SEQUENCE LISTING (1406 sequences)

SEQ ID NO: 1 - DNA, 44 bp, synthetic construct
  source 1..44: mol_type = other DNA
ctaaagaggagaaaggatctnnkgacaaaaagtactctattggc

SEQ ID NO: 2 - DNA, 41 bp, synthetic construct
  source 1..41: mol_type = other DNA
agaggagaaaggatctatgnnkaaaaagtactctattggcc

SEQ ID NO: 3 - DNA, 38 bp, synthetic construct
  source 1..38: mol_type = other DNA
agaaaggatctatggacnnkaagtactctattggcctg

SEQ ID NO: 4 - DNA, 37 bp, synthetic construct
  source 1..37: mol_type = other DNA
aaggatctatggacaaannktactctattggcctgga

SEQ ID NO: 5 - DNA, 39 bp, synthetic construct
  source 1..39: mol_type = other DNA
ggatctatggacaaaaagnnktctattggcctggatatc

SEQ ID NO: 6 - DNA, 37 bp, synthetic construct
  source 1..37: mol_type = other DNA
ctatggacaaaaagtacnnkattggcctggatatcgg

SEQ ID NO: 7 - DNA, 33 bp, synthetic construct
  source 1..33: mol_type = other DNA
gacaaaaagtactctnnkggcctggatatcggg

SEQ ID NO: 8 - DNA, 39 bp, synthetic construct
  source 1..39: mol_type = other DNA
gacaaaaagtactctattnnkctggatatcgggaccaac

SEQ ID NO: 9 - DNA, 35 bp, synthetic construct
  source 1..35: mol_type = other DNA
aaagtactctattggcnnkgatatcgggaccaaca

SEQ ID NO: 10 - DNA, 30 bp, synthetic construct
  source 1..30: mol_type = other DNA
ctctattggcctgnnkatcgggaccaacag

SEQ ID NO: 11 - DNA, 25 bp, synthetic construct
  source 1..25: mol_type = other DNA
ttggcctggatnnkgggaccaacag

SEQ ID NO: 12 - DNA, 27 bp, synthetic construct
  source 1..27: mol_type = other DNA
ggcctggatatcnnkaccaacagcgtc

SEQ ID NO: 13 - DNA, 27 bp, synthetic construct
  source 1..27: mol_type = other DNA
ctggatatcgggnnkaacagcgtcggg

SEQ ID NO: 14 - DNA, 25 bp, synthetic construct
  source 1..25: mol_type = other DNA
atatcgggaccnnkagcgtcgggtg

SEQ ID NO: 15 - DNA, 25 bp, synthetic construct
  source 1..25: mol_type = other DNA
tcgggaccaacnnkgtcgggtgggc

SEQ ID NO: 16 - DNA, 25 bp, synthetic construct
  source 1..25: mol_type = other DNA
ggaccaacagcnnkgggtgggctgt

SEQ ID NO: 17 - DNA, 29 bp, synthetic construct
  source 1..29: mol_type = other DNA
gaccaacagcgtcnnktgggctgttatca

SEQ ID NO: 18 - DNA, 25 bp, synthetic construct
  source 1..25: mol_type = other DNA
acagcgtcgggnnkgctgttatcac

SEQ ID NO: 19 - DNA, 25 bp, synthetic construct
  source 1..25: mol_type = other DNA
gcgtcgggtggnnkgttatcaccga

SEQ ID NO: 20 - DNA, 25 bp, synthetic construct
  source 1..25: mol_type = other DNA
tcgggtgggctnnkatcaccgacga

SEQ ID NO: 21 - DNA, 28 bp, synthetic construct
  source 1..28: mol_type = other DNA
gggtgggctgttnnkaccgacgagtata

SEQ ID NO: 22 - DNA, 34 bp, synthetic construct
  source 1..34: mol_type = other DNA
gggtgggctgttatcnnkgacgagtataaagtac

SEQ ID NO: 23 - DNA, 36 bp, synthetic construct
  source 1..36: mol_type = other DNA
gtgggctgttatcaccnnkgagtataaagtaccttc

SEQ ID NO: 24 - DNA, 36 bp, synthetic construct
  source 1..36: mol_type = other DNA
ggctgttatcaccgacnnktataaagtaccttcgaa

SEQ ID NO: 25 - DNA, 37 bp, synthetic construct
  source 1..37: mol_type = other DNA
ctgttatcaccgacgagnnkaaagtaccttcgaaaaa

SEQ ID NO: 26 - DNA, 38 bp, synthetic construct
  source 1..38: mol_type = other DNA
ttatcaccgacgagtatnnkgtaccttcgaaaaagttc

SEQ ID NO: 27 - DNA, 37 bp, synthetic construct
  source 1..37: mol_type = other DNA
tcaccgacgagtataaannkccttcgaaaaagttcaa

SEQ ID NO: 28 - DNA, 40 bp, synthetic construct
  source 1..40: mol_type = other DNA
accgacgagtataaagtannktcgaaaaagttcaaagtgc

SEQ ID NO: 29 - DNA, 41 bp, synthetic construct
  source 1..41: mol_type = other DNA
cgacgagtataaagtacctnnkaaaaagttcaaagtgctgg

SEQ ID NO: 30 - DNA, 37 bp, synthetic construct
  source 1..37: mol_type = other DNA
agtataaagtaccttcgnnkaagttcaaagtgctggg

SEQ ID NO: 31 - DNA, 37 bp, synthetic construct
  source 1..37: mol_type = other DNA
ataaagtaccttcgaaannkttcaaagtgctgggcaa

SEQ ID NO: 32 - DNA, 33 bp, synthetic construct
  source 1..33: mol_type = other DNA
gtaccttcgaaaaagnnkaaagtgctgggcaac

SEQ ID NO: 33 - DNA, 30 bp, synthetic construct
  source 1..30: mol_type = other DNA
ttcgaaaaagttcnnkgtgctgggcaacac

SEQ ID NO: 34 - DNA, 32 bp, synthetic construct
  source 1..32: mol_type = other DNA
cgaaaaagttcaaannkctgggcaacaccgat

SEQ ID NO: 35 - DNA, 31 bp, synthetic construct
  source 1..31: mol_type = other DNA
aaaagttcaaagtgnnkggcaacaccgatcg

SEQ ID NO: 36 - DNA, 29 bp, synthetic construct
  source 1..29: mol_type = other DNA
gttcaaagtgctgnnkaacaccgatcgcc

SEQ ID NO: 37 - DNA, 25 bp, synthetic construct
  source 1..25: mol_type = other DNA
aagtgctgggcnnkaccgatcgcca

SEQ ID NO: 38 - DNA, 25 bp, synthetic construct
  source 1..25: mol_type = other DNA
tgctgggcaacnnkgatcgccattc

SEQ ID NO: 39 - DNA, 27 bp, synthetic construct
  source 1..27: mol_type = other DNA
ctgggcaacaccnnkcgccattcaatc

SEQ ID NO: 40 - DNA, 34 bp, synthetic construct
  source 1..34: mol_type = other DNA
ctgggcaacaccgatnnkcattcaatcaaaaaga

SEQ ID NO: 41 - DNA, 32 bp, synthetic construct
  source 1..32: mol_type = other DNA
gcaacaccgatcgcnnktcaatcaaaaagaac

SEQ ID NO: 42 - DNA, 36 bp, synthetic construct
  source 1..36: mol_type = other DNA
caacaccgatcgccatnnkatcaaaaagaacttgat

SEQ ID NO: 43 - DNA, 35 bp, synthetic construct
  source 1..35: mol_type = other DNA
caccgatcgccattcannkaaaaagaacttgattg

SEQ ID NO: 44 - DNA, 35 bp, synthetic construct
  source 1..35: mol_type = other DNA
cgatcgccattcaatcnnkaagaacttgattggtg

SEQ ID NO: 45 - DNA, 33 bp, synthetic construct
  source 1..33: mol_type = other DNA
cgccattcaatcaaannkaacttgattggtgcg

SEQ ID NO: 46 - DNA, 34 bp, synthetic construct
  source 1..34: mol_type = other DNA
cattcaatcaaaaagnnkttgattggtgcgctgt

SEQ ID NO: 47 - DNA, 37 bp, synthetic construct
  source 1..37: mol_type = other DNA
attcaatcaaaaagaacnnkattggtgcgctgttgtt

SEQ ID NO: 48 - DNA, 33 bp, synthetic construct
  source 1..33: mol_type = other DNA
atcaaaaagaacttgnnkggtgcgctgttgttt

SEQ ID NO: 49 - DNA, 37 bp, synthetic construct
  source 1..37: mol_type = other DNA
tcaaaaagaacttgattnnkgcgctgttgtttgactc

SEQ ID NO: 50 - DNA, 35 bp, synthetic construct
  source 1..35: mol_type = other DNA
aaagaacttgattggtnnkctgttgtttgactccg

SEQ ID NO: 51 - DNA, 30 bp, synthetic construct
  source 1..30: mol_type = other DNA
cttgattggtgcgnnkttgtttgactccgg

SEQ ID NO: 52 - DNA, 25 bp, synthetic construct
  source 1..25: mol_type = other DNA
ttggtgcgctgnnktttgactccgg

SEQ ID NO: 53 - DNA, 25 bp, synthetic construct
  source 1..25: mol_type = other DNA
gtgcgctgttgnnkgactccgggga

SEQ ID NO: 54 - DNA, 26 bp, synthetic construct
  source 1..26: mol_type = other DNA
cgctgttgtttnnktccggggaaacc

SEQ ID NO: 55 - DNA, 27 bp, synthetic construct
  source 1..27: mol_type = other DNA
ctgttgtttgacnnkggggaaaccgcc

SEQ ID NO: 56 - DNA, 27 bp, synthetic construct
  source 1..27: mol_type = other DNA
ttgtttgactccnnkgaaaccgccgag

SEQ ID NO: 57 - DNA, 25 bp, synthetic construct
  source 1..25: mol_type = other DNA
ttgactccgggnnkaccgccgaggc

SEQ ID NO: 58 - DNA, 25 bp, synthetic construct
  source 1..25: mol_type = other DNA
actccggggaannkgccgaggcgac

SEQ ID NO: 59 - DNA, 25 bp, synthetic construct
  source 1..25: mol_type = other DNA
ccggggaaaccnnkgaggcgactcg

SEQ ID NO: 60 - DNA, 25 bp, synthetic construct
  source 1..25: mol_type = other DNA
gggaaaccgccnnkgcgactcgcct

SEQ ID NO: 61 - DNA, 28 bp, synthetic construct
  source 1..28: mol_type = other DNA
gaaaccgccgagnnkactcgccttaaac

SEQ ID NO: 62 - DNA, 25 bp, synthetic construct
  source 1..25: mol_type = other DNA
ccgccgaggcgnnkcgccttaaacg

SEQ ID NO: 63 - DNA, 28 bp, synthetic construct
  source 1..28: mol_type = other DNA
gccgaggcgactnnkcttaaacgtacag

SEQ ID NO: 64 - DNA, 25 bp, synthetic construct
  source 1..25: mol_type = other DNA
aggcgactcgcnnkaaacgtacagc

SEQ ID NO: 65 - DNA, 25 bp, synthetic construct
  source 1..25: mol_type = other DNA
cgactcgccttnnkcgtacagcacg

SEQ ID NO: 66 - DNA, 27 bp, synthetic construct
  source 1..27: mol_type = other DNA
actcgccttaaannkacagcacgtcgc

SEQ ID NO: 67 - DNA, 25 bp, synthetic construct
  source 1..25: mol_type = other DNA
gccttaaacgtnnkgcacgtcgccg

SEQ ID NO: 68 - DNA, 29 bp, synthetic construct
  source 1..29: mol_type = other DNA
ccttaaacgtacannkcgtcgccggtaca

SEQ ID NO: 69 - DNA, 29 bp, synthetic construct
  source 1..29: mol_type = other DNA
taaacgtacagcannkcgccggtacactc

SEQ ID NO: 70 - DNA, 26 bp, synthetic construct
  source 1..26: mol_type = other DNA
gtacagcacgtnnkcggtacactcgg

SEQ ID NO: 71 - DNA, 25 bp, synthetic construct
  source 1..25: mol_type = other DNA
cagcacgtcgcnnktacactcggcg

SEQ ID NO: 72 - DNA, 25 bp, synthetic construct
  source 1..25: mol_type = other DNA
cacgtcgccggnnkactcggcgtaa

SEQ ID NO: 73 - DNA, 25 bp, synthetic construct
  source 1..25: mol_type = other DNA
gtcgccggtacnnkcggcgtaagaa

SEQ ID NO: 74 - DNA, 27 bp, synthetic construct
  source 1..27: mol_type = other DNA
cgccggtacactnnkcgtaagaatcgc

SEQ ID NO: 75 - DNA, 30 bp, synthetic construct
  source 1..30: mol_type = other DNA
ccggtacactcggnnkaagaatcgcatttg

SEQ ID NO: 76 - DNA, 30 bp, synthetic construct
  source 1..30: mol_type = other DNA
gtacactcggcgtnnkaatcgcatttgcta

SEQ ID NO: 77 - DNA, 30 bp, synthetic construct
  source 1..30: mol_type = other DNA
cactcggcgtaagnnkcgcatttgctattt

SEQ ID NO: 78 - DNA, 35 bp, synthetic construct
  source 1..35: mol_type = other DNA
cactcggcgtaagaatnnkatttgctatttgcagg

SEQ ID NO: 79 - DNA, 30 bp, synthetic construct
  source 1..30: mol_type = other DNA
gcgtaagaatcgcnnktgctatttgcagga

SEQ ID NO: 80 - DNA, 37 bp, synthetic construct
  source 1..37: mol_type = other DNA
ggcgtaagaatcgcattnnktatttgcaggaaatctt

SEQ ID NO: 81 - DNA, 38 bp, synthetic construct
  source 1..38: mol_type = other DNA
gtaagaatcgcatttgcnnkttgcaggaaatctttagc

SEQ ID NO: 82 - DNA, 38 bp, synthetic construct
  source 1..38: mol_type = other DNA
agaatcgcatttgctatnnkcaggaaatctttagcaac

SEQ ID NO: 83 - DNA, 37 bp, synthetic construct
  source 1..37: mol_type = other DNA
atcgcatttgctatttgnnkgaaatctttagcaacga

SEQ ID NO: 84 - DNA, 37 bp, synthetic construct
  source 1..37: mol_type = other DNA
gcatttgctatttgcagnnkatctttagcaacgagat

SEQ ID NO: 85 - DNA, 35 bp, synthetic construct
  source 1..35: mol_type = other DNA
ttgctatttgcaggaannktttagcaacgagatgg

SEQ ID NO: 86 - DNA, 31 bp, synthetic construct
  source 1..31: mol_type = other DNA
atttgcaggaaatcnnkagcaacgagatggc

SEQ ID NO: 87 - DNA, 37 bp, synthetic construct
  source 1..37: mol_type = other DNA
atttgcaggaaatctttnnkaacgagatggcaaaagt

SEQ ID NO: 88 - DNA, 34 bp, synthetic construct
  source 1..34: mol_type = other DNA
caggaaatctttagcnnkgagatggcaaaagtcg

SEQ ID NO: 89 - DNA, 37 bp, synthetic construct
  source 1..37: mol_type = other DNA
aggaaatctttagcaacnnkatggcaaaagtcgatga

SEQ ID NO: 90 - DNA, 35 bp, synthetic construct
  source 1..35: mol_type = other DNA
aatctttagcaacgagnnkgcaaaagtcgatgact

SEQ ID NO: 91 - DNA, 36 bp, synthetic construct
  source 1..36: mol_type = other DNA
ctttagcaacgagatgnnkaaagtcgatgactcgtt

SEQ ID NO: 92 - DNA, 30 bp, synthetic construct
  source 1..30: mol_type = other DNA
caacgagatggcannkgtcgatgactcgtt

SEQ ID NO: 93 - DNA, 35 bp, synthetic construct
  source 1..35: mol_type = other DNA
caacgagatggcaaaannkgatgactcgtttttcc

SEQ ID NO: 94 - DNA, 33 bp, synthetic construct
  source 1..33: mol_type = other DNA
gagatggcaaaagtcnnkgactcgtttttccac

SEQ ID NO: 95 - DNA, 31 bp, synthetic construct
  source 1..31: mol_type = other DNA
tggcaaaagtcgatnnktcgtttttccaccg

SEQ ID NO: 96 - DNA, 32 bp, synthetic construct
  source 1..32: mol_type = other DNA
caaaagtcgatgacnnktttttccaccgcctc

SEQ ID NO: 97 - DNA, 26 bp, synthetic construct
  source 1..26: mol_type = other DNA
tcgatgactcgnnkttccaccgcctc

SEQ ID NO: 98 - DNA, 27 bp, synthetic construct
  source 1..27: mol_type = other DNA
gatgactcgtttnnkcaccgcctcgag

SEQ ID NO: 99 - DNA, 29 bp, synthetic construct
  source 1..29: mol_type = other DNA
tgactcgtttttcnnkcgcctcgaggaaa

SEQ ID NO: 100 - DNA, 32 bp, synthetic construct
  source 1..32: mol_type = other DNA
actcgtttttccacnnkctcgaggaaagcttt

SEQ ID NO: 101 - DNA, 30 bp, synthetic construct
  source 1..30: mol_type = other DNA
gtttttccaccgcnnkgaggaaagctttct

SEQ ID NO: 102 - DNA, 27 bp, synthetic construct
  source 1..27: mol_type = other DNA
ttccaccgcctcnnkgaaagctttctg

SEQ ID NO: 103 - DNA, 26 bp, synthetic construct
  source 1..26: mol_type = other DNA
accgcctcgagnnkagctttctggtg

SEQ ID NO: 104 - DNA, 27 bp, synthetic construct
  source 1..27: mol_type = other DNA
cgcctcgaggaannktttctggtggag

SEQ ID NO: 105 - DNA, 28 bp, synthetic construct
  source 1..28: mol_type = other DNA
ctcgaggaaagcnnkctggtggaggaag

SEQ ID NO: 106 - DNA, 34 bp, synthetic construct
  source 1..34: mol_type = other DNA
ctcgaggaaagctttnnkgtggaggaagacaaaa

SEQ ID NO: 107 - DNA, 34 bp, synthetic construct
  source 1..34: mol_type = other DNA
gaggaaagctttctgnnkgaggaagacaaaaagc

SEQ ID NO: 108 - DNA, 35 bp, synthetic construct
  source 1..35: mol_type = other DNA
ggaaagctttctggtgnnkgaagacaaaaagcatg

SEQ ID NO: 109 - DNA, 32 bp, synthetic construct
  source 1..32: mol_type = other DNA
gctttctggtggagnnkgacaaaaagcatgag

SEQ ID NO: 110 - DNA, 31 bp, synthetic construct
  source 1..31: mol_type = other DNA
ttctggtggaggaannkaaaaagcatgagcg

SEQ ID NO: 111 - DNA, 28 bp, synthetic construct
  source 1..28: mol_type = other DNA
gtggaggaagacnnkaagcatgagcgtc

SEQ ID NO: 112 - DNA, 29 bp, synthetic construct
  source 1..29: mol_type = other DNA
ggaggaagacaaannkcatgagcgtcacc

SEQ ID NO: 113 - DNA, 28 bp, synthetic construct
  source 1..28: mol_type = other DNA
gaagacaaaaagnnkgagcgtcacccga

SEQ ID NO: 114 - DNA, 33 bp, synthetic construct
  source 1..33: mol_type = other DNA
gaagacaaaaagcatnnkcgtcacccgatcttc

SEQ ID NO: 115 - DNA, 30 bp, synthetic construct
  source 1..30: mol_type = other DNA
caaaaagcatgagnnkcacccgatcttcgg

SEQ ID NO: 116 - DNA, 25 bp, synthetic construct
  source 1..25: mol_type = other DNA
agcatgagcgtnnkccgatcttcgg

SEQ ID NO: 117 - DNA, 28 bp, synthetic construct
  source 1..28: mol_type = other DNA
catgagcgtcacnnkatcttcggcaaca

SEQ ID NO: 118 - DNA, 25 bp, synthetic construct
  source 1..25: mol_type = other DNA
agcgtcacccgnnkttcggcaacat

SEQ ID NO: 119 - DNA, 27 bp, synthetic construct
  source 1..27: mol_type = other DNA
cgtcacccgatcnnkggcaacattgtc

SEQ ID NO: 120 - DNA, 32 bp, synthetic construct
  source 1..32: mol_type = other DNA
gtcacccgatcttcnnkaacattgtcgatgaa

SEQ ID NO: 121 - DNA, 29 bp, synthetic construct
  source 1..29: mol_type = other DNA
cccgatcttcggcnnkattgtcgatgaag

SEQ ID NO: 122 - DNA, 30 bp, synthetic construct
  source 1..30: mol_type = other DNA
gatcttcggcaacnnkgtcgatgaagtagc

SEQ ID NO: 123 - DNA, 36 bp, synthetic construct
  source 1..36: mol_type = other DNA
gatcttcggcaacattnnkgatgaagtagcgtatca

SEQ ID NO: 124 - DNA, 33 bp, synthetic construct
  source 1..33: mol_type = other DNA
ttcggcaacattgtcnnkgaagtagcgtatcat

SEQ ID NO: 125 - DNA, 34 bp, synthetic construct
  source 1..34: mol_type = other DNA
ggcaacattgtcgatnnkgtagcgtatcatgaaa

SEQ ID NO: 126 - DNA, 39 bp, synthetic construct
  source 1..39: mol_type = other DNA
ggcaacattgtcgatgaannkgcgtatcatgaaaaatac

SEQ ID NO: 127 - DNA, 43 bp, synthetic construct
  source 1..43: mol_type = other DNA
gcaacattgtcgatgaagtannktatcatgaaaaatacccaac

SEQ ID NO: 128 - DNA, 35 bp, synthetic construct
  source 1..35: mol_type = other DNA
tgtcgatgaagtagcgnnkcatgaaaaatacccaa

SEQ ID NO: 129 - DNA, 37 bp, synthetic construct
  source 1..37: mol_type = other DNA
tcgatgaagtagcgtatnnkgaaaaatacccaaccat

SEQ ID NO: 130 - DNA, 41 bp, synthetic construct
  source 1..41: mol_type = other DNA
cgatgaagtagcgtatcatnnkaaatacccaaccatttacc

SEQ ID NO: 131 - DNA, 38 bp, synthetic construct
  source 1..38: mol_type = other DNA
aagtagcgtatcatgaannktacccaaccatttaccac

SEQ ID NO: 132 - DNA, 40 bp, synthetic construct
  source 1..40: mol_type = other DNA
gtagcgtatcatgaaaaannkccaaccatttaccacttac

SEQ ID NO: 133 - DNA, 39 bp, synthetic construct
  source 1..39: mol_type = other DNA
gcgtatcatgaaaaatacnnkaccatttaccacttacgc

SEQ ID NO: 134 - DNA, 43 bp, synthetic construct
  source 1..43: mol_type = other DNA
cgtatcatgaaaaatacccannkatttaccacttacgcaaaaa

SEQ ID NO: 135 - DNA, 38 bp, synthetic construct
  source 1..38: mol_type = other DNA
atgaaaaatacccaaccnnktaccacttacgcaaaaag

SEQ ID NO: 136 - DNA, 37 bp, synthetic construct
  source 1..37: mol_type = other DNA
aaaaatacccaaccattnnkcacttacgcaaaaagct

SEQ ID NO: 137 - DNA, 36 bp, synthetic construct
  source 1..36: mol_type = other DNA
atacccaaccatttacnnkttacgcaaaaagctggt

SEQ ID NO: 138 - DNA, 31 bp, synthetic construct
  source 1..31: mol_type = other DNA
caaccatttaccacnnkcgcaaaaagctggt

SEQ ID NO: 139 - DNA, 37 bp, synthetic construct
  source 1..37: mol_type = other DNA
caaccatttaccacttannkaaaaagctggtggacag

SEQ ID NO: 140 - DNA, 30 bp, synthetic construct
  source 1..30: mol_type = other DNA
ttaccacttacgcnnkaagctggtggacag

SEQ ID NO: 141 - DNA, 28 bp, synthetic construct
  source 1..28: mol_type = other DNA
cacttacgcaaannkctggtggacagca

SEQ ID NO: 142 - DNA, 31 bp, synthetic construct
  source 1..31: mol_type = other DNA
acttacgcaaaaagnnkgtggacagcactga

SEQ ID NO: 143 - DNA, 28 bp, synthetic construct
  source 1..28: mol_type = other DNA
cgcaaaaagctgnnkgacagcactgaca

SEQ ID NO: 144 - DNA, 30 bp, synthetic construct
  source 1..30: mol_type = other DNA
caaaaagctggtgnnkagcactgacaaagc

SEQ ID NO: 145 - DNA, 32 bp, synthetic construct
  source 1..32: mol_type = other DNA
aaaagctggtggacnnkactgacaaagctgat

SEQ ID NO: 146 - DNA, 28 bp, synthetic construct
  source 1..28: mol_type = other DNA
ctggtggacagcnnkgacaaagctgatt

SEQ ID NO: 147 - DNA, 30 bp, synthetic construct
  source 1..30: mol_type = other DNA
ggtggacagcactnnkaaagctgatttgcg

SEQ ID NO: 148 - DNA, 25 bp, synthetic construct
  source 1..25: mol_type = other DNA
acagcactgacnnkgctgatttgcg

SEQ ID NO: 149 - DNA, 33 bp, synthetic construct
  source 1..33: mol_type = other DNA
gacagcactgacaaannkgatttgcgccttatc

SEQ ID NO: 150 - DNA, 33 bp, synthetic construct
  source 1..33: mol_type = other DNA
agcactgacaaagctnnkttgcgccttatctat

SEQ ID NO: 151 - DNA, 36 bp, synthetic construct
  source 1..36: mol_type = other DNA
cactgacaaagctgatnnkcgccttatctatttagc

SEQ ID NO: 152 - DNA, 39 bp, synthetic construct
  source 1..39: mol_type = other DNA
actgacaaagctgatttgnnkcttatctatttagccctg

SEQ ID NO: 153 - DNA, 33 bp, synthetic construct
  source 1..33: mol_type = other DNA
aaagctgatttgcgcnnkatctatttagccctg

SEQ ID NO: 154 - DNA, 31 bp, synthetic construct
  source 1..31: mol_type = other DNA
ctgatttgcgccttnnktatttagccctggc

SEQ ID NO: 155 - DNA, 27 bp, synthetic construct
  source 1..27: mol_type = other DNA
ttgcgccttatcnnkttagccctggca

SEQ ID NO: 156 - DNA, 30 bp, synthetic construct
  source 1..30: mol_type = other DNA
gcgccttatctatnnkgccctggcacatat

SEQ ID NO: 157 - DNA, 40 bp, synthetic construct
  source 1..40: mol_type = other DNA
ttgcgccttatctatttannkctggcacatatgattaagt

SEQ ID NO: 158 - DNA, 41 bp, synthetic construct
  source 1..41: mol_type = other DNA
gcgccttatctatttagccnnkgcacatatgattaagtttc

SEQ ID NO: 159 - DNA, 42 bp, synthetic construct
  source 1..42: mol_type = other DNA
ccttatctatttagccctgnnkcatatgattaagtttcgtgg

SEQ ID NO: 160 - DNA, 37 bp, synthetic construct
  source 1..37: mol_type = other DNA
tctatttagccctggcannkatgattaagtttcgtgg

SEQ ID NO: 161 - DNA, 35 bp, synthetic construct
  source 1..35: mol_type = other DNA
tttagccctggcacatnnkattaagtttcgtggtc

SEQ ID NO: 162 - DNA, 31 bp, synthetic construct
  source 1..31: mol_type = other DNA
ccctggcacatatgnnkaagtttcgtggtca

SEQ ID NO: 163 - DNA, 34 bp, synthetic construct
  source 1..34: mol_type = other DNA
ctggcacatatgattnnktttcgtggtcacttcc

SEQ ID NO: 164 - DNA, 34 bp, synthetic construct
  source 1..34: mol_type = other DNA
gcacatatgattaagnnkcgtggtcacttcctga

SEQ ID NO: 165 - DNA, 39 bp, synthetic construct
  source 1..39: mol_type = other DNA
gcacatatgattaagtttnnkggtcacttcctgatcgaa

SEQ ID NO: 166 - DNA, 39 bp, synthetic construct
  source 1..39: mol_type = other DNA
catatgattaagtttcgtnnkcacttcctgatcgaagga

SEQ ID NO: 167 - DNA, 36 bp, synthetic construct
  source 1..36: mol_type = other DNA
gattaagtttcgtggtnnkttcctgatcgaaggaga

SEQ ID NO: 168 - DNA, 30 bp, synthetic construct
  source 1..30: mol_type = other DNA
gtttcgtggtcacnnkctgatcgaaggaga

SEQ ID NO: 169 - DNA, 36 bp, synthetic construct
  source 1..36: mol_type = other DNA
gtttcgtggtcacttcnnkatcgaaggagacttaaa

SEQ ID NO: 170 - DNA, 34 bp, synthetic construct
  source 1..34: mol_type = other DNA
cgtggtcacttcctgnnkgaaggagacttaaatc

SEQ ID NO: 171 - DNA, 33 bp, synthetic construct
  source 1..33: mol_type = other DNA
ggtcacttcctgatcnnkggagacttaaatccc

SEQ ID NO: 172 - DNA, 36 bp, synthetic construct
  source 1..36: mol_type = other DNA
tcacttcctgatcgaannkgacttaaatcccgacaa

SEQ ID NO: 173 - DNA, 36 bp, synthetic construct
  source 1..36: mol_type = other DNA
cttcctgatcgaaggannkttaaatcccgacaacag

SEQ ID NO: 174 - DNA, 31 bp, synthetic construct
  source 1..31: mol_type = other DNA
tgatcgaaggagacnnkaatcccgacaacag

SEQ ID NO: 175 - DNA, 33 bp, synthetic construct
  source 1..33: mol_type = other DNA
atcgaaggagacttannkcccgacaacagtgat

SEQ ID NO: 176 - DNA, 38 bp, synthetic construct
  source 1..38: mol_type = other DNA
tcgaaggagacttaaatnnkgacaacagtgatgttgat

SEQ ID NO: 177 - DNA, 41 bp, synthetic construct
  source 1..41: mol_type = other DNA
cgaaggagacttaaatcccnnkaacagtgatgttgataaat

SEQ ID NO: 178 - DNA, 40 bp, synthetic construct
  source 1..40: mol_type = other DNA
ggagacttaaatcccgacnnkagtgatgttgataaattgt

SEQ ID NO: 179 - DNA, 45 bp, synthetic construct
  source 1..45: mol_type = other DNA
ggagacttaaatcccgacaacnnkgatgttgataaattgtttatt

SEQ ID NO: 180 - DNA, 45 bp, synthetic construct
  source 1..45: mol_type = other DNA
gacttaaatcccgacaacagtnnkgttgataaattgtttattcag

SEQ ID NO: 181 - DNA, 41 bp, synthetic construct
  source 1..41: mol_type = other DNA
aaatcccgacaacagtgatnnkgataaattgtttattcagc

SEQ ID NO: 182 - DNA, 39 bp, synthetic construct
  source 1..39: mol_type = other DNA
cccgacaacagtgatgttnnkaaattgtttattcagctt

SEQ ID NO: 183 - DNA, 40 bp, synthetic construct
  source 1..40: mol_type = other DNA
gacaacagtgatgttgatnnkttgtttattcagcttgtcc

SEQ ID NO: 184 - DNA, 43 bp, synthetic construct
  source 1..43: mol_type = other DNA
acaacagtgatgttgataaannktttattcagcttgtccaaac

SEQ ID NO: 185 - DNA, 41 bp, synthetic construct
  source 1..41: mol_type = other DNA
cagtgatgttgataaattgnnkattcagcttgtccaaactt

SEQ ID NO: 186 - DNA, 43 bp, synthetic construct
  source 1..43: mol_type = other DNA
gtgatgttgataaattgtttnnkcagcttgtccaaacttacaa

SEQ ID NO: 187 - DNA, 49 bp, synthetic construct
  source 1..49: mol_type = other DNA
gtgatgttgataaattgtttattnnkcttgtccaaacttacaatcaact

SEQ ID NO: 188 - DNA, 46 bp, synthetic construct
  source 1..46: mol_type = other DNA
gttgataaattgtttattcagnnkgtccaaacttacaatcaactgt

SEQ ID NO: 189 - DNA, 47 bp, synthetic construct
  source 1..47: mol_type = other DNA
tgataaattgtttattcagcttnnkcaaacttacaatcaactgttcg

SEQ ID NO: 190 - DNA, 40 bp, synthetic construct
  source 1..40: mol_type = other DNA
ttgtttattcagcttgtcnnkacttacaatcaactgttcg

SEQ ID NO: 191 - DNA, 40 bp, synthetic construct
  source 1..40: mol_type = other DNA
tttattcagcttgtccaannktacaatcaactgttcgagg

SEQ ID NO: 192 - DNA, 35 bp, synthetic construct
  source 1..35: mol_type = other DNA
tcagcttgtccaaactnnkaatcaactgttcgagg

SEQ ID NO: 193 - DNA, 34 bp, synthetic construct
  source 1..34: mol_type = other DNA
cttgtccaaacttacnnkcaactgttcgaggaaa

SEQ ID NO: 194 - DNA, 37 bp, synthetic construct
  source 1..37: mol_type = other DNA
ttgtccaaacttacaatnnkctgttcgaggaaaaccc

SEQ ID NO: 195 - DNA, 37 bp, synthetic construct
  source 1..37: mol_type = other DNA
tccaaacttacaatcaannkttcgaggaaaacccgat

SEQ ID NO: 196 - DNA, 37 bp, synthetic construct
  source 1..37: mol_type = other DNA
aaacttacaatcaactgnnkgaggaaaacccgatcaa

SEQ ID NO: 197 - DNA, 37 bp, synthetic construct
  source 1..37: mol_type = other DNA
cttacaatcaactgttcnnkgaaaacccgatcaatgc

SEQ ID NO: 198 - DNA, 31 bp, synthetic construct
  source 1..31: mol_type = other DNA
atcaactgttcgagnnkaacccgatcaatgc

SEQ ID NO: 199 - DNA, 31 bp, synthetic construct
  source 1..31: mol_type = other DNA
aactgttcgaggaannkccgatcaatgcctc

SEQ ID NO: 200 - DNA, 30 bp, synthetic construct
  source 1..30: mol_type = other DNA
gttcgaggaaaacnnkatcaatgcctccgg

SEQ ID NO: 201 - DNA, 27 bp, synthetic construct
  source 1..27: mol_type = other DNA
gaggaaaacccgnnkaatgcctccggt

SEQ ID NO: 202 - DNA, 25 bp, synthetic construct
  source 1..25: mol_type = other DNA
aaaacccgatcnnkgcctccggtgt

SEQ ID NO: 203 - DNA, 29 bp, synthetic construct
  source 1..29: mol_type = other DNA
aaacccgatcaatnnktccggtgtggatg

SEQ ID NO: 204 - DNA, 26 bp, synthetic construct
  source 1..26: mol_type = other DNA
cgatcaatgccnnkggtgtggatgca

SEQ ID NO: 205 - DNA, 29 bp, synthetic construct
  source 1..29: mol_type = other DNA
gatcaatgcctccnnkgtggatgcaaaag

SEQ ID NO: 206 - DNA, 27 bp, synthetic construct
  source 1..27: mol_type = other DNA
aatgcctccggtnnkgatgcaaaagcc

SEQ ID NO: 207 - DNA, 26 bp, synthetic construct
  source 1..26: mol_type = other DNA
cctccggtgtgnnkgcaaaagccatt

SEQ ID NO: 208 - DNA, 32 bp, synthetic construct
  source 1..32: mol_type = other DNA
cctccggtgtggatnnkaaagccattttaagt

SEQ ID NO: 209 - DNA, 29 bp, synthetic construct
  source 1..29: mol_type = other DNA
cggtgtggatgcannkgccattttaagtg

SEQ ID NO: 210 - DNA, 33 bp, synthetic construct
  source 1..33: mol_type = other DNA
ggtgtggatgcaaaannkattttaagtgcacgc

SEQ ID NO: 211 - DNA, 28 bp, synthetic construct
  source 1..28: mol_type = other DNA
gatgcaaaagccnnkttaagtgcacgcc

SEQ ID NO: 212 - DNA, 31 bp, synthetic construct
  source 1..31: mol_type = other DNA
atgcaaaagccattnnkagtgcacgccttag

SEQ ID NO: 213 - DNA, 32 bp, synthetic construct
  source 1..32: mol_type = other DNA
caaaagccattttannkgcacgccttagcaag

SEQ ID NO: 214 - DNA, 33 bp, synthetic construct
  source 1..33: mol_type = other DNA
aaagccattttaagtnnkcgccttagcaagtcc

SEQ ID NO: 215 - DNA, 32 bp, synthetic construct
  source 1..32: mol_type = other DNA
ccattttaagtgcannkcttagcaagtcccgt

SEQ ID NO: 216 - DNA, 25 bp, synthetic construct
  source 1..25: mol_type = other DNA
taagtgcacgcnnkagcaagtcccg

SEQ ID NO: 217 - DNA, 25 bp, synthetic construct
  source 1..25: mol_type = other DNA
gtgcacgccttnnkaagtcccgtcg

SEQ ID NO: 218 - DNA, 26 bp, synthetic construct
  source 1..26: mol_type = other DNA
cacgccttagcnnktcccgtcgctta

SEQ ID NO: 219 - DNA, 28 bp, synthetic construct
  source 1..28: mol_type = other DNA
cgccttagcaagnnkcgtcgcttagaaa

SEQ ID NO: 220 - DNA, 31 bp, synthetic construct
  source 1..31: mol_type = other DNA
gccttagcaagtccnnkcgcttagaaaacct

SEQ ID NO: 221 - DNA, 35 bp, synthetic construct
  source 1..35: mol_type = other DNA
ccttagcaagtcccgtnnkttagaaaaccttatcg

SEQ ID NO: 222 - DNA, 29 bp, synthetic construct
  source 1..29: mol_type = other DNA
caagtcccgtcgcnnkgaaaaccttatcg

SEQ ID NO: 223 - DNA, 27 bp, synthetic construct
  source 1..27: mol_type = other DNA
tcccgtcgcttannkaaccttatcgcg

SEQ ID NO: 224 - DNA, 28 bp, synthetic construct
  source 1..28: mol_type = other DNA
cgtcgcttagaannkcttatcgcgcagc

SEQ ID NO: 225 - DNA, 27 bp, synthetic construct
  source 1..27: mol_type = other DNA
cgcttagaaaacnnkatcgcgcagctg

SEQ ID NO: 226 - DNA, 27 bp, synthetic construct
  source 1..27: mol_type = other DNA
ttagaaaaccttnnkgcgcagctgccc

SEQ ID NO: 227 - DNA, 27 bp, synthetic construct
  source 1..27: mol_type = other DNA
gaaaaccttatcnnkcagctgcccggc

SEQ ID NO: 228 - DNA, 25 bp, synthetic construct
  source 1..25: mol_type = other DNA
accttatcgcgnnkctgcccggcga

SEQ ID NO: 229 - DNA, 25 bp, synthetic construct
  source 1..25: mol_type = other DNA
ttatcgcgcagnnkcccggcgagaa

SEQ ID NO: 230 - DNA, 25 bp, synthetic construct
  source 1..25: mol_type = other DNA
tcgcgcagctgnnkggcgagaaaaa

SEQ ID NO: 231 - DNA, 27 bp, synthetic construct
  source 1..27: mol_type = other DNA
gcgcagctgcccnnkgagaaaaagaat

SEQ ID NO: 232 - DNA, 28 bp, synthetic construct
  source 1..28: mol_type = other DNA
cagctgcccggcnnkaaaaagaatggtt

SEQ ID NO: 233 - DNA, 27 bp, synthetic construct
  source 1..27: mol_type = other DNA
ctgcccggcgagnnkaagaatggtttg

SEQ ID NO: 234 - DNA, 29 bp, synthetic construct
  source 1..29: mol_type = other DNA
gcccggcgagaaannkaatggtttgtttg

SEQ ID NO: 235 - DNA, 30 bp, synthetic construct
  source 1..30: mol_type = other DNA
cggcgagaaaaagnnkggtttgtttgggaa

SEQ ID NO: 236 - DNA, 36 bp, synthetic construct
  source 1..36: mol_type = other DNA
cggcgagaaaaagaatnnkttgtttgggaaccttat

SEQ ID NO: 237 - DNA, 36 bp, synthetic construct
  source 1..36: mol_type = other DNA
cgagaaaaagaatggtnnktttgggaaccttattgc

SEQ ID NO: 238 - DNA, 37 bp, synthetic construct
  source 1..37: mol_type = other DNA
agaaaaagaatggtttgnnkgggaaccttattgcctt

SEQ ID NO: 239 - DNA, 40 bp, synthetic construct
  source 1..40: mol_type = other DNA
aaaaagaatggtttgtttnnkaaccttattgccttgagct

SEQ ID NO: 240 - DNA, 33 bp, synthetic construct
  source 1..33: mol_type = other DNA
aatggtttgtttgggnnkcttattgccttgagc

SEQ ID NO: 241 - DNA, 34 bp, synthetic construct
  source 1..34: mol_type = other DNA
ggtttgtttgggaacnnkattgccttgagcttag

SEQ ID NO: 242 - DNA, 31 bp, synthetic construct
  source 1..31: mol_type = other DNA
tgtttgggaaccttnnkgccttgagcttagg

SEQ ID NO: 243 - DNA, 35 bp, synthetic construct
  source 1..35: mol_type = other DNA
gtttgggaaccttattnnkttgagcttaggcctca

SEQ ID NO: 244 - DNA, 30 bp, synthetic construct
  source 1..30: mol_type = other DNA
gaaccttattgccnnkagcttaggcctcac

SEQ ID NO: 245 - DNA, 30 bp, synthetic construct
  source 1..30: mol_type = other DNA
ccttattgccttgnnkttaggcctcacccc

SEQ ID NO: 246 - DNA, 25 bp, synthetic construct
  source 1..25: mol_type = other DNA
ttgccttgagcnnkggcctcacccc

SEQ ID NO: 247 - DNA, 31 bp, synthetic construct
  source 1..31: mol_type = other DNA
ttgccttgagcttannkctcaccccgaattt

SEQ ID NO: 248 - DNA, 30 bp, synthetic construct
  source 1..30: mol_type = other DNA
cttgagcttaggcnnkaccccgaatttcaa

SEQ ID NO: 249 - DNA, 36 bp, synthetic construct
  source 1..36: mol_type = other DNA
cttgagcttaggcctcnnkccgaatttcaaaagtaa

SEQ ID NO: 250 - DNA, 39 bp, synthetic construct
  source 1..39: mol_type = other DNA
ttgagcttaggcctcaccnnkaatttcaaaagtaatttc

SEQ ID NO: 251 - DNA, 34 bp, synthetic construct
  source 1..34: mol_type = other DNA
ttaggcctcaccccgnnkttcaaaagtaatttcg

SEQ ID NO: 252 - DNA, 37 bp, synthetic construct
  source 1..37: mol_type = other DNA
taggcctcaccccgaatnnkaaaagtaatttcgatct

SEQ ID NO: 253 - DNA, 35 bp, synthetic construct
  source 1..35: mol_type = other DNA
cctcaccccgaatttcnnkagtaatttcgatcttg

SEQ ID NO: 254 - DNA, 36 bp, synthetic construct
  source 1..36: mol_type = other DNA
caccccgaatttcaaannkaatttcgatcttgcaga

SEQ ID NO: 255 - DNA, 36 bp, synthetic construct
  source 1..36: mol_type = other DNA
cccgaatttcaaaagtnnkttcgatcttgcagaaga

SEQ ID NO: 256 - DNA, 38 bp, synthetic construct
  source 1..38: mol_type = other DNA
cgaatttcaaaagtaatnnkgatcttgcagaagacgcc

SEQ ID NO: 257 - DNA, 37 bp, synthetic construct
  source 1..37: mol_type = other DNA
atttcaaaagtaatttcnnkcttgcagaagacgccaa

SEQ ID NO: 258 - DNA, 37 bp, synthetic construct
  source 1..37: mol_type = other DNA
tcaaaagtaatttcgatnnkgcagaagacgccaaatt

SEQ ID NO: 259 - DNA, 42 bp, synthetic construct
  source 1..42: mol_type = other DNA
caaaagtaatttcgatcttnnkgaagacgccaaattacaact

SEQ ID NO: 260 - DNA, 37 bp, synthetic construct
  source 1..37: mol_type = other DNA
gtaatttcgatcttgcannkgacgccaaattacaact

SEQ ID NO: 261 - DNA, 37 bp, synthetic construct
  source 1..37: mol_type = other DNA
atttcgatcttgcagaannkgccaaattacaactgtc

SEQ ID NO: 262 - DNA, 37 bp, synthetic construct
  source 1..37: mol_type = other DNA
tcgatcttgcagaagacnnkaaattacaactgtcgaa

SEQ ID NO: 263 - DNA, 31 bp, synthetic construct
  source 1..31: mol_type = other DNA
ttgcagaagacgccnnkttacaactgtcgaa

SEQ ID NO: 264 - DNA, 30 bp, synthetic construct
  source 1..30: mol_type = other DNA
agaagacgccaaannkcaactgtcgaagga

SEQ ID NO: 265 - DNA, 37 bp, synthetic construct
  source 1..37: mol_type = other DNA
cagaagacgccaaattannkctgtcgaaggatactta

SEQ ID NO: 266 - DNA, 41 bp, synthetic construct
  source 1..41: mol_type = other DNA
agaagacgccaaattacaannktcgaaggatacttatgatg

SEQ ID NO: 267 - DNA, 40 bp, synthetic construct
  source 1..40: mol_type = other DNA
gacgccaaattacaactgnnkaaggatacttatgatgacg

SEQ ID NO: 268 - DNA, 39 bp, synthetic construct
  source 1..39: mol_type = other DNA
gccaaattacaactgtcgnnkgatacttatgatgacgat

SEQ ID NO: 269 - DNA, 40 bp, synthetic construct
  source 1..40: mol_type = other DNA
aaattacaactgtcgaagnnkacttatgatgacgatctcg

SEQ ID NO: 270 - DNA, 37 bp, synthetic construct
  source 1..37: mol_type = other DNA
tacaactgtcgaaggatnnktatgatgacgatctcga

SEQ ID NO: 271 - DNA, 37 bp, synthetic construct
  source 1..37: mol_type = other DNA
aactgtcgaaggatactnnkgatgacgatctcgataa

SEQ ID NO: 272 - DNA, 40 bp, synthetic construct
  source 1..40: mol_type = other DNA
ctgtcgaaggatacttatnnkgacgatctcgataatctgt

SEQ ID NO: 273 - DNA, 43 bp, synthetic construct
  source 1..43: mol_type = other DNA
tgtcgaaggatacttatgatnnkgatctcgataatctgttagc

SEQ ID NO: 274 - DNA, 40 bp, synthetic construct
  source 1..40: mol_type = other DNA
aaggatacttatgatgacnnkctcgataatctgttagcgc

SEQ ID NO: 275 - DNA, 41 bp, synthetic construct
  source 1..41: mol_type = other DNA
ggatacttatgatgacgatnnkgataatctgttagcgcaga

SEQ ID NO: 276 - DNA, 37 bp, synthetic construct
  source 1..37: mol_type = other DNA
cttatgatgacgatctcnnkaatctgttagcgcagat

SEQ ID NO: 277 - DNA, 33 bp, synthetic construct
  source 1..33: mol_type = other DNA
gatgacgatctcgatnnkctgttagcgcagatt

SEQ ID NO: 278 - DNA, 35 bp, synthetic construct
  source 1..35: mol_type = other DNA
tgacgatctcgataatnnkttagcgcagattggtg

SEQ ID NO: 279 - DNA, 32 bp, synthetic construct
  source 1..32: mol_type = other DNA
atctcgataatctgnnkgcgcagattggtgac

SEQ ID NO: 280 - DNA, 40 bp, synthetic construct
  source 1..40: mol_type = other DNA
gatctcgataatctgttannkcagattggtgaccaatacg

SEQ ID NO: 281 - DNA, 35 bp, synthetic construct
  source 1..35: mol_type = other DNA
cgataatctgttagcgnnkattggtgaccaatacg

SEQ ID NO: 282 - DNA, 29 bp, synthetic construct
  source 1..29: mol_type = other DNA
tctgttagcgcagnnkggtgaccaatacg

SEQ ID NO: 283 - DNA, 29 bp, synthetic construct
  source 1..29: mol_type = other DNA
gttagcgcagattnnkgaccaatacgccg

SEQ ID NO: 284 - DNA, 28 bp, synthetic construct
  source 1..28: mol_type = other DNA
gcgcagattggtnnkcaatacgccgatc

SEQ ID NO: 285 - DNA, 30 bp, synthetic construct
  source 1..30: mol_type = other DNA
gcagattggtgacnnktacgccgatctttt

SEQ ID NO: 286 - DNA, 33 bp, synthetic construct
  source 1..33: mol_type = other DNA
cagattggtgaccaannkgccgatctttttctg

SEQ ID NO: 287 - DNA, 33 bp, synthetic construct
  source 1..33: mol_type = other DNA
attggtgaccaatacnnkgatctttttctggcg

SEQ ID NO: 288 - DNA, 28 bp, synthetic construct
  source 1..28: mol_type = other DNA
gaccaatacgccnnkctttttctggcgg

SEQ ID NO: 289 - DNA, 28 bp, synthetic construct
  source 1..28: mol_type = other DNA
caatacgccgatnnktttctggcggcta

SEQ ID NO: 290 - DNA, 32 bp, synthetic construct
  source 1..32: mol_type = other DNA
aatacgccgatcttnnkctggcggctaaaaat

SEQ ID NO: 291 - DNA, 33 bp, synthetic construct
  source 1..33: mol_type = other DNA
tacgccgatctttttnnkgcggctaaaaatctg

SEQ ID NO: 292 - DNA, 34 bp, synthetic construct
  source 1..34: mol_type = other DNA
gccgatctttttctgnnkgctaaaaatctgagcg

SEQ ID NO: 293 - DNA, 34 bp, synthetic construct
  source 1..34: mol_type = other DNA
gatctttttctggcgnnkaaaaatctgagcgacg

SEQ ID NO: 294 - DNA, 27 bp, synthetic construct
  source 1..27: mol_type = other DNA
tttctggcggctnnkaatctgagcgac

SEQ ID NO: 295 - DNA, 25 bp, synthetic construct
  source 1..25: mol_type = other DNA
tggcggctaaannkctgagcgacgc

SEQ ID NO: 296 - DNA, 28 bp, synthetic construct
  source 1..28: mol_type = other DNA
gcggctaaaaatnnkagcgacgccatct

SEQ ID NO: 297 - DNA, 30 bp, synthetic construct
  source 1..30: mol_type = other DNA
ggctaaaaatctgnnkgacgccatcttgct

SEQ ID NO: 298 - DNA, 32 bp, synthetic construct
  source 1..32: mol_type = other DNA
ctaaaaatctgagcnnkgccatcttgctttcg

SEQ ID NO: 299 - DNA, 39 bp, synthetic construct
  source 1..39: mol_type = other DNA
gctaaaaatctgagcgacnnkatcttgctttcggatatt

SEQ ID NO: 300 - DNA, 25 bp, synthetic construct
  source 1..25: mol_type = other DNA
tgagcgacgccnnkttgctttcgga

SEQ ID NO: 301 - DNA, 30 bp, synthetic construct
  source 1..30: mol_type = other DNA
gagcgacgccatcnnkctttcggatattct

SEQ ID NO: 302 - DNA, 29 bp, synthetic construct
  source 1..29: mol_type = other DNA
cgacgccatcttgnnktcggatattctcc

SEQ ID NO: 303 - DNA, 29 bp, synthetic construct
  source 1..29: mol_type = other DNA
cgccatcttgcttnnkgatattctccgcg

SEQ ID NO: 304 - DNA, 32 bp, synthetic construct
  source 1..32: mol_type = other DNA
ccatcttgctttcgnnkattctccgcgttaac

SEQ ID NO: 305 - DNA, 30 bp, synthetic construct
  source 1..30: mol_type = other DNA
cttgctttcggatnnkctccgcgttaacac

SEQ ID NO: 306 - DNA, 31 bp, synthetic construct
  source 1..31: mol_type = other DNA
tgctttcggatattnnkcgcgttaacaccga

SEQ ID NO: 307 - DNA, 36 bp, synthetic construct
  source 1..36: mol_type = other DNA
gctttcggatattctcnnkgttaacaccgaaatcac

SEQ ID NO: 308 - DNA, 31 bp, synthetic construct
  source 1..31: mol_type = other DNA
cggatattctccgcnnkaacaccgaaatcac

SEQ ID NO: 309 - DNA, 34 bp, synthetic construct
  source 1..34: mol_type = other DNA
gatattctccgcgttnnkaccgaaatcacgaaag

SEQ ID NO: 310 - DNA, 31 bp, synthetic construct
  source 1..31: mol_type = other DNA
ttctccgcgttaacnnkgaaatcacgaaagc

SEQ ID NO: 311 - DNA, 27 bp, synthetic construct
  source 1..27: mol_type = other DNA
cgcgttaacaccnnkatcacgaaagcg

SEQ ID NO: 312 - DNA, 28 bp, synthetic construct
  source 1..28: mol_type = other DNA
gttaacaccgaannkacgaaagcgcctc

SEQ ID NO: 313 - DNA, 34 bp, synthetic construct
  source 1..34: mol_type = other DNA
gttaacaccgaaatcnnkaaagcgcctcttagtg

SEQ ID NO: 314 - DNA, 27 bp, synthetic construct
  source 1..27: mol_type = other DNA
accgaaatcacgnnkgcgcctcttagt

SEQ ID NO: 315 - DNA, 31 bp, synthetic construct
  source 1..31: mol_type = other DNA
ccgaaatcacgaaannkcctcttagtgccag

SEQ ID NO: 316 - DNA, 27 bp, synthetic construct
  source 1..27: mol_type = other DNA
atcacgaaagcgnnkcttagtgccagc

SEQ ID NO: 317 - DNA, 26 bp, synthetic construct
  source 1..26: mol_type = other DNA
cgaaagcgcctnnkagtgccagcatg

SEQ ID NO: 318 - DNA, 30 bp, synthetic construct
  source 1..30: mol_type = other DNA
gaaagcgcctcttnnkgccagcatgattaa

SEQ ID NO: 319 - DNA, 37 bp, synthetic construct
  source 1..37: mol_type = other DNA
cgaaagcgcctcttagtnnkagcatgattaaacgtta

SEQ ID NO: 320 - DNA, 34 bp, synthetic construct
  source 1..34: mol_type = other DNA
gcgcctcttagtgccnnkatgattaaacgttatg

SEQ ID NO: 321 - DNA, 36 bp, synthetic construct
  source 1..36: mol_type = other DNA
gcctcttagtgccagcnnkattaaacgttatgatga

SEQ ID NO: 322 - DNA, 37 bp, synthetic construct
  source 1..37: mol_type = other DNA
ctcttagtgccagcatgnnkaaacgttatgatgaaca

SEQ ID NO: 323 - DNA, 35 bp, synthetic construct
  source 1..35: mol_type = other DNA
tagtgccagcatgattnnkcgttatgatgaacacc

SEQ ID NO: 324 - DNA, 36 bp, synthetic construct
  source 1..36: mol_type = other DNA
tgccagcatgattaaannktatgatgaacaccacca

SEQ ID NO: 325 - DNA, 33 bp, synthetic construct
  source 1..33: mol_type = other DNA
agcatgattaaacgtnnkgatgaacaccaccag

SEQ ID NO: 326 - DNA, 36 bp, synthetic construct
  source 1..36: mol_type = other DNA
catgattaaacgttatnnkgaacaccaccaggacct

SEQ ID NO: 327 - DNA, 36 bp, synthetic construct
  source 1..36: mol_type = other DNA
gattaaacgttatgatnnkcaccaccaggacctgac

SEQ ID NO: 328 - DNA, 35 bp, synthetic construct
  source 1..35: mol_type = other DNA
taaacgttatgatgaannkcaccaggacctgacct

SEQ ID NO: 329 - DNA, 34 bp, synthetic construct
  source 1..34: mol_type = other DNA
cgttatgatgaacacnnkcaggacctgaccttac

SEQ ID NO: 330 - DNA, 36 bp, synthetic construct
  source 1..36: mol_type = other DNA
ttatgatgaacaccacnnkgacctgaccttactcaa

SEQ ID NO: 331 - DNA, 34 bp, synthetic construct
  source 1..34: mol_type = other DNA
gatgaacaccaccagnnkctgaccttactcaaag

SEQ ID NO: 332 - DNA, 31 bp, synthetic construct
  source 1..31: mol_type = other DNA
aacaccaccaggacnnkaccttactcaaagc

SEQ ID NO: 333 - DNA, 30 bp, synthetic construct
  source 1..30: mol_type = other DNA
ccaccaggacctgnnkttactcaaagcgtt

SEQ ID NO: 334 - DNA, 27 bp, synthetic construct
  source 1..27: mol_type = other DNA
caggacctgaccnnkctcaaagcgttg

SEQ ID NO: 335 - DNA, 30 bp, synthetic construct
  source 1..30: mol_type = other DNA
ggacctgaccttannkaaagcgttggttcg

SEQ ID NO: 336 - DNA, 27 bp, synthetic construct
  source 1..27: mol_type = other DNA
ctgaccttactcnnkgcgttggttcgc

SEQ ID NO: 337 - DNA, 32 bp, synthetic construct
  source 1..32: mol_type = other DNA
tgaccttactcaaannkttggttcgccagcaa

SEQ ID NO: 338 - DNA, 29 bp, synthetic construct
  source 1..29: mol_type = other DNA
cttactcaaagcgnnkgttcgccagcaac

SEQ ID NO: 339 - DNA, 25 bp, synthetic construct
  source 1..25: mol_type = other DNA
tcaaagcgttgnnkcgccagcaact

SEQ ID NO: 340 - DNA, 28 bp, synthetic construct
  source 1..28: mol_type = other DNA
aaagcgttggttnnkcagcaactgccag

SEQ ID NO: 341 - DNA, 26 bp, synthetic construct
  source 1..26: mol_type = other DNA
cgttggttcgcnnkcaactgccagag

SEQ ID NO: 342 - DNA, 25 bp, synthetic construct
  source 1..25: mol_type = other DNA
tggttcgccagnnkctgccagagaa

SEQ ID NO: 343 - DNA, 29 bp, synthetic construct
  source 1..29: mol_type = other DNA
ggttcgccagcaannkccagagaagtaca

SEQ ID NO: 344 - DNA, 32 bp, synthetic construct
  source 1..32: mol_type = other DNA
ttcgccagcaactgnnkgagaagtacaaagaa

SEQ ID NO: 345 - DNA, 32 bp, synthetic construct
  source 1..32: mol_type = other DNA
gccagcaactgccannkaagtacaaagaaatc

SEQ ID NO: 346 - DNA, 36 bp, synthetic construct
  source 1..36: mol_type = other DNA
ccagcaactgccagagnnktacaaagaaatcttctt

SEQ ID NO: 347 - DNA, 37 bp, synthetic construct
  source 1..37: mol_type = other DNA
agcaactgccagagaagnnkaaagaaatcttctttga

SEQ ID NO: 348 - DNA, 37 bp, synthetic construct
  source 1..37: mol_type = other DNA
aactgccagagaagtacnnkgaaatcttctttgatca

SEQ ID NO: 349 - DNA, 39 bp, synthetic construct
  source 1..39: mol_type = other DNA
ctgccagagaagtacaaannkatcttctttgatcagtca

SEQ ID NO: 350 - DNA, 41 bp, synthetic construct
  source 1..41: mol_type = other DNA
gccagagaagtacaaagaannkttctttgatcagtcaaaga

SEQ ID NO: 351 - DNA, 44 bp, synthetic construct
  source 1..44: mol_type = other DNA
cagagaagtacaaagaaatcnnktttgatcagtcaaagaatggt

SEQ ID NO: 352 - DNA, 45 bp, synthetic construct
  source 1..45: mol_type = other DNA
gagaagtacaaagaaatcttcnnkgatcagtcaaagaatggttat

SEQ ID NO: 353 - DNA, 44 bp, synthetic construct
  source 1..44: mol_type = other DNA
agtacaaagaaatcttctttnnkcagtcaaagaatggttatgcc

SEQ ID NO: 354 - DNA, 42 bp, synthetic construct
  source 1..42: mol_type = other DNA
caaagaaatcttctttgatnnktcaaagaatggttatgccgg

SEQ ID NO: 355 - DNA, 38 bp, synthetic construct
  source 1..38: mol_type = other DNA
aaatcttctttgatcagnnkaagaatggttatgccggc

SEQ ID NO: 356 - DNA, 36 bp, synthetic construct
  source 1..36: mol_type = other DNA
cttctttgatcagtcannkaatggttatgccggcta

SEQ ID NO: 357 - DNA, 36 bp, synthetic construct
  source 1..36: mol_type = other DNA
ctttgatcagtcaaagnnkggttatgccggctatat

SEQ ID NO: 358 - DNA, 40 bp, synthetic construct
  source 1..40: mol_type = other DNA
tttgatcagtcaaagaatnnktatgccggctatattgacg

SEQ ID NO: 359 - DNA, 33 bp, synthetic construct
  source 1..33: mol_type = other DNA
cagtcaaagaatggtnnkgccggctatattgac

SEQ ID NO: 360 - DNA, 36 bp, synthetic construct
  source 1..36: mol_type = other DNA
gtcaaagaatggttatnnkggctatattgacggggg

SEQ ID NO: 361 - DNA, 35 bp, synthetic construct
  source 1..35: mol_type = other DNA
aaagaatggttatgccnnktatattgacgggggtg

SEQ ID NO: 362 - DNA, 25 bp, synthetic construct
  source 1..25: mol_type = other DNA
gttatgccggcnnkattgacggggg

SEQ ID NO: 363 - DNA, 25 bp, synthetic construct
  source 1..25: mol_type = other DNA
atgccggctatnnkgacgggggtgc

SEQ ID NO: 364 - DNA, 26 bp, synthetic construct
  source 1..26: mol_type = other DNA
ccggctatattnnkgggggtgcaagc

SEQ ID NO: 365 - DNA, 29 bp, synthetic construct
  source 1..29: mol_type = other DNA
cggctatattgacnnkggtgcaagccaag

SEQ ID NO: 366 - DNA, 30 bp, synthetic construct
  source 1..30: mol_type = other DNA
ctatattgacgggnnkgcaagccaagagga

SEQ ID NO: 367 - DNA, 36 bp, synthetic construct
  source 1..36: mol_type = other DNA
ctatattgacgggggtnnkagccaagaggaattcta

SEQ ID NO: 368 - DNA, 28 bp, synthetic construct
  source 1..28: mol_type = other DNA
gacgggggtgcannkcaagaggaattct

SEQ ID NO: 369 - DNA, 28 bp, synthetic construct
  source 1..28: mol_type = other DNA
gggggtgcaagcnnkgaggaattctaca

SEQ ID NO: 370 - DNA, 34 bp, synthetic construct
  source 1..34: mol_type = other DNA
gggggtgcaagccaannkgaattctacaaattta

SEQ ID NO: 371 - DNA, 36 bp, synthetic construct
  source 1..36: mol_type = other DNA
gggtgcaagccaagagnnkttctacaaatttatcaa

SEQ ID NO: 372 - DNA, 37 bp, synthetic construct
  source 1..37: mol_type = other DNA
gtgcaagccaagaggaannktacaaatttatcaagcc

SEQ ID NO: 373 - DNA, 39 bp, synthetic construct
  source 1..39: mol_type = other DNA
gcaagccaagaggaattcnnkaaatttatcaagcctatt

SEQ ID NO: 374 - DNA, 40 bp, synthetic construct
  source 1..40: mol_type = other DNA
agccaagaggaattctacnnktttatcaagcctattctgg

SEQ ID NO: 375 - DNA, 41 bp, synthetic construct
  source 1..41: mol_type = other DNA
ccaagaggaattctacaaannkatcaagcctattctggaga

SEQ ID NO: 376 - DNA, 46 bp, synthetic construct
  source 1..46: mol_type = other DNA
caagaggaattctacaaatttnnkaagcctattctggagaaaatgg

SEQ ID NO: 377 - DNA, 46 bp, synthetic construct
  source 1..46: mol_type = other DNA
gaggaattctacaaatttatcnnkcctattctggagaaaatggatg

SEQ ID NO: 378 - DNA, 45 bp, synthetic construct
  source 1..45: mol_type = other DNA
gaattctacaaatttatcaagnnkattctggagaaaatggatggc

SEQ ID NO: 379 - DNA, 38 bp, synthetic construct
  source 1..38: mol_type = other DNA
acaaatttatcaagcctnnkctggagaaaatggatggc

SEQ ID NO: 380 - DNA, 40 bp, synthetic construct
  source 1..40: mol_type = other DNA
aaatttatcaagcctattnnkgagaaaatggatggcaccg

SEQ ID NO: 381 - DNA, 35 bp, synthetic construct
  source 1..35: mol_type = other DNA
tatcaagcctattctgnnkaaaatggatggcaccg

SEQ ID NO: 382 - DNA, 29 bp, synthetic construct
  source 1..29: mol_type = other DNA
gcctattctggagnnkatggatggcaccg

SEQ ID NO: 383 - DNA, 33 bp, synthetic construct
  source 1..33: mol_type = other DNA
cctattctggagaaannkgatggcaccgaagag

SEQ ID NO: 384 - DNA, 37 bp, synthetic construct
  source 1..37: mol_type = other DNA
ctattctggagaaaatgnnkggcaccgaagagttatt

SEQ ID NO: 385 - DNA, 38 bp, synthetic construct
  source 1..38: mol_type = other DNA
ttctggagaaaatggatnnkaccgaagagttattggtg

SEQ ID NO: 386 - DNA, 36 bp, synthetic construct
  source 1..36: mol_type = other DNA
ggagaaaatggatggcnnkgaagagttattggtgaa

SEQ ID NO: 387 - DNA, 33 bp, synthetic construct
  source 1..33: mol_type = other DNA
aaaatggatggcaccnnkgagttattggtgaag

SEQ ID NO: 388 - DNA, 37 bp, synthetic construct
  source 1..37: mol_type = other DNA
aaatggatggcaccgaannkttattggtgaagcttaa

SEQ ID NO: 389 - DNA, 31 bp, synthetic construct
  source 1..31: mol_type = other DNA
atggcaccgaagagnnkttggtgaagcttaa

SEQ ID NO: 390 - DNA, 32 bp, synthetic construct
  source 1..32: mol_type = other DNA
gcaccgaagagttannkgtgaagcttaaccgt

SEQ ID NO: 391 - DNA, 36 bp, synthetic construct
  source 1..36: mol_type = other DNA
caccgaagagttattgnnkaagcttaaccgtgaaga

SEQ ID NO: 392 - DNA, 34 bp, synthetic construct
  source 1..34: mol_type = other DNA
gaagagttattggtgnnkcttaaccgtgaagacc

SEQ ID NO: 393 - DNA, 34 bp, synthetic construct
  source 1..34: mol_type = other DNA
gagttattggtgaagnnkaaccgtgaagacctcc

SEQ ID NO: 394 - DNA, 33 bp, synthetic construct
  source 1..33: mol_type = other DNA
ttattggtgaagcttnnkcgtgaagacctcctg

SEQ ID NO: 395 - DNA, 29 bp, synthetic construct
  source 1..29: mol_type = other DNA
ggtgaagcttaacnnkgaagacctcctgc

SEQ ID NO: 396 - DNA, 27 bp, synthetic construct
  source 1..27: mol_type = other DNA
aagcttaaccgtnnkgacctcctgcgg

SEQ ID NO: 397 - DNA, 29 bp, synthetic construct
  source 1..29: mol_type = other DNA
gcttaaccgtgaannkctcctgcggaaac

SEQ ID NO: 398 - DNA, 27 bp, synthetic construct
  source 1..27: mol_type = other DNA
aaccgtgaagacnnkctgcggaaacag

SEQ ID NO: 399 - DNA, 26 bp, synthetic construct
  source 1..26: mol_type = other DNA
gtgaagacctcnnkcggaaacagcgc

SEQ ID NO: 400 - DNA, 30 bp, synthetic construct
  source 1..30: mol_type = other DNA
tgaagacctcctgnnkaaacagcgcacatt

SEQ ID NO: 401 - DNA, 25 bp, synthetic construct
  source 1..25: mol_type = other DNA
acctcctgcggnnkcagcgcacatt

SEQ ID NO: 402 - DNA, 28 bp, synthetic construct
  source 1..28: mol_type = other DNA
ctcctgcggaaannkcgcacattcgata

SEQ ID NO: 403 - DNA, 34 bp, synthetic construct
  source 1..34: mol_type = other DNA
ctcctgcggaaacagnnkacattcgataatggtt

SEQ ID NO: 404 - DNA, 29 bp, synthetic construct
  source 1..29: mol_type = other DNA
gcggaaacagcgcnnkttcgataatggtt

SEQ ID NO: 405 - DNA, 32 bp, synthetic construct
  source 1..32: mol_type = other DNA
ggaaacagcgcacannkgataatggttcgatc

SEQ ID NO: 406 - DNA, 29 bp, synthetic construct
  source 1..29: mol_type = other DNA
acagcgcacattcnnkaatggttcgatcc

SEQ ID NO: 407 - DNA, 28 bp, synthetic construct
  source 1..28: mol_type = other DNA
cgcacattcgatnnkggttcgatcccac

SEQ ID NO: 408 - DNA, 32 bp, synthetic construct
  source 1..32: mol_type = other DNA
gcacattcgataatnnktcgatcccacaccaa

SEQ ID NO: 409 - DNA, 36 bp, synthetic construct
  source 1..36: mol_type = other DNA
cacattcgataatggtnnkatcccacaccaaatcca

SEQ ID NO: 410 - DNA, 33 bp, synthetic construct
  source 1..33: mol_type = other DNA
ttcgataatggttcgnnkccacaccaaatccat

SEQ ID NO: 411 - DNA, 35 bp, synthetic construct
  source 1..35: mol_type = other DNA
cgataatggttcgatcnnkcaccaaatccatttgg

SEQ ID NO: 412 - DNA, 31 bp, synthetic construct
  source 1..31: mol_type = other DNA
atggttcgatcccannkcaaatccatttggg

SEQ ID NO: 413 - DNA, 29 bp, synthetic construct
  source 1..29: mol_type = other DNA
ttcgatcccacacnnkatccatttggggg

SEQ ID NO: 414 - DNA, 30 bp, synthetic construct
  source 1..30: mol_type = other DNA
gatcccacaccaannkcatttgggggagtt

SEQ ID NO: 415 - DNA, 30 bp, synthetic construct
  source 1..30: mol_type = other DNA
cccacaccaaatcnnkttgggggagttaca

SEQ ID NO: 416 - DNA, 29 bp, synthetic construct
  source 1..29: mol_type = other DNA
acaccaaatccatnnkggggagttacacg

SEQ ID NO: 417 - DNA, 37 bp, synthetic construct
  source 1..37: mol_type = other DNA
cacaccaaatccatttgnnkgagttacacgctatttt

SEQ ID NO: 418 - DNA, 34 bp, synthetic construct
  source 1..34: mol_type = other DNA
caaatccatttggggnnkttacacgctattttgc

SEQ ID NO: 419 - DNA, 28 bp, synthetic construct
  source 1..28: mol_type = other DNA
catttgggggagnnkcacgctattttgc

SEQ ID NO: 420 - DNA, 31 bp, synthetic construct
  source 1..31: mol_type = other DNA
atttgggggagttannkgctattttgcgtcg

SEQ ID NO: 421 - DNA, 28 bp, synthetic construct
  source 1..28: mol_type = other DNA
ggggagttacacnnkattttgcgtcgcc

SEQ ID NO: 422 - DNA, 26 bp, synthetic construct
  source 1..26: mol_type = other DNA
agttacacgctnnkttgcgtcgccag

SEQ ID NO: 423 - DNA, 30 bp, synthetic construct
  source 1..30: mol_type = other DNA
gttacacgctattnnkcgtcgccaggaaga

SEQ ID NO: 424 - DNA, 31 bp, synthetic construct
  source 1..31: mol_type = other DNA
tacacgctattttgnnkcgccaggaagactt

SEQ ID NO: 425 - DNA, 34 bp, synthetic construct
  source 1..34: mol_type = other DNA
cacgctattttgcgtnnkcaggaagacttttacc

SEQ ID NO: 426 - DNA, 32 bp, synthetic construct
  source 1..32: mol_type = other DNA
ctattttgcgtcgcnnkgaagacttttaccct

SEQ ID NO: 427 - DNA, 28 bp, synthetic construct
  source 1..28: mol_type = other DNA
ttgcgtcgccagnnkgacttttaccctt

SEQ ID NO: 428 - DNA, 29 bp, synthetic construct
  source 1..29: mol_type = other DNA
gcgtcgccaggaannkttttaccctttcc

SEQ ID NO: 429 - DNA, 30 bp, synthetic construct
  source 1..30: mol_type = other DNA
tcgccaggaagacnnktaccctttcctgaa

SEQ ID NO: 430 - DNA, 32 bp, synthetic construct
  source 1..32: mol_type = other DNA
gccaggaagactttnnkcctttcctgaaggat

SEQ ID NO: 431 - DNA, 36 bp, synthetic construct
  source 1..36: mol_type = other DNA
ccaggaagacttttacnnkttcctgaaggataaccg

SEQ ID NO: 432 - DNA, 34 bp, synthetic construct
  source 1..34: mol_type = other DNA
gaagacttttaccctnnkctgaaggataaccggg

SEQ ID NO: 433 - DNA, 37 bp, synthetic construct
  source 1..37: mol_type = other DNA
aagacttttaccctttcnnkaaggataaccgggagaa

SEQ ID NO: 434 - DNA, 36 bp, synthetic construct
  source 1..36: mol_type = other DNA
cttttaccctttcctgnnkgataaccgggagaaaat

SEQ ID NO: 435 - DNA, 35 bp, synthetic construct
  source 1..35: mol_type = other DNA
ttaccctttcctgaagnnkaaccgggagaaaattg

SEQ ID NO: 436 - DNA, 34 bp, synthetic construct
  source 1..34: mol_type = other DNA
cctttcctgaaggatnnkcgggagaaaattgaga

SEQ ID NO: 437 - DNA, 40 bp, synthetic construct
  source 1..40: mol_type = other DNA
cctttcctgaaggataacnnkgagaaaattgagaagatcc

SEQ ID NO: 438 - DNA, 37 bp, synthetic construct
  source 1..37: mol_type = other DNA
tcctgaaggataaccggnnkaaaattgagaagatcct

SEQ ID NO: 439 - DNA, 37 bp, synthetic construct
  source 1..37: mol_type = other DNA
tgaaggataaccgggagnnkattgagaagatccttac

SEQ ID NO: 440 - DNA, 36 bp, synthetic construct
  source 1..36: mol_type = other DNA
ggataaccgggagaaannkgagaagatccttacctt

SEQ ID NO: 441 - DNA, 41 bp, synthetic construct
  source 1..41: mol_type = other DNA
ggataaccgggagaaaattnnkaagatccttacctttcgta

SEQ ID NO: 442 - DNA, 37 bp, synthetic construct
  source 1..37: mol_type = other DNA
accgggagaaaattgagnnkatccttacctttcgtat

SEQ ID NO: 443 - DNA, 38 bp, synthetic construct
  source 1..38: mol_type = other DNA
gggagaaaattgagaagnnkcttacctttcgtattccg

SEQ ID NO: 444 - DNA, 41 bp, synthetic construct
  source 1..41: mol_type = other DNA
ggagaaaattgagaagatcnnkacctttcgtattccgtatt

SEQ ID NO: 445 - DNA, 43 bp, synthetic construct
  source 1..43: mol_type = other DNA
agaaaattgagaagatccttnnktttcgtattccgtattacgt

SEQ ID NO: 446 - DNA, 37 bp, synthetic construct
  source 1..37: mol_type = other DNA
ttgagaagatccttaccnnkcgtattccgtattacgt

SEQ ID NO: 447 - DNA, 40 bp, synthetic construct
  source 1..40: mol_type = other DNA
gagaagatccttacctttnnkattccgtattacgtaggcc

SEQ ID NO: 448 - DNA, 33 bp, synthetic construct
  source 1..33: mol_type = other DNA
atccttacctttcgtnnkccgtattacgtaggc

SEQ ID NO: 449 - DNA, 37 bp, synthetic construct
  source 1..37: mol_type = other DNA
tccttacctttcgtattnnktattacgtaggcccctt

SEQ ID NO: 450 - DNA, 30 bp, synthetic construct
  source 1..30: mol_type = other DNA
ctttcgtattccgnnktacgtaggcccctt

SEQ ID NO: 451 - DNA, 33 bp, synthetic construct
  source 1..33: mol_type = other DNA
tttcgtattccgtatnnkgtaggccccttagca

SEQ ID NO: 452 - DNA, 31 bp, synthetic construct
  source 1..31: mol_type = other DNA
gtattccgtattacnnkggccccttagcacg

SEQ ID NO: 453 - DNA, 29 bp, synthetic construct
  source 1..29: mol_type = other DNA
tccgtattacgtannkcccttagcacggg

SEQ ID NO: 454 - DNA, 32 bp, synthetic construct
  source 1..32: mol_type = other DNA
cgtattacgtaggcnnkttagcacggggtaat

SEQ ID NO: 455 - DNA, 25 bp, synthetic construct
  source 1..25: mol_type = other DNA
acgtaggccccnnkgcacggggtaa

SEQ ID NO: 456 - DNA, 28 bp, synthetic construct
  source 1..28: mol_type = other DNA
gtaggccccttannkcggggtaatagcc

SEQ ID NO: 457 - DNA, 31 bp, synthetic construct
  source 1..31: mol_type = other DNA
taggccccttagcannkggtaatagccgttt

SEQ ID NO: 458 - DNA, 28 bp, synthetic construct
  source 1..28: mol_type = other DNA
cccttagcacggnnkaatagccgtttcg

SEQ ID NO: 459 - DNA, 25 bp, synthetic construct
  source 1..25: mol_type = other DNA
tagcacggggtnnkagccgtttcgc

SEQ ID NO: 460 - DNA, 26 bp, synthetic construct
  source 1..26: mol_type = other DNA
cacggggtaatnnkcgtttcgcgtgg

SEQ ID NO: 461 - DNA, 28 bp, synthetic construct
  source 1..28: mol_type = other DNA
cggggtaatagcnnkttcgcgtggatga

SEQ ID NO: 462 - DNA, 28 bp, synthetic construct
  source 1..28: mol_type = other DNA
ggtaatagccgtnnkgcgtggatgacac

SEQ ID NO: 463 - DNA, 33 bp, synthetic construct
  source 1..33: mol_type = other DNA
ggtaatagccgtttcnnktggatgacacggaag

SEQ ID NO: 464 - DNA, 25 bp, synthetic construct
  source 1..25: mol_type = other DNA
gccgtttcgcgnnkatgacacggaa

SEQ ID NO: 465 - DNA, 26 bp, synthetic construct
  source 1..26: mol_type = other DNA
gtttcgcgtggnnkacacggaagtcg

SEQ ID NO: 466 - DNA, 25 bp, synthetic construct
  source 1..25: mol_type = other DNA
tcgcgtggatgnnkcggaagtcgga

SEQ ID NO: 467 - DNA, 29 bp, synthetic construct
  source 1..29: mol_type = other DNA
cgcgtggatgacannkaagtcggaagaga

SEQ ID NO: 468 - DNA, 27 bp, synthetic construct
  source 1..27: mol_type = other DNA
tggatgacacggnnktcggaagagacg

SEQ ID NO: 469 - DNA, 31 bp, synthetic construct
  source 1..31: mol_type = other DNA
ggatgacacggaagnnkgaagagacgatcac

SEQ ID NO: 470 - DNA, 27 bp, synthetic construct
  source 1..27: mol_type = other DNA
acacggaagtcgnnkgagacgatcacc

SEQ ID NO: 471 - DNA, 26 bp, synthetic construct
  source 1..26: mol_type = other DNA
ggaagtcggaannkacgatcaccccg

SEQ ID NO: 472 - DNA, 28 bp, synthetic construct
  source 1..28: mol_type = other DNA
aagtcggaagagnnkatcaccccgtgga

SEQ ID NO: 473 - DNA, 25 bp, synthetic construct
  source 1..25: mol_type = other DNA
cggaagagacgnnkaccccgtggaa

SEQ ID NO: 474 - DNA, 29 bp, synthetic construct
  source 1..29: mol_type = other DNA
ggaagagacgatcnnkccgtggaacttcg

SEQ ID NO: 475 - DNA, 33 bp, synthetic construct
  source 1..33: mol_type = other DNA
gaagagacgatcaccnnktggaacttcgaagag

SEQ ID NO: 476 - DNA, 29 bp, synthetic construct
  source 1..29: mol_type = other DNA
gacgatcaccccgnnkaacttcgaagagg

SEQ ID NO: 477 - DNA, 29 bp, synthetic construct
  source 1..29: mol_type = other DNA
gatcaccccgtggnnkttcgaagaggtag

SEQ ID NO: 478 - DNA, 27 bp, synthetic construct
  source 1..27: mol_type = other DNA
accccgtggaacnnkgaagaggtagtc

SEQ ID NO: 479 - DNA, 28 bp, synthetic construct
  source 1..28: mol_type = other DNA
ccgtggaacttcnnkgaggtagtcgaca

SEQ ID NO: 480 - DNA, 31 bp, synthetic construct
  source 1..31: mol_type = other DNA
cgtggaacttcgaannkgtagtcgacaaggg

SEQ ID NO: 481 - DNA, 28 bp, synthetic construct
  source 1..28: mol_type = other DNA
aacttcgaagagnnkgtcgacaagggcg

SEQ ID NO: 482 - DNA, 29 bp, synthetic construct
  source 1..29: mol_type = other DNA
cttcgaagaggtannkgacaagggcgcat

SEQ ID NO: 483 - DNA, 28 bp, synthetic construct
  source 1..28: mol_type = other DNA
gaagaggtagtcnnkaagggcgcatcag

SEQ ID NO: 484 - DNA, 25 bp, synthetic construct
  source 1..25: mol_type = other DNA
aggtagtcgacnnkggcgcatcagc

SEQ ID NO: 485 - DNA, 27 bp, synthetic construct
  source 1..27: mol_type = other DNA
gtagtcgacaagnnkgcatcagcgcag

SEQ ID NO: 486 - DNA, 25 bp, synthetic construct
  source 1..25: mol_type = other DNA
tcgacaagggcnnktcagcgcagtc

SEQ ID NO: 487 - DNA, 25 bp, synthetic construct
  source 1..25: mol_type = other DNA
acaagggcgcannkgcgcagtcttt

SEQ ID NO: 488 - DNA, 34 bp, synthetic construct
  source 1..34: mol_type = other DNA
gacaagggcgcatcannkcagtcttttattgaac

SEQ ID NO: 489 - DNA, 29 bp, synthetic construct
  source 1..29: mol_type = other DNA
gggcgcatcagcgnnktcttttattgaac

SEQ ID NO: 490 - DNA, 31 bp, synthetic construct
  source 1..31: mol_type = other DNA
gcgcatcagcgcagnnktttattgaacgtat

SEQ ID NO: 491 - DNA, 32 bp, synthetic construct
  source 1..32: mol_type = other DNA
catcagcgcagtctnnkattgaacgtatgacg

SEQ ID NO: 492 - DNA, 32 bp, synthetic construct
  source 1..32: mol_type = other DNA
cagcgcagtcttttnnkgaacgtatgacgaat

SEQ ID NO: 493 - DNA, 36 bp, synthetic construct
  source 1..36: mol_type = other DNA
agcgcagtcttttattnnkcgtatgacgaatttcga

SEQ ID NO: 494 - DNA, 41 bp, synthetic construct
  source 1..41: mol_type = other DNA
agcgcagtcttttattgaannkatgacgaatttcgataaaa

SEQ ID NO: 495 - DNA, 43 bp, synthetic construct
  source 1..43: mol_type = other DNA
cgcagtcttttattgaacgtnnkacgaatttcgataaaaactt

SEQ ID NO: 496 - DNA, 44 bp, synthetic construct
  source 1..44: mol_type = other DNA
agtcttttattgaacgtatgnnkaatttcgataaaaacttgccc

SEQ ID NO: 497 - DNA, 38 bp, synthetic construct
  source 1..38: mol_type = other DNA
ttattgaacgtatgacgnnkttcgataaaaacttgccc

SEQ ID NO: 498 - DNA, 40 bp, synthetic construct
  source 1..40: mol_type = other DNA
attgaacgtatgacgaatnnkgataaaaacttgcccaatg

SEQ ID NO: 499 - DNA, 37 bp, synthetic construct
  source 1..37: mol_type = other DNA
aacgtatgacgaatttcnnkaaaaacttgcccaatga

SEQ ID NO: 500 - DNA, 38 bp, synthetic construct
  source 1..38: mol_type = other DNA
gtatgacgaatttcgatnnkaacttgcccaatgagaag

SEQ ID NO: 501 - DNA, 37 bp, synthetic construct
  source 1..37: mol_type = other DNA
tgacgaatttcgataaannkttgcccaatgagaaggt

SEQ ID NO: 502 - DNA, 36 bp, synthetic construct
  source 1..36: mol_type = other DNA
gaatttcgataaaaacnnkcccaatgagaaggtgct

SEQ ID NO: 503 - DNA, 38 bp, synthetic construct
  source 1..38: mol_type = other DNA
atttcgataaaaacttgnnkaatgagaaggtgcttccg

SEQ ID NO: 504 - DNA, 31 bp, synthetic construct
  source 1..31: mol_type = other DNA
ataaaaacttgcccnnkgagaaggtgcttcc

SEQ ID NO: 505 - DNA, 35 bp, synthetic construct
  source 1..35: mol_type = other DNA
taaaaacttgcccaatnnkaaggtgcttccgaaac

SEQ ID NO: 506 - DNA, 29 bp, synthetic construct
  source 1..29: mol_type = other DNA
cttgcccaatgagnnkgtgcttccgaaac

SEQ ID NO: 507 - DNA, 31 bp, synthetic construct
  source 1..31: mol_type = other DNA
tgcccaatgagaagnnkcttccgaaacattc

SEQ ID NO: 508 - DNA, 32 bp, synthetic construct
  source 1..32: mol_type = other DNA
ccaatgagaaggtgnnkccgaaacattccttg

SEQ ID NO: 509 - DNA, 41 bp, synthetic construct
  source 1..41: mol_type = other DNA
gcccaatgagaaggtgcttnnkaaacattccttgttatatg

SEQ ID NO: 510 - DNA, 35 bp, synthetic construct
  source 1..35: mol_type = other DNA
tgagaaggtgcttccgnnkcattccttgttatatg

SEQ ID NO: 511 - DNA, 43 bp, synthetic construct
  source 1..43: mol_type = other DNA
atgagaaggtgcttccgaaannktccttgttatatgaatattt

SEQ ID NO: 512 - DNA, 46 bp, synthetic construct
  source 1..46: mol_type = other DNA
gagaaggtgcttccgaaacatnnkttgttatatgaatattttacag

SEQ ID NO: 513 - DNA, 44 bp, synthetic construct
  source 1..44: mol_type = other DNA
aggtgcttccgaaacattccnnkttatatgaatattttacagtt

SEQ ID NO: 514 - DNA, 45 bp, synthetic construct
  source 1..45: mol_type = other DNA
gtgcttccgaaacattccttgnnktatgaatattttacagtttac

SEQ ID NO: 515 - DNA, 46 bp, synthetic construct
  source 1..46: mol_type = other DNA
cttccgaaacattccttgttannkgaatattttacagtttacaacg

SEQ ID NO: 516 - DNA, 46 bp, synthetic construct
  source 1..46: mol_type = other DNA
ccgaaacattccttgttatatnnktattttacagtttacaacgagc

SEQ ID NO: 517 - DNA, 46 bp, synthetic construct
  source 1..46: mol_type = other DNA
aaacattccttgttatatgaannktttacagtttacaacgagctga

SEQ ID NO: 518 - DNA, 44 bp, synthetic construct
  source 1..44: mol_type = other DNA
attccttgttatatgaatatnnkacagtttacaacgagctgacc

SEQ ID NO: 519 - DNA, 44 bp, synthetic construct
  source 1..44: mol_type = other DNA
ccttgttatatgaatattttnnkgtttacaacgagctgaccaag

SEQ ID NO: 520 - DNA, 45 bp, synthetic construct
  source 1..45: mol_type = other DNA
ttgttatatgaatattttacannktacaacgagctgaccaaggtt

SEQ ID NO: 521 - DNA, 50 bp, synthetic construct
  source 1..50: mol_type = other DNA
tgttatatgaatattttacagttnnkaacgagctgaccaaggttaaatac

SEQ ID NO: 522 - DNA, 46 bp, synthetic construct
  source 1..46: mol_type = other DNA
tatgaatattttacagtttacnnkgagctgaccaaggttaaatacg

SEQ ID NO: 523 - DNA, 46 bp, synthetic construct
  source 1..46: mol_type = other DNA
gaatattttacagtttacaacnnkctgaccaaggttaaatacgtga

SEQ ID NO: 524 - DNA, 40 bp, synthetic construct
  source 1..40: mol_type = other DNA
tttacagtttacaacgagnnkaccaaggttaaatacgtga

SEQ ID NO: 525 - DNA, 37 bp, synthetic construct
  source 1..37: mol_type = other DNA
cagtttacaacgagctgnnkaaggttaaatacgtgac

SEQ ID NO: 526 - DNA, 33 bp, synthetic construct
  source 1..33: mol_type = other DNA
tacaacgagctgaccnnkgttaaatacgtgacg

SEQ ID NO: 527 - DNA, 30 bp, synthetic construct
  source 1..30: mol_type = other DNA
cgagctgaccaagnnkaaatacgtgacgga

SEQ ID NO: 528 - DNA, 29 bp, synthetic construct
  source 1..29: mol_type = other DNA
gctgaccaaggttnnktacgtgacggaag

SEQ ID NO: 529 - DNA, 33 bp, synthetic construct
  source 1..33: mol_type = other DNA
ctgaccaaggttaaannkgtgacggaaggaatg

SEQ ID NO: 530 - DNA, 32 bp, synthetic construct
  source 1..32: mol_type = other DNA
ccaaggttaaatacnnkacggaaggaatgcgc

SEQ ID NO: 531 - DNA, 33 bp, synthetic construct
  source 1..33: mol_type = other DNA
aaggttaaatacgtgnnkgaaggaatgcgcaag

SEQ ID NO: 532 - DNA, 29 bp, synthetic construct
  source 1..29: mol_type = other DNA
taaatacgtgacgnnkggaatgcgcaagc

SEQ ID NO: 533 - DNA, 25 bp, synthetic construct
  source 1..25: mol_type = other DNA
acgtgacggaannkatgcgcaagcc

SEQ ID NO: 534 - DNA, 25 bp, synthetic construct
  source 1..25: mol_type = other DNA
tgacggaaggannkcgcaagcccgc

SEQ ID NO: 535 - DNA, 29 bp, synthetic construct
  source 1..29: mol_type = other DNA
gacggaaggaatgnnkaagcccgcttttc

SEQ ID NO: 536 - DNA, 28 bp, synthetic construct
  source 1..28: mol_type = other DNA
gaaggaatgcgcnnkcccgcttttctta

SEQ ID NO: 537 - DNA, 29 bp, synthetic construct
  source 1..29: mol_type = other DNA
aggaatgcgcaagnnkgcttttcttagcg

SEQ ID NO: 538 - DNA, 25 bp, synthetic construct
  source 1..25: mol_type = other DNA
tgcgcaagcccnnktttcttagcgg

SEQ ID NO: 539 - DNA, 25 bp, synthetic construct
  source 1..25: mol_type = other DNA
gcaagcccgctnnkcttagcggtga

SEQ ID NO: 540 - DNA, 25 bp, synthetic construct
  source 1..25: mol_type = other DNA
agcccgcttttnnkagcggtgagca

SEQ ID NO: 541 - DNA, 31 bp, synthetic construct
  source 1..31: mol_type = other DNA
agcccgcttttcttnnkggtgagcaaaaaaa

SEQ ID NO: 542 - DNA, 31 bp, synthetic construct
  source 1..31: mol_type = other DNA
ccgcttttcttagcnnkgagcaaaaaaaggc

SEQ ID NO: 543 - DNA, 33 bp, synthetic construct
  source 1..33: mol_type = other DNA
gcttttcttagcggtnnkcaaaaaaaggcgatc

SEQ ID NO: 544 - DNA, 31 bp, synthetic construct
  source 1..31: mol_type = other DNA
ttcttagcggtgagnnkaaaaaggcgatcgt

SEQ ID NO: 545 - DNA, 25 bp, synthetic construct
  source 1..25: mol_type = other DNA
gcggtgagcaannkaaggcgatcgt

SEQ ID NO: 546 - DNA, 28 bp, synthetic construct
  source 1..28: mol_type = other DNA
ggtgagcaaaaannkgcgatcgtcgacc

SEQ ID NO: 547 - DNA, 34 bp, synthetic construct
  source 1..34: mol_type = other DNA
ggtgagcaaaaaaagnnkatcgtcgacctgttat

SEQ ID NO: 548 - DNA, 33 bp, synthetic construct
  source 1..33: mol_type = other DNA
gagcaaaaaaaggcgnnkgtcgacctgttattc

SEQ ID NO: 549 - DNA, 37 bp, synthetic construct
  source 1..37: mol_type = other DNA
agcaaaaaaaggcgatcnnkgacctgttattcaaaac

SEQ ID NO: 550 - DNA, 37 bp, synthetic construct
  source 1..37: mol_type = other DNA
aaaaaaaggcgatcgtcnnkctgttattcaaaacgaa

SEQ ID NO: 551 - DNA, 35 bp, synthetic construct
  source 1..35: mol_type = other DNA
aaaggcgatcgtcgacnnkttattcaaaacgaatc

SEQ ID NO: 552 - DNA, 31 bp, synthetic construct
  source 1..31: mol_type = other DNA
cgatcgtcgacctgnnkttcaaaacgaatcg

SEQ ID NO: 553 - DNA, 36 bp, synthetic construct
  source 1..36: mol_type = other DNA
gatcgtcgacctgttannkaaaacgaatcgtaaggt

SEQ ID NO: 554 - DNA, 34 bp, synthetic construct
  source 1..34: mol_type = other DNA
gtcgacctgttattcnnkacgaatcgtaaggtga

SEQ ID NO: 555 - DNA, 37 bp, synthetic construct
  source 1..37: mol_type = other DNA
tcgacctgttattcaaannkaatcgtaaggtgactgt

SEQ ID NO: 556 - DNA, 37 bp, synthetic construct
  source 1..37: mol_type = other DNA
acctgttattcaaaacgnnkcgtaaggtgactgtaaa

SEQ ID NO: 557 - DNA, 41 bp, synthetic construct
  source 1..41: mol_type = other DNA
cctgttattcaaaacgaatnnkaaggtgactgtaaagcaac

SEQ ID NO: 558 - DNA, 38 bp, synthetic construct
  source 1..38: mol_type = other DNA
tattcaaaacgaatcgtnnkgtgactgtaaagcaactc

SEQ ID NO: 559 - DNA, 40 bp, synthetic construct
  source 1..40: mol_type = other DNA
ttcaaaacgaatcgtaagnnkactgtaaagcaactcaaag

SEQ ID NO: 560 - DNA, 37 bp, synthetic construct
  source 1..37: mol_type = other DNA
aaacgaatcgtaaggtgnnkgtaaagcaactcaaaga

SEQ ID NO: 561 - DNA, 37 bp, synthetic construct
  source 1..37: mol_type = other DNA
cgaatcgtaaggtgactnnkaagcaactcaaagaaga

SEQ ID NO: 562 - DNA, 42 bp, synthetic construct
  source 1..42: mol_type = other DNA
gaatcgtaaggtgactgtannkcaactcaaagaagattactt

SEQ ID NO: 563 - DNA, 40 bp, synthetic construct
  source 1..40: mol_type = other DNA
cgtaaggtgactgtaaagnnkctcaaagaagattacttca

SEQ ID NO: 564 - DNA, 45 bp, synthetic construct
  source 1..45: mol_type = other DNA
cgtaaggtgactgtaaagcaannkaaagaagattacttcaaaaag

SEQ ID NO: 565 - DNA, 41 bp, synthetic construct
  source 1..41: mol_type = other DNA
ggtgactgtaaagcaactcnnkgaagattacttcaaaaaga

SEQ ID NO: 566 - DNA, 45 bp, synthetic construct
  source 1..45: mol_type = other DNA
gtgactgtaaagcaactcaaannkgattacttcaaaaagattgag

SEQ ID NO: 567 - DNA, 44 bp, synthetic construct
  source 1..44: mol_type = other DNA
ctgtaaagcaactcaaagaannktacttcaaaaagattgagtgc

SEQ ID NO: 568 - DNA, 40 bp, synthetic construct
  source 1..40: mol_type = other DNA
aagcaactcaaagaagatnnkttcaaaaagattgagtgct

SEQ ID NO: 569 - DNA, 41 bp, synthetic construct
  source 1..41: mol_type = other DNA
gcaactcaaagaagattacnnkaaaaagattgagtgcttcg

SEQ ID NO: 570 - DNA, 40 bp, synthetic construct
  source 1..40: mol_type = other DNA
ctcaaagaagattacttcnnkaagattgagtgcttcgaca

SEQ ID NO: 571 - DNA, 38 bp, synthetic construct
  source 1..38: mol_type = other DNA
aagaagattacttcaaannkattgagtgcttcgacagc

SEQ ID NO: 572 - DNA, 34 bp, synthetic construct
  source 1..34: mol_type = other DNA
gattacttcaaaaagnnkgagtgcttcgacagcg

SEQ ID NO: 573 - DNA, 35 bp, synthetic construct
  source 1..35: mol_type = other DNA
ttacttcaaaaagattnnktgcttcgacagcgtcg

SEQ ID NO: 574 - DNA, 36 bp, synthetic construct
  source 1..36: mol_type = other DNA
cttcaaaaagattgagnnkttcgacagcgtcgaaat

SEQ ID NO: 575 - DNA, 33 bp, synthetic construct
  source 1..33: mol_type = other DNA
aaaaagattgagtgcnnkgacagcgtcgaaatc

SEQ ID NO: 576 - DNA, 33 bp, synthetic construct
  source 1..33: mol_type = other DNA
aagattgagtgcttcnnkagcgtcgaaatctct

SEQ ID NO: 577 - DNA, 31 bp, synthetic construct
  source 1..31: mol_type = other DNA
ttgagtgcttcgacnnkgtcgaaatctctgg

SEQ ID NO: 578 - DNA, 27 bp, synthetic construct
  source 1..27: mol_type = other DNA
tgcttcgacagcnnkgaaatctctggg

SEQ ID NO: 579 - DNA, 29 bp, synthetic construct
  source 1..29: mol_type = other DNA
cttcgacagcgtcnnkatctctggggtag

SEQ ID NO: 580 - DNA, 28 bp, synthetic construct
  source 1..28: mol_type = other DNA
gacagcgtcgaannktctggggtagagg

SEQ ID NO: 581 - DNA, 29 bp, synthetic construct
  source 1..29: mol_type = other DNA
cagcgtcgaaatcnnkggggtagaggatc

SEQ ID NO: 582 - DNA, 34 bp, synthetic construct
  source 1..34: mol_type = other DNA
agcgtcgaaatctctnnkgtagaggatcggttta

SEQ ID NO: 583 - DNA, 33 bp, synthetic construct
  source 1..33: mol_type = other DNA
gtcgaaatctctgggnnkgaggatcggtttaac

SEQ ID NO: 584 - DNA, 34 bp, synthetic construct
  source 1..34: mol_type = other DNA
gaaatctctggggtannkgatcggtttaacgcaa

SEQ ID NO: 585 - DNA, 29 bp, synthetic construct
  source 1..29: mol_type = other DNA
ctctggggtagagnnkcggtttaacgcaa

SEQ ID NO: 586 - DNA, 36 bp, synthetic construct
  source 1..36: mol_type = other DNA
ctctggggtagaggatnnktttaacgcaagtttagg

SEQ ID NO: 587 - DNA, 31 bp, synthetic construct
  source 1..31: mol_type = other DNA
gggtagaggatcggnnkaacgcaagtttagg

SEQ ID NO: 588 - DNA, 36 bp, synthetic construct
  source 1..36: mol_type = other DNA
ggtagaggatcggtttnnkgcaagtttaggtaccta

SEQ ID NO: 589 - DNA, 39 bp, synthetic construct
  source 1..39: mol_type = other DNA
gtagaggatcggtttaacnnkagtttaggtacctaccat

SEQ ID NO: 590 - DNA, 35 bp, synthetic construct
  source 1..35: mol_type = other DNA
ggatcggtttaacgcannkttaggtacctaccatg

SEQ ID NO: 591 - DNA, 32 bp, synthetic construct
  source 1..32: mol_type = other DNA
ggtttaacgcaagtnnkggtacctaccatgac

SEQ ID NO: 592 - DNA, 35 bp, synthetic construct
  source 1..35: mol_type = other DNA
gtttaacgcaagtttannkacctaccatgacctgc

SEQ ID NO: 593 - DNA, 37 bp, synthetic construct
  source 1..37: mol_type = other DNA
ttaacgcaagtttaggtnnktaccatgacctgcttaa

SEQ ID NO: 594 - DNA, 36 bp, synthetic construct
  source 1..36: mol_type = other DNA
cgcaagtttaggtaccnnkcatgacctgcttaaaat

SEQ ID NO: 595 - DNA, 39 bp, synthetic construct
  source 1..39: mol_type = other DNA
gcaagtttaggtacctacnnkgacctgcttaaaatcatt

SEQ ID NO: 596 - DNA, 45 bp, synthetic construct
  source 1..45: mol_type = other DNA
gcaagtttaggtacctaccatnnkctgcttaaaatcattaaggat

SEQ ID NO: 597 - DNA, 48 bp, synthetic construct
  source 1..48: mol_type = other DNA
aagtttaggtacctaccatgacnnkcttaaaatcattaaggataaaga

SEQ ID NO: 598 - DNA, 42 bp, synthetic construct
  source 1..42: mol_type = other DNA
aggtacctaccatgacctgnnkaaaatcattaaggataaaga

SEQ ID NO: 599 - DNA, 40 bp, synthetic construct
  source 1..40: mol_type = other DNA
acctaccatgacctgcttnnkatcattaaggataaagact

SEQ ID NO: 600 - DNA, 45 bp, synthetic construct
  source 1..45: mol_type = other DNA
acctaccatgacctgcttaaannkattaaggataaagacttctta

SEQ ID NO: 601 - DNA, 43 bp, synthetic construct
  source 1..43: mol_type = other DNA
accatgacctgcttaaaatcnnkaaggataaagacttcttaga

SEQ ID NO: 602 - DNA, 48 bp, synthetic construct
  source 1..48: mol_type = other DNA
ccatgacctgcttaaaatcattnnkgataaagacttcttagataatga

SEQ ID NO: 603 - DNA, 51 bp, synthetic construct
  source 1..51: mol_type = other DNA
catgacctgcttaaaatcattaagnnkaaagacttcttagataatgaagag

SEQ ID NO: 604 - DNA, 49 bp, synthetic construct
  source 1..49: mol_type = other DNA
acctgcttaaaatcattaaggatnnkgacttcttagataatgaagagaa

SEQ ID NO: 605 - DNA, 51 bp, synthetic construct
  source 1..51: mol_type = other DNA
ctgcttaaaatcattaaggataaannkttcttagataatgaagagaacgaa

SEQ ID NO: 606 - DNA, 51 bp, synthetic construct
  source 1..51: mol_type = other DNA
cttaaaatcattaaggataaagacnnkttagataatgaagagaacgaagat

SEQ ID NO: 607 - DNA, 51 bp, synthetic construct
  source 1..51: mol_type = other DNA
aaaatcattaaggataaagacttcnnkgataatgaagagaacgaagatatt

SEQ ID NO: 608 - DNA, 51 bp, synthetic construct
  source 1..51: mol_type = other DNA
atcattaaggataaagacttcttannkaatgaagagaacgaagatattctc

SEQ ID NO: 609 - DNA, 46 bp, synthetic construct
  source 1..46: mol_type = other DNA
aaggataaagacttcttagatnnkgaagagaacgaagatattctcg

SEQ ID NO: 610 - DNA, 47 bp, synthetic construct
  source 1..47: mol_type = other DNA
ggataaagacttcttagataatnnkgagaacgaagatattctcgagg

SEQ ID NO: 611 - DNA, 46 bp, synthetic construct
  source 1..46: mol_type = other DNA
aaagacttcttagataatgaannkaacgaagatattctcgaggaca

SEQ ID NO: 612 - DNA, 45 bp, synthetic construct
  source 1..45: mol_type = other DNA
gacttcttagataatgaagagnnkgaagatattctcgaggacatc

SEQ ID NO: 613 - DNA, 42 bp, synthetic construct
  source 1..42: mol_type = other DNA
cttagataatgaagagaacnnkgatattctcgaggacatcgt

SEQ ID NO: 614 - DNA, 38 bp, synthetic construct
  source 1..38: mol_type = other DNA
ataatgaagagaacgaannkattctcgaggacatcgtc

SEQ ID NO: 615 - DNA, 34 bp, synthetic construct
  source 1..34: mol_type = other DNA
gaagagaacgaagatnnkctcgaggacatcgtct

SEQ ID NO: 616 - DNA, 38 bp, synthetic construct
  source 1..38: mol_type = other DNA
aagagaacgaagatattnnkgaggacatcgtcttgacg

SEQ ID NO: 617 - DNA, 36 bp, synthetic construct
  source 1..36: mol_type = other DNA
gaacgaagatattctcnnkgacatcgtcttgacgtt

SEQ ID NO: 618 - DNA, 37 bp, synthetic construct
  source 1..37: mol_type = other DNA
acgaagatattctcgagnnkatcgtcttgacgttaac

SEQ ID NO: 619 - DNA, 37 bp, synthetic construct
  source 1..37: mol_type = other DNA
aagatattctcgaggacnnkgtcttgacgttaacctt

SEQ ID NO: 620 - DNA, 42 bp, synthetic construct
  source 1..42: mol_type = other DNA
agatattctcgaggacatcnnkttgacgttaaccttatttga

SEQ ID NO: 621 - DNA, 36 bp, synthetic construct
  source 1..36: mol_type = other DNA
tctcgaggacatcgtcnnkacgttaaccttatttga

SEQ ID NO: 622 - DNA, 37 bp, synthetic construct
  source 1..37: mol_type = other DNA
tcgaggacatcgtcttgnnkttaaccttatttgagga

SEQ ID NO: 623 - DNA, 35 bp, synthetic construct
  source 1..35: mol_type = other DNA
ggacatcgtcttgacgnnkaccttatttgaggatc

SEQ ID NO: 624 - DNA, 38 bp, synthetic construct
  source 1..38: mol_type = other DNA
acatcgtcttgacgttannkttatttgaggatcgtgaa

SEQ ID NO: 625 - DNA, 36 bp, synthetic construct
  source 1..36: mol_type = other DNA
cgtcttgacgttaaccnnktttgaggatcgtgaaat

SEQ ID NO: 626 - DNA, 39 bp, synthetic construct
  source 1..39: mol_type = other DNA
gtcttgacgttaaccttannkgaggatcgtgaaatgatt

SEQ ID NO: 627 - DNA, 41 bp, synthetic construct
  source 1..41: mol_type = other DNA
cttgacgttaaccttatttnnkgatcgtgaaatgattgagg

SEQ ID NO: 628 - DNA, 39 bp, synthetic construct
  source 1..39: mol_type = other DNA
acgttaaccttatttgagnnkcgtgaaatgattgaggaa

SEQ ID NO: 629 - DNA, 40 bp, synthetic construct
  source 1..40: mol_type = other DNA
ttaaccttatttgaggatnnkgaaatgattgaggaacgcc

SEQ ID NO: 630 - DNA, 36 bp, synthetic construct
  source 1..36: mol_type = other DNA
cttatttgaggatcgtnnkatgattgaggaacgcct

SEQ ID NO: 631 - DNA, 32 bp, synthetic construct
  source 1..32: mol_type = other DNA
ttgaggatcgtgaannkattgaggaacgcctc

SEQ ID NO: 632 - DNA, 30 bp, synthetic construct
  source 1..30: mol_type = other DNA
ggatcgtgaaatgnnkgaggaacgcctcaa

SEQ ID NO: 633 - DNA, 37 bp, synthetic construct
  source 1..37: mol_type = other DNA
aggatcgtgaaatgattnnkgaacgcctcaaaactta

SEQ ID NO: 634 - DNA, 35 bp, synthetic construct
  source 1..35: mol_type = other DNA
tcgtgaaatgattgagnnkcgcctcaaaacttatg

SEQ ID NO: 635 - DNA, 38 bp, synthetic construct
  source 1..38: mol_type = other DNA
gtgaaatgattgaggaannkctcaaaacttatgcccac

SEQ ID NO: 636 - DNA, 33 bp, synthetic construct
  source 1..33: mol_type = other DNA
atgattgaggaacgcnnkaaaacttatgcccac

SEQ ID NO: 637 - DNA, 27 bp, synthetic construct
  source 1..27: mol_type = other DNA
gaggaacgcctcnnkacttatgcccac

SEQ ID NO: 638 - DNA, 28 bp, synthetic construct
  source 1..28: mol_type = other DNA
gaacgcctcaaannktatgcccacctgt

SEQ ID NO: 639 - DNA, 27 bp, synthetic construct
  source 1..27: mol_type = other DNA
cgcctcaaaactnnkgcccacctgttc

SEQ ID NO: 640 - DNA, 34 bp, synthetic construct
  source 1..34: mol_type = other DNA
cgcctcaaaacttatnnkcacctgttcgacgata

SEQ ID NO: 641 - DNA, 34 bp, synthetic construct
  source 1..34: mol_type = other DNA
ctcaaaacttatgccnnkctgttcgacgataagg

SEQ ID NO: 642 - DNA, 36 bp, synthetic construct
  source 1..36: mol_type = other DNA
caaaacttatgcccacnnkttcgacgataaggtgat

SEQ ID NO: 643 - DNA, 32 bp, synthetic construct
  source 1..32: mol_type = other DNA
cttatgcccacctgnnkgacgataaggtgatg

SEQ ID NO: 644 - DNA, 33 bp, synthetic construct
  source 1..33: mol_type = other DNA
tatgcccacctgttcnnkgataaggtgatgaag

SEQ ID NO: 645 - DNA, 29 bp, synthetic construct
  source 1..29: mol_type = other DNA
ccacctgttcgacnnkaaggtgatgaagc

SEQ ID NO: 646 - DNA, 29 bp, synthetic construct
  source 1..29: mol_type = other DNA
cctgttcgacgatnnkgtgatgaagcagc

SEQ ID NO: 647 - DNA, 34 bp, synthetic construct
  source 1..34: mol_type = other DNA
ctgttcgacgataagnnkatgaagcagctgaaac

SEQ ID NO: 648 - DNA, 30 bp, synthetic construct
  source 1..30: mol_type = other DNA
cgacgataaggtgnnkaagcagctgaaacg

SEQ ID NO: 649 - DNA, 30 bp, synthetic construct
  source 1..30: mol_type = other DNA
cgataaggtgatgnnkcagctgaaacgtcg

SEQ ID NO: 650 - DNA, 29 bp, synthetic construct
  source 1..29: mol_type = other DNA
taaggtgatgaagnnkctgaaacgtcggc

SEQ ID NO: 651 - DNA, 26 bp, synthetic construct
  source 1..26: mol_type = other DNA
tgatgaagcagnnkaaacgtcggcgc

SEQ ID NO: 652 - DNA, 25 bp, synthetic construct
  source 1..25: mol_type = other DNA
tgaagcagctgnnkcgtcggcgcta

SEQ ID NO: 653 - DNA, 25 bp, synthetic construct
  source 1..25: mol_type = other DNA
agcagctgaaannkcggcgctacac

SEQ ID NO: 654 - DNA, 28 bp, synthetic construct
  source 1..28: mol_type = other DNA
cagctgaaacgtnnkcgctacacaggat

SEQ ID NO: 655 - DNA, 28 bp, synthetic construct
  source 1..28: mol_type = other DNA
ctgaaacgtcggnnktacacaggatggg

SEQ ID NO: 656 - DNA, 25 bp, synthetic construct
  source 1..25: mol_type = other DNA
aacgtcggcgcnnkacaggatgggg

SEQ ID NO: 657 - DNA, 25 bp, synthetic construct
  source 1..25: mol_type = other DNA
gtcggcgctacnnkggatggggccg

SEQ ID NO: 658 - DNA, 25 bp, synthetic construct
  source 1..25: mol_type = other DNA
ggcgctacacannktggggccgctt

SEQ ID NO: 659 - DNA, 26 bp, synthetic construct
  source 1..26: mol_type = other DNA
gctacacaggannkggccgcttgagt

SEQ ID NO: 660 - DNA, 25 bp, synthetic construct
  source 1..25: mol_type = other DNA
acacaggatggnnkcgcttgagtcg

SEQ ID NO: 661 - DNA, 27 bp, synthetic construct
  source 1..27: mol_type = other DNA
acaggatggggcnnkttgagtcgcaaa

SEQ ID NO: 662 - DNA, 25 bp, synthetic construct
  source 1..25: mol_type = other DNA
gatggggccgcnnkagtcgcaaact

SEQ ID NO: 663 - DNA, 25 bp, synthetic construct
  source 1..25: mol_type = other DNA
ggggccgcttgnnkcgcaaacttat

SEQ ID NO: 664 - DNA, 32 bp, synthetic construct
  source 1..32: mol_type = other DNA
ggggccgcttgagtnnkaaacttattaacgga

SEQ ID NO: 665 - DNA, 30 bp, synthetic construct
  source 1..30: mol_type = other DNA
ccgcttgagtcgcnnkcttattaacggaat

SEQ ID NO: 666 - DNA, 32 bp, synthetic construct
  source 1..32: mol_type = other DNA
gcttgagtcgcaaannkattaacggaatccgt

SEQ ID NO: 667 - DNA, 31 bp, synthetic construct
  source 1..31: mol_type = other DNA
tgagtcgcaaacttnnkaacggaatccgtga

SEQ ID NO: 668 - DNA, 33 bp, synthetic construct
  source 1..33: mol_type = other DNA
agtcgcaaacttattnnkggaatccgtgacaag

SEQ ID NO: 669 - DNA, 37 bp, synthetic construct
  source 1..37: mol_type = other DNA
gtcgcaaacttattaacnnkatccgtgacaagcaatc

SEQ ID NO: 670 - DNA, 35 bp, synthetic construct
  source 1..35: mol_type = other DNA
caaacttattaacggannkcgtgacaagcaatccg

SEQ ID NO: 671 - DNA, 33 bp, synthetic construct
  source 1..33: mol_type = other DNA
cttattaacggaatcnnkgacaagcaatccggc

SEQ ID NO: 672 - DNA, 35 bp, synthetic construct
  source 1..35: mol_type = other DNA
tattaacggaatccgtnnkaagcaatccggcaaaa

SEQ ID NO: 673 - DNA, 28 bp, synthetic construct
  source 1..28: mol_type = other DNA
ggaatccgtgacnnkcaatccggcaaaa

SEQ ID NO: 674 - DNA, 27 bp, synthetic construct
  source 1..27: mol_type = other DNA
atccgtgacaagnnktccggcaaaacg

SEQ ID NO: 675 - DNA, 30 bp, synthetic construct
  source 1..30: mol_type = other DNA
ccgtgacaagcaannkggcaaaacgattct

SEQ ID NO: 676 - DNA, 34 bp, synthetic construct
  source 1..34: mol_type = other DNA
cgtgacaagcaatccnnkaaaacgattctggatt

SEQ ID NO: 677 - DNA, 30 bp, synthetic construct
  source 1..30: mol_type = other DNA
caagcaatccggcnnkacgattctggattt

SEQ ID NO: 678 - DNA, 36 bp, synthetic construct
  source 1..36: mol_type = other DNA
caagcaatccggcaaannkattctggatttcttgaa

SEQ ID NO: 679 - DNA, 32 bp, synthetic construct
  source 1..32: mol_type = other DNA
aatccggcaaaacgnnkctggatttcttgaag

SEQ ID NO: 680 - DNA, 33 bp, synthetic construct
  source 1..33: mol_type = other DNA
tccggcaaaacgattnnkgatttcttgaagtcg

SEQ ID NO: 681 - DNA, 33 bp, synthetic construct
  source 1..33: mol_type = other DNA
ggcaaaacgattctgnnkttcttgaagtcggac

SEQ ID NO: 682 - DNA, 34 bp, synthetic construct
  source 1..34: mol_type = other DNA
aaaacgattctggatnnkttgaagtcggacggat

SEQ ID NO: 683 - DNA, 33 bp, synthetic construct
  source 1..33: mol_type = other DNA
acgattctggatttcnnkaagtcggacggattt

SEQ ID NO: 684 - DNA, 36 bp, synthetic construct
  source 1..36: mol_type = other DNA
gattctggatttcttgnnktcggacggatttgctaa

SEQ ID NO: 685 - DNA, 37 bp, synthetic construct
  source 1..37: mol_type = other DNA
ttctggatttcttgaagnnkgacggatttgctaatcg

SEQ ID NO: 686 - DNA, 34 bp, synthetic construct
  source 1..34: mol_type = other DNA
gatttcttgaagtcgnnkggatttgctaatcgca

SEQ ID NO: 687 - DNA, 35 bp, synthetic construct
  source 1..35: mol_type = other DNA
tttcttgaagtcggacnnktttgctaatcgcaact

SEQ ID NO: 688 - DNA, 30 bp, synthetic construct
  source 1..30: mol_type = other DNA
gaagtcggacggannkgctaatcgcaactt

SEQ ID NO: 689 - DNA, 33 bp, synthetic construct
  source 1..33: mol_type = other DNA
aagtcggacggatttnnkaatcgcaacttcatg

SEQ ID NO: 690 - DNA, 28 bp, synthetic construct
  source 1..28: mol_type = other DNA
gacggatttgctnnkcgcaacttcatgc

SEQ ID NO: 691 - DNA, 36 bp, synthetic construct
  source 1..36: mol_type = other DNA
ggacggatttgctaatnnkaacttcatgcagttgat

SEQ ID NO: 692 - DNA, 34 bp, synthetic construct
  source 1..34: mol_type = other DNA
ggatttgctaatcgcnnkttcatgcagttgatcc

SEQ ID NO: 693 - DNA, 35 bp, synthetic construct
  source 1..35: mol_type = other DNA
atttgctaatcgcaacnnkatgcagttgatccatg

SEQ ID NO: 694 - DNA, 34 bp, synthetic construct
  source 1..34: mol_type = other DNA
gctaatcgcaacttcnnkcagttgatccatgatg

SEQ ID NO: 695 - DNA, 38 bp, synthetic construct
  source 1..38: mol_type = other DNA
ctaatcgcaacttcatgnnkttgatccatgatgactcc

SEQ ID NO: 696 - DNA, 32 bp, synthetic construct
  source 1..32: mol_type = other DNA
gcaacttcatgcagnnkatccatgatgactcc

SEQ ID NO: 697 - DNA, 31 bp, synthetic construct
  source 1..31: mol_type = other DNA
acttcatgcagttgnnkcatgatgactccct

SEQ ID NO: 698 - DNA, 31 bp, synthetic construct
  source 1..31: mol_type = other DNA
tcatgcagttgatcnnkgatgactccctgac

SEQ ID NO: 699 - DNA, 36 bp, synthetic construct
  source 1..36: mol_type = other DNA
catgcagttgatccatnnkgactccctgacttttaa

SEQ ID NO: 700 - DNA, 37 bp, synthetic construct
  source 1..37: mol_type = other DNA
tgcagttgatccatgatnnktccctgacttttaaaga

SEQ ID NO: 701 - DNA, 39 bp, synthetic construct
  source 1..39: mol_type = other DNA
cagttgatccatgatgacnnkctgacttttaaagaggat

SEQ ID NO: 702 - DNA, 43 bp, synthetic construct
  source 1..43: mol_type = other DNA
agttgatccatgatgactccnnkacttttaaagaggatattca

SEQ ID NO: 703 - DNA, 43 bp, synthetic construct
  source 1..43: mol_type = other DNA
tgatccatgatgactccctgnnktttaaagaggatattcaaaa

SEQ ID NO: 704 - DNA, 40 bp, synthetic construct
  source 1..40: mol_type = other DNA
catgatgactccctgactnnkaaagaggatattcaaaagg

SEQ ID NO: 705 - DNA, 37 bp, synthetic construct
  source 1..37: mol_type = other DNA
atgactccctgacttttnnkgaggatattcaaaaggc

SEQ ID NO: 706 - DNA, 37 bp, synthetic construct
  source 1..37: mol_type = other DNA
actccctgacttttaaannkgatattcaaaaggcgca

SEQ ID NO: 707 - DNA, 35 bp, synthetic construct
  source 1..35: mol_type = other DNA
cctgacttttaaagagnnkattcaaaaggcgcagg

SEQ ID NO: 708 - DNA, 37 bp, synthetic construct
  source 1..37: mol_type = other DNA
tgacttttaaagaggatnnkcaaaaggcgcaggttag

SEQ ID NO: 709 - DNA, 38 bp, synthetic construct
  source 1..38: mol_type = other DNA
cttttaaagaggatattnnkaaggcgcaggttagtggt

SEQ ID NO: 710 - DNA, 36 bp, synthetic construct
  source 1..36: mol_type = other DNA
taaagaggatattcaannkgcgcaggttagtggtca

SEQ ID NO: 711 - DNA, 37 bp, synthetic construct
  source 1..37: mol_type = other DNA
aagaggatattcaaaagnnkcaggttagtggtcaagg

SEQ ID NO: 712 - DNA, 33 bp, synthetic construct
  source 1..33: mol_type = other DNA
gatattcaaaaggcgnnkgttagtggtcaaggc

SEQ ID NO: 713 - DNA, 27 bp, synthetic construct
  source 1..27: mol_type = other DNA
caaaaggcgcagnnkagtggtcaaggc

SEQ ID NO: 714 - DNA, 25 bp, synthetic construct
  source 1..25: mol_type = other DNA
aggcgcaggttnnkggtcaaggcga

SEQ ID NO: 715 - DNA, 26 bp, synthetic construct
  source 1..26: mol_type = other DNA
cgcaggttagtnnkcaaggcgacagc

SEQ ID NO: 716 - DNA, 28 bp, synthetic construct
  source 1..28: mol_type = other DNA
caggttagtggtnnkggcgacagcttac

SEQ ID NO: 717 - DNA, 34 bp, synthetic construct
  source 1..34: mol_type = other DNA
caggttagtggtcaannkgacagcttacacgaac

SEQ ID NO: 718 - DNA, 32 bp, synthetic construct
  source 1..32: mol_type = other DNA
ttagtggtcaaggcnnkagcttacacgaacac

SEQ ID NO: 719 - DNA, 30 bp, synthetic construct
  source 1..30: mol_type = other DNA
tggtcaaggcgacnnkttacacgaacacat

SEQ ID NO: 720 - DNA, 27 bp, synthetic construct
  source 1..27: mol_type = other DNA
caaggcgacagcnnkcacgaacacatc

SEQ ID NO: 721 - DNA, 29 bp, synthetic construct
  source 1..29: mol_type = other DNA
aggcgacagcttannkgaacacatcgcaa

SEQ ID NO: 722 - DNA, 31 bp, synthetic construct
  source 1..31: mol_type = other DNA
gcgacagcttacacnnkcacatcgcaaattt

SEQ ID NO: 723 - DNA, 33 bp, synthetic construct
  source 1..33: mol_type = other DNA
gacagcttacacgaannkatcgcaaatttggct

SEQ ID NO: 724 - DNA, 30 bp, synthetic construct
  source 1..30: mol_type = other DNA
cttacacgaacacnnkgcaaatttggctgg

SEQ ID NO: 725 - DNA, 33 bp, synthetic construct
  source 1..33: mol_type = other DNA
ttacacgaacacatcnnkaatttggctggttcg

SEQ ID NO: 726 - DNA, 27 bp, synthetic construct
  source 1..27: mol_type = other DNA
gaacacatcgcannkttggctggttcg

SEQ ID NO: 727 - DNA, 25 bp, synthetic construct
  source 1..25: mol_type = other DNA
acatcgcaaatnnkgctggttcgcc

SEQ ID NO: 728 - DNA, 25 bp, synthetic construct
  source 1..25: mol_type = other DNA
tcgcaaatttgnnkggttcgccggc

SEQ ID NO: 729 - DNA, 26 bp, synthetic construct
  source 1..26: mol_type = other DNA
caaatttggctnnktcgccggccatt

SEQ ID NO: 730 - DNA, 32 bp, synthetic construct
  source 1..32: mol_type = other DNA
caaatttggctggtnnkccggccattaaaaag

SEQ ID NO: 731 - DNA, 31 bp, synthetic construct
  source 1..31: mol_type = other DNA
atttggctggttcgnnkgccattaaaaaggg

SEQ ID NO: 732 - DNA, 28 bp, synthetic construct
  source 1..28: mol_type = other DNA
gctggttcgccgnnkattaaaaagggga

SEQ ID NO: 733 - DNA, 26 bp, synthetic construct
  source 1..26: mol_type = other DNA
gttcgccggccnnkaaaaaggggatc

SEQ ID NO: 734 - DNA, 25 bp, synthetic construct
  source 1..25: mol_type = other DNA
cgccggccattnnkaaggggatcct

SEQ ID NO: 735 - DNA, 28 bp, synthetic construct
  source 1..28: mol_type = other DNA
ccggccattaaannkgggatcctccaga

SEQ ID NO: 736 - DNA, 31 bp, synthetic construct
  source 1..31: mol_type = other DNA
cggccattaaaaagnnkatcctccagaccgt

SEQ ID NO: 737 - DNA, 31 bp, synthetic construct
  source 1..31: mol_type = other DNA
ccattaaaaaggggnnkctccagaccgtgaa

SEQ ID NO: 738 - DNA, 36 bp, synthetic construct
  source 1..36: mol_type = other DNA
cattaaaaaggggatcnnkcagaccgtgaaagttgt

SEQ ID NO: 739 - DNA, 36 bp, synthetic construct
  source 1..36: mol_type = other DNA
taaaaaggggatcctcnnkaccgtgaaagttgtaga

SEQ ID NO: 740 - DNA, 33 bp, synthetic construct
  source 1..33: mol_type = other DNA
aaggggatcctccagnnkgtgaaagttgtagat

SEQ ID NO: 741 - DNA, 33 bp, synthetic construct
  source 1..33: mol_type = other DNA
gggatcctccagaccnnkaaagttgtagatgag

SEQ ID NO: 742 - DNA, 29 bp, synthetic construct
  source 1..29: mol_type = other DNA
cctccagaccgtgnnkgttgtagatgagc

SEQ ID NO: 743 - DNA, 34 bp, synthetic construct
  source 1..34: mol_type = other DNA
ctccagaccgtgaaannkgtagatgagcttgtta

SEQ ID NO: 744 - DNA, 34 bp, synthetic construct
  source 1..34: mol_type = other DNA
cagaccgtgaaagttnnkgatgagcttgttaagg

SEQ ID NO: 745 - DNA, 36 bp, synthetic construct
  source 1..36: mol_type = other DNA
gaccgtgaaagttgtannkgagcttgttaaggtcat

SEQ ID NO: 746 - DNA, 37 bp, synthetic construct
  source 1..37: mol_type = other DNA
ccgtgaaagttgtagatnnkcttgttaaggtcatggg

SEQ ID NO: 747 - DNA, 36 bp, synthetic construct
  source 1..36: mol_type = other DNA
gaaagttgtagatgagnnkgttaaggtcatgggtcg

SEQ ID NO: 748 - DNA, 34 bp, synthetic construct
  source 1..34: mol_type = other DNA
gttgtagatgagcttnnkaaggtcatgggtcgtc

SEQ ID NO: 749 - DNA, 34 bp, synthetic construct
  source 1..34: mol_type = other DNA
gtagatgagcttgttnnkgtcatgggtcgtcata

SEQ ID NO: 750 - DNA, 35 bp, synthetic construct
  source 1..35: mol_type = other DNA
agatgagcttgttaagnnkatgggtcgtcataagc

SEQ ID NO: 751 - DNA, 31 bp, synthetic construct
  source 1..31: mol_type = other DNA
agcttgttaaggtcnnkggtcgtcataagcc

SEQ ID NO: 752 - DNA, 32 bp, synthetic construct
  source 1..32: mol_type = other DNA
ttgttaaggtcatgnnkcgtcataagcccgaa

SEQ ID NO: 753 - DNA, 34 bp, synthetic construct
  source 1..34: mol_type = other DNA
gttaaggtcatgggtnnkcataagcccgaaaaca

SEQ ID NO: 754 - DNA, 28 bp, synthetic construct
  source 1..28: mol_type = other DNA
gtcatgggtcgtnnkaagcccgaaaaca

SEQ ID NO: 755 - DNA, 29 bp, synthetic construct
  source 1..29: mol_type = other DNA
catgggtcgtcatnnkcccgaaaacatcg

SEQ ID NO: 756 - DNA, 36 bp, synthetic construct
  source 1..36: mol_type = other DNA
catgggtcgtcataagnnkgaaaacatcgtgattga

SEQ ID NO: 757 - DNA, 32 bp, synthetic construct
  source 1..32: mol_type = other DNA
gtcgtcataagcccnnkaacatcgtgattgaa

SEQ ID NO: 758 - DNA, 34 bp, synthetic construct
  source 1..34: mol_type = other DNA
cgtcataagcccgaannkatcgtgattgaaatgg

SEQ ID NO: 759 - DNA, 32 bp, synthetic construct
  source 1..32: mol_type = other DNA
ataagcccgaaaacnnkgtgattgaaatggcg

SEQ ID NO: 760 - DNA, 29 bp, synthetic construct
  source 1..29: mol_type = other DNA
gcccgaaaacatcnnkattgaaatggcgc

SEQ ID NO: 761 - DNA, 27 bp, synthetic construct
  source 1..27: mol_type = other DNA
gaaaacatcgtgnnkgaaatggcgcgg

SEQ ID NO: 762 - DNA, 30 bp, synthetic construct
  source 1..30: mol_type = other DNA
aaacatcgtgattnnkatggcgcgggagaa

SEQ ID NO: 763 - DNA, 29 bp, synthetic construct
  source 1..29: mol_type = other DNA
catcgtgattgaannkgcgcgggagaatc

SEQ ID NO: 764 - DNA, 33 bp, synthetic construct
  source 1..33: mol_type = other DNA
atcgtgattgaaatgnnkcgggagaatcaaacg

SEQ ID NO: 765 - DNA, 33 bp, synthetic construct
  source 1..33: mol_type = other DNA
gtgattgaaatggcgnnkgagaatcaaacgacc

SEQ ID NO: 766 - DNA, 27 bp, synthetic construct
  source 1..27: mol_type = other DNA
gaaatggcgcggnnkaatcaaacgacc

SEQ ID NO: 767 - DNA, 25 bp, synthetic construct
  source 1..25: mol_type = other DNA
tggcgcgggagnnkcaaacgaccca

SEQ ID NO: 768 - DNA, 26 bp, synthetic construct
  source 1..26: mol_type = other DNA
cgcgggagaatnnkacgacccagaaa

SEQ ID NO: 769 - DNA, 30 bp, synthetic construct
  source 1..30: mol_type = other DNA
gcgggagaatcaannkacccagaaaggaca

SEQ ID NO: 770 - DNA, 33 bp, synthetic construct
  source 1..33: mol_type = other DNA
cgggagaatcaaacgnnkcagaaaggacaaaag

SEQ ID NO: 771 - DNA, 38 bp, synthetic construct
  source 1..38: mol_type = other DNA
gggagaatcaaacgaccnnkaaaggacaaaagaatagc

SEQ ID NO: 772 - DNA, 34 bp, synthetic construct
  source 1..34: mol_type = other DNA
aatcaaacgacccagnnkggacaaaagaatagcc

SEQ ID NO: 773 - DNA, 34 bp, synthetic construct
  source 1..34: mol_type = other DNA
caaacgacccagaaannkcaaaagaatagccgtg

SEQ ID NO: 774 - DNA, 32 bp, synthetic construct
  source 1..32: mol_type = other DNA
cgacccagaaaggannkaagaatagccgtgaa

SEQ ID NO: 775 - DNA, 31 bp, synthetic construct
  source 1..31: mol_type = other DNA
cccagaaaggacaannkaatagccgtgaacg

SEQ ID NO: 776 - DNA, 31 bp, synthetic construct
  source 1..31: mol_type = other DNA
agaaaggacaaaagnnkagccgtgaacggat

SEQ ID NO: 777 - DNA, 35 bp, synthetic construct
  source 1..35: mol_type = other DNA
gaaaggacaaaagaatnnkcgtgaacggatgaagc

SEQ ID NO: 778 - DNA, 32 bp, synthetic construct
  source 1..32: mol_type = other DNA
gacaaaagaatagcnnkgaacggatgaagcgg

SEQ ID NO: 779 - DNA, 31 bp, synthetic construct
  source 1..31: mol_type = other DNA
aaaagaatagccgtnnkcggatgaagcggat

SEQ ID NO: 780 - DNA, 32 bp, synthetic construct
  source 1..32: mol_type = other DNA
agaatagccgtgaannkatgaagcggatcgag

SEQ ID NO: 781 - DNA, 25 bp, synthetic construct
  source 1..25: mol_type = other DNA
gccgtgaacggnnkaagcggatcga

SEQ ID NO: 782 - DNA, 26 bp, synthetic construct
  source 1..26: mol_type = other DNA
gtgaacggatgnnkcggatcgaggaa

SEQ ID NO: 783 - DNA, 32 bp, synthetic construct
  source 1..32: mol_type = other DNA
gtgaacggatgaagnnkatcgaggaaggcatt

SEQ ID NO: 784 - DNA, 28 bp, synthetic construct
  source 1..28: mol_type = other DNA
cggatgaagcggnnkgaggaaggcatta

SEQ ID NO: 785 - DNA, 32 bp, synthetic construct
  source 1..32: mol_type = other DNA
ggatgaagcggatcnnkgaaggcattaaagag

SEQ ID NO: 786 - DNA, 30 bp, synthetic construct
  source 1..30: mol_type = other DNA
gaagcggatcgagnnkggcattaaagagct

SEQ ID NO: 787 - DNA, 31 bp, synthetic construct
  source 1..31: mol_type = other DNA
agcggatcgaggaannkattaaagagctggg

SEQ ID NO: 788 - DNA, 27 bp, synthetic construct
  source 1..27: mol_type = other DNA
atcgaggaaggcnnkaaagagctgggg

SEQ ID NO: 789 - DNA, 28 bp, synthetic construct
  source 1..28: mol_type = other DNA
gaggaaggcattnnkgagctggggtctc

SEQ ID NO: 790 - DNA, 34 bp, synthetic construct
  source 1..34: mol_type = other DNA
gaggaaggcattaaannkctggggtctcaaatct

SEQ ID NO: 791 - DNA, 36 bp, synthetic construct
  source 1..36: mol_type = other DNA
ggaaggcattaaagagnnkgggtctcaaatcttgaa

SEQ ID NO: 792 - DNA, 37 bp, synthetic construct
  source 1..37: mol_type = other DNA
aaggcattaaagagctgnnktctcaaatcttgaagga

SEQ ID NO: 793 - DNA, 35 bp, synthetic construct
  source 1..35: mol_type = other DNA
cattaaagagctggggnnkcaaatcttgaaggaac

SEQ ID NO: 794 - DNA, 35 bp, synthetic construct
  source 1..35: mol_type = other DNA
taaagagctggggtctnnkatcttgaaggaacacc

SEQ ID NO: 795 - DNA, 29 bp, synthetic construct
  source 1..29: mol_type = other DNA
gctggggtctcaannkttgaaggaacacc

SEQ ID NO: 796 - DNA, 30 bp, synthetic construct
  source 1..30: mol_type = other DNA
ggggtctcaaatcnnkaaggaacaccctgt

SEQ ID NO: 797 - DNA, 32 bp, synthetic construct
  source 1..32: mol_type = other DNA
ggtctcaaatcttgnnkgaacaccctgtggag

SEQ ID NO: 798 - DNA, 33 bp, synthetic construct
  source 1..33: mol_type = other DNA
tctcaaatcttgaagnnkcaccctgtggagaac

SEQ ID NO: 799 - DNA, 35 bp, synthetic construct
  source 1..35: mol_type = other DNA
tcaaatcttgaaggaannkcctgtggagaacactc

SEQ ID NO: 800 - DNA, 33 bp, synthetic construct
  source 1..33: mol_type = other DNA
atcttgaaggaacacnnkgtggagaacactcag

SEQ ID NO: 801 - DNA, 30 bp, synthetic construct
  source 1..30: mol_type = other DNA
gaaggaacaccctnnkgagaacactcagct

SEQ ID NO: 802 - DNA, 28 bp, synthetic construct
  source 1..28: mol_type = other DNA
gaacaccctgtgnnkaacactcagctcc

SEQ ID NO: 803 - DNA, 28 bp, synthetic construct
  source 1..28: mol_type = other DNA
caccctgtggagnnkactcagctccaaa

SEQ ID NO: 804 - DNA, 30 bp, synthetic construct
  source 1..30: mol_type = other DNA
ccctgtggagaacnnkcagctccaaaatga

SEQ ID NO: 805 - DNA, 36 bp, synthetic construct
  source 1..36: mol_type = other DNA
ccctgtggagaacactnnkctccaaaatgaaaaact

SEQ ID NO: 806 - DNA, 39 bp, synthetic construct
  source 1..39: mol_type = other DNA
cctgtggagaacactcagnnkcaaaatgaaaaactttac

SEQ ID NO: 807 - DNA, 37 bp, synthetic construct
  source 1..37: mol_type = other DNA
tggagaacactcagctcnnkaatgaaaaactttacct

SEQ ID NO: 808 - DNA, 38 bp, synthetic construct
  source 1..38: mol_type = other DNA
agaacactcagctccaannkgaaaaactttacctgtac

SEQ ID NO: 809 - DNA, 44 bp, synthetic construct
  source 1..44: mol_type = other DNA
agaacactcagctccaaaatnnkaaactttacctgtactatttg

SEQ ID NO: 810 - DNA, 40 bp, synthetic construct
  source 1..40: mol_type = other DNA
actcagctccaaaatgaannkctttacctgtactatttgc

SEQ ID NO: 811 - DNA, 41 bp, synthetic construct
  source 1..41: mol_type = other DNA
tcagctccaaaatgaaaaannktacctgtactatttgcaga

SEQ ID NO: 812 - DNA, 41 bp, synthetic construct
  source 1..41: mol_type = other DNA
gctccaaaatgaaaaacttnnkctgtactatttgcagaacg

SEQ ID NO: 813 - DNA, 42 bp, synthetic construct
  source 1..42: mol_type = other DNA
ccaaaatgaaaaactttacnnktactatttgcagaacggacg

SEQ ID NO: 814 - DNA, 37 bp, synthetic construct
  source 1..37: mol_type = other DNA
atgaaaaactttacctgnnktatttgcagaacggacg

SEQ ID NO: 815 - DNA, 31 bp, synthetic construct
  source 1..31: mol_type = other DNA
aactttacctgtacnnkttgcagaacggacg

SEQ ID NO: 816 - DNA, 37 bp, synthetic construct
  source 1..37: mol_type = other DNA
aactttacctgtactatnnkcagaacggacgcgatat

SEQ ID NO: 817 - DNA, 38 bp, synthetic construct
  source 1..38: mol_type = other DNA
tttacctgtactatttgnnkaacggacgcgatatgtac

SEQ ID NO: 818 - DNA, 34 bp, synthetic construct
  source 1..34: mol_type = other DNA
ctgtactatttgcagnnkggacgcgatatgtacg

SEQ ID NO: 819 - DNA, 34 bp, synthetic construct
  source 1..34: mol_type = other DNA
tactatttgcagaacnnkcgcgatatgtacgtgg

SEQ ID NO: 820 - DNA, 35 bp, synthetic construct
  source 1..35: mol_type = other DNA
ctatttgcagaacggannkgatatgtacgtggacc

SEQ ID NO: 821 - DNA, 27 bp, synthetic construct
  source 1..27: mol_type = other DNA
cagaacggacgcnnkatgtacgtggac

SEQ ID NO: 822 - DNA, 27 bp, synthetic construct
  source 1..27: mol_type = other DNA
aacggacgcgatnnktacgtggaccaa

SEQ ID NO: 823 - DNA, 28 bp, synthetic construct
  source 1..28: mol_type = other DNA
ggacgcgatatgnnkgtggaccaagagt

SEQ ID NO: 824 - DNA, 32 bp, synthetic construct
  source 1..32: mol_type = other DNA
gacgcgatatgtacnnkgaccaagagttggat

SEQ ID NO: 825 - DNA, 37 bp, synthetic construct
  source 1..37: mol_type = other DNA
gacgcgatatgtacgtgnnkcaagagttggatattaa

SEQ ID NO: 826 - DNA, 37 bp, synthetic construct
  source 1..37: mol_type = other DNA
gcgatatgtacgtggacnnkgagttggatattaatcg

SEQ ID NO: 827 - DNA, 38 bp, synthetic construct
  source 1..38: mol_type = other DNA
atatgtacgtggaccaannkttggatattaatcggctg

SEQ ID NO: 828 - DNA, 34 bp, synthetic construct
  source 1..34: mol_type = other DNA
tacgtggaccaagagnnkgatattaatcggctga

SEQ ID NO: 829 - DNA, 33 bp, synthetic construct
  source 1..33: mol_type = other DNA
gtggaccaagagttgnnkattaatcggctgagt

SEQ ID NO: 830 - DNA, 33 bp, synthetic construct
  source 1..33: mol_type = other DNA
gaccaagagttggatnnkaatcggctgagtgac

SEQ ID NO: 831 - DNA, 34 bp, synthetic construct
  source 1..34: mol_type = other DNA
caagagttggatattnnkcggctgagtgactacg

SEQ ID NO: 832 - DNA, 40 bp, synthetic construct
  source 1..40: mol_type = other DNA
caagagttggatattaatnnkctgagtgactacgacgttg

SEQ ID NO: 833 - DNA, 37 bp, synthetic construct
  source 1..37: mol_type = other DNA
agttggatattaatcggnnkagtgactacgacgttga

SEQ ID NO: 834 - DNA, 35 bp, synthetic construct
  source 1..35: mol_type = other DNA
ggatattaatcggctgnnkgactacgacgttgatg

SEQ ID NO: 835 - DNA, 31 bp, synthetic construct
  source 1..31: mol_type = other DNA
ttaatcggctgagtnnktacgacgttgatgc

SEQ ID NO: 836 - DNA, 26 bp, synthetic construct
  source 1..26: mol_type = other DNA
ggctgagtgacnnkgacgttgatgcg

SEQ ID NO: 837 - DNA, 29 bp, synthetic construct
  source 1..29: mol_type = other DNA
gctgagtgactacnnkgttgatgcgatcg

SEQ ID NO: 838 - DNA, 29 bp, synthetic construct
  source 1..29: mol_type = other DNA
gagtgactacgacnnkgatgcgatcgtcc

SEQ ID NO: 839 - DNA, 25 bp, synthetic construct
  source 1..25: mol_type = other DNA
actacgacgttnnkgcgatcgtccc

SEQ ID NO: 840 - DNA, 30 bp, synthetic construct
  source 1..30: mol_type = other DNA
ctacgacgttgatnnkatcgtcccgcagag

SEQ ID NO: 841 - DNA, 25 bp, synthetic construct
  source 1..25: mol_type = other DNA
acgttgatgcgnnkgtcccgcagag

SEQ ID NO: 842 - DNA, 27 bp, synthetic construct
  source 1..27: mol_type = other DNA
gttgatgcgatcnnkccgcagagcttc

SEQ ID NO: 843 - DNA, 28 bp, synthetic construct
  source 1..28: mol_type = other DNA
gatgcgatcgtcnnkcagagcttcctca

SEQ ID NO: 844 - DNA, 26 bp, synthetic construct
  source 1..26: mol_type = other DNA
cgatcgtcccgnnkagcttcctcaaa

SEQ ID NO: 845 - DNA, 27 bp, synthetic construct
  source 1..27: mol_type = other DNA
atcgtcccgcagnnkttcctcaaagac

SEQ ID NO: 846 - DNA, 27 bp, synthetic construct
  source 1..27: mol_type = other DNA
gtcccgcagagcnnkctcaaagacgat

SEQ ID NO: 847 - DNA, 33 bp, synthetic construct
  source 1..33: mol_type = other DNA
gtcccgcagagcttcnnkaaagacgattctatt

SEQ ID NO: 848 - DNA, 31 bp, synthetic construct
  source 1..31: mol_type = other DNA
cgcagagcttcctcnnkgacgattctattga

SEQ ID NO: 849 - DNA, 39 bp, synthetic construct
  source 1..39: mol_type = other DNA
ccgcagagcttcctcaaannkgattctattgacaataag

SEQ ID NO: 850 - DNA, 41 bp, synthetic construct
  source 1..41: mol_type = other DNA
gcagagcttcctcaaagacnnktctattgacaataaggtac

SEQ ID NO: 851 - DNA, 40 bp, synthetic construct
  source 1..40: mol_type = other DNA
agcttcctcaaagacgatnnkattgacaataaggtactga

SEQ ID NO: 852 - DNA, 38 bp, synthetic construct
  source 1..38: mol_type = other DNA
tcctcaaagacgattctnnkgacaataaggtactgacg

SEQ ID NO: 853 - DNA, 38 bp, synthetic construct
  source 1..38: mol_type = other DNA
tcaaagacgattctattnnkaataaggtactgacgcgc

SEQ ID NO: 854 - DNA, 32 bp, synthetic construct
  source 1..32: mol_type = other DNA
acgattctattgacnnkaaggtactgacgcgc

SEQ ID NO: 855 - DNA, 34 bp, synthetic construct
  source 1..34: mol_type = other DNA
gattctattgacaatnnkgtactgacgcgctctg

SEQ ID NO: 856 - DNA, 37 bp, synthetic construct
  source 1..37: mol_type = other DNA
attctattgacaataagnnkctgacgcgctctgataa

SEQ ID NO: 857 - DNA, 40 bp, synthetic construct
  source 1..40: mol_type = other DNA
tctattgacaataaggtannkacgcgctctgataaaaacc

SEQ ID NO: 858 - DNA, 37 bp, synthetic construct
  source 1..37: mol_type = other DNA
ttgacaataaggtactgnnkcgctctgataaaaaccg

SEQ ID NO: 859 - DNA, 37 bp, synthetic construct
  source 1..37: mol_type = other DNA
acaataaggtactgacgnnktctgataaaaaccgtgg

SEQ ID NO: 860 - DNA, 29 bp, synthetic construct
  source 1..29: mol_type = other DNA
ggtactgacgcgcnnkgataaaaaccgtg

SEQ ID NO: 861 - DNA, 32 bp, synthetic construct
  source 1..32: mol_type = other DNA
tactgacgcgctctnnkaaaaaccgtggtaag

SEQ ID NO: 862 - DNA, 29 bp, synthetic construct
  source 1..29: mol_type = other DNA
gacgcgctctgatnnkaaccgtggtaagt

SEQ ID NO: 863 - DNA, 30 bp, synthetic construct
  source 1..30: mol_type = other DNA
gcgctctgataaannkcgtggtaagtcgga

SEQ ID NO: 864 - DNA, 33 bp, synthetic construct
  source 1..33: mol_type = other DNA
cgctctgataaaaacnnkggtaagtcggacaac

SEQ ID NO: 865 - DNA, 33 bp, synthetic construct
  source 1..33: mol_type = other DNA
tctgataaaaaccgtnnkaagtcggacaacgtg

SEQ ID NO: 866 - DNA, 29 bp, synthetic construct
  source 1..29: mol_type = other DNA
taaaaaccgtggtnnktcggacaacgtgc

SEQ ID NO: 867 - DNA, 25 bp, synthetic construct
  source 1..25: mol_type = other DNA
accgtggtaagnnkgacaacgtgcc

SEQ ID NO: 868 - DNA, 26 bp, synthetic construct
  source 1..26: mol_type = other DNA
gtggtaagtcgnnkaacgtgccctcc

SEQ ID NO: 869 - DNA, 26 bp, synthetic construct
  source 1..26: mol_type = other DNA
gtaagtcggacnnkgtgccctccgaa

SEQ ID NO: 870 - DNA, 27 bp, synthetic construct
  source 1..27: mol_type = other DNA
aagtcggacaacnnkccctccgaagag

SEQ ID NO: 871 - DNA, 28 bp, synthetic construct
  source 1..28: mol_type = other DNA
tcggacaacgtgnnktccgaagaggttg

SEQ ID NO: 872 - DNA, 27 bp, synthetic construct
  source 1..27: mol_type = other DNA
gacaacgtgcccnnkgaagaggttgtg

SEQ ID NO: 873 - DNA, 27 bp, synthetic construct
  source 1..27: mol_type = other DNA
aacgtgccctccnnkgaggttgtgaaa

SEQ ID NO: 874 - DNA, 31 bp, synthetic construct
  source 1..31: mol_type = other DNA
acgtgccctccgaannkgttgtgaaaaagat

SEQ ID NO: 875 - DNA, 33 bp, synthetic construct
  source 1..33: mol_type = other DNA
gtgccctccgaagagnnkgtgaaaaagatgaaa

SEQ ID NO: 876 - DNA, 41 bp, synthetic construct
  source 1..41: mol_type = other DNA
cgtgccctccgaagaggttnnkaaaaagatgaaaaattatt

SEQ ID NO: 877 - DNA, 39 bp, synthetic construct
  source 1..39: mol_type = other DNA
ccctccgaagaggttgtgnnkaagatgaaaaattattgg

SEQ ID NO: 878 - DNA, 38 bp, synthetic construct
  source 1..38: mol_type = other DNA
ccgaagaggttgtgaaannkatgaaaaattattggcgc

SEQ ID NO: 879 - DNA, 37 bp, synthetic construct
  source 1..37: mol_type = other DNA
aagaggttgtgaaaaagnnkaaaaattattggcgcca

SEQ ID NO: 880 - DNA, 34 bp, synthetic construct
  source 1..34: mol_type = other DNA
gttgtgaaaaagatgnnkaattattggcgccagc

SEQ ID NO: 881 - DNA, 37 bp, synthetic construct
  source 1..37: mol_type = other DNA
ttgtgaaaaagatgaaannktattggcgccagctttt

SEQ ID NO: 882 - DNA, 38 bp, synthetic construct
  source 1..38: mol_type = other DNA
tgaaaaagatgaaaaatnnktggcgccagcttttaaac

SEQ ID NO: 883 - DNA, 38 bp, synthetic construct
  source 1..38: mol_type = other DNA
aaaagatgaaaaattatnnkcgccagcttttaaacgcg

SEQ ID NO: 884 - DNA, 40 bp, synthetic construct
  source 1..40: mol_type = other DNA
aagatgaaaaattattggnnkcagcttttaaacgcgaagc

SEQ ID NO: 885 - DNA, 35 bp, synthetic construct
  source 1..35: mol_type = other DNA
gaaaaattattggcgcnnkcttttaaacgcgaagc

SEQ ID NO: 886 - DNA, 28 bp, synthetic construct
  source 1..28: mol_type = other DNA
tattggcgccagnnkttaaacgcgaagc

SEQ ID NO: 887 - DNA, 25 bp, synthetic construct
  source 1..25: mol_type = other DNA
ggcgccagcttnnkaacgcgaagct

SEQ ID NO: 888 - DNA, 28 bp, synthetic construct
  source 1..28: mol_type = other DNA
cgccagcttttannkgcgaagctgatca

SEQ ID NO: 889 - DNA, 33 bp, synthetic construct
  source 1..33: mol_type = other DNA
cgccagcttttaaacnnkaagctgatcacacaa

SEQ ID NO: 890 - DNA, 30 bp, synthetic construct
  source 1..30: mol_type = other DNA
gcttttaaacgcgnnkctgatcacacaacg

SEQ ID NO: 891 - DNA, 37 bp, synthetic construct
  source 1..37: mol_type = other DNA
agcttttaaacgcgaagnnkatcacacaacgtaaatt

SEQ ID NO: 892 - DNA, 35 bp, synthetic construct
  source 1..35: mol_type = other DNA
tttaaacgcgaagctgnnkacacaacgtaaattcg

SEQ ID NO: 893 - DNA, 37 bp, synthetic construct
  source 1..37: mol_type = other DNA
taaacgcgaagctgatcnnkcaacgtaaattcgataa

SEQ ID NO: 894 - DNA, 36 bp, synthetic construct
  source 1..36: mol_type = other DNA
cgcgaagctgatcacannkcgtaaattcgataattt

SEQ ID NO: 895 - DNA, 39 bp, synthetic construct
  source 1..39: mol_type = other DNA
gcgaagctgatcacacaannkaaattcgataatttgacc

SEQ ID NO: 896 - DNA, 36 bp, synthetic construct
  source 1..36: mol_type = other DNA
gctgatcacacaacgtnnkttcgataatttgaccaa

SEQ ID NO: 897 - DNA, 38 bp, synthetic construct
  source 1..38: mol_type = other DNA
tgatcacacaacgtaaannkgataatttgaccaaggct

SEQ ID NO: 898 - DNA, 36 bp, synthetic construct
  source 1..36: mol_type = other DNA
cacacaacgtaaattcnnkaatttgaccaaggctga

SEQ ID NO: 899 - DNA, 35 bp, synthetic construct
  source 1..35: mol_type = other DNA
acaacgtaaattcgatnnkttgaccaaggctgaac

SEQ ID NO: 900 - DNA, 35 bp, synthetic construct
  source 1..35: mol_type = other DNA
acgtaaattcgataatnnkaccaaggctgaacggg

SEQ ID NO: 901 - DNA, 35 bp, synthetic construct
  source 1..35: mol_type = other DNA
taaattcgataatttgnnkaaggctgaacggggtg

SEQ ID NO: 902 - DNA, 29 bp, synthetic construct
  source 1..29: mol_type = other DNA
cgataatttgaccnnkgctgaacggggtg

SEQ ID NO: 903 - DNA, 27 bp, synthetic construct
  source 1..27: mol_type = other DNA
aatttgaccaagnnkgaacggggtggc

SEQ ID NO: 904 - DNA, 25 bp, synthetic construct
  source 1..25: mol_type = other DNA
tgaccaaggctnnkcggggtggcct

SEQ ID NO: 905 - DNA, 25 bp, synthetic construct
  source 1..25: mol_type = other DNA
ccaaggctgaannkggtggcctgag

SEQ ID NO: 906 - DNA, 25 bp, synthetic construct
  source 1..25: mol_type = other DNA
aggctgaacggnnkggcctgagcga

SEQ ID NO: 907 - DNA, 27 bp, synthetic construct
  source 1..27: mol_type = other DNA
gctgaacggggtnnkctgagcgagtta

SEQ ID NO: 908 - DNA, 28 bp, synthetic construct
  source 1..28: mol_type = other DNA
gaacggggtggcnnkagcgagttagata

SEQ ID NO: 909 - DNA, 28 bp, synthetic construct
  source 1..28: mol_type = other DNA
cggggtggcctgnnkgagttagataagg

SEQ ID NO: 910 - DNA, 29 bp, synthetic construct
  source 1..29: mol_type = other DNA
gggtggcctgagcnnkttagataaggcag

SEQ ID NO: 911 - DNA, 26 bp, synthetic construct
  source 1..26: mol_type = other DNA
gcctgagcgagnnkgataaggcagga

SEQ ID NO: 912 - DNA, 34 bp, synthetic construct
  source 1..34: mol_type = other DNA
ggcctgagcgagttannkaaggcaggatttatta

SEQ ID NO: 913 - DNA, 36 bp, synthetic construct
  source 1..36: mol_type = other DNA
cctgagcgagttagatnnkgcaggatttattaaacg

SEQ ID NO: 914 - DNA, 38 bp, synthetic construct
  source 1..38: mol_type = other DNA
tgagcgagttagataagnnkggatttattaaacgccag

SEQ ID NO: 915 - DNA, 39 bp, synthetic construct
  source 1..39: mol_type = other DNA
agcgagttagataaggcannktttattaaacgccagtta

SEQ ID NO: 916 - DNA, 38 bp, synthetic construct
  source 1..38: mol_type = other DNA
agttagataaggcaggannkattaaacgccagttagtg

SEQ ID NO: 917 - DNA, 37 bp, synthetic construct
  source 1..37: mol_type = other DNA
tagataaggcaggatttnnkaaacgccagttagtgga

SEQ ID NO: 918 - DNA, 35 bp, synthetic construct
  source 1..35: mol_type = other DNA
taaggcaggatttattnnkcgccagttagtggaga

SEQ ID NO: 919 - DNA, 38 bp, synthetic construct
  source 1..38: mol_type = other DNA
aggcaggatttattaaannkcagttagtggagactcgt

SEQ ID NO: 920 - DNA, 37 bp, synthetic construct
  source 1..37: mol_type = other DNA
caggatttattaaacgcnnkttagtggagactcgtca

SEQ ID NO: 921 - DNA, 37 bp, synthetic construct
  source 1..37: mol_type = other DNA
gatttattaaacgccagnnkgtggagactcgtcaaat

SEQ ID NO: 922 - DNA, 38 bp, synthetic construct
  source 1..38: mol_type = other DNA
ttattaaacgccagttannkgagactcgtcaaatcacc

SEQ ID NO: 923 - DNA, 31 bp, synthetic construct
  source 1..31: mol_type = other DNA
aacgccagttagtgnnkactcgtcaaatcac

SEQ ID NO: 924 - DNA, 30 bp, synthetic construct
  source 1..30: mol_type = other DNA
ccagttagtggagnnkcgtcaaatcaccaa

SEQ ID NO: 925 - DNA, 36 bp, synthetic construct
  source 1..36: mol_type = other DNA
ccagttagtggagactnnkcaaatcaccaaacatgt

SEQ ID NO: 926 - DNA, 35 bp, synthetic construct
  source 1..35: mol_type = other DNA
gttagtggagactcgtnnkatcaccaaacatgtcg

SEQ ID NO: 927 - DNA, 30 bp, synthetic construct
  source 1..30: mol_type = other DNA
ggagactcgtcaannkaccaaacatgtcgc

SEQ ID NO: 928 - DNA, 30 bp, synthetic construct
  source 1..30: mol_type = other DNA
gactcgtcaaatcnnkaaacatgtcgcgca

SEQ ID NO: 929 - DNA, 26 bp, synthetic construct
  source 1..26: mol_type = other DNA
gtcaaatcaccnnkcatgtcgcgcag

SEQ ID NO: 930 - DNA, 32 bp, synthetic construct
  source 1..32: mol_type = other DNA
gtcaaatcaccaaannkgtcgcgcagattttg

SEQ ID NO: 931 - DNA, 33 bp, synthetic construct
  source 1..33: mol_type = other DNA
caaatcaccaaacatnnkgcgcagattttggac

SEQ ID NO: 932 - DNA, 33 bp, synthetic construct
  source 1..33: mol_type = other DNA
atcaccaaacatgtcnnkcagattttggacagc

SEQ ID NO: 933 - DNA, 29 bp, synthetic construct
  source 1..29: mol_type = other DNA
caaacatgtcgcgnnkattttggacagcc

SEQ ID NO: 934 - DNA, 25 bp, synthetic construct
  source 1..25: mol_type = other DNA
atgtcgcgcagnnkttggacagccg

SEQ ID NO: 935 - DNA, 25 bp, synthetic construct
  source 1..25: mol_type = other DNA
tcgcgcagattnnkgacagccggat

SEQ ID NO: 936 - DNA, 28 bp, synthetic construct
  source 1..28: mol_type = other DNA
gcgcagattttgnnkagccggatgaaca

SEQ ID NO: 937 - DNA, 30 bp, synthetic construct
  source 1..30: mol_type = other DNA
gcagattttggacnnkcggatgaacaccaa

SEQ ID NO: 938 - DNA, 34 bp, synthetic construct
  source 1..34: mol_type = other DNA
cagattttggacagcnnkatgaacaccaagtacg

SEQ ID NO: 939 - DNA, 27 bp, synthetic construct
  source 1..27: mol_type = other DNA
ttggacagccggnnkaacaccaagtac

SEQ ID NO: 940 - DNA, 28 bp, synthetic construct
  source 1..28: mol_type = other DNA
gacagccggatgnnkaccaagtacgatg

SEQ ID NO: 941 - DNA, 34 bp, synthetic construct
  source 1..34: mol_type = other DNA
gacagccggatgaacnnkaagtacgatgaaaatg

SEQ ID NO: 942 - DNA, 33 bp, synthetic construct
  source 1..33: mol_type = other DNA
agccggatgaacaccnnktacgatgaaaatgac

SEQ ID NO: 943 - DNA, 35 bp, synthetic construct
  source 1..35: mol_type = other DNA
ccggatgaacaccaagnnkgatgaaaatgacaaac

SEQ ID NO: 944 - DNA, 38 bp, synthetic construct
  source 1..38: mol_type = other DNA
ggatgaacaccaagtacnnkgaaaatgacaaactgatc

SEQ ID NO: 945 - DNA, 37 bp, synthetic construct
  source 1..37: mol_type = other DNA
tgaacaccaagtacgatnnkaatgacaaactgatccg

SEQ ID NO: 946 - DNA, 34 bp, synthetic construct
  source 1..34: mol_type = other DNA
accaagtacgatgaannkgacaaactgatccgtg

SEQ ID NO: 947 - DNA, 37 bp, synthetic construct
  source 1..37: mol_type = other DNA
ccaagtacgatgaaaatnnkaaactgatccgtgaggt

SEQ ID NO: 948 - DNA, 33 bp, synthetic construct
  source 1..33: mol_type = other DNA
tacgatgaaaatgacnnkctgatccgtgaggtg

SEQ ID NO: 949 - DNA, 36 bp, synthetic construct
  source 1..36: mol_type = other DNA
cgatgaaaatgacaaannkatccgtgaggtgaaagt

SEQ ID NO: 950 - DNA, 34 bp, synthetic construct
  source 1..34: mol_type = other DNA
gaaaatgacaaactgnnkcgtgaggtgaaagtca

SEQ ID NO: 951 - DNA, 41 bp, synthetic construct
  source 1..41: mol_type = other DNA
tgaaaatgacaaactgatcnnkgaggtgaaagtcattactc

SEQ ID NO: 952 - DNA, 37 bp, synthetic construct
  source 1..37: mol_type = other DNA
atgacaaactgatccgtnnkgtgaaagtcattactct

SEQ ID NO: 953 - DNA, 38 bp, synthetic construct
  source 1..38: mol_type = other DNA
acaaactgatccgtgagnnkaaagtcattactctgaag

SEQ ID NO: 954 - DNA, 33 bp, synthetic construct
  source 1..33: mol_type = other DNA
ctgatccgtgaggtgnnkgtcattactctgaag

SEQ ID NO: 955 - DNA, 36 bp, synthetic construct
  source 1..36: mol_type = other DNA
gatccgtgaggtgaaannkattactctgaagtccaa

SEQ ID NO: 956 - DNA, 34 bp, synthetic construct
  source 1..34: mol_type = other DNA
cgtgaggtgaaagtcnnkactctgaagtccaaat

SEQ ID NO: 957 - DNA, 39 bp, synthetic construct
  source 1..39: mol_type = other DNA
cgtgaggtgaaagtcattnnkctgaagtccaaattagtt

SEQ ID NO: 958 - DNA, 45 bp, synthetic construct
  source 1..45: mol_type = other DNA
cgtgaggtgaaagtcattactnnkaagtccaaattagttagtgat

SEQ ID NO: 959 - DNA, 44 bp, synthetic construct
  source 1..44: mol_type = other DNA
aggtgaaagtcattactctgnnktccaaattagttagtgatttc

SEQ ID NO: 960 - DNA, 44 bp, synthetic construct
  source 1..44: mol_type = other DNA
tgaaagtcattactctgaagnnkaaattagttagtgatttccgg

SEQ ID NO: 961 - DNA, 40 bp, synthetic construct
  source 1..40: mol_type = other DNA
gtcattactctgaagtccnnkttagttagtgatttccgga

SEQ ID NO: 962 - DNA, 38 bp, synthetic construct
  source 1..38: mol_type = other DNA
ttactctgaagtccaaannkgttagtgatttccggaag

SEQ ID NO: 963 - DNA, 37 bp, synthetic construct
  source 1..37: mol_type = other DNA
ctctgaagtccaaattannkagtgatttccggaagga

SEQ ID NO: 964 - DNA, 37 bp, synthetic construct
  source 1..37: mol_type = other DNA
tgaagtccaaattagttnnkgatttccggaaggactt

SEQ ID NO: 965 - DNA, 39 bp, synthetic construct
  source 1..39: mol_type = other DNA
aagtccaaattagttagtnnkttccggaaggactttcaa

SEQ ID NO: 966 - DNA, 41 bp, synthetic construct
  source 1..41: mol_type = other DNA
gtccaaattagttagtgatnnkcggaaggactttcaattct

SEQ ID NO: 967 - DNA, 48 bp, synthetic construct
  source 1..48: mol_type = other DNA
gtccaaattagttagtgatttcnnkaaggactttcaattctacaaagt

SEQ ID NO: 968 - DNA, 43 bp, synthetic construct
  source 1..43: mol_type = other DNA
aattagttagtgatttccggnnkgactttcaattctacaaagt

SEQ ID NO: 969 - DNA, 41 bp, synthetic construct
  source 1..41: mol_type = other DNA
agttagtgatttccggaagnnktttcaattctacaaagtcc

SEQ ID NO: 970 - DNA, 35 bp, synthetic construct
  source 1..35: mol_type = other DNA
tgatttccggaaggacnnkcaattctacaaagtcc

SEQ ID NO: 971 - DNA, 35 bp, synthetic construct
  source 1..35: mol_type = other DNA
tttccggaaggactttnnkttctacaaagtccgtg

SEQ ID NO: 972 - DNA, 36 bp, synthetic construct
  source 1..36: mol_type = other DNA
ccggaaggactttcaannktacaaagtccgtgaaat

SEQ ID NO: 973 - DNA, 39 bp, synthetic construct
  source 1..39: mol_type = other DNA
cggaaggactttcaattcnnkaaagtccgtgaaattaat

SEQ ID NO: 974 - DNA, 43 bp, synthetic construct
  source 1..43: mol_type = other DNA
ggaaggactttcaattctacnnkgtccgtgaaattaataacta

SEQ ID NO: 975 - DNA, 49 bp, synthetic construct
  source 1..49: mol_type = other DNA
ggaaggactttcaattctacaaannkcgtgaaattaataactatcatca

SEQ ID NO: 976 - DNA, 48 bp, synthetic construct
  source 1..48: mol_type = other DNA
ggactttcaattctacaaagtcnnkgaaattaataactatcatcacgc

SEQ ID NO: 977 - DNA, 45 bp, synthetic construct
  source 1..45: mol_type = other DNA
tttcaattctacaaagtccgtnnkattaataactatcatcacgca

SEQ ID NO: 978 - DNA, 38 bp, synthetic construct
  source 1..38: mol_type = other DNA
tctacaaagtccgtgaannkaataactatcatcacgca

SEQ ID NO: 979 - DNA, 37 bp, synthetic construct
  source 1..37: mol_type = other DNA
acaaagtccgtgaaattnnkaactatcatcacgcaca

SEQ ID NO: 980 - DNA, 38 bp, synthetic construct
  source 1..38: mol_type = other DNA
aagtccgtgaaattaatnnktatcatcacgcacatgac

SEQ ID NO: 981 - DNA, 35 bp, synthetic construct
  source 1..35: mol_type = other DNA
ccgtgaaattaataacnnkcatcacgcacatgacg

SEQ ID NO: 982 - DNA, 38 bp, synthetic construct
  source 1..38: mol_type = other DNA
gtgaaattaataactatnnkcacgcacatgacgcgtac

SEQ ID NO: 983 - DNA, 39 bp, synthetic construct
  source 1..39: mol_type = other DNA
gaaattaataactatcatnnkgcacatgacgcgtacctg

SEQ ID NO: 984 - DNA, 40 bp, synthetic construct
  source 1..40: mol_type = other DNA
attaataactatcatcacnnkcatgacgcgtacctgaatg

SEQ ID NO: 985 - DNA, 30 bp, synthetic construct
  source 1..30: mol_type = other DNA
ctatcatcacgcannkgacgcgtacctgaa

SEQ ID NO: 986 - DNA, 29 bp, synthetic construct
  source 1..29: mol_type = other DNA
tcatcacgcacatnnkgcgtacctgaatg

SEQ ID NO: 987 - DNA, 31 bp, synthetic construct
  source 1..31: mol_type = other DNA
atcacgcacatgacnnktacctgaatgcagt

SEQ ID NO: 988 - DNA, 26 bp, synthetic construct
  source 1..26: mol_type = other DNA
cacatgacgcgnnkctgaatgcagtg

SEQ ID NO: 989 - DNA, 29 bp, synthetic construct
  source 1..29: mol_type = other DNA
acatgacgcgtacnnkaatgcagtggttg

SEQ ID NO: 990 - DNA, 25 bp, synthetic construct
  source 1..25: mol_type = other DNA
acgcgtacctgnnkgcagtggttgg

SEQ ID NO: 991 - DNA, 27 bp, synthetic construct
  source 1..27: mol_type = other DNA
gcgtacctgaatnnkgtggttgggacc

SEQ ID NO: 992 - DNA, 27 bp, synthetic construct
  source 1..27: mol_type = other DNA
tacctgaatgcannkgttgggaccgcc

SEQ ID NO: 993 - DNA, 25 bp, synthetic construct
  source 1..25: mol_type = other DNA
tgaatgcagtgnnkgggaccgccct

SEQ ID NO: 994 - DNA, 32 bp, synthetic construct
  source 1..32: mol_type = other DNA
tgaatgcagtggttnnkaccgcccttatcaag

SEQ ID NO: 995 - DNA, 28 bp, synthetic construct
  source 1..28: mol_type = other DNA
gcagtggttgggnnkgcccttatcaaga

SEQ ID NO: 996 - DNA, 35 bp, synthetic construct
  source 1..35: mol_type = other DNA
tgcagtggttgggaccnnkcttatcaagaaatatc

SEQ ID NO: 997 - DNA, 31 bp, synthetic construct
  source 1..31: mol_type = other DNA
tggttgggaccgccnnkatcaagaaatatcc

SEQ ID NO: 998 - DNA, 34 bp, synthetic construct
  source 1..34: mol_type = other DNA
gttgggaccgcccttnnkaagaaatatcctaagc

SEQ ID NO: 999 - DNA, 33 bp, synthetic construct
  source 1..33: mol_type = other DNA
gggaccgcccttatcnnkaaatatcctaagctg

SEQ ID NO: 1000 - DNA, 33 bp, synthetic construct
  source 1..33: mol_type = other DNA
accgcccttatcaagnnktatcctaagctggag

SEQ ID NO: 1001 - DNA, 33 bp, synthetic construct
  source 1..33: mol_type = other DNA
gcccttatcaagaaannkcctaagctggagtcg

SEQ ID NO: 1002 - DNA, 39 bp, synthetic construct
  source 1..39: mol_type = other DNA
gcccttatcaagaaatatnnkaagctggagtcggagttt

SEQ ID NO: 1003 - DNA, 39 bp, synthetic construct
  source 1..39: mol_type = other DNA
cttatcaagaaatatcctnnkctggagtcggagtttgtc

SEQ ID NO: 1004 - DNA, 42 bp, synthetic construct
  source 1..42: mol_type = other DNA
tatcaagaaatatcctaagnnkgagtcggagtttgtctatgg

SEQ ID NO: 1005 - DNA, 38 bp, synthetic construct
  source 1..38: mol_type = other DNA
agaaatatcctaagctgnnktcggagtttgtctatggc

SEQ ID NO: 1006 - DNA, 35 bp, synthetic construct
  source 1..35: mol_type = other DNA
atatcctaagctggagnnkgagtttgtctatggcg

SEQ ID NO: 1007 - DNA, 32 bp, synthetic construct
  source 1..32: mol_type = other DNA
ctaagctggagtcgnnktttgtctatggcgac

SEQ ID NO: 1008 - DNA, 27 bp, synthetic construct
  source 1..27: mol_type = other DNA
ctggagtcggagnnkgtctatggcgac

SEQ ID NO: 1009 - DNA, 34 bp, synthetic construct
  source 1..34: mol_type = other DNA
ctggagtcggagtttnnktatggcgactataagg

SEQ ID NO: 1010 - DNA, 32 bp, synthetic construct
  source 1..32: mol_type = other DNA
agtcggagtttgtcnnkggcgactataaggta

SEQ ID NO: 1011 - DNA, 41 bp, synthetic construct
  source 1..41: mol_type = other DNA
ggagtcggagtttgtctatnnkgactataaggtatacgatg

SEQ ID NO: 1012 - DNA, 39 bp, synthetic construct
  source 1..39: mol_type = other DNA
tcggagtttgtctatggcnnktataaggtatacgatgtt

SEQ ID NO: 1013 - DNA, 35 bp, synthetic construct
  source 1..35: mol_type = other DNA
gtttgtctatggcgacnnkaaggtatacgatgttc

SEQ ID NO: 1014 - DNA, 35 bp, synthetic construct
  source 1..35: mol_type = other DNA
tgtctatggcgactatnnkgtatacgatgttcgca

SEQ ID NO: 1015 - DNA, 37 bp, synthetic construct
  source 1..37: mol_type = other DNA
tctatggcgactataagnnktacgatgttcgcaaaat

SEQ ID NO: 1016 - DNA, 37 bp, synthetic construct
  source 1..37: mol_type = other DNA
atggcgactataaggtannkgatgttcgcaaaatgat

SEQ ID NO: 1017 - DNA, 37 bp, synthetic construct
  source 1..37: mol_type = other DNA
gcgactataaggtatacnnkgttcgcaaaatgattgc

SEQ ID NO: 1018 - DNA, 41 bp, synthetic construct
  source 1..41: mol_type = other DNA
cgactataaggtatacgatnnkcgcaaaatgattgcgaaat

SEQ ID NO: 1019 - DNA, 46 bp, synthetic construct
  source 1..46: mol_type = other DNA
gactataaggtatacgatgttnnkaaaatgattgcgaaatctgagc

SEQ ID NO: 1020 - DNA, 35 bp, synthetic construct
  source 1..35: mol_type = other DNA
ggtatacgatgttcgcnnkatgattgcgaaatctg

SEQ ID NO: 1021 - DNA, 35 bp, synthetic construct
  source 1..35: mol_type = other DNA
atacgatgttcgcaaannkattgcgaaatctgagc

SEQ ID NO: 1022 - DNA, 32 bp, synthetic construct
  source 1..32: mol_type = other DNA
atgttcgcaaaatgnnkgcgaaatctgagcag

SEQ ID NO: 1023 - DNA, 39 bp, synthetic construct
  source 1..39: mol_type = other DNA
gatgttcgcaaaatgattnnkaaatctgagcaggagatc

SEQ ID NO: 1024 - DNA, 32 bp, synthetic construct
  source 1..32: mol_type = other DNA
gcaaaatgattgcgnnktctgagcaggagatc

SEQ ID NO: 1025 - DNA, 34 bp, synthetic construct
  source 1..34: mol_type = other DNA
aaaatgattgcgaaannkgagcaggagatcggta

SEQ ID NO: 1026 - DNA, 35 bp, synthetic construct
  source 1..35: mol_type = other DNA
aatgattgcgaaatctnnkcaggagatcggtaagg

SEQ ID NO: 1027 - DNA, 31 bp, synthetic construct
  source 1..31: mol_type = other DNA
ttgcgaaatctgagnnkgagatcggtaaggc

SEQ ID NO: 1028 - DNA, 31 bp, synthetic construct
  source 1..31: mol_type = other DNA
cgaaatctgagcagnnkatcggtaaggcaac

SEQ ID NO: 1029 - DNA, 27 bp, synthetic construct
  source 1..27: mol_type = other DNA
tctgagcaggagnnkggtaaggcaacc

SEQ ID NO: 1030 - DNA, 26 bp, synthetic construct
  source 1..26: mol_type = other DNA
agcaggagatcnnkaaggcaaccgca

SEQ ID NO: 1031 - DNA, 28 bp, synthetic construct
  source 1..28: mol_type = other DNA
caggagatcggtnnkgcaaccgcaaaat

SEQ ID NO: 1032 - DNA, 36 bp, synthetic construct
  source 1..36: mol_type = other DNA
gcaggagatcggtaagnnkaccgcaaaatatttctt

SEQ ID NO: 1033 - DNA, 39 bp, synthetic construct
  source 1..39: mol_type = other DNA
caggagatcggtaaggcannkgcaaaatatttcttttac

SEQ ID NO: 1034 - DNA, 41 bp, synthetic construct
  source 1..41: mol_type = other DNA
ggagatcggtaaggcaaccnnkaaatatttcttttactcaa

SEQ ID NO: 1035 - DNA, 37 bp, synthetic construct
  source 1..37: mol_type = other DNA
tcggtaaggcaaccgcannktatttcttttactcaaa

SEQ ID NO: 1036 - DNA, 39 bp, synthetic construct
  source 1..39: mol_type = other DNA
ggtaaggcaaccgcaaaannkttcttttactcaaacatt

SEQ ID NO: 1037 - DNA, 45 bp, synthetic construct
  source 1..45: mol_type = other DNA
ggtaaggcaaccgcaaaatatnnkttttactcaaacattatgaat

SEQ ID NO: 1038 - DNA, 43 bp, synthetic construct
  source 1..43: mol_type = other DNA
aggcaaccgcaaaatatttcnnktactcaaacattatgaattt

SEQ ID NO: 1039 - DNA, 47 bp, synthetic construct
  source 1..47: mol_type = other DNA
ggcaaccgcaaaatatttctttnnktcaaacattatgaatttcttta

SEQ ID NO: 1040 - DNA, 51 bp, synthetic construct
  source 1..51: mol_type = other DNA
gcaaccgcaaaatatttcttttacnnkaacattatgaatttctttaagaca

SEQ ID NO: 1041 - DNA, 51 bp, synthetic construct
  source 1..51: mol_type = other DNA
accgcaaaatatttcttttactcannkattatgaatttctttaagacagaa

SEQ ID NO: 1042 - DNA, 51 bp, synthetic construct
  source 1..51: mol_type = other DNA
gcaaaatatttcttttactcaaacnnkatgaatttctttaagacagaaatc

SEQ ID NO: 1043 - DNA, 51 bp, synthetic construct
  source 1..51: mol_type = other DNA
aaatatttcttttactcaaacattnnkaatttctttaagacagaaatcact

SEQ ID NO: 1044 - DNA, 51 bp, synthetic construct
  source 1..51: mol_type = other DNA
tatttcttttactcaaacattatgnnkttctttaagacagaaatcactctg

SEQ ID NO: 1045 - DNA, 48 bp, synthetic construct
  source 1..48: mol_type = other DNA
cttttactcaaacattatgaatnnktttaagacagaaatcactctggc

SEQ ID NO: 1046 - DNA, 42 bp, synthetic construct
  source 1..42: mol_type = other DNA
ctcaaacattatgaatttcnnkaagacagaaatcactctggc

SEQ ID NO: 1047 - DNA, 43 bp, synthetic construct
  source 1..43: mol_type = other DNA
caaacattatgaatttctttnnkacagaaatcactctggccaa

SEQ ID NO: 1048 - DNA, 41 bp, synthetic construct
  source 1..41: mol_type = other DNA
cattatgaatttctttaagnnkgaaatcactctggccaacg

SEQ ID NO: 1049 - DNA, 36 bp, synthetic construct
  source 1..36: mol_type = other DNA
gaatttctttaagacannkatcactctggccaacgg

SEQ ID NO: 1050 - DNA, 33 bp, synthetic construct
  source 1..33: mol_type = other DNA
ttctttaagacagaannkactctggccaacggg

SEQ ID NO: 1051 - DNA, 29 bp, synthetic construct
  source 1..29: mol_type = other DNA
taagacagaaatcnnkctggccaacgggg

SEQ ID NO: 1052 - DNA, 30 bp, synthetic construct
  source 1..30: mol_type = other DNA
gacagaaatcactnnkgccaacggggagat

SEQ ID NO: 1053 - DNA, 31 bp, synthetic construct
  source 1..31: mol_type = other DNA
cagaaatcactctgnnkaacggggagattcg

SEQ ID NO: 1054 - DNA, 25 bp, synthetic construct
  source 1..25: mol_type = other DNA
tcactctggccnnkggggagattcg

SEQ ID NO: 1055 - DNA, 29 bp, synthetic construct
  source 1..29: mol_type = other DNA
cactctggccaacnnkgagattcgcaaac

SEQ ID NO: 1056 - DNA, 25 bp, synthetic construct
  source 1..25: mol_type = other DNA
tggccaacgggnnkattcgcaaacg

SEQ ID NO: 1057 - DNA, 25 bp, synthetic construct
  source 1..25: mol_type = other DNA
ccaacggggagnnkcgcaaacgtcc

SEQ ID NO: 1058 - DNA, 31 bp, synthetic construct
  source 1..31: mol_type = other DNA
ccaacggggagattnnkaaacgtccgttgat

SEQ ID NO: 1059 - DNA, 26 bp, synthetic construct
  source 1..26: mol_type = other DNA
gggagattcgcnnkcgtccgttgatc

SEQ ID NO: 1060 - DNA, 31 bp, synthetic construct
  source 1..31: mol_type = other DNA
gggagattcgcaaannkccgttgatcgaaac

SEQ ID NO: 1061 - DNA, 34 bp, synthetic construct
  source 1..34: mol_type = other DNA
gagattcgcaaacgtnnkttgatcgaaacaaacg

SEQ ID NO: 1062 - DNA, 29 bp, synthetic construct
  source 1..29: mol_type = other DNA
tcgcaaacgtccgnnkatcgaaacaaacg

SEQ ID NO: 1063 - DNA, 27 bp, synthetic construct
  source 1..27: mol_type = other DNA
aaacgtccgttgnnkgaaacaaacggc

SEQ ID NO: 1064 - DNA, 27 bp, synthetic construct
  source 1..27: mol_type = other DNA
cgtccgttgatcnnkacaaacggcgag

SEQ ID NO: 1065 - DNA, 28 bp, synthetic construct
  source 1..28: mol_type = other DNA
ccgttgatcgaannkaacggcgagactg

SEQ ID NO: 1066 - DNA, 27 bp, synthetic construct
  source 1..27: mol_type = other DNA
ttgatcgaaacannkggcgagactggc

SEQ ID NO: 1067 - DNA, 34 bp, synthetic construct
  source 1..34: mol_type = other DNA
ttgatcgaaacaaacnnkgagactggcgaaattg

SEQ ID NO: 1068 - DNA, 29 bp, synthetic construct
  source 1..29: mol_type = other DNA
cgaaacaaacggcnnkactggcgaaattg

SEQ ID NO: 1069 - DNA, 29 bp, synthetic construct
  source 1..29: mol_type = other DNA
aacaaacggcgagnnkggcgaaattgttt

SEQ ID NO: 1070 - DNA, 31 bp, synthetic construct
  source 1..31: mol_type = other DNA
caaacggcgagactnnkgaaattgtttggga

SEQ ID NO: 1071 - DNA, 27 bp, synthetic construct
  source 1..27: mol_type = other DNA
ggcgagactggcnnkattgtttgggac

SEQ ID NO: 1072 - DNA, 29 bp, synthetic construct
  source 1..29: mol_type = other DNA
cgagactggcgaannkgtttgggacaaag

SEQ ID NO: 1073 - DNA, 29 bp, synthetic construct
  source 1..29: mol_type = other DNA
gactggcgaaattnnktgggacaaagggc

SEQ ID NO: 1074 - DNA, 28 bp, synthetic construct
  source 1..28: mol_type = other DNA
ggcgaaattgttnnkgacaaagggcgtg

SEQ ID NO: 1075 - DNA, 32 bp, synthetic construct
  source 1..32: mol_type = other DNA
gcgaaattgtttggnnkaaagggcgtgatttc

SEQ ID NO: 1076 - DNA, 29 bp, synthetic construct
  source 1..29: mol_type = other DNA
aattgtttgggacnnkgggcgtgatttcg

SEQ ID NO: 1077 - DNA, 30 bp, synthetic construct
  source 1..30: mol_type = other DNA
tgtttgggacaaannkcgtgatttcgcgac

SEQ ID NO: 1078 - DNA, 26 bp, synthetic construct
  source 1..26: mol_type = other DNA
gggacaaagggnnkgatttcgcgacg

SEQ ID NO: 1079 - DNA, 25 bp, synthetic construct
  source 1..25: mol_type = other DNA
acaaagggcgtnnkttcgcgacggt

SEQ ID NO: 1080 - DNA, 25 bp, synthetic construct
  source 1..25: mol_type = other DNA
aagggcgtgatnnkgcgacggtgcg

SEQ ID NO: 1081 - DNA, 25 bp, synthetic construct
  source 1..25: mol_type = other DNA
ggcgtgatttcnnkacggtgcgcaa

SEQ ID NO: 1082 - DNA, 26 bp, synthetic construct
  source 1..26: mol_type = other DNA
gtgatttcgcgnnkgtgcgcaaggta

SEQ ID NO: 1083 - DNA, 26 bp, synthetic construct
  source 1..26: mol_type = other DNA
atttcgcgacgnnkcgcaaggtactg

SEQ ID NO: 1084 - DNA, 26 bp, synthetic construct
  source 1..26: mol_type = other DNA
tcgcgacggtgnnkaaggtactgagc

SEQ ID NO: 1085 - DNA, 25 bp, synthetic construct
  source 1..25: mol_type = other DNA
cgacggtgcgcnnkgtactgagcat

SEQ ID NO: 1086 - DNA, 25 bp, synthetic construct
  source 1..25: mol_type = other DNA
cggtgcgcaagnnkctgagcatgcc

SEQ ID NO: 1087 - DNA, 27 bp, synthetic construct
  source 1..27: mol_type = other DNA
gtgcgcaaggtannkagcatgcctcaa

SEQ ID NO: 1088 - DNA, 28 bp, synthetic construct
  source 1..28: mol_type = other DNA
cgcaaggtactgnnkatgcctcaagtca

SEQ ID NO: 1089 - DNA, 33 bp, synthetic construct
  source 1..33: mol_type = other DNA
cgcaaggtactgagcnnkcctcaagtcaatatt

SEQ ID NO: 1090 - DNA, 38 bp, synthetic construct
  source 1..38: mol_type = other DNA
gcaaggtactgagcatgnnkcaagtcaatattgttaag

SEQ ID NO: 1091 - DNA, 38 bp, synthetic construct
  source 1..38: mol_type = other DNA
aggtactgagcatgcctnnkgtcaatattgttaagaaa

SEQ ID NO: 1092 - DNA, 40 bp, synthetic construct
  source 1..40: mol_type = other DNA
gtactgagcatgcctcaannkaatattgttaagaaaaccg

SEQ ID NO: 1093 - DNA, 35 bp, synthetic construct
  source 1..35: mol_type = other DNA
gagcatgcctcaagtcnnkattgttaagaaaaccg

SEQ ID NO: 1094 - DNA, 36 bp, synthetic construct
  source 1..36: mol_type = other DNA
catgcctcaagtcaatnnkgttaagaaaaccgaagt

SEQ ID NO: 1095 - DNA, 37 bp, synthetic construct
  source 1..37: mol_type = other DNA
tgcctcaagtcaatattnnkaagaaaaccgaagtgca

SEQ ID NO: 1096 - DNA, 38 bp, synthetic construct
  source 1..38: mol_type = other DNA
ctcaagtcaatattgttnnkaaaaccgaagtgcagacg

SEQ ID NO: 1097 - DNA, 35 bp, synthetic construct
  source 1..35: mol_type = other DNA
agtcaatattgttaagnnkaccgaagtgcagacgg

SEQ ID NO: 1098 - DNA, 35 bp, synthetic construct
  source 1..35: mol_type = other DNA
caatattgttaagaaannkgaagtgcagacgggcg

SEQ ID NO: 1099 - DNA, 28 bp, synthetic construct
  source 1..28: mol_type = other DNA
gttaagaaaaccnnkgtgcagacgggcg

SEQ ID NO: 1100 - DNA, 25 bp, synthetic construct
  source 1..25: mol_type = other DNA
agaaaaccgaannkcagacgggcgg

SEQ ID NO: 1101 - DNA, 25 bp, synthetic construct
  source 1..25: mol_type = other DNA
aaaccgaagtgnnkacgggcgggtt

SEQ ID NO: 1102 - DNA, 25 bp, synthetic construct
  source 1..25: mol_type = other DNA
ccgaagtgcagnnkggcgggttttc

SEQ ID NO: 1103 - DNA, 28 bp, synthetic construct
  source 1..28: mol_type = other DNA
gaagtgcagacgnnkgggttttccaagg

SEQ ID NO: 1104 - DNA, 29 bp, synthetic construct
  source 1..29: mol_type = other DNA
agtgcagacgggcnnkttttccaaggaaa

SEQ ID NO: 1105 - DNA, 25 bp, synthetic construct
  source 1..25: mol_type = other DNA
agacgggcgggnnktccaaggaaag

SEQ ID NO: 1106 - DNA, 28 bp, synthetic construct
  source 1..28: mol_type = other DNA
acgggcgggtttnnkaaggaaagcatct

SEQ ID NO: 1107 - DNA, 29 bp, synthetic construct
  source 1..29: mol_type = other DNA
gggcgggttttccnnkgaaagcatcttac

SEQ ID NO: 1108 - DNA, 30 bp, synthetic construct
  source 1..30: mol_type = other DNA
cgggttttccaagnnkagcatcttacccaa

SEQ ID NO: 1109 - DNA, 35 bp, synthetic construct
  source 1..35: mol_type = other DNA
cgggttttccaaggaannkatcttacccaaacgta

SEQ ID NO: 1110 - DNA, 37 bp, synthetic construct
  source 1..37: mol_type = other DNA
ggttttccaaggaaagcnnkttacccaaacgtaattc

SEQ ID NO: 1111 - DNA, 34 bp, synthetic construct
  source 1..34: mol_type = other DNA
tccaaggaaagcatcnnkcccaaacgtaattcag

SEQ ID NO: 1112 - DNA, 49 bp, synthetic construct
  source 1..49: mol_type = other DNA
ggttttccaaggaaagcatcttannkaaacgtaattcagataaacttat

SEQ ID NO: 1113 - DNA, 41 bp, synthetic construct
  source 1..41: mol_type = other DNA
caaggaaagcatcttacccnnkcgtaattcagataaactta

SEQ ID NO: 1114 - DNA, 44 bp, synthetic construct
  source 1..44: mol_type = other DNA
aggaaagcatcttacccaaannkaattcagataaacttattgca

SEQ ID NO: 1115 - DNA, 39 bp, synthetic construct
  source 1..39: mol_type = other DNA
agcatcttacccaaacgtnnktcagataaacttattgca

SEQ ID NO: 1116 - DNA, 39 bp, synthetic construct
  source 1..39: mol_type = other DNA
atcttacccaaacgtaatnnkgataaacttattgcacgc

SEQ ID NO: 1117 - DNA, 37 bp, synthetic construct
  source 1..37: mol_type = other DNA
tacccaaacgtaattcannkaaacttattgcacgcaa

SEQ ID NO: 1118 - DNA, 39 bp, synthetic construct
  source 1..39: mol_type = other DNA
cccaaacgtaattcagatnnkcttattgcacgcaaaaag

SEQ ID NO: 1119 - DNA, 40 bp, synthetic construct
  source 1..40: mol_type = other DNA
aaacgtaattcagataaannkattgcacgcaaaaaggact

SEQ ID NO: 1120 - DNA, 38 bp, synthetic construct
  source 1..38: mol_type = other DNA
gtaattcagataaacttnnkgcacgcaaaaaggactgg

SEQ ID NO: 1121 - DNA, 42 bp, synthetic construct
  source 1..42: mol_type = other DNA
taattcagataaacttattnnkcgcaaaaaggactgggatcc

SEQ ID NO: 1122 - DNA, 38 bp, synthetic construct
  source 1..38: mol_type = other DNA
cagataaacttattgcannkaaaaaggactgggatccg

SEQ ID NO: 1123 - DNA, 30 bp, synthetic construct
  source 1..30: mol_type = other DNA
acttattgcacgcnnkaaggactgggatcc

SEQ ID NO: 1124 - DNA, 31 bp, synthetic construct
  source 1..31: mol_type = other DNA
ttattgcacgcaaannkgactgggatccgaa

SEQ ID NO: 1125 - DNA, 31 bp, synthetic construct
  source 1..31: mol_type = other DNA
ttgcacgcaaaaagnnktgggatccgaaaaa

SEQ ID NO: 1126 - DNA, 33 bp, synthetic construct
  source 1..33: mol_type = other DNA
gcacgcaaaaaggacnnkgatccgaaaaagtat

SEQ ID NO: 1127 - DNA, 32 bp, synthetic construct
  source 1..32: mol_type = other DNA
gcaaaaaggactggnnkccgaaaaagtatgga

SEQ ID NO: 1128 - DNA, 37 bp, synthetic construct
  source 1..37: mol_type = other DNA
gcaaaaaggactgggatnnkaaaaagtatggaggctt

SEQ ID NO: 1129 - DNA, 28 bp, synthetic construct
  source 1..28: mol_type = other DNA
gactgggatccgnnkaagtatggaggct

SEQ ID NO: 1130 - DNA, 31 bp, synthetic construct
  source 1..31: mol_type = other DNA
actgggatccgaaannktatggaggcttcga

SEQ ID NO: 1131 - DNA, 29 bp, synthetic construct
  source 1..29: mol_type = other DNA
ggatccgaaaaagnnkggaggcttcgaca

SEQ ID NO: 1132 - DNA, 34 bp, synthetic construct
  source 1..34: mol_type = other DNA
gatccgaaaaagtatnnkggcttcgacagtccaa

SEQ ID NO: 1133 - DNA, 33 bp, synthetic construct
  source 1..33: mol_type = other DNA
ccgaaaaagtatggannkttcgacagtccaacc

SEQ ID NO: 1134 - DNA, 29 bp, synthetic construct
  source 1..29: mol_type = other DNA
aaagtatggaggcnnkgacagtccaaccg

SEQ ID NO: 1135 - DNA, 30 bp, synthetic construct
  source 1..30: mol_type = other DNA
gtatggaggcttcnnkagtccaaccgtagc

SEQ ID NO: 1136 - DNA, 25 bp, synthetic construct
  source 1..25: mol_type = other DNA
gaggcttcgacnnkccaaccgtagc

SEQ ID NO: 1137 - DNA, 29 bp, synthetic construct
  source 1..29: mol_type = other DNA
aggcttcgacagtnnkaccgtagcctact

SEQ ID NO: 1138 - DNA, 30 bp, synthetic construct
  source 1..30: mol_type = other DNA
cttcgacagtccannkgtagcctactctgt

SEQ ID NO: 1139 - DNA, 29 bp, synthetic construct
  source 1..29: mol_type = other DNA
cgacagtccaaccnnkgcctactctgttc

SEQ ID NO: 1140 - DNA, 34 bp, synthetic construct
  source 1..34: mol_type = other DNA
gacagtccaaccgtannktactctgttctcgttg

SEQ ID NO: 1141 - DNA, 29 bp, synthetic construct
  source 1..29: mol_type = other DNA
tccaaccgtagccnnktctgttctcgttg

SEQ ID NO: 1142 - DNA, 30 bp, synthetic construct
  source 1..30: mol_type = other DNA
aaccgtagcctacnnkgttctcgttgtagc

SEQ ID NO: 1143 - DNA, 30 bp, synthetic construct
  source 1..30: mol_type = other DNA
cgtagcctactctnnkctcgttgtagcgaa

SEQ ID NO: 1144 - DNA, 35 bp, synthetic construct
  source 1..35: mol_type = other DNA
cgtagcctactctgttnnkgttgtagcgaaagtag

SEQ ID NO: 1145 - DNA, 37 bp, synthetic construct
  source 1..37: mol_type = other DNA
tagcctactctgttctcnnkgtagcgaaagtagaaaa

SEQ ID NO: 1146 - DNA, 36 bp, synthetic construct
  source 1..36: mol_type = other DNA
ctactctgttctcgttnnkgcgaaagtagaaaaggg

SEQ ID NO: 1147 - DNA, 43 bp, synthetic construct
  source 1..43: mol_type = other DNA
cctactctgttctcgttgtannkaaagtagaaaagggtaaatc

SEQ ID NO: 1148 - DNA, 37 bp, synthetic construct
  source 1..37: mol_type = other DNA
ctgttctcgttgtagcgnnkgtagaaaagggtaaatc

SEQ ID NO: 1149 - DNA, 37 bp, synthetic construct
  source 1..37: mol_type = other DNA
ttctcgttgtagcgaaannkgaaaagggtaaatccaa

SEQ ID NO: 1150 - DNA, 37 bp, synthetic construct
  source 1..37: mol_type = other DNA
tcgttgtagcgaaagtannkaagggtaaatccaagaa

SEQ ID NO: 1151 - DNA, 40 bp, synthetic construct
  source 1..40: mol_type = other DNA
gttgtagcgaaagtagaannkggtaaatccaagaaactga

SEQ ID NO: 1152 - DNA, 44 bp, synthetic construct
  source 1..44: mol_type = other DNA
ttgtagcgaaagtagaaaagnnkaaatccaagaaactgaaatct

SEQ ID NO: 1153 - DNA, 39 bp, synthetic construct
  source 1..39: mol_type = other DNA
gcgaaagtagaaaagggtnnktccaagaaactgaaatct

SEQ ID NO: 1154 - DNA, 44 bp, synthetic construct
  source 1..44: mol_type = other DNA
cgaaagtagaaaagggtaaannkaagaaactgaaatctgtcaag

SEQ ID NO: 1155 - DNA, 41 bp, synthetic construct
  source 1..41: mol_type = other DNA
agtagaaaagggtaaatccnnkaaactgaaatctgtcaagg

SEQ ID NO: 1156 - DNA, 37 bp, synthetic construct
  source 1..37: mol_type = other DNA
aaaagggtaaatccaagnnkctgaaatctgtcaagga

SEQ ID NO: 1157 - DNA, 40 bp, synthetic construct
  source 1..40: mol_type = other DNA
aagggtaaatccaagaaannkaaatctgtcaaggagttgc

SEQ ID NO: 1158 - DNA, 37 bp, synthetic construct
  source 1..37: mol_type = other DNA
gtaaatccaagaaactgnnktctgtcaaggagttgct

SEQ ID NO: 1159 - DNA, 35 bp, synthetic construct
  source 1..35: mol_type = other DNA
atccaagaaactgaaannkgtcaaggagttgcttg

SEQ ID NO: 1160 - DNA, 39 bp, synthetic construct
  source 1..39: mol_type = other DNA
tccaagaaactgaaatctnnkaaggagttgcttggaatc

SEQ ID NO: 1161 - DNA, 36 bp, synthetic construct
  source 1..36: mol_type = other DNA
gaaactgaaatctgtcnnkgagttgcttggaatcac

SEQ ID NO: 1162 - DNA, 37 bp, synthetic construct
  source 1..37: mol_type = other DNA
aactgaaatctgtcaagnnkttgcttggaatcaccat

SEQ ID NO: 1163 - DNA, 37 bp, synthetic construct
  source 1..37: mol_type = other DNA
tgaaatctgtcaaggagnnkcttggaatcaccattat

SEQ ID NO: 1164 - DNA, 37 bp, synthetic construct
  source 1..37: mol_type = other DNA
aatctgtcaaggagttgnnkggaatcaccattatgga

SEQ ID NO: 1165 - DNA, 35 bp, synthetic construct
  source 1..35: mol_type = other DNA
tgtcaaggagttgcttnnkatcaccattatggagc

SEQ ID NO: 1166 - DNA, 31 bp, synthetic construct
  source 1..31: mol_type = other DNA
aggagttgcttggannkaccattatggagcg

SEQ ID NO: 1167 - DNA, 34 bp, synthetic construct
  source 1..34: mol_type = other DNA
gagttgcttggaatcnnkattatggagcgtagct

SEQ ID NO: 1168 - DNA, 28 bp, synthetic construct
  source 1..28: mol_type = other DNA
cttggaatcaccnnkatggagcgtagct

SEQ ID NO: 1169 - DNA, 32 bp, synthetic construct
  source 1..32: mol_type = other DNA
ttggaatcaccattnnkgagcgtagctccttc

SEQ ID NO: 1170 - DNA, 33 bp, synthetic construct
  source 1..33: mol_type = other DNA
ggaatcaccattatgnnkcgtagctccttcgag

SEQ ID NO: 1171 - DNA, 36 bp, synthetic construct
  source 1..36: mol_type = other DNA
aatcaccattatggagnnkagctccttcgagaagaa

SEQ ID NO: 1172 - DNA, 33 bp, synthetic construct
  source 1..33: mol_type = other DNA
accattatggagcgtnnktccttcgagaagaac

SEQ ID NO: 1173 - DNA, 31 bp, synthetic construct
  source 1..31: mol_type = other DNA
ttatggagcgtagcnnkttcgagaagaaccc

SEQ ID NO: 1174 - DNA, 27 bp, synthetic construct
  source 1..27: mol_type = other DNA
gagcgtagctccnnkgagaagaacccg

SEQ ID NO: 1175 - DNA, 30 bp, synthetic construct
  source 1..30: mol_type = other DNA
gcgtagctccttcnnkaagaacccgattga

SEQ ID NO: 1176 - DNA, 32 bp, synthetic construct
  source 1..32: mol_type = other DNA
gtagctccttcgagnnkaacccgattgacttt

SEQ ID NO: 1177 - DNA, 31 bp, synthetic construct
  source 1..31: mol_type = other DNA
gctccttcgagaagnnkccgattgactttct

SEQ ID NO: 1178 - DNA, 36 bp, synthetic construct
  source 1..36: mol_type = other DNA
ctccttcgagaagaacnnkattgactttctggaagc

SEQ ID NO: 1179 - DNA, 29 bp, synthetic construct
  source 1..29: mol_type = other DNA
cgagaagaacccgnnkgactttctggaag

SEQ ID NO: 1180 - DNA, 34 bp, synthetic construct
  source 1..34: mol_type = other DNA
gagaagaacccgattnnktttctggaagccaaag

SEQ ID NO: 1181 - DNA, 29 bp, synthetic construct
  source 1..29: mol_type = other DNA
gaacccgattgacnnkctggaagccaaag

SEQ ID NO: 1182 - DNA, 37 bp, synthetic construct
  source 1..37: mol_type = other DNA
agaacccgattgactttnnkgaagccaaaggatataa

SEQ ID NO: 1183 - DNA, 36 bp, synthetic construct
  source 1..36: mol_type = other DNA
cccgattgactttctgnnkgccaaaggatataaaga

SEQ ID NO: 1184 - DNA, 40 bp, synthetic construct
  source 1..40: mol_type = other DNA
ccgattgactttctggaannkaaaggatataaagaggtca

SEQ ID NO: 1185 - DNA, 37 bp, synthetic construct
  source 1..37: mol_type = other DNA
ttgactttctggaagccnnkggatataaagaggtcaa

SEQ ID NO: 1186 - DNA, 41 bp, synthetic construct
  source 1..41: mol_type = other DNA
tgactttctggaagccaaannktataaagaggtcaagaaag

SEQ ID NO: 1187 - DNA, 35 bp, synthetic construct
  source 1..35: mol_type = other DNA
tctggaagccaaaggannkaaagaggtcaagaaag

SEQ ID NO: 1188 - DNA, 37 bp, synthetic construct
  source 1..37: mol_type = other DNA
tggaagccaaaggatatnnkgaggtcaagaaagatct

SEQ ID NO: 1189 - DNA, 45 bp, synthetic construct
  source 1..45: mol_type = other DNA
ctggaagccaaaggatataaannkgtcaagaaagatcttatcatt

SEQ ID NO: 1190 - DNA, 47 bp, synthetic construct
  source 1..47: mol_type = other DNA
ggaagccaaaggatataaagagnnkaagaaagatcttatcattaagc

SEQ ID NO: 1191 - DNA, 45 bp, synthetic construct
  source 1..45: mol_type = other DNA
gccaaaggatataaagaggtcnnkaaagatcttatcattaagctg

SEQ ID NO: 1192 - DNA, 42 bp, synthetic construct
  source 1..42: mol_type = other DNA
aggatataaagaggtcaagnnkgatcttatcattaagctgcc

SEQ ID NO: 1193 - DNA, 45 bp, synthetic construct
  source 1..45: mol_type = other DNA
ggatataaagaggtcaagaaannkcttatcattaagctgcctaag

SEQ ID NO: 1194 - DNA, 50 bp, synthetic construct
  source 1..50: mol_type = other DNA
gatataaagaggtcaagaaagatnnkatcattaagctgcctaagtattca

SEQ ID NO: 1195 - DNA, 44 bp, synthetic construct
  source 1..44: mol_type = other DNA
aagaggtcaagaaagatcttnnkattaagctgcctaagtattca

SEQ ID NO: 1196 - DNA, 41 bp, synthetic construct
  source 1..41: mol_type = other DNA
ggtcaagaaagatcttatcnnkaagctgcctaagtattcac

SEQ ID NO: 1197 - DNA, 45 bp, synthetic construct
  source 1..45: mol_type = other DNA
gtcaagaaagatcttatcattnnkctgcctaagtattcactcttc

SEQ ID NO: 1198 - DNA, 46 bp, synthetic construct
  source 1..46: mol_type = other DNA
aagaaagatcttatcattaagnnkcctaagtattcactcttcgagc

SEQ ID NO: 1199 - DNA, 42 bp, synthetic construct
  source 1..42: mol_type = other DNA
agatcttatcattaagctgnnkaagtattcactcttcgagct

SEQ ID NO: 1200 - DNA, 38 bp, synthetic construct
  source 1..38: mol_type = other DNA
ttatcattaagctgcctnnktattcactcttcgagctg

SEQ ID NO: 1201 - DNA, 33 bp, synthetic construct
  source 1..33: mol_type = other DNA
attaagctgcctaagnnktcactcttcgagctg

SEQ ID NO: 1202 - DNA, 37 bp, synthetic construct
  source 1..37: mol_type = other DNA
ttaagctgcctaagtatnnkctcttcgagctggaaaa

SEQ ID NO: 1203 - DNA, 35 bp, synthetic construct
  source 1..35: mol_type = other DNA
gctgcctaagtattcannkttcgagctggaaaatg

SEQ ID NO: 1204 - DNA, 35 bp, synthetic construct
  source 1..35: mol_type = other DNA
gcctaagtattcactcnnkgagctggaaaatggtc

SEQ ID NO: 1205 - DNA, 40 bp, synthetic construct
  source 1..40: mol_type = other DNA
cctaagtattcactcttcnnkctggaaaatggtcgtaaac

SEQ ID NO: 1206 - DNA, 37 bp, synthetic construct
  source 1..37: mol_type = other DNA
agtattcactcttcgagnnkgaaaatggtcgtaaacg

SEQ ID NO: 1207 - DNA, 31 bp, synthetic construct
  source 1..31: mol_type = other DNA
cactcttcgagctgnnkaatggtcgtaaacg

SEQ ID NO: 1208 - DNA, 28 bp, synthetic construct
  source 1..28: mol_type = other DNA
ttcgagctggaannkggtcgtaaacgca

SEQ ID NO: 1209 - DNA, 30 bp, synthetic construct
  source 1..30: mol_type = other DNA
cgagctggaaaatnnkcgtaaacgcatgct

SEQ ID NO: 1210 - DNA, 29 bp, synthetic construct
  source 1..29: mol_type = other DNA
gctggaaaatggtnnkaaacgcatgctcg

SEQ ID NO: 1211 - DNA, 28 bp, synthetic construct
  source 1..28: mol_type = other DNA
gaaaatggtcgtnnkcgcatgctcgctt

SEQ ID NO: 1212 - DNA, 32 bp, synthetic construct
  source 1..32: mol_type = other DNA
aaaatggtcgtaaannkatgctcgcttctgcc

SEQ ID NO: 1213 - DNA, 26 bp, synthetic construct
  source 1..26: mol_type = other DNA
gtcgtaaacgcnnkctcgcttctgcc

SEQ ID NO: 1214 - DNA, 25 bp, synthetic construct
  source 1..25: mol_type = other DNA
gtaaacgcatgnnkgcttctgccgg

SEQ ID NO: 1215 - DNA, 25 bp, synthetic construct
  source 1..25: mol_type = other DNA
aacgcatgctcnnktctgccggcga

SEQ ID NO: 1216 - DNA, 25 bp, synthetic construct
  source 1..25: mol_type = other DNA
gcatgctcgctnnkgccggcgagtt

SEQ ID NO: 1217 - DNA, 25 bp, synthetic construct
  source 1..25: mol_type = other DNA
tgctcgcttctnnkggcgagttgca

SEQ ID NO: 1218 - DNA, 27 bp, synthetic construct
  source 1..27: mol_type = other DNA
ctcgcttctgccnnkgagttgcagaag

SEQ ID NO: 1219 - DNA, 25 bp, synthetic construct
  source 1..25: mol_type = other DNA
cttctgccggcnnkttgcagaaggg

SEQ ID NO: 1220 - DNA, 25 bp, synthetic construct
  source 1..25: mol_type = other DNA
ctgccggcgagnnkcagaagggcaa

SEQ ID NO: 1221 - DNA, 26 bp, synthetic construct
  source 1..26: mol_type = other DNA
ccggcgagttgnnkaagggcaatgaa

SEQ ID NO: 1222 - DNA, 29 bp, synthetic construct
  source 1..29: mol_type = other DNA
cggcgagttgcagnnkggcaatgaattag

SEQ ID NO: 1223 - DNA, 34 bp, synthetic construct
  source 1..34: mol_type = other DNA
ggcgagttgcagaagnnkaatgaattagcacttc

SEQ ID NO: 1224 - DNA, 30 bp, synthetic construct
  source 1..30: mol_type = other DNA
gttgcagaagggcnnkgaattagcacttcc

SEQ ID NO: 1225 - DNA, 33 bp, synthetic construct
  source 1..33: mol_type = other DNA
ttgcagaagggcaatnnkttagcacttccatca

SEQ ID NO: 1226 - DNA, 33 bp, synthetic construct
  source 1..33: mol_type = other DNA
cagaagggcaatgaannkgcacttccatcaaag

SEQ ID NO: 1227 - DNA, 37 bp, synthetic construct
  source 1..37: mol_type = other DNA
agaagggcaatgaattannkcttccatcaaagtacgt

SEQ ID NO: 1228 - DNA, 37 bp, synthetic construct
  source 1..37: mol_type = other DNA
agggcaatgaattagcannkccatcaaagtacgttaa

SEQ ID NO: 1229 - DNA, 40 bp, synthetic construct
  source 1..40: mol_type = other DNA
ggcaatgaattagcacttnnktcaaagtacgttaacttcc

SEQ ID NO: 1230 - DNA, 40 bp, synthetic construct
  source 1..40: mol_type = other DNA
aatgaattagcacttccannkaagtacgttaacttcctgt

SEQ ID NO: 1231 - DNA, 44 bp, synthetic construct
  source 1..44: mol_type = other DNA
atgaattagcacttccatcannktacgttaacttcctgtatttg

SEQ ID NO: 1232 - DNA, 40 bp, synthetic construct
  source 1..40: mol_type = other DNA
ttagcacttccatcaaagnnkgttaacttcctgtatttgg

SEQ ID NO: 1233 - DNA, 37 bp, synthetic construct
  source 1..37: mol_type = other DNA
cacttccatcaaagtacnnkaacttcctgtatttggc

SEQ ID NO: 1234 - DNA, 36 bp, synthetic construct
  source 1..36: mol_type = other DNA
tccatcaaagtacgttnnkttcctgtatttggccag

SEQ ID NO: 1235 - DNA, 34 bp, synthetic construct
  source 1..34: mol_type = other DNA
tcaaagtacgttaacnnkctgtatttggccagcc

SEQ ID NO: 1236 - DNA, 38 bp, synthetic construct
  source 1..38: mol_type = other DNA
caaagtacgttaacttcnnktatttggccagccattac

SEQ ID NO: 1237 - DNA, 33 bp, synthetic construct
  source 1..33: mol_type = other DNA
tacgttaacttcctgnnkttggccagccattac

SEQ ID NO: 1238 - DNA, 34 bp, synthetic construct
  source 1..34: mol_type = other DNA
gttaacttcctgtatnnkgccagccattacgaga

SEQ ID NO: 1239 - DNA, 40 bp, synthetic construct
  source 1..40: mol_type = other DNA
gttaacttcctgtatttgnnkagccattacgagaaactga

SEQ ID NO: 1240 - DNA, 36 bp, synthetic construct
  source 1..36: mol_type = other DNA
cttcctgtatttggccnnkcattacgagaaactgaa

SEQ ID NO: 1241 - DNA, 33 bp, synthetic construct
  source 1..33: mol_type = other DNA
ctgtatttggccagcnnktacgagaaactgaag

SEQ ID NO: 1242 - DNA, 29 bp, synthetic construct
  source 1..29: mol_type = other DNA
tttggccagccatnnkgagaaactgaagg

SEQ ID NO: 1243 - DNA, 28 bp, synthetic construct
  source 1..28: mol_type = other DNA
gccagccattacnnkaaactgaaggggt

SEQ ID NO: 1244 - DNA, 29 bp, synthetic construct
  source 1..29: mol_type = other DNA
cagccattacgagnnkctgaaggggtctc

SEQ ID NO: 1245 - DNA, 32 bp, synthetic construct
  source 1..32: mol_type = other DNA
gccattacgagaaannkaaggggtctccagag

SEQ ID NO: 1246 - DNA, 32 bp, synthetic construct
  source 1..32: mol_type = other DNA
attacgagaaactgnnkgggtctccagaggac

SEQ ID NO: 1247 - DNA, 33 bp, synthetic construct
  source 1..33: mol_type = other DNA
tacgagaaactgaagnnktctccagaggacaac

SEQ ID NO: 1248 - DNA, 28 bp, synthetic construct
  source 1..28: mol_type = other DNA
aaactgaaggggnnkccagaggacaacg

SEQ ID NO: 1249 - DNA, 29 bp, synthetic construct
  source 1..29: mol_type = other DNA
actgaaggggtctnnkgaggacaacgaac

SEQ ID NO: 1250 - DNA, 30 bp, synthetic construct
  source 1..30: mol_type = other DNA
gaaggggtctccannkgacaacgaacagaa

SEQ ID NO: 1251 - DNA, 31 bp, synthetic construct
  source 1..31: mol_type = other DNA
aggggtctccagagnnkaacgaacagaaaca

SEQ ID NO: 1252 - DNA, 34 bp, synthetic construct
  source 1..34: mol_type = other DNA
gggtctccagaggacnnkgaacagaaacaattat

SEQ ID NO: 1253 - DNA, 37 bp, synthetic construct
  source 1..37: mol_type = other DNA
ggtctccagaggacaacnnkcagaaacaattatttgt

SEQ ID NO: 1254 - DNA, 40 bp, synthetic construct
  source 1..40: mol_type = other DNA
tctccagaggacaacgaannkaaacaattatttgtagagc

SEQ ID NO: 1255 - DNA, 37 bp, synthetic construct
  source 1..37: mol_type = other DNA
cagaggacaacgaacagnnkcaattatttgtagagca

SEQ ID NO: 1256 - DNA, 37 bp, synthetic construct
  source 1..37: mol_type = other DNA
aggacaacgaacagaaannkttatttgtagagcagca

SEQ ID NO: 1257 - DNA, 36 bp, synthetic construct
  source 1..36: mol_type = other DNA
caacgaacagaaacaannktttgtagagcagcacaa

SEQ ID NO: 1258 - DNA, 35 bp, synthetic construct
  source 1..35: mol_type = other DNA
cgaacagaaacaattannkgtagagcagcacaagc

SEQ ID NO: 1259 - DNA, 41 bp, synthetic construct
  source 1..41: mol_type = other DNA
cgaacagaaacaattatttnnkgagcagcacaagcattatc

SEQ ID NO: 1260 - DNA, 45 bp, synthetic construct
  source 1..45: mol_type = other DNA
gaacagaaacaattatttgtannkcagcacaagcattatcttgat

SEQ ID NO: 1261 - DNA, 46 bp, synthetic construct
  source 1..46: mol_type = other DNA
cagaaacaattatttgtagagnnkcacaagcattatcttgatgaaa

SEQ ID NO: 1262 - DNA, 49 bp, synthetic construct
  source 1..49: mol_type = other DNA
agaaacaattatttgtagagcagnnkaagcattatcttgatgaaatcat

SEQ ID NO: 1263 - DNA, 46 bp, synthetic construct
  source 1..46: mol_type = other DNA
caattatttgtagagcagcacnnkcattatcttgatgaaatcattg

SEQ ID NO: 1264 - DNA, 42 bp, synthetic construct
  source 1..42: mol_type = other DNA
atttgtagagcagcacaagnnktatcttgatgaaatcattga

SEQ ID NO: 1265 - DNA, 37 bp, synthetic construct
  source 1..37: mol_type = other DNA
tagagcagcacaagcatnnkcttgatgaaatcattga

SEQ ID NO: 1266 - DNA, 36 bp, synthetic construct
  source 1..36: mol_type = other DNA
gcagcacaagcattatnnkgatgaaatcattgagca

SEQ ID NO: 1267 - DNA, 41 bp, synthetic construct
  source 1..41: mol_type = other DNA
gcagcacaagcattatcttnnkgaaatcattgagcaaattt

SEQ ID NO: 1268 - DNA, 41 bp, synthetic construct
  source 1..41: mol_type = other DNA
gcacaagcattatcttgatnnkatcattgagcaaatttccg

SEQ ID NO: 1269 - DNA, 43 bp, synthetic construct
  source 1..43: mol_type = other DNA
acaagcattatcttgatgaannkattgagcaaatttccgaatt

SEQ ID NO: 1270 - DNA, 41 bp, synthetic construct
  source 1..41: mol_type = other DNA
gcattatcttgatgaaatcnnkgagcaaatttccgaattca

SEQ ID NO: 1271 - DNA, 48 bp, synthetic construct
  source 1..48: mol_type = other DNA
gcattatcttgatgaaatcattnnkcaaatttccgaattcagtaaacg

SEQ ID NO: 1272 - DNA, 44 bp, synthetic construct
  source 1..44: mol_type = other DNA
atcttgatgaaatcattgagnnkatttccgaattcagtaaacgc

SEQ ID NO: 1273 - DNA, 38 bp, synthetic construct
  source 1..38: mol_type = other DNA
atgaaatcattgagcaannktccgaattcagtaaacgc

SEQ ID NO: 1274 - DNA, 41 bp, synthetic construct
  source 1..41: mol_type = other DNA
tgaaatcattgagcaaattnnkgaattcagtaaacgcgtaa

SEQ ID NO: 1275 - DNA, 38 bp, synthetic construct
  source 1..38: mol_type = other DNA
tcattgagcaaatttccnnkttcagtaaacgcgtaatc

SEQ ID NO: 1276 - DNA, 35 bp, synthetic construct
  source 1..35: mol_type = other DNA
tgagcaaatttccgaannkagtaaacgcgtaatcc

SEQ ID NO: 1277 - DNA, 34 bp, synthetic construct
  source 1..34: mol_type = other DNA
caaatttccgaattcnnkaaacgcgtaatcctgg

SEQ ID NO: 1278 - DNA, 29 bp, synthetic construct
  source 1..29: mol_type = other DNA
ttccgaattcagtnnkcgcgtaatcctgg

SEQ ID NO: 1279 - DNA, 35 bp, synthetic construct
  source 1..35: mol_type = other DNA
ttccgaattcagtaaannkgtaatcctggccgatg

SEQ ID NO: 1280 - DNA, 29 bp, synthetic construct
  source 1..29: mol_type = other DNA
attcagtaaacgcnnkatcctggccgatg

SEQ ID NO: 1281 - DNA, 26 bp, synthetic construct
  source 1..26: mol_type = other DNA
gtaaacgcgtannkctggccgatgca

SEQ ID NO: 1282 - DNA, 29 bp, synthetic construct
  source 1..29: mol_type = other DNA
taaacgcgtaatcnnkgccgatgcaaacc

SEQ ID NO: 1283 - DNA, 28 bp, synthetic construct
  source 1..28: mol_type = other DNA
cgcgtaatcctgnnkgatgcaaacctcg

SEQ ID NO: 1284 - DNA, 27 bp, synthetic construct
  source 1..27: mol_type = other DNA
gtaatcctggccnnkgcaaacctcgac

SEQ ID NO: 1285 - DNA, 31 bp, synthetic construct
  source 1..31: mol_type = other DNA
taatcctggccgatnnkaacctcgacaaggt

SEQ ID NO: 1286 - DNA, 25 bp, synthetic construct
  source 1..25: mol_type = other DNA
tggccgatgcannkctcgacaaggt

SEQ ID NO: 1287 - DNA, 26 bp, synthetic construct
  source 1..26: mol_type = other DNA
ccgatgcaaacnnkgacaaggtgctg

SEQ ID NO: 1288 - DNA, 28 bp, synthetic construct
  source 1..28: mol_type = other DNA
gatgcaaacctcnnkaaggtgctgagcg

SEQ ID NO: 1289 - DNA, 25 bp, synthetic construct
  source 1..25: mol_type = other DNA
caaacctcgacnnkgtgctgagcgc

SEQ ID NO: 1290 - DNA, 29 bp, synthetic construct
  source 1..29: mol_type = other DNA
aaacctcgacaagnnkctgagcgcttaca

SEQ ID NO: 1291 - DNA, 33 bp, synthetic construct
  source 1..33: mol_type = other DNA
aacctcgacaaggtgnnkagcgcttacaataag

SEQ ID NO: 1292 - DNA, 32 bp, synthetic construct
  source 1..32: mol_type = other DNA
tcgacaaggtgctgnnkgcttacaataagcat

SEQ ID NO: 1293 - DNA, 31 bp, synthetic construct
  source 1..31: mol_type = other DNA
acaaggtgctgagcnnktacaataagcatcg

SEQ ID NO: 1294 - DNA, 27 bp, synthetic construct
  source 1..27: mol_type = other DNA
gtgctgagcgctnnkaataagcatcgc

SEQ ID NO: 1295 - DNA, 27 bp, synthetic construct
  source 1..27: mol_type = other DNA
ctgagcgcttacnnkaagcatcgcgac

SEQ ID NO: 1296 - DNA, 29 bp, synthetic construct
  source 1..29: mol_type = other DNA
gagcgcttacaatnnkcatcgcgacaaac

SEQ ID NO: 1297 - DNA, 32 bp, synthetic construct
  source 1..32: mol_type = other DNA
gcgcttacaataagnnkcgcgacaaacctatc

SEQ ID NO: 1298 - DNA, 36 bp, synthetic construct
  source 1..36: mol_type = other DNA
cgcttacaataagcatnnkgacaaacctatccgtga

SEQ ID NO: 1299 - DNA, 34 bp, synthetic construct
  source 1..34: mol_type = other DNA
tacaataagcatcgcnnkaaacctatccgtgagc

SEQ ID NO: 1300 - DNA, 27 bp, synthetic construct
  source 1..27: mol_type = other DNA
aagcatcgcgacnnkcctatccgtgag

SEQ ID NO: 1301 - DNA, 28 bp, synthetic construct
  source 1..28: mol_type = other DNA
catcgcgacaaannkatccgtgagcagg

SEQ ID NO: 1302 - DNA, 25 bp, synthetic construct
  source 1..25: mol_type = other DNA
gcgacaaacctnnkcgtgagcaggc

SEQ ID NO: 1303 - DNA, 30 bp, synthetic construct
  source 1..30: mol_type = other DNA
cgacaaacctatcnnkgagcaggctgaaaa

SEQ ID NO: 1304 - DNA, 36 bp, synthetic construct
  source 1..36: mol_type = other DNA
cgacaaacctatccgtnnkcaggctgaaaatatcat

SEQ ID NO: 1305 - DNA, 37 bp, synthetic construct
  source 1..37: mol_type = other DNA
acaaacctatccgtgagnnkgctgaaaatatcattca

SEQ ID NO: 1306 - DNA, 36 bp, synthetic construct
  source 1..36: mol_type = other DNA
acctatccgtgagcagnnkgaaaatatcattcacct

SEQ ID NO: 1307 - DNA, 36 bp, synthetic construct
  source 1..36: mol_type = other DNA
tatccgtgagcaggctnnkaatatcattcacctgtt

SEQ ID NO: 1308 - DNA, 32 bp, synthetic construct
  source 1..32: mol_type = other DNA
gtgagcaggctgaannkatcattcacctgttc

SEQ ID NO: 1309 - DNA, 36 bp, synthetic construct
  source 1..36: mol_type = other DNA
tgagcaggctgaaaatnnkattcacctgttcacatt

SEQ ID NO: 1310 - DNA, 37 bp, synthetic construct
  source 1..37: mol_type = other DNA
agcaggctgaaaatatcnnkcacctgttcacattaac

SEQ ID NO: 1311 - DNA, 40 bp, synthetic construct
  source 1..40: mol_type = other DNA
caggctgaaaatatcattnnkctgttcacattaacgaacc

SEQ ID NO: 1312 - DNA, 40 bp, synthetic construct
  source 1..40: mol_type = other DNA
gctgaaaatatcattcacnnkttcacattaacgaacctgg

SEQ ID NO: 1313 - DNA, 38 bp, synthetic construct
  source 1..38: mol_type = other DNA
aaaatatcattcacctgnnkacattaacgaacctgggc

SEQ ID NO: 1314 - DNA, 33 bp, synthetic construct
  source 1..33: mol_type = other DNA
atcattcacctgttcnnkttaacgaacctgggc

SEQ ID NO: 1315 - DNA, 27 bp, synthetic construct
  source 1..27: mol_type = other DNA
cacctgttcacannkacgaacctgggc

SEQ ID NO: 1316 - DNA, 29 bp, synthetic construct
  source 1..29: mol_type = other DNA
cctgttcacattannkaacctgggcgctc

SEQ ID NO: 1317 - DNA, 25 bp, synthetic construct
  source 1..25: mol_type = other DNA
tcacattaacgnnkctgggcgctcc

SEQ ID NO: 1318 - DNA, 25 bp, synthetic construct
  source 1..25: mol_type = other DNA
cattaacgaacnnkggcgctccggc

SEQ ID NO: 1319 - DNA, 25 bp, synthetic construct
  source 1..25: mol_type = other DNA
taacgaacctgnnkgctccggccgc

SEQ ID NO: 1320 - DNA, 25 bp, synthetic construct
  source 1..25: mol_type = other DNA
cgaacctgggcnnkccggccgcttt

SEQ ID NO: 1321 - DNA, 25 bp, synthetic construct
  source 1..25: mol_type = other DNA
acctgggcgctnnkgccgcttttaa

SEQ ID NO: 1322 - DNA, 30 bp, synthetic construct
  source 1..30: mol_type = other DNA
cctgggcgctccgnnkgcttttaaatattt

SEQ ID NO: 1323 - DNA, 29 bp, synthetic construct
  source 1..29: mol_type = other DNA
gggcgctccggccnnktttaaatatttcg

SEQ ID NO: 1324 - DNA, 28 bp, synthetic construct
  source 1..28: mol_type = other DNA
gctccggccgctnnkaaatatttcgaca

SEQ ID NO: 1325 - DNA, 28 bp, synthetic construct
  source 1..28: mol_type = other DNA
ccggccgcttttnnktatttcgacacga

SEQ ID NO: 1326 - DNA, 31 bp, synthetic construct
  source 1..31: mol_type = other DNA
cggccgcttttaaannkttcgacacgacaat

SEQ ID NO: 1327 - DNA, 34 bp, synthetic construct
  source 1..34: mol_type = other DNA
gccgcttttaaatatnnkgacacgacaatcgacc

SEQ ID NO: 1328 - DNA, 37 bp, synthetic construct
  source 1..37: mol_type = other DNA
ccgcttttaaatatttcnnkacgacaatcgaccgtaa

SEQ ID NO: 1329 - DNA, 38 bp, synthetic construct
  source 1..38: mol_type = other DNA
cttttaaatatttcgacnnkacaatcgaccgtaagcgc

SEQ ID NO: 1330 - DNA, 31 bp, synthetic construct
  source 1..31: mol_type = other DNA
aatatttcgacacgnnkatcgaccgtaagcg

SEQ ID NO: 1331 - DNA, 25 bp, synthetic construct
  source 1..25: mol_type = other DNA
tcgacacgacannkgaccgtaagcg

SEQ ID NO: 1332 - DNA, 31 bp, synthetic construct
  source 1..31: mol_type = other DNA
tcgacacgacaatcnnkcgtaagcgctatac

SEQ ID NO: 1333 - DNA, 30 bp, synthetic construct
  source 1..30: mol_type = other DNA
cacgacaatcgacnnkaagcgctataccag

SEQ ID NO: 1334 - DNA, 31 bp, synthetic construct
  source 1..31: mol_type = other DNA
cgacaatcgaccgtnnkcgctataccagtac

SEQ ID NO: 1335 - DNA, 36 bp, synthetic construct
  source 1..36: mol_type = other DNA
gacaatcgaccgtaagnnktataccagtacgaaaga

SEQ ID NO: 1336 - DNA, 28 bp, synthetic construct
  source 1..28: mol_type = other DNA
gaccgtaagcgcnnkaccagtacgaaag

SEQ ID NO: 1337 - DNA, 34 bp, synthetic construct
  source 1..34: mol_type = other DNA
gaccgtaagcgctatnnkagtacgaaagaagtgt

SEQ ID NO: 1338 - DNA, 33 bp, synthetic construct
  source 1..33: mol_type = other DNA
cgtaagcgctataccnnkacgaaagaagtgttg

SEQ ID NO: 1339 - DNA, 36 bp, synthetic construct
  source 1..36: mol_type = other DNA
taagcgctataccagtnnkaaagaagtgttggatgc

SEQ ID NO: 1340 - DNA, 31 bp, synthetic construct
  source 1..31: mol_type = other DNA
gctataccagtacgnnkgaagtgttggatgc

SEQ ID NO: 1341 - DNA, 33 bp, synthetic construct
  source 1..33: mol_type = other DNA
tataccagtacgaaannkgtgttggatgcgacc

SEQ ID NO: 1342 - DNA, 32 bp, synthetic construct
  source 1..32: mol_type = other DNA
ccagtacgaaagaannkttggatgcgaccctt

SEQ ID NO: 1343 - DNA, 35 bp, synthetic construct
  source 1..35: mol_type = other DNA
cagtacgaaagaagtgnnkgatgcgacccttattc

SEQ ID NO: 1344 - DNA, 32 bp, synthetic construct
  source 1..32: mol_type = other DNA
cgaaagaagtgttgnnkgcgacccttattcac

SEQ ID NO: 1345 - DNA, 37 bp, synthetic construct
  source 1..37: mol_type = other DNA
cgaaagaagtgttggatnnkacccttattcaccagtc

SEQ ID NO: 1346 - DNA, 32 bp, synthetic construct
  source 1..32: mol_type = other DNA
aagtgttggatgcgnnkcttattcaccagtca

SEQ ID NO: 1347 - DNA, 31 bp, synthetic construct
  source 1..31: mol_type = other DNA
tgttggatgcgaccnnkattcaccagtcaat

SEQ ID NO: 1348 - DNA, 31 bp, synthetic construct
  source 1..31: mol_type = other DNA
tggatgcgacccttnnkcaccagtcaattac

SEQ ID NO: 1349 - DNA, 36 bp, synthetic construct
  source 1..36: mol_type = other DNA
ggatgcgacccttattnnkcagtcaattacaggatt

SEQ ID NO: 1350 - DNA, 41 bp, synthetic construct
  source 1..41: mol_type = other DNA
ggatgcgacccttattcacnnktcaattacaggattatatg

SEQ ID NO: 1351 - DNA, 40 bp, synthetic construct
  source 1..40: mol_type = other DNA
gcgacccttattcaccagnnkattacaggattatatgaga

SEQ ID NO: 1352 - DNA, 39 bp, synthetic construct
  source 1..39: mol_type = other DNA
acccttattcaccagtcannkacaggattatatgagacc

SEQ ID NO: 1353 - DNA, 41 bp, synthetic construct
  source 1..41: mol_type = other DNA
ccttattcaccagtcaattnnkggattatatgagacccgta

SEQ ID NO: 1354 - DNA, 42 bp, synthetic construct
  source 1..42: mol_type = other DNA
tattcaccagtcaattacannkttatatgagacccgtatcga

SEQ ID NO: 1355 - DNA, 36 bp, synthetic construct
  source 1..36: mol_type = other DNA
ccagtcaattacaggannktatgagacccgtatcga

SEQ ID NO: 1356 - DNA, 36 bp, synthetic construct
  source 1..36: mol_type = other DNA
gtcaattacaggattannkgagacccgtatcgacct

SEQ ID NO: 1357 - DNA, 40 bp, synthetic construct
  source 1..40: mol_type = other DNA
tcaattacaggattatatnnkacccgtatcgaccttagcc

SEQ ID NO: 1358 - DNA, 37 bp, synthetic construct
  source 1..37: mol_type = other DNA
ttacaggattatatgagnnkcgtatcgaccttagcca

SEQ ID NO: 1359 - DNA, 39 bp, synthetic construct
  source 1..39: mol_type = other DNA
acaggattatatgagaccnnkatcgaccttagccaatta

SEQ ID NO: 1360 - DNA, 38 bp, synthetic construct
  source 1..38: mol_type = other DNA
gattatatgagacccgtnnkgaccttagccaattaggt

SEQ ID NO: 1361 - DNA, 37 bp, synthetic construct
  source 1..37: mol_type = other DNA
tatatgagacccgtatcnnkcttagccaattaggtgg

SEQ ID NO: 1362 - DNA, 30 bp, synthetic construct
  source 1..30: mol_type = other DNA
gacccgtatcgacnnkagccaattaggtgg

SEQ ID NO: 1363 - DNA, 30 bp, synthetic construct
  source 1..30: mol_type = other DNA
ccgtatcgaccttnnkcaattaggtgggga

SEQ ID NO: 1364 - DNA, 32 bp, synthetic construct
  source 1..32: mol_type = other DNA
gtatcgaccttagcnnkttaggtggggatagc

SEQ ID NO: 1365 - DNA, 28 bp, synthetic construct
  source 1..28: mol_type = other DNA
gaccttagccaannkggtggggatagcg

SEQ ID NO: 1366 - DNA, 30 bp, synthetic construct
  source 1..30: mol_type = other DNA
ccttagccaattannkggggatagcggtgg

SEQ ID NO: 1367 - DNA, 33 bp, synthetic construct
  source 1..33: mol_type = other DNA
cttagccaattaggtnnkgatagcggtggtagc

SEQ ID NO: 1368 - DNA, 28 bp, synthetic construct
  source 1..28: mol_type = other DNA
caattaggtgggnnkagcggtggtagca

SEQ ID NO: 1369 - DNA, 27 bp, synthetic construct
  source 1..27: mol_type = other DNA
ttaggtggggatnnkggtggtagcagc

SEQ ID NO: 1370 - DNA, 25 bp, synthetic construct
  source 1..25: mol_type = other DNA
gtggggatagcnnkggtagcagcgg

SEQ ID NO: 1371 - DNA, 25 bp, synthetic construct
  source 1..25: mol_type = other DNA
gggatagcggtnnkagcagcggtgg

SEQ ID NO: 1372 - DNA, 28 bp, synthetic construct
  source 1..28: mol_type = other DNA
gatagcggtggtnnkagcggtggttcaa

SEQ ID NO: 1373 - DNA, 25 bp, synthetic construct
  source 1..25: mol_type = other DNA
gcggtggtagcnnkggtggttcaag

SEQ ID NO: 1374 - DNA, 25 bp, synthetic construct
  source 1..25: mol_type = other DNA
gtggtagcagcnnkggttcaagcgg

SEQ ID NO: 1375 - DNA, 26 bp, synthetic construct
  source 1..26: mol_type = other DNA
gtagcagcggtnnktcaagcggtagc

SEQ ID NO: 1376 - DNA, 25 bp, synthetic construct
  source 1..25: mol_type = other DNA
gcagcggtggtnnkagcggtagcga

SEQ ID NO: 1377 - DNA, 27 bp, synthetic construct
  source 1..27: mol_type = other DNA
agcggtggttcannkggtagcgaaaca

SEQ ID NO: 1378 - DNA, 26 bp, synthetic construct
  source 1..26: mol_type = other DNA
gtggttcaagcnnkagcgaaacaccg

SEQ ID NO: 1379 - DNA, 26 bp, synthetic construct
  source 1..26: mol_type = other DNA
gttcaagcggtnnkgaaacaccgggt

SEQ ID NO: 1380 - DNA, 25 bp, synthetic construct
  source 1..25: mol_type = other DNA
caagcggtagcnnkacaccgggtac

SEQ ID NO: 1381 - DNA, 25 bp, synthetic construct
  source 1..25: mol_type = other DNA
gcggtagcgaannkccgggtacaag

SEQ ID NO: 1382 - DNA, 30 bp, synthetic construct
  source 1..30: mol_type = other DNA
cggtagcgaaacannkggtacaagcgaaag

SEQ ID NO: 1383 - DNA, 27 bp, synthetic construct
  source 1..27: mol_type = other DNA
agcgaaacaccgnnkacaagcgaaagc

SEQ ID NO: 1384 - DNA, 25 bp, synthetic construct
  source 1..25: mol_type = other DNA
aaacaccgggtnnkagcgaaagcgc

SEQ ID NO: 1385 - DNA, 26 bp, synthetic construct
  source 1..26: mol_type = other DNA
caccgggtacannkgaaagcgcaaca

SEQ ID NO: 1386 - DNA, 25 bp, synthetic construct
  source 1..25: mol_type = other DNA
cgggtacaagcnnkagcgcaacacc

SEQ ID NO: 1387 - DNA, 28 bp, synthetic construct
  source 1..28: mol_type = other DNA
ggtacaagcgaannkgcaacaccggaaa

SEQ ID NO: 1388 - DNA, 27 bp, synthetic construct
  source 1..27: mol_type = other DNA
acaagcgaaagcnnkacaccggaaagc

SEQ ID NO: 1389 - DNA, 25 bp, synthetic construct
  source 1..25: mol_type = other DNA
gcgaaagcgcannkccggaaagcag

SEQ ID NO: 1390 - DNA, 28 bp, synthetic construct
  source 1..28: mol_type = other DNA
gaaagcgcaacannkgaaagcagtggtg

SEQ ID NO: 1391 - DNA, 25 bp, synthetic construct
  source 1..25: mol_type = other DNA
gcgcaacaccgnnkagcagtggtgg

SEQ ID NO: 1392 - DNA, 27 bp, synthetic construct
  source 1..27: mol_type = other DNA
gcaacaccggaannkagtggtggtagc

SEQ ID NO: 1393 - DNA, 25 bp, synthetic construct
  source 1..25: mol_type = other DNA
caccggaaagcnnkggtggtagctc

SEQ ID NO: 1394 - DNA, 27 bp, synthetic construct
  source 1..27: mol_type = other DNA
ccggaaagcagtnnkggtagctcaggt

SEQ ID NO: 1395 - DNA, 30 bp, synthetic construct
  source 1..30: mol_type = other DNA
ggaaagcagtggtnnkagctcaggtggtag

SEQ ID NO: 1396 - DNA, 31 bp, synthetic construct
  source 1..31: mol_type = other DNA
aaagcagtggtggtnnktcaggtggtagtag

SEQ ID NO: 1397 - DNA, 28 bp, synthetic construct
  source 1..28: mol_type = other DNA
agtggtggtagcnnkggtggtagtagca

SEQ ID NO: 1398 - DNA, 29 bp, synthetic construct
  source 1..29: mol_type = other DNA
tggtggtagctcannkggtagtagcaccc

SEQ ID NO: 1399 - DNA, 32 bp, synthetic construct
  source 1..32: mol_type = other DNA
gtggtagctcaggtnnkagtagcaccctgaat

SEQ ID NO: 1400 - DNA, 32 bp, synthetic construct
  source 1..32: mol_type = other DNA
gtagctcaggtggtnnkagcaccctgaatatt

SEQ ID NO: 1401 - protein, 1368 aa, Streptococcus pyogenes
MDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 1402 - protein, 1368 aa, Streptococcus pyogenes [sequence omitted]

SEQ ID NO: 1403 - RNA, 76 nt, synthetic construct
  source 1..76: mol_type = other RNA
gttttagagctagaaatagcaagttaaaataaggctagtccgttatcaacttgaaaaagtggcaccgagtcggtgc

SEQ ID NO: 1404 - RNA, 44 nt, synthetic construct
  source 1..44: mol_type = other RNA
gttttagagctagaaatagcaagttaaaataaggctagtccgtt

SEQ ID NO: 1405 - RNA, 119 nt, synthetic construct
  source 1..119: mol_type = other RNA
nnnnnnnnnnnnnnnnnnnngttttagagctagaaatagcaagttaaaataaggctagtccgttatcaacttgaaaaagtggcaccgagtcggtgcnnnnnnnnnnnnnnnnnnnnnnn

SEQ ID NO: 1406 - RNA, 87 nt, synthetic construct
  source 1..87: mol_type = other RNA
nnnnnnnnnnnnnnnnnnnngttttagagctagaaatagcaagttaaaataaggctagtccgttnnnnnnnnnnnnnnnnnnnnnnn

(19) United States
(12) Patent Application Publication
(10) Pub. No.: US 2026/0176655 A1
(43) Pub. Date: Jun. 25, 2026

(54) CAS9 Mutants with Improved Activity with Truncated PegRNA in Prime Editing Applications

(71) Applicant: Integrated DNA Technologies, Inc., Coralville, IA (US)

(72) Inventors: Christopher Anthony VAKULSKAS, North Liberty, IA (US); Sarah Franz BEAUDOIN, Iowa City, IA (US); Michael Allen COLLINGWOOD, Solon, IA (US); Diane DEZWAAN, Swisher, IA (US); Adam BRAINARD, Cedar Rapids, IA (US); Katherine KEOGH, Coralville, IA (US)

(21) Appl. No.: 19/421,036

(22) Filed: Dec. 16, 2025

Related U.S. Application Data
(60) Provisional application No. 63/735,978, filed on Dec. 19, 2024.

(51) Int. Cl. C12N 15/90; C12N 9/22; C12N 15/11

(52) U.S. Cl. CPC C12N 15/902; C12N 9/226; C12N 15/11; C12N 2310/20

(57) ABSTRACT

Provided herein are Cas9 mutations with enhanced prime editing activity relative to wild type for use in prime editing using a truncated prime editing guide RNA.

This application claims the benefit of U.S. Ser. No. 63/735,978, filed Dec. 19, 2024, the entirety of which is incorporated herein by reference.

### REFERENCE TO AN ELECTRONIC SEQUENCE LISTING

The contents of the electronic sequence listing 6391-0013PV01_12182024.xml; Size: 1,233 bytes; and Date of Creation: Dec. 18, 2024 is herein incorporated by reference in its entirety.

### BACKGROUND

This disclosure pertains to prime editor variants having novel mutations that would enable prime editing with increased overall potency using a truncated pegRNA, and further, to the ability of a nickase CRISPR/Cas9 mutant to cleave double-stranded DNA on one strand in a targeted manner in living cells when complexed with sgRNAs. SpCas9 is an RNA guided endonuclease from the Clustered Regularly Interspaced Short Palindromic Repeat (CRISPR)-Cas (CRISPR-associated) bacterial adaptive immune system of Streptococcus pyogenes[1]. Cas9 is guided to a 23-nt DNA target sequence by a target site-specific 20-nt complementary RNA (part of the 44-nt crRNA) and a universal 89-nt tracrRNA, collectively referred to as the guide RNA (gRNA) complex. The Cas9-gRNA ribonucleoprotein (RNP) complex mediates double-stranded DNA breaks (DSBs) which are then typically repaired by the non-homologous end joining (NHEJ), microhomology mediated end joining (MMEJ), or homology-directed repair (HDR) system if a suitable template nucleic acid is present.

S. pyogenes Cas9 protein contains two endonuclease domains that function together to generate a double-strand DNA break by cleaving both the target (guide complementary) and non-target (guide noncomplementary) strands of a double-stranded DNA (dsDNA). These conserved domains are the RuvC and HNH domains. There are two known mutations that can alter Cas9, which produces double-stranded cleavage, into a ‘nickase’ that results in single-stranded cleavage. Cas9 D10A variant, in the RuvC domain, generates the nick on the targeted strand, while the Cas9 H840A variant, in the HNH domain, generates the nick on the non-targeted strand[1]. The nickase Cas9 variants have been used to facilitate CRISPR-targeted genome editing approaches that do not rely on the introduction of a dsDNA break, examples of which include cytosine/adenine base editors[2,3] and more recently the Cas9 prime editor[4].

Prime Editing is a technology that utilizes a Cas9 (e.g., a Cas9 nickase) fused to an engineered reverse transcriptase, thereby forming a prime editor. The prime editor is then coupled with a prime editing guide RNA (“pegRNA”) that recognizes the target site and contains the desired edit. The pegRNA can range in length depending on the edit (minimally around 125 bps), which makes these oligos difficult to chemically synthesize. The prime editor was developed by David Liu and it contains the Cas9 nickase, H840A, and a highly mutagenized reverse transcriptase from Moloney Murine Leukemia Virus (MMLV RTase). [4].

Combining saturation mutagenesis and a bacterial prime editing selection approach will facilitate the isolation of mutations within the Cas9 nickase that will result in the most potent prime editor variants with a truncated pegRNA. Previously, the limitation of inserted cargo was much smaller as the pegRNA was already near the limits of chemical synthesis. This invention will allow for shorter pegRNAs and thus larger cargoes and ease of manufacturing.

All references cited herein are incorporated herein by reference in their entireties.

### BRIEF SUMMARY

In exemplary embodiments, the disclosure provides a prime editor variant, comprising: a. a modified Cas9 having one or more mutations listed in Table 2; and b. an engineered reverse transcriptase, wherein the prime editor variant binds to a truncated pegRNA, and exhibits enhanced prime editing activity relative to wild-type Cas9 with a full-length pegRNA. The disclosure provides a prime editor variant wherein the one or more mutations comprises at least one mutation selected from Table 2 that exhibits greater than 2-fold enrichment relative to the wild-type Cas9 amino acid sequence of SEQ ID NO: 1402. The disclosure provides a prime editor variant wherein the one or more mutations comprises at least one mutation selected from the group consisting of: E1071R, E1071K, T1065R, N758Y, A1034E, D180H, H420Q, Q894A, R40L, I122P, R215L, K961E, E1007L, K31Y, D364S, K382S, R457S, S42E, N193M, K649N, Q1364S, and F1008V relative to the wild-type Cas9 amino acid sequence of SEQ ID NO: 1402. The disclosure provides a prime editor variant the modified Cas9 further comprises a H840A mutation relative to the wild-type Cas9 amino acid sequence of SEQ ID NO: 1402. The disclosure provides a prime editor variant wherein the modified Cas9 comprises a Cas protein selected from the group consisting of wild-type Cas9, dCas9, and Cas9 nickase. The disclosure provides a prime editor variant wherein the truncated pegRNA comprises a target sequence, sgRNA scaffold, reverse transcriptase template (RTT), and a primer binding site (PBS) domains, wherein the truncated pegRNA comprises the sgRNA scaffold domain that is shorter from the 3′ end by at least the number of bases selected from the group consisting of 30 bases, 31 bases, 32 bases, 33 bases, 34 bases, 35 bases, 36 bases, 37 bases, 38 bases, 39 bases, and 40 bases relative to the sgRNA scaffold domain of a full-length pegRNA represented by SEQ ID NO: 1403. The disclosure provides a prime editor variant wherein the truncated pegRNA is 31 bases shorter from the 3′ end. In some embodiments, the truncated pegRNA comprises a sgRNA scaffold domain comprising the sequence of SEQ ID NO: 1404. In some embodiments, the truncated pegRNA comprises a sgRNA scaffold domain having the sequence of SEQ ID NO: 1404. The disclosure provides a prime editor variant the modified Cas9 further comprises at least one nuclear localization signal.

The disclosure provides an isolated nucleic acid encoding the prime editor variant as disclosed herein. The disclosure provides an isolated nucleic acid encoding the modified Cas9 as disclosed herein. The disclosure provides an mRNA encoding the prime editor variant as disclosed herein. The disclosure provides a prime editing system, comprising: a. a prime editor variant as disclosed herein; and b. a truncated pegRNA, wherein the prime editing system exhibits enhanced prime editing activity relative to wild-type Cas9 with a full-length pegRNA.

The disclosure provides a method for isolating a modified Cas9 that exhibits enhanced prime editing activity with a truncated prime editing guide RNA (“pegRNA”) relative to a wild-type Cas9 and a respective full-length pegRNA, the method comprising: (a) generating a library of Cas9 variants through saturation mutagenesis of one or more target codons within the nucleic acid sequence encoding Cas9; (b) cloning the mutated nucleic acid encoding Cas9 along with a nucleic acid encoding a truncated pegRNA which targets an antibiotic resistance gene into the same or different plasmid vector; (c) introducing the plasmid vector(s) into a host cell population that has a deactivated antibiotic resistance gene; (d) subjecting the host cell population to antibiotic selection; and (e) isolating modified Cas9 variants that exhibit enhanced prime editing activity with truncated pegRNA to restore antibiotic resistance, thereby isolating a modified Cas9 that functions with a truncated pegRNA that exhibits enhanced prime editing activity relative to a wild-type Cas9 with a respective full-length pegRNA.

The disclosure provides a method for isolating a modified Cas9 that exhibits enhanced prime editing activity with a truncated prime editing guide RNA (“pegRNA”) relative to a wild-type Cas9 and a respective full-length pegRNA wherein the host cell is selected from the group consisting of bacterial cells, insect cells, plant cells, or mammalian cells.

The disclosure provides a method for isolating a modified Cas9 that exhibits enhanced prime editing activity with a truncated prime editing guide RNA (“pegRNA”) relative to a wild-type Cas9 and a respective full-length pegRNA wherein the modified Cas9 that functions with a truncated pegRNA exhibits prime editing activity enhanced by at least an amount selected from the group consisting of 5%, 10%, 15%, 20%, 25%, and 30%, relative to a wild-type Cas9 with a full-length pegRNA.

The disclosure provides a method for isolating a modified Cas9 that exhibits enhanced prime editing activity with a truncated prime editing guide RNA (“pegRNA”) relative to a wild-type Cas9 and a respective full-length pegRNA wherein the truncated pegRNA targets a gene of interest (e.g., kanamycin resistance gene). The disclosure provides a method for isolating a modified Cas9 that exhibits enhanced prime editing activity with a truncated prime editing guide RNA (“pegRNA”) relative to a wild-type Cas9 and a respective full-length pegRNA wherein the truncated pegRNA comprises a target sequence, sgRNA scaffold, reverse transcriptase template (RTT), and a primer binding site (PBS) domains, wherein the truncated pegRNA comprises the sgRNA scaffold domain that is shorter from the 3′ end by a number of bases selected from the group consisting of 30 bases, 31 bases, 32 bases, 33 bases, 34 bases, 35 bases, 36 bases, 37 bases, 38 bases, 39 bases, and 40 bases relative to the sgRNA scaffold domain of a full-length pegRNA represented by SEQ ID NO: 1403. The disclosure provides a method for isolating a modified Cas9 that exhibits enhanced prime editing activity with a truncated prime editing guide RNA (“pegRNA”) relative to a wild-type Cas9 and a respective full-length pegRNA wherein the truncated pegRNA is 31 bases shorter from the 3′ end. The disclosure provides a method for isolating a modified Cas9 that exhibits enhanced prime editing activity with a truncated pegRNA relative to a wild-type Cas9 and a respective full-length pegRNA wherein the truncated pegRNA comprises a sgRNA scaffold domain having the sequence of SEQ ID NO: 1404. The disclosure provides a method for isolating a modified Cas9 that exhibits enhanced prime editing activity with a truncated prime editing guide RNA (“pegRNA”) relative to a wild-type Cas9 and a respective full-length pegRNA wherein the saturation mutagenesis targets specific codons within a Cas9 selected from the group consisting of the REC Lobe; the Bridge Helix; the NUC Lobe; the RuvC Domain; the HNH Domain; the PAM-Interacting Domain; and the C Terminal Domain. The disclosure provides a method for isolating a modified Cas9 that exhibits enhanced prime editing activity with a truncated prime editing guide RNA (“pegRNA”) relative to a wild-type Cas9 and a respective full-length pegRNA wherein the mutations are located in a Cas9 selected from the group consisting of REC1, REC2, REC3, and combinations thereof.

The disclosure provides a method for producing a modified Cas9 that exhibits enhanced prime editing activity with a truncated pegRNA relative to wild-type Cas9 with a respective full-length pegRNA, the method comprising: (a) subjecting a nucleic acid sequence encoding Cas9 to saturation mutagenesis; (b) cloning the mutated nucleic acid encoding Cas9 along with a nucleic acid encoding truncated pegRNA which targets an antibiotic resistance gene into the same or different plasmid vector; (c) introducing the plasmid vector(s) into a host cell population that has a deactivated antibiotic resistance gene; (d) subjecting the host cell population to antibiotic selection; (d) isolating modified Cas9 variants that exhibit enhanced prime editing activity functional interaction with truncated pegRNA which restores antibiotic resistance, thereby producing a modified Cas9 that functions with a truncated pegRNA that exhibits enhanced prime editing activity relative to wild-type Cas9 with a respective full-length pegRNA.

The disclosure provides a method for producing a modified Cas9 that exhibits enhanced prime editing activity with a truncated pegRNA relative to wild-type Cas9 with a respective full-length pegRNA wherein the host cell is selected from the group consisting of bacterial cells, insect cells, plant cells, or mammalian cells. The disclosure provides a method for producing a modified Cas9 that exhibits enhanced prime editing activity with a truncated pegRNA relative to wild-type Cas9 with a respective full-length pegRNA wherein the modified Cas9 that functions with a truncated pegRNA exhibits prime editing activity enhanced by at least an amount selected from the group consisting of 5%, 10%, 15%, 20%, 25%, and 30%, relative to a wild-type Cas9 with a full-length pegRNA. The disclosure provides a method for producing a modified Cas9 that exhibits enhanced prime editing activity with a truncated pegRNA relative to wild-type Cas9 with a respective full-length pegRNA wherein the truncated pegRNA targets a kanamycin resistance gene. The disclosure provides a method for producing a modified Cas9 that exhibits enhanced prime editing activity with a truncated pegRNA relative to wild-type Cas9 with a respective full-length pegRNA wherein the truncated pegRNA comprises a target sequence, sgRNA scaffold, reverse transcriptase template (RTT), and a primer binding site (PBS) domains, wherein the truncated pegRNA comprises the sgRNA scaffold domain that is shorter from the 3′ end by a number of bases selected from the group consisting of 30 bases, 31 bases, 32 bases, 33 bases, 34 bases, 35 bases, 36 bases, 37 bases, 38 bases, 39 bases, and 40 bases relative to the sgRNA scaffold domain of a full-length pegRNA represented by SEQ ID NO: 1403. The disclosure provides a method for producing a modified Cas9 that exhibits enhanced prime editing activity with a truncated pegRNA relative to wild-type Cas9 with a respective full-length pegRNA wherein the truncated pegRNA is 31 bases shorter from the 3′ end relative to the sgRNA scaffold domain of a full-length pegRNA represented by SEQ ID NO: 1403. The disclosure provides a method for producing a modified Cas9 that exhibits enhanced prime editing activity with a truncated pegRNA relative to wild-type Cas9 with a respective full-length pegRNA wherein the bacterial host cell expresses a reporter gene under the control of a promoter responsive to Cas9 activity, wherein expression of the antibiotic resistance gene indicates enhanced potency of the Cas9 variant. The disclosure provides a method for producing a modified Cas9 that exhibits enhanced prime editing activity with a truncated pegRNA relative to wild-type Cas9 with a respective full-length pegRNA wherein the saturation mutagenesis targets specific codons within a Cas9 selected from the group consisting of the REC Lobe; the Bridge Helix; the NUC Lobe; the RuvC Domain; the HNH Domain; the PAM-Interacting Domain; and the C Terminal Domain. The disclosure provides a method for producing a modified Cas9 that exhibits enhanced prime editing activity with a truncated pegRNA relative to wild-type Cas9 with a respective full-length pegRNA wherein the mutations are located in a Cas9 selected from the group consisting of REC1, REC2, REC3, and combinations thereof.

The disclosure provides a modified Cas9, comprising one or more mutations selected from Table 2 relative to the wild-type Cas9 amino acid sequence of SEQ ID NO: 1402, wherein the modified Cas9 binds to a truncated pegRNA to form a complex, and wherein the complex exhibits enhanced prime editing activity relative to wild-type Cas9 with a full-length pegRNA.

The disclosure provides a modified Cas9 wherein the one or more mutations comprises at least one mutation selected from the group consisting of: E1071R, E1071K, T1065R, N758Y, A1034E, D180H, H420Q, Q894A, R40L, I122P, R215L, K961E, E1007L, K31Y, D364S, K382S, R457S, S42E, N193M, K649N, Q1364S, and F1008V relative to the wild-type Cas9 amino acid sequence of SEQ ID NO: 1402.

The disclosure provides a modified Cas9 wherein the modified Cas9 that functions with a truncated pegRNA exhibits prime editing activity enhanced by at least an amount selected from the group consisting of 5%, 10%, 15%, 20%, 25%, and 30%, relative to a wild-type Cas9 with a full-length pegRNA. The disclosure provides a modified Cas9 wherein the truncated pegRNA comprises a sgRNA scaffold domain that is shorter from the 3′ end by a number of bases selected from the group consisting of 30 bases, 31 bases, 32 bases, 33 bases, 34 bases, 35 bases, 36 bases, 37 bases, 38 bases, 39 bases, and 40 bases relative to the sgRNA scaffold domain of a full-length pegRNA represented by SEQ ID NO: 1403. The disclosure provides a modified Cas9 wherein the truncated pegRNA is 31 bases shorter from the 3′ end relative to the sgRNA scaffold domain of a full-length pegRNA represented by SEQ ID NO: 1403. The disclosure provides a modified Cas9 that binds to a truncated pegRNA, wherein the truncated pegRNA comprises a sgRNA scaffold domain having the sequence of SEQ ID NO: 1404. The disclosure provides a modified Cas9 wherein the saturation mutagenesis targets specific codons within the active site of Cas9 to increase affinity for its substrate. The disclosure provides a modified Cas9 wherein the mutations are located in the active site, substrate-binding region, or regulatory region of the modified Cas9. The disclosure provides a modified Cas9 wherein the mutations are located in the active site, substrate-binding region, or regulatory region of the modified Cas9.

The disclosure provides a host cell comprising a nucleic acid encoding the modified Cas9 variant as disclosed herein wherein the host cell is configured to express the potent Cas9 variant. The disclosure provides a composition comprising the potent Cas9 variant as disclosed herein formulated for use in biochemical assays, industrial processes, or therapeutic applications. The disclosure provides a method of using the potent Cas9 variant as disclosed herein to increase prime editing activity in an in vitro, in vivo, or ex vivo system.

The disclosure provides a method of delivering a Cas9 truncated pegRNA system as disclosed herein to a mammalian cell, the method comprising the steps of: (a) providing a first viral vector component encoding a truncated pegRNA that hybridizes with a target sequence; (b) providing a second viral vector component encoding a Cas9 variant; wherein components (a) and (b) are located on same or different vectors of the system; and (c) transducing the mammalian cell with the viral vector(s) under conditions sufficient to express the Cas9-Truncated pegRNA, wherein the Cas9-Truncated pegRNA complex form a complex that binds to and edits the target sequence.

The disclosure provides a method for delivering a Cas9 truncated pegRNA system as disclosed herein to a mammalian cell, comprising: (a) providing lipid nanoparticles encapsulating a first mRNA which is a truncated pegRNA that hybridizes with a target sequence; (b) providing lipid nanoparticles encapsulating a second mRNA encoding a Cas9 variant as disclosed herein; wherein components (a) and (b) are located on same or different vectors of the system, (c) transducing the mammalian cell with the lipid nanoparticles under conditions sufficient to express the Cas9-truncated pegRNA, wherein the Cas9-truncated pegRNA are expressed and form a complex to edit a specific target sequence in the cell's genome.

The disclosure provides a method of performing gene editing having reduced off-target editing activity and/or increased on-target editing activity, comprising: (a) providing a ribonucleoprotein (RNP) complex comprising the Cas9-truncated pegRNA as disclosed herein and a truncated pegRNA; and (b) introducing the RNP complex into the mammalian cell using electroporation, wherein the Cas9-truncated pegRNA system binds to and edits a target sequence within the genome of the mammalian cell.

The disclosure provides a method of delivering the Cas9-truncated pegRNA system as disclosed herein to a mammalian cell, comprising: preparing a lipofection reagent comprising a first regulatory element operable in a eukaryotic cell operably linked to at least one nucleotide sequence encoding a truncated pegRNA that hybridizes with a target sequence; a second regulatory element operable in a eukaryotic cell operably linked to a nucleotide sequence encoding a Cas9 variant; wherein components (a) and (b) are located on the same or different vectors of the system, and applying the lipofection reagent to the mammalian cell, wherein the Cas9-truncated pegRNA system are expressed and form a complex to edit a specific target sequence in the cell's genome.

The disclosure provides a method of targeted delivery of the Cas9-truncated pegRNA system as disclosed herein to a mammalian cell, comprising: (a) preparing exosomes encapsulating a first nucleotide sequence encoding a truncated pegRNA that hybridizes with a target sequence; and (b) preparing exosomes encapsulating a second nucleotide sequence encoding a Cas9 variant; wherein components (a) and (b) are located on same or different vectors of the system; and (c) delivering the engineered exosomes to the mammalian cell under conditions that allow the Cas9-truncated pegRNA system to edit the target sequence.

### BRIEF DESCRIPTION OF SEVERAL VIEWS OF THE DRAWINGS

The invention will be described in conjunction with the following drawings in which like reference numerals designate like elements and wherein:

FIG. 1 is a graphic illustration of representative domains of a full length pegRNA (SEQ ID NO: 1405).

FIG. 2 is a graphic illustration of representative domains of a truncated pegRNA (SEQ ID NO: 1406).

### DETAILED DESCRIPTION

This disclosure provides using a bacterial prime editing selection approach to isolate mutations within the Cas9 of a prime editor variant to uncover the most potent prime editor variant using a truncated pegRNA. The prime editor variant as provided herein comprises a Cas9 and an engineered reverse transcriptase. To our knowledge, no comprehensive screen with selection has been performed for the prime editor variant using a truncated pegRNA. The inventors made 14 mutant libraries of every possible amino acid substitution spanning the open reading frame of the Cas9 portion of the prime editor variant. The libraries were screened for substitutions that would enable prime editing in bacteria with the highest overall potency using a truncated pegRNA.

The prime editor variants as provided herein can be used for any application that requires Cas9, nickase Cas9, or dead Cas9 (aka dCas9 or catalytically inactive Cas9) to function with a truncated guide RNA, but more aptly as a prime editor variant to function with a shorter, truncated pegRNA, which are easier to chemically synthesize and enable larger insertion cargoes. The prime editor variants as provided herein can be used, for example, in live cells or in a cell free context, and could be expressed from a virus, plasmid, or mRNA, or alternatively used directly in an RNP as a purified nuclease.

The Clustered Regularly Interspaced Short Palindromic Repeats and CRISPR-associated protein 9 (CRISPR-Cas9) system is a tool in genetic engineering to precisely edit genes within organisms. CRISPR-Cas systems are native to bacteria and Archaea to provide adaptive immunity against viruses and plasmids. CRISPR are sequences of DNA in bacterial genomes that store fragments of viral DNA. If the bacterium survives a viral attack, it incorporates a piece of the virus's genetic material into its own DNA. These stored sequences help the bacteria recognize and destroy a virus or plasmid. Cas9 is an enzyme that cuts DNA at specific sites. It's guided by a piece of RNA, known as a guide RNA (gRNA), which matches a specific DNA sequence that scientists want to modify. The gRNA is complementary to the target DNA sequence. This gRNA will guide the Cas9 protein to the exact location in the genome. Once guided to the target location, the Cas9 enzyme cuts the DNA at that precise spot. After Cas9 cuts the DNA, the cell tries to repair the break. However, the cell's repair process is often error-prone, leading to mutations that can disrupt the function of the targeted gene. By providing a template with the desired DNA sequence, scientists can instruct the cell to incorporate this new sequence during the repair process.

There are three classes of CRISPR-Cas systems that could potentially be adapted for research and therapeutic reagents, but Type-II CRISPR systems have a desirable characteristic in utilizing a single CRISPR associated (Cas) nuclease (e.g., Cas9) in a complex with the appropriate guide RNAs—either a 2-part RNA system similar to the natural complex in bacteria comprising a CRISPR-activating RNA: trans-activating crRNA (crRNA: tracrRNA) pair or an artificial chimeric single-guide-RNA (sgRNA)—to mediate double-stranded cleavage of target DNA. In mammalian systems, these RNAs have been introduced by transfection of DNA cassettes containing RNA Pol III promoters (such as U6 or H1) driving RNA transcription, viral vectors, and single-stranded RNA following in vitro transcription (see Xu, T., et al., Appl Environ Microbiol, 2014. 80(5): p. 1544-52).

Cas9 nickase is a modified version of the Cas9 protein. While the wild-type Cas9 enzyme introduces double-strand breaks (DSBs) in DNA, Cas9 nickase is designed to make a single-strand break, or “nick,” at a specific location on the DNA. This modified version offers more precision and reduces the risk of unintended genomic alterations (off-target effects). Cas9 nickase only cleaves one strand of the DNA, creating a nick rather than a complete break. This happens by mutating one of Cas9's two nuclease domains, either RuvC or HNH, which renders it inactive. As a result, Cas9 retains the ability to cut only one DNA strand. For genome editing, two Cas9 nickases can be used in combination to target opposite strands of DNA close to each other. This creates two single-strand breaks, which can then be treated as a DSB by the cell's repair machinery. The advantage of this method is that it requires two closely spaced, correctly targeted nicks to achieve a DSB, reducing the likelihood of off-target effects. DSBs are typically repaired by non-homologous end joining (NHEJ), which is error-prone and can lead to insertions or deletions (indels). Nicks, on the other hand, are primarily repaired through homology-directed repair (HDR), a more accurate repair process if a homologous DNA template is available. The nickase system allows for more precise editing with fewer chances of errors or unwanted mutations, especially when using HDR for precise gene modifications.

Cas9 has two nuclease domains, RuvC and HNH, that are responsible for cleaving the two strands of DNA. To create a nickase, one of these domains is mutated: Mutating the RuvC domain (e.g., D10A mutation) leads to the cutting of the complementary strand only. Mutating the HNH domain (e.g., H840A mutation) results in the nicking of the noncomplementary strand only. This modification makes Cas9 a versatile tool for more controlled genome editing, especially when combined with other components to enhance precision.

Paired nickases can be used to introduce highly accurate modifications by ensuring both strands are cut only at the intended target sites. Since two nicks are required to create a DSB, the probability of off-target double-strand breaks is significantly reduced compared to wild-type Cas9. Cas9 nickase can be combined with deaminases for base editing, allowing the precise conversion of one base to another (e.g., C to T) without inducing a DSB.

The Cas9 protein consists of several functional regions or domains, each with specific roles in the CRISPR-Cas9 gene-editing process, including the REC Lobe (Recognition Lobe), which contains several subdomains (REC1, REC2, REC3) that are responsible for recognizing and binding the guide RNA (gRNA); the Bridge Helix, which is a helical structure that connects the REC and NUC lobes and plays a role in the allosteric activation of Cas9. When the gRNA binds, the bridge helix undergoes a conformational change that activates the nuclease activity, preparing Cas9 to cleave DNA; The NUC Lobe (Nuclease Lobe) which contains the active nuclease domains and is responsible for cutting the target DNA. The two primary nuclease domains include the RuvC Domain which cleaves the DNA strand that does not pair with the guide RNA (the non-target strand) and the HNH Domain which cleaves the DNA strand that is complementary to the guide RNA (the target strand). The PAM-Interacting Domain (PID) recognizes the protospacer adjacent motif (PAM) sequence, a short DNA sequence adjacent to the target site required for Cas9 binding. Different Cas9 variants recognize different PAM sequences, which determines the target specificity of each Cas9 protein. PAM recognition is a critical step because Cas9 will not bind or cut DNA without the correct PAM sequence. The CTD (C-Terminal Domain) is involved in stabilizing the Cas9-DNA complex.

The modified Cas9-truncated pegRNA system components as disclosed herein can be introduced into the cell, together or separately, using various approaches. Examples include plasmid or viral expression vectors (which lead to endogenous expression), Cas9 mRNA with separate pegRNA transfection, or delivery of the modified Cas9 protein with the pegRNA as a ribonucleoprotein (RNP) complex (see Kouranova et al., Hum Gen Ther (2016) 27(6):464-475). Effective strategies for introducing, for example, the Cas9-truncated pegRNA system as disclosed herein into target cells include, for example, Viral Vectors, such as Adeno-Associated Virus (AAV) which are widely used for CRISPR delivery because they are generally safe, induce minimal immune response, and have been approved in some gene therapy applications; Lentivirus and Retrovirus; and Adenovirus.

Additional methods for introducing the modified Cas9-truncated pegRNA system as disclosed herein into target cells includes, for example, Lipid Nanoparticles (LNPs) which are commonly used for delivering RNA-based therapies; Electroporation, which involves applying an electrical field to create temporary pores in the cell membrane, allowing CRISPR components (like plasmids, ribonucleoprotein complexes, or mRNA) to enter the cell. Additional methods for introducing the modified Cas9-truncated pegRNA system as disclosed herein into target cells includes, for example, Ribonucleoprotein (RNP) Complexes which involves directly delivering the Cas9 protein pre-complexed with guide RNA (gRNA) into cells, usually via electroporation or lipid-based transfection; Lipid-Based Transfection Agents (lipofection) uses lipid-based reagents to encapsulate CRISPR plasmids or RNP complexes and facilitate their uptake by cells. Other methods for introducing the Modified Cas9-truncated pegRNA system as disclosed herein into target cells includes, for example, Physical Methods such as Microinjection to directly injects CRISPR components into cells, typically used in single-cell embryos or zygotes for generating transgenic animals; Nanoneedles and Microfluidics which can introduce CRISPR components with minimal damage to cells; and Exosome-Mediated Delivery, which can be engineered to carry CRISPR/Cas components and target them to specific cells.

Prime editing is an advanced genome editing technique derived from CRISPR technology which offers the ability to precisely insert, delete, or alter specific DNA sequences without the need for double-strand breaks (DSBs) or donor DNA templates. Prime editing was developed to address some of the limitations of earlier CRISPR systems, such as imprecise modifications, off-target effects, and the need for an external DNA template for homologous recombination. The Components of Prime Editing include a Cas9 Nickase (Cas9n). Instead of cutting both strands of DNA to create a double-strand break, Cas9 nickase cuts only one strand, producing a single-strand break or “nick.” This significantly reduces the risks associated with double-strand breaks. Other components include Reverse Transcriptase (RT). In prime editing, the Cas9 nickase is fused to a reverse transcriptase enzyme, which synthesizes a new DNA strand using an RNA template provided by the pegRNA (prime editing guide RNA). This allows for precise edits, including base changes, insertions, or deletions, directly at or near the nicked site. The Prime Editing Guide RNA (pegRNA) is an extended version of the traditional guide RNA (gRNA) used in CRISPR-Cas9. It has two main parts: A guide sequence that directs the Cas9 nickase to the correct location in the genome, ensuring the cut happens at the desired site; and a reverse transcriptase template and a primer binding site that encode the desired DNA sequence changes. This template provides the instructions for the reverse transcriptase to make the new DNA sequence, which will replace the original DNA at the target site.

The pegRNA guides the Cas9 nickase to a specific location in the genome, where it makes a single-strand nick in the DNA. After the DNA is nicked, the reverse transcriptase enzyme uses the reverse transcription template in the pegRNA to synthesize a complementary DNA sequence. This new DNA strand incorporates the desired genetic change (such as a base substitution, insertion, or deletion). The newly synthesized DNA is integrated into the genome through the cell's natural repair processes. Because this process doesn't involve double-strand breaks, the cell avoids using error-prone repair mechanisms like non-homologous end joining (NHEJ). Instead, it typically uses more accurate repair pathways, such as homology-directed repair (HDR). The edited DNA sequence replaces the original sequence at the targeted site, creating a precise, intended change without introducing indels or off-target mutations.

Prime editing allows for very precise genome modifications, including base substitutions, small insertions, deletions, and even complex modifications like transversions (e.g., changing a C-G pair to a G-C pair). Unlike traditional CRISPR-Cas9, which relies on DSBs followed by error-prone repair, prime editing uses a single-strand break. This minimizes the chances of large insertions, deletions, or chromosomal rearrangements. Prime editing doesn't require an external DNA template for homology-directed repair (HDR), as the pegRNA provides the instructions for the modification directly. By using a Cas9 nickase and a single-strand break, the risk of off-target effects is significantly lower compared to conventional CRISPR-Cas9 systems that rely on DSBs.

Prime editing can make a variety of precise genetic changes, including small indels, all types of base substitutions, and even more complex alterations like insertions of longer sequences. Prime editing is limited to a specific “editing window” near the nicking site, meaning the changes that can be made are localized to a certain area around the cut. Prime editing has great potential for correcting disease-causing mutations at their precise location in the genome without causing unintended damage.

The Prime Editing Guide RNA (pegRNA) is a specially designed guide RNA used in the prime editing system, which directs the prime editor variant (Cas9, including a Cas9 nickase, fused to a reverse transcriptase enzyme) to the target DNA and provides a template for the desired DNA change. pegRNA is more complex than a standard guide RNA (gRNA) because it has additional elements that enable precise editing. Like traditional CRISPR gRNA, the pegRNA contains an about 20-nucleotide sequence guide RNA sequence that matches the target DNA site. This sequence directs the Cas9 component of the prime editor variant to the specific region of the genome where the desired edit will occur. The pegRNA also includes an extended sequence that acts as a template for reverse transcription. This template encodes the desired DNA modification (e.g., base substitution, insertion, or deletion). It also contains a primer binding site, which allows the reverse transcriptase enzyme to use the nicked strand of DNA as a starting point for synthesizing the edited DNA strand according to the instructions in the pegRNA.

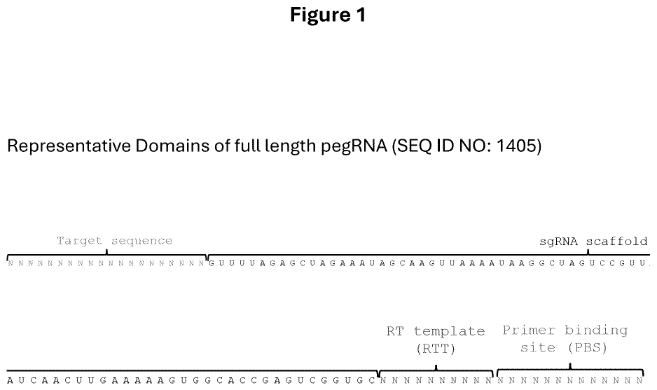

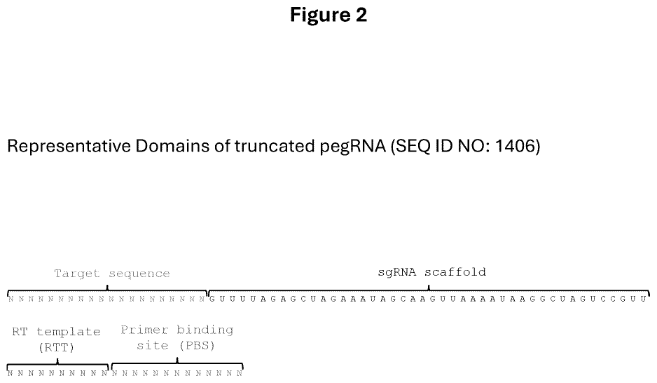

Certain embodiments as disclosed herein utilize a truncated pegRNA with a shorter sgRNA scaffold domain. A full-length pegRNA comprises a target sequence, sgRNA scaffold, reverse transcriptase template (RTT), and a primer binding site (PBS) (FIG. 1). The truncated pegRNA as disclosed is a modified version of a full-length prime editing guide RNA (pegRNA), which has a shortened sgRNA scaffold domain (FIG. 2). In certain exemplary embodiments as disclosed herein the truncated pegRNA comprises a sgRNA scaffold domain that is shorter from the 3′ end by 5 bases, 10 bases, 15 bases, 20 bases, 25 bases, 30 bases, 35 bases, 40 bases, 45 bases, or 50 bases. In certain exemplary embodiments as disclosed herein the truncated pegRNA comprises a sgRNA scaffold domain that is shorter from the 3′ end by 30 bases, 31 bases, 32 bases, 33 bases, 34 bases, 35 bases, 36 bases, 37 bases, 38 bases, 39 bases, or 40 bases. In certain exemplary embodiments as disclosed herein the truncated pegRNA comprises a sgRNA scaffold domain is 31 bases shorter from the 3′ end. In some embodiments, the truncated pegRNA comprises a sgRNA scaffold domain comprising the sequence of SEQ ID NO: 1404.

Next Generation Sequencing (NGS) allows rapid and high-throughput sequencing of DNA and RNA. Unlike earlier methods such as Sanger sequencing, which sequences one DNA fragment at a time, NGS enables the simultaneous sequencing of millions of DNA fragments, making it much faster, cheaper, and more efficient. In NGS, a DNA or RNA from the sample is extracted and fragmented into smaller pieces. These fragments are then attached to short synthetic DNA sequences called adapters, which are needed for binding to the sequencing platform. The DNA fragments with adapters are amplified (copied many times) to create a “library” of DNA fragments. This increases the amount of DNA available for sequencing. Most NGS platforms, like Illumina, use a method called “sequencing by synthesis.” Each fragment is attached to a solid surface and copied in place. Fluorescently-labeled nucleotides (A, T, C, and G) are added one by one. As they bind to the complementary strand, the machine detects the fluorescent signal, allowing the sequence of bases to be read. The massive amount of sequencing data is analyzed using bioinformatics tools. The overlapping DNA fragments are assembled back into their original sequence by aligning them to a reference genome or constructing new genomes (de novo sequencing). NGS allows for High Throughput, since millions to billions of DNA fragments can be sequenced in parallel, producing vast amounts of data, is cost-effective, and can sequence entire genomes or large sets of genes in days, making it much faster than older sequencing methods.

Saturation mutagenesis is used to introduce mutations at every possible site within a specific region of a gene. This method systematically replaces one or more amino acids in a protein with all other possible amino acids (or nucleotides in a DNA sequence) to explore the effects of these changes on the protein's function. The goal is to understand which mutations enhance or diminish the activity, stability, or other characteristics of a protein, and it is often used in protein engineering. The target selected for saturation mutagenesis can be a specific codon or a set of codons within a protein that are crucial for function. A set of oligonucleotides with randomized nucleotides at the positions where mutations are intended is created. The mutagenized oligonucleotides are incorporated into the target gene via polymerase chain reaction (PCR). The mutagenized gene is then cloned into an expression vector. After introducing the mutated genes into a host organism (such as bacteria), the resulting proteins are expressed. These proteins are then tested for desired properties such as improved enzymatic activity, binding affinity, or stability. The most promising mutants are sequenced to identify which amino acid substitutions are responsible for any observed changes in protein function. By generating a diverse library of mutants, saturation mutagenesis can be used for optimizing protein properties or gaining deeper insight into protein function.

A variety of host cells can serve as platforms for CRISPR expression, such as: Bacterial Cells: Escherichia coli is widely used for CRISPR applications like plasmid construction, cloning, and CRISPR screens; Yeast: Saccharomyces cerevisiae and other yeast species can be genetically modified to express CRISPR systems, especially in studies focused on gene function and genome screening in eukaryotic systems; Insect cells can serve as hosts for CRISPR expression. In particular, insect cell lines such as Sf9 (from Spodoptera frugiperda) and S2 (from Drosophila melanogaster); Mammalian Cells: Various mammalian cell lines, including HEK293, HeLa, and CHO cells, are commonly used, which are compatible with more complex CRISPR modifications, such as large gene insertions, knock-ins, or base editing, due to their complex regulatory machinery; Primary Cells and Stem Cells: Primary cells, like human or animal-derived cells, and induced pluripotent stem cells (iPSCs) can also be used as host cells for CRISPR systems, especially for therapeutic studies and disease modeling; Plant Cells: Plants like Arabidopsis thaliana, tobacco, and rice can serve as CRISPR host cells. Plant cells are often transformed with CRISPR machinery to study gene function, enhance traits, or improve resistance to pathogens. Various delivery systems for CRISPR components, such as plasmids, viral vectors, or ribonucleoprotein complexes, and specific promoters can be optimized for the host's transcriptional machinery.

As used herein, the terms “subject” and “patient” are used interchangeably. As used herein, the term “patient” refers to an animal, preferably a mammal such as a non-primate (e.g., cows, pigs, horses, cats, dogs, rats etc.) and a primate (e.g., monkey and human), and most preferably a human. In some embodiments, the subject is a non-human animal such as a farm animal (e.g., a horse, pig, or cow) or a pet (e.g., a dog or cat). In a specific embodiment, the subject is an elderly human. In another embodiment, the subject is a human adult. In another embodiment, the subject is a human child. In yet another embodiment, the subject is a human infant. The patient or subject to be medicated according to the compositions and methods as disclosed herein may be any animal or human. In certain embodiments, animals may include vertebrates. The terms vertebrate or animals in this context is understood to comprise, for example fish, amphibians, reptiles, birds, and mammals including humans. One preferred group of vertebrates or animals according to the invention comprises warm-blooded animals including farm animals, such as cattle, horses, pigs, sheep and goats, poultry such as chickens, turkeys, guinea fowls and geese, fur-bearing animals such as mink, foxes, chinchillas, rabbits and the like, as well as companion animals such as ferrets, guinea pigs, rats, hamster, cats and dogs. A further group of preferred vertebrates or animals according to the disclosure comprises fish including salmonids, for example salmon, trout or whitefish. The subject is preferably mammalian. In some embodiments the subject is a human. In other embodiments the subject is an animal, more preferably a non-human mammal. The non-human mammal may be a domestic pet, or animal kept for commercial purposes, e.g., a racehorse, or farming livestock or animals such as pigs, sheep or cattle. As such the disclosure may have veterinary applications. Non-human mammals include rabbits, guinea pigs, rats, mice or other rodents (including any animal in the order Rodentia), cats, dogs, pigs, sheep, goats, cattle (including cows or any animal in the order Bos), horse (including any animal in the order Equidae), donkey, and non-human primates. The subject may be male or female. The subject may be an adult or a child. The subject may be a patient.

As used herein, the term “about” when used in conjunction with a stated numerical value or range has the meaning reasonably ascribed to it by a person skilled in the art, i.e., denoting somewhat more or somewhat less than the stated value or range.

When a group of substituents is disclosed herein, it is understood that all individual members of those groups and all subgroups and classes that can be formed using the substituents are disclosed separately. When a Markush group or other grouping is used herein, all individual members of the group and all combinations and subcombinations possible of the group are intended to be individually included in the disclosure. As used herein, “and/or” means that one, all, or any combination of items in a list separated by “and/or” are included in the list; for example, “1, 2 and/or 3” is equivalent to “1, 2, 3, 1 and 2, 1 and 3, 2 and 3, or 1, 2, and 3”.

Every formulation or combination of components described or exemplified can be used to practice the disclosure, unless otherwise stated. Specific names of materials are intended to be exemplary, as it is known that one of ordinary skill in the art can name the same material differently. It will be appreciated that methods, device elements, starting materials, and synthetic methods other than those specifically exemplified can be employed in the practice as disclosed herein without resort to undue experimentation. All art-known functional equivalents, of any such methods, device elements, starting materials, and synthetic methods are intended to be included in this disclosure. Whenever a range is given in the specification, for example, a temperature range, a time range, or a composition range, all intermediate ranges and subranges, as well as all individual values included in the ranges given are intended to be included in the disclosure. It is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed. The upper and lower limits of these smaller ranges may independently be included or excluded in the range, and each range where either, neither, or both limits are included in the smaller ranges is also encompassed within the disclosure, subject to any specifically excluded limit in the stated range. Where the stated range includes one or both of the limits, ranges excluding either or both of those included limits are also included.

As used herein, “comprising” is synonymous with “including,” “containing,” or “characterized by,” and is inclusive or open-ended and does not exclude additional, unrecited elements or method steps. As used herein, “consisting of” excludes any element, step, or ingredient not specified in the claim element. As used herein, “consisting essentially of” does not exclude materials or steps that do not materially affect the basic and novel characteristics of the claim. Any recitation herein of the term “comprising”, particularly in a description of components of a composition, in a description of a method, or in a description of elements of a device, is understood to encompass those compositions, methods, or devices consisting essentially of and consisting of the recited components or elements, optionally in addition to other components or elements. The disclosure as illustratively described herein suitably may be practiced in the absence of any element, elements, limitation, or limitations which is not specifically disclosed herein.

As used herein and in the appended claims, the singular forms “a”, “an”, and “the” include plural referents unless the context clearly dictates otherwise. Thus, for example, reference to “a method” includes a plurality of such methods and reference to “the nanoparticle” includes reference to one or more nanoparticles and equivalents thereof known to those skilled in the art, and so forth. The terms and expressions which have been employed are used as terms of description and not of limitation, and there is no intention in the use of such terms and expressions of excluding any equivalents of the features shown and described or portions thereof, but it is recognized that various modifications are possible within the scope as disclosed herein claimed. Thus, it should be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

All references throughout this application, for example patent documents, including issued or granted patents or equivalents and patent application publications, and non-patent literature documents or other source material are hereby incorporated by reference herein in their entireties, as though individually incorporated by reference. None is admitted to being prior art.

The invention will be illustrated in more detail with reference to the following Examples, but it should be understood that the present invention is not deemed to be limited thereto.

### EXAMPLES

Example 1—A bacterial prime editing selection and enrichment strategy reveals Cas9 and RTase mutations that facilitate the most potent prime editor variant.

Functional prime editing has recently been described in E. coli using a variety of insertions/deletions and substitutions [5]. We used expression of a prime editor variant and pegRNA that targets a kanamycin resistance gene that contains a 10-base disruption between codons 1 and 2. This prime editing strategy is designed to remove the disrupting 10-base element and restore kanamycin resistance gene expression, and thus, confer resistance to the antibiotic kanamycin. The screen is setup such that a library of Cas9 prime editor variants and a truncated pegRNA (where the truncated pegRNA comprises a sgRNA scaffold domain having the sequence of SEQ ID NO: 1404) are expressed from one plasmid, and the target site and non-functional kanamycin resistance gene is present on the chromosome. Practically, bacterial cells that repair and stably replicate the kanamycin resistance gene are made competent, transformed using Cas9 prime editor variant and the truncated pegRNA plasmid, and selected on Kanamycin-containing solid media.

Fourteen saturation mutagenesis libraries (Table 1) spanning the Cas9 nickase portion of the prime editor variant were generated with QuikChange Multi Site-Directed Mutagenesis Kit (Agilent Technologies Inc.) and the resulting library complexity was analyzed with tiled-amplicon NGS. These libraries were delivered into E. coli cells as described above to be greater than 99% confident that each possible codon change was observed at least once. The resulting colonies were pooled, plasmids from this pool were purified, and supplied back into the same E. coli cells for subsequent rounds and pooling, until E. coli cells reached saturation (up to five rounds). The resulting pools from each round were sequenced with overlapping tiled amplicon NGS. Pre- and post-enrichment pools were sequenced and compared simultaneously with total read count normalization to determine enrichment for each substitution within the pool. Paired end reads were merged by fastp [6], trimmed to keep the desired mutated region by Cutadapt [7], and filtered out undesired reads with wrong length or multiple codon mutation. Codon frequency and enrichment analysis were completed by in-house python script. The results included over 3,600 amino acid substitutions that are at equal or greater frequency than the identical substitution observed in the pre-enriched pool (Table 2).

TABLE 1

| SEQ ID | Sequence (5′-3′) |
|---|---|
| SEQ ID NO: 1 | ctaaagaggagaaaggatctNNKGACAAAAAGTACTCTATTGGC |
| SEQ ID NO: 2 | agaggagaaaggatctATGNNKAAAAAGTACTCTATTGGCC |
| SEQ ID NO: 3 | agaaaggatctATGGACNNKAAGTACTCTATTGGCCTG |
| SEQ ID NO: 4 | aaggatctATGGACAAANNKTACTCTATTGGCCTGGA |
| SEQ ID NO: 5 | ggatctATGGACAAAAAGNNKTCTATTGGCCTGGATATC |
| SEQ ID NO: 6 | ctATGGACAAAAAGTACNNKATTGGCCTGGATATCGG |
| SEQ ID NO: 7 | GACAAAAAGTACTCTNNKGGCCTGGATATCGGG |
| SEQ ID NO: 8 | GACAAAAAGTACTCTATTNNKCTGGATATCGGGACCAAC |
| SEQ ID NO: 9 | AAAGTACTCTATTGGCNNKGATATCGGGACCAACA |
| SEQ ID NO: 10 | CTCTATTGGCCTGNNKATCGGGACCAACAG |
| SEQ ID NO: 11 | TTGGCCTGGATNNKGGGACCAACAG |
| SEQ ID NO: 12 | GGCCTGGATATCNNKACCAACAGCGTC |
| SEQ ID NO: 13 | CTGGATATCGGGNNKAACAGCGTCGGG |
| SEQ ID NO: 14 | ATATCGGGACCNNKAGCGTCGGGTG |
| SEQ ID NO: 15 | TCGGGACCAACNNKGTCGGGTGGGC |
| SEQ ID NO: 16 | GGACCAACAGCNNKGGGTGGGCTGT |
| SEQ ID NO: 17 | GACCAACAGCGTCNNKTGGGCTGTTATCA |
| SEQ ID NO: 18 | ACAGCGTCGGGNNKGCTGTTATCAC |
| SEQ ID NO: 19 | GCGTCGGGTGGNNKGTTATCACCGA |
| SEQ ID NO: 20 | TCGGGTGGGCTNNKATCACCGACGA |
| SEQ ID NO: 21 | GGGTGGGCTGTTNNKACCGACGAGTATA |
| SEQ ID NO: 22 | GGGTGGGCTGTTATCNNKGACGAGTATAAAGTAC |
| SEQ ID NO: 23 | GTGGGCTGTTATCACCNNKGAGTATAAAGTACCTTC |
| SEQ ID NO: 24 | GGCTGTTATCACCGACNNKTATAAAGTACCTTCGAA |
| SEQ ID NO: 25 | CTGTTATCACCGACGAGNNKAAAGTACCTTCGAAAAA |
| SEQ ID NO: 26 | TTATCACCGACGAGTATNNKGTACCTTCGAAAAAGTTC |
| SEQ ID NO: 27 | TCACCGACGAGTATAAANNKCCTTCGAAAAAGTTCAA |
| SEQ ID NO: 28 | ACCGACGAGTATAAAGTANNKTCGAAAAAGTTCAAAGTGC |
| SEQ ID NO: 29 | CGACGAGTATAAAGTACCTNNKAAAAAGTTCAAAGTGCTGG |
| SEQ ID NO: 30 | AGTATAAAGTACCTTCGNNKAAGTTCAAAGTGCTGGG |
| SEQ ID NO: 31 | ATAAAGTACCTTCGAAANNKTTCAAAGTGCTGGGCAA |
| SEQ ID NO: 32 | GTACCTTCGAAAAAGNNKAAAGTGCTGGGCAAC |
| SEQ ID NO: 33 | TTCGAAAAAGTTCNNKGTGCTGGGCAACAC |
| SEQ ID NO: 34 | CGAAAAAGTTCAAANNKCTGGGCAACACCGAT |
| SEQ ID NO: 35 | AAAAGTTCAAAGTGNNKGGCAACACCGATCG |
| SEQ ID NO: 36 | GTTCAAAGTGCTGNNKAACACCGATCGCC |
| SEQ ID NO: 37 | AAGTGCTGGGCNNKACCGATCGCCA |
| SEQ ID NO: 38 | TGCTGGGCAACNNKGATCGCCATTC |
| SEQ ID NO: 39 | CTGGGCAACACCNNKCGCCATTCAATC |
| SEQ ID NO: 40 | CTGGGCAACACCGATNNKCATTCAATCAAAAAGA |
| SEQ ID NO: 41 | GCAACACCGATCGCNNKTCAATCAAAAAGAAC |
| SEQ ID NO: 42 | CAACACCGATCGCCATNNKATCAAAAAGAACTTGAT |
| SEQ ID NO: 43 | CACCGATCGCCATTCANNKAAAAAGAACTTGATTG |
| SEQ ID NO: 44 | CGATCGCCATTCAATCNNKAAGAACTTGATTGGTG |
| SEQ ID NO: 45 | CGCCATTCAATCAAANNKAACTTGATTGGTGCG |
| SEQ ID NO: 46 | CATTCAATCAAAAAGNNKTTGATTGGTGCGCTGT |
| SEQ ID NO: 47 | ATTCAATCAAAAAGAACNNKATTGGTGCGCTGTTGTT |
| SEQ ID NO: 48 | ATCAAAAAGAACTTGNNKGGTGCGCTGTTGTTT |
| SEQ ID NO: 49 | TCAAAAAGAACTTGATTNNKGCGCTGTTGTTTGACTC |
| SEQ ID NO: 50 | AAAGAACTTGATTGGTNNKCTGTTGTTTGACTCCG |
| SEQ ID NO: 51 | CTTGATTGGTGCGNNKTTGTTTGACTCCGG |
| SEQ ID NO: 52 | TTGGTGCGCTGNNKTTTGACTCCGG |
| SEQ ID NO: 53 | GTGCGCTGTTGNNKGACTCCGGGGA |
| SEQ ID NO: 54 | CGCTGTTGTTTNNKTCCGGGGAAACC |
| SEQ ID NO: 55 | CTGTTGTTTGACNNKGGGGAAACCGCC |
| SEQ ID NO: 56 | TTGTTTGACTCCNNKGAAACCGCCGAG |
| SEQ ID NO: 57 | TTGACTCCGGGNNKACCGCCGAGGC |
| SEQ ID NO: 58 | ACTCCGGGGAANNKGCCGAGGCGAC |
| SEQ ID NO: 59 | CCGGGGAAACCNNKGAGGCGACTCG |
| SEQ ID NO: 60 | GGGAAACCGCCNNKGCGACTCGCCT |
| SEQ ID NO: 61 | GAAACCGCCGAGNNKACTCGCCTTAAAC |
| SEQ ID NO: 62 | CCGCCGAGGCGNNKCGCCTTAAACG |
| SEQ ID NO: 63 | GCCGAGGCGACTNNKCTTAAACGTACAG |
| SEQ ID NO: 64 | AGGCGACTCGCNNKAAACGTACAGC |
| SEQ ID NO: 65 | CGACTCGCCTTNNKCGTACAGCACG |
| SEQ ID NO: 66 | ACTCGCCTTAAANNKACAGCACGTCGC |
| SEQ ID NO: 67 | GCCTTAAACGTNNKGCACGTCGCCG |
| SEQ ID NO: 68 | CCTTAAACGTACANNKCGTCGCCGGTACA |
| SEQ ID NO: 69 | TAAACGTACAGCANNKCGCCGGTACACTC |
| SEQ ID NO: 70 | GTACAGCACGTNNKCGGTACACTCGG |
| SEQ ID NO: 71 | CAGCACGTCGCNNKTACACTCGGCG |
| SEQ ID NO: 72 | CACGTCGCCGGNNKACTCGGCGTAA |
| SEQ ID NO: 73 | GTCGCCGGTACNNKCGGCGTAAGAA |
| SEQ ID NO: 74 | CGCCGGTACACTNNKCGTAAGAATCGC |
| SEQ ID NO: 75 | CCGGTACACTCGGNNKAAGAATCGCATTTG |
| SEQ ID NO: 76 | GTACACTCGGCGTNNKAATCGCATTTGCTA |
| SEQ ID NO: 77 | CACTCGGCGTAAGNNKCGCATTTGCTATTT |
| SEQ ID NO: 78 | CACTCGGCGTAAGAATNNKATTTGCTATTTGCAGG |
| SEQ ID NO: 79 | GCGTAAGAATCGCNNKTGCTATTTGCAGGA |
| SEQ ID NO: 80 | GGCGTAAGAATCGCATTNNKTATTTGCAGGAAATCTT |
| SEQ ID NO: 81 | GTAAGAATCGCATTTGCNNKTTGCAGGAAATCTTTAGC |
| SEQ ID NO: 82 | AGAATCGCATTTGCTATNNKCAGGAAATCTTTAGCAAC |
| SEQ ID NO: 83 | ATCGCATTTGCTATTTGNNKGAAATCTTTAGCAACGA |
| SEQ ID NO: 84 | GCATTTGCTATTTGCAGNNKATCTTTAGCAACGAGAT |
| SEQ ID NO: 85 | TTGCTATTTGCAGGAANNKTTTAGCAACGAGATGG |
| SEQ ID NO: 86 | ATTTGCAGGAAATCNNKAGCAACGAGATGGC |
| SEQ ID NO: 87 | ATTTGCAGGAAATCTTTNNKAACGAGATGGCAAAAGT |
| SEQ ID NO: 88 | CAGGAAATCTTTAGCNNKGAGATGGCAAAAGTCG |
| SEQ ID NO: 89 | AGGAAATCTTTAGCAACNNKATGGCAAAAGTCGATGA |
| SEQ ID NO: 90 | AATCTTTAGCAACGAGNNKGCAAAAGTCGATGACT |
| SEQ ID NO: 91 | CTTTAGCAACGAGATGNNKAAAGTCGATGACTCGTT |
| SEQ ID NO: 92 | CAACGAGATGGCANNKGTCGATGACTCGTT |
| SEQ ID NO: 93 | CAACGAGATGGCAAAANNKGATGACTCGTTTTTCC |
| SEQ ID NO: 94 | GAGATGGCAAAAGTCNNKGACTCGTTTTTCCAC |
| SEQ ID NO: 95 | TGGCAAAAGTCGATNNKTCGTTTTTCCACCG |
| SEQ ID NO: 96 | CAAAAGTCGATGACNNKTTTTTCCACCGCCTC |
| SEQ ID NO: 97 | TCGATGACTCGNNKTTCCACCGCCTC |
| SEQ ID NO: 98 | GATGACTCGTTTNNKCACCGCCTCGAG |
| SEQ ID NO: 99 | TGACTCGTTTTTCNNKCGCCTCGAGGAAA |
| SEQ ID NO: 100 | ACTCGTTTTTCCACNNKCTCGAGGAAAGCTTT |
| SEQ ID NO: 101 | GTTTTTCCACCGCNNKGAGGAAAGCTTTCT |
| SEQ ID NO: 102 | TTCCACCGCCTCNNKGAAAGCTTTCTG |
| SEQ ID NO: 103 | ACCGCCTCGAGNNKAGCTTTCTGGTG |
| SEQ ID NO: 104 | CGCCTCGAGGAANNKTTTCTGGTGGAG |
| SEQ ID NO: 105 | CTCGAGGAAAGCNNKCTGGTGGAGGAAG |
| SEQ ID NO: 106 | CTCGAGGAAAGCTTTNNKGTGGAGGAAGACAAAA |
| SEQ ID NO: 107 | GAGGAAAGCTTTCTGNNKGAGGAAGACAAAAAGC |
| SEQ ID NO: 108 | GGAAAGCTTTCTGGTGNNKGAAGACAAAAAGCATG |
| SEQ ID NO: 109 | GCTTTCTGGTGGAGNNKGACAAAAAGCATGAG |
| SEQ ID NO: 110 | TTCTGGTGGAGGAANNKAAAAAGCATGAGCG |
| SEQ ID NO: 111 | GTGGAGGAAGACNNKAAGCATGAGCGTC |
| SEQ ID NO: 112 | GGAGGAAGACAAANNKCATGAGCGTCACC |
| SEQ ID NO: 113 | GAAGACAAAAAGNNKGAGCGTCACCCGA |
| SEQ ID NO: 114 | GAAGACAAAAAGCATNNKCGTCACCCGATCTTC |
| SEQ ID NO: 115 | CAAAAAGCATGAGNNKCACCCGATCTTCGG |
| SEQ ID NO: 116 | AGCATGAGCGTNNKCCGATCTTCGG |
| SEQ ID NO: 117 | CATGAGCGTCACNNKATCTTCGGCAACA |
| SEQ ID NO: 118 | AGCGTCACCCGNNKTTCGGCAACAT |
| SEQ ID NO: 119 | CGTCACCCGATCNNKGGCAACATTGTC |
| SEQ ID NO: 120 | GTCACCCGATCTTCNNKAACATTGTCGATGAA |
| SEQ ID NO: 121 | CCCGATCTTCGGCNNKATTGTCGATGAAG |
| SEQ ID NO: 122 | GATCTTCGGCAACNNKGTCGATGAAGTAGC |
| SEQ ID NO: 123 | GATCTTCGGCAACATTNNKGATGAAGTAGCGTATCA |
| SEQ ID NO: 124 | TTCGGCAACATTGTCNNKGAAGTAGCGTATCAT |
| SEQ ID NO: 125 | GGCAACATTGTCGATNNKGTAGCGTATCATGAAA |
| SEQ ID NO: 126 | GGCAACATTGTCGATGAANNKGCGTATCATGAAAAATAC |
| SEQ ID NO: 127 | GCAACATTGTCGATGAAGTANNKTATCATGAAAAATACCCAAC |
| SEQ ID NO: 128 | TGTCGATGAAGTAGCGNNKCATGAAAAATACCCAA |
| SEQ ID NO: 129 | TCGATGAAGTAGCGTATNNKGAAAAATACCCAACCAT |
| SEQ ID NO: 130 | CGATGAAGTAGCGTATCATNNKAAATACCCAACCATTTACC |
| SEQ ID NO: 131 | AAGTAGCGTATCATGAANNKTACCCAACCATTTACCAC |
| SEQ ID NO: 132 | GTAGCGTATCATGAAAAANNKCCAACCATTTACCACTTAC |
| SEQ ID NO: 133 | GCGTATCATGAAAAATACNNKACCATTTACCACTTACGC |
| SEQ ID NO: 134 | CGTATCATGAAAAATACCCANNKATTTACCACTTACGCAAAAA |
| SEQ ID NO: 135 | ATGAAAAATACCCAACCNNKTACCACTTACGCAAAAAG |
| SEQ ID NO: 136 | AAAAATACCCAACCATTNNKCACTTACGCAAAAAGCT |
| SEQ ID NO: 137 | ATACCCAACCATTTACNNKTTACGCAAAAAGCTGGT |
| SEQ ID NO: 138 | CAACCATTTACCACNNKCGCAAAAAGCTGGT |
| SEQ ID NO: 139 | CAACCATTTACCACTTANNKAAAAAGCTGGTGGACAG |
| SEQ ID NO: 140 | TTACCACTTACGCNNKAAGCTGGTGGACAG |
| SEQ ID NO: 141 | CACTTACGCAAANNKCTGGTGGACAGCA |
| SEQ ID NO: 142 | ACTTACGCAAAAAGNNKGTGGACAGCACTGA |
| SEQ ID NO: 143 | CGCAAAAAGCTGNNKGACAGCACTGACA |
| SEQ ID NO: 144 | CAAAAAGCTGGTGNNKAGCACTGACAAAGC |
| SEQ ID NO: 145 | AAAAGCTGGTGGACNNKACTGACAAAGCTGAT |
| SEQ ID NO: 146 | CTGGTGGACAGCNNKGACAAAGCTGATT |
| SEQ ID NO: 147 | GGTGGACAGCACTNNKAAAGCTGATTTGCG |
| SEQ ID NO: 148 | ACAGCACTGACNNKGCTGATTTGCG |
| SEQ ID NO: 149 | GACAGCACTGACAAANNKGATTTGCGCCTTATC |
| SEQ ID NO: 150 | AGCACTGACAAAGCTNNKTTGCGCCTTATCTAT |
| SEQ ID NO: 151 | CACTGACAAAGCTGATNNKCGCCTTATCTATTTAGC |
| SEQ ID NO: 152 | ACTGACAAAGCTGATTTGNNKCTTATCTATTTAGCCCTG |
| SEQ ID NO: 153 | AAAGCTGATTTGCGCNNKATCTATTTAGCCCTG |
| SEQ ID NO: 154 | CTGATTTGCGCCTTNNKTATTTAGCCCTGGC |
| SEQ ID NO: 155 | TTGCGCCTTATCNNKTTAGCCCTGGCA |
| SEQ ID NO: 156 | GCGCCTTATCTATNNKGCCCTGGCACATAT |
| SEQ ID NO: 157 | TTGCGCCTTATCTATTTANNKCTGGCACATATGATTAAGT |
| SEQ ID NO: 158 | GCGCCTTATCTATTTAGCCNNKGCACATATGATTAAGTTTC |
| SEQ ID NO: 159 | CCTTATCTATTTAGCCCTGNNKCATATGATTAAGTTTCGTGG |
| SEQ ID NO: 160 | TCTATTTAGCCCTGGCANNKATGATTAAGTTTCGTGG |
| SEQ ID NO: 161 | TTTAGCCCTGGCACATNNKATTAAGTTTCGTGGTC |
| SEQ ID NO: 162 | CCCTGGCACATATGNNKAAGTTTCGTGGTCA |
| SEQ ID NO: 163 | CTGGCACATATGATTNNKTTTCGTGGTCACTTCC |
| SEQ ID NO: 164 | GCACATATGATTAAGNNKCGTGGTCACTTCCTGA |
| SEQ ID NO: 165 | GCACATATGATTAAGTTTNNKGGTCACTTCCTGATCGAA |
| SEQ ID NO: 166 | CATATGATTAAGTTTCGTNNKCACTTCCTGATCGAAGGA |
| SEQ ID NO: 167 | GATTAAGTTTCGTGGTNNKTTCCTGATCGAAGGAGA |
| SEQ ID NO: 168 | GTTTCGTGGTCACNNKCTGATCGAAGGAGA |
| SEQ ID NO: 169 | GTTTCGTGGTCACTTCNNKATCGAAGGAGACTTAAA |
| SEQ ID NO: 170 | CGTGGTCACTTCCTGNNKGAAGGAGACTTAAATC |
| SEQ ID NO: 171 | GGTCACTTCCTGATCNNKGGAGACTTAAATCCC |
| SEQ ID NO: 172 | TCACTTCCTGATCGAANNKGACTTAAATCCCGACAA |
| SEQ ID NO: 173 | CTTCCTGATCGAAGGANNKTTAAATCCCGACAACAG |
| SEQ ID NO: 174 | TGATCGAAGGAGACNNKAATCCCGACAACAG |
| SEQ ID NO: 175 | ATCGAAGGAGACTTANNKCCCGACAACAGTGAT |
| SEQ ID NO: 176 | TCGAAGGAGACTTAAATNNKGACAACAGTGATGTTGAT |
| SEQ ID NO: 177 | CGAAGGAGACTTAAATCCCNNKAACAGTGATGTTGATAAAT |
| SEQ ID NO: 178 | GGAGACTTAAATCCCGACNNKAGTGATGTTGATAAATTGT |
| SEQ ID NO: 179 | GGAGACTTAAATCCCGACAACNNKGATGTTGATAAATTGTTTATT |
| SEQ ID NO: 180 | GACTTAAATCCCGACAACAGTNNKGTTGATAAATTGTTTATTCAG |
| SEQ ID NO: 181 | AAATCCCGACAACAGTGATNNKGATAAATTGTTTATTCAGC |
| SEQ ID NO: 182 | CCCGACAACAGTGATGTTNNKAAATTGTTTATTCAGCTT |
| SEQ ID NO: 183 | GACAACAGTGATGTTGATNNKTTGTTTATTCAGCTTGTCC |
| SEQ ID NO: 184 | ACAACAGTGATGTTGATAAANNKTTTATTCAGCTTGTCCAAAC |
| SEQ ID NO: 185 | CAGTGATGTTGATAAATTGNNKATTCAGCTTGTCCAAACTT |
| SEQ ID NO: 186 | GTGATGTTGATAAATTGTTTNNKCAGCTTGTCCAAACTTACAA |
| SEQ ID NO: 187 | GTGATGTTGATAAATTGTTTATTNNKCTTGTCCAAACTTACAATCAACT |
| SEQ ID NO: 188 | GTTGATAAATTGTTTATTCAGNNKGTCCAAACTTACAATCAACTGT |
| SEQ ID NO: 189 | TGATAAATTGTTTATTCAGCTTNNKCAAACTTACAATCAACTGTTCG |
| SEQ ID NO: 190 | TTGTTTATTCAGCTTGTCNNKACTTACAATCAACTGTTCG |
| SEQ ID NO: 191 | TTTATTCAGCTTGTCCAANNKTACAATCAACTGTTCGAGG |
| SEQ ID NO: 192 | TCAGCTTGTCCAAACTNNKAATCAACTGTTCGAGG |
| SEQ ID NO: 193 | CTTGTCCAAACTTACNNKCAACTGTTCGAGGAAA |
| SEQ ID NO: 194 | TTGTCCAAACTTACAATNNKCTGTTCGAGGAAAACCC |
| SEQ ID NO: 195 | TCCAAACTTACAATCAANNKTTCGAGGAAAACCCGAT |
| SEQ ID NO: 196 | AAACTTACAATCAACTGNNKGAGGAAAACCCGATCAA |
| SEQ ID NO: 197 | CTTACAATCAACTGTTCNNKGAAAACCCGATCAATGC |
| SEQ ID NO: 198 | ATCAACTGTTCGAGNNKAACCCGATCAATGC |
| SEQ ID NO: 199 | AACTGTTCGAGGAANNKCCGATCAATGCCTC |
| SEQ ID NO: 200 | GTTCGAGGAAAACNNKATCAATGCCTCCGG |
| SEQ ID NO: 201 | GAGGAAAACCCGNNKAATGCCTCCGGT |
| SEQ ID NO: 202 | AAAACCCGATCNNKGCCTCCGGTGT |
| SEQ ID NO: 203 | AAACCCGATCAATNNKTCCGGTGTGGATG |
| SEQ ID NO: 204 | CGATCAATGCCNNKGGTGTGGATGCA |
| SEQ ID NO: 205 | GATCAATGCCTCCNNKGTGGATGCAAAAG |
| SEQ ID NO: 206 | AATGCCTCCGGTNNKGATGCAAAAGCC |
| SEQ ID NO: 207 | CCTCCGGTGTGNNKGCAAAAGCCATT |
| SEQ ID NO: 208 | CCTCCGGTGTGGATNNKAAAGCCATTTTAAGT |
| SEQ ID NO: 209 | CGGTGTGGATGCANNKGCCATTTTAAGTG |
| SEQ ID NO: 210 | GGTGTGGATGCAAAANNKATTTTAAGTGCACGC |
| SEQ ID NO: 211 | GATGCAAAAGCCNNKTTAAGTGCACGCC |
| SEQ ID NO: 212 | ATGCAAAAGCCATTNNKAGTGCACGCCTTAG |
| SEQ ID NO: 213 | CAAAAGCCATTTTANNKGCACGCCTTAGCAAG |
| SEQ ID NO: 214 | AAAGCCATTTTAAGTNNKCGCCTTAGCAAGTCC |
| SEQ ID NO: 215 | CCATTTTAAGTGCANNKCTTAGCAAGTCCCGT |
| SEQ ID NO: 216 | TAAGTGCACGCNNKAGCAAGTCCCG |
| SEQ ID NO: 217 | GTGCACGCCTTNNKAAGTCCCGTCG |
| SEQ ID NO: 218 | CACGCCTTAGCNNKTCCCGTCGCTTA |
| SEQ ID NO: 219 | CGCCTTAGCAAGNNKCGTCGCTTAGAAA |
| SEQ ID NO: 220 | GCCTTAGCAAGTCCNNKCGCTTAGAAAACCT |
| SEQ ID NO: 221 | CCTTAGCAAGTCCCGTNNKTTAGAAAACCTTATCG |
| SEQ ID NO: 222 | CAAGTCCCGTCGCNNKGAAAACCTTATCG |
| SEQ ID NO: 223 | TCCCGTCGCTTANNKAACCTTATCGCG |
| SEQ ID NO: 224 | CGTCGCTTAGAANNKCTTATCGCGCAGC |
| SEQ ID NO: 225 | CGCTTAGAAAACNNKATCGCGCAGCTG |
| SEQ ID NO: 226 | TTAGAAAACCTTNNKGCGCAGCTGCCC |
| SEQ ID NO: 227 | GAAAACCTTATCNNKCAGCTGCCCGGC |
| SEQ ID NO: 228 | ACCTTATCGCGNNKCTGCCCGGCGA |
| SEQ ID NO: 229 | TTATCGCGCAGNNKCCCGGCGAGAA |
| SEQ ID NO: 230 | TCGCGCAGCTGNNKGGCGAGAAAAA |
| SEQ ID NO: 231 | GCGCAGCTGCCCNNKGAGAAAAAGAAT |
| SEQ ID NO: 232 | CAGCTGCCCGGCNNKAAAAAGAATGGTT |
| SEQ ID NO: 233 | CTGCCCGGCGAGNNKAAGAATGGTTTG |
| SEQ ID NO: 234 | GCCCGGCGAGAAANNKAATGGTTTGTTTG |
| SEQ ID NO: 235 | CGGCGAGAAAAAGNNKGGTTTGTTTGGGAA |
| SEQ ID NO: 236 | CGGCGAGAAAAAGAATNNKTTGTTTGGGAACCTTAT |
| SEQ ID NO: 237 | CGAGAAAAAGAATGGTNNKTTTGGGAACCTTATTGC |
| SEQ ID NO: 238 | AGAAAAAGAATGGTTTGNNKGGGAACCTTATTGCCTT |
| SEQ ID NO: 239 | AAAAAGAATGGTTTGTTTNNKAACCTTATTGCCTTGAGCT |
| SEQ ID NO: 240 | AATGGTTTGTTTGGGNNKCTTATTGCCTTGAGC |
| SEQ ID NO: 241 | GGTTTGTTTGGGAACNNKATTGCCTTGAGCTTAG |
| SEQ ID NO: 242 | TGTTTGGGAACCTTNNKGCCTTGAGCTTAGG |
| SEQ ID NO: 243 | GTTTGGGAACCTTATTNNKTTGAGCTTAGGCCTCA |
| SEQ ID NO: 244 | GAACCTTATTGCCNNKAGCTTAGGCCTCAC |
| SEQ ID NO: 245 | CCTTATTGCCTTGNNKTTAGGCCTCACCCC |
| SEQ ID NO: 246 | TTGCCTTGAGCNNKGGCCTCACCCC |
| SEQ ID NO: 247 | TTGCCTTGAGCTTANNKCTCACCCCGAATTT |
| SEQ ID NO: 248 | CTTGAGCTTAGGCNNKACCCCGAATTTCAA |
| SEQ ID NO: 249 | CTTGAGCTTAGGCCTCNNKCCGAATTTCAAAAGTAA |
| SEQ ID NO: 250 | TTGAGCTTAGGCCTCACCNNKAATTTCAAAAGTAATTTC |
| SEQ ID NO: 251 | TTAGGCCTCACCCCGNNKTTCAAAAGTAATTTCG |
| SEQ ID NO: 252 | TAGGCCTCACCCCGAATNNKAAAAGTAATTTCGATCT |
| SEQ ID NO: 253 | CCTCACCCCGAATTTCNNKAGTAATTTCGATCTTG |
| SEQ ID NO: 254 | CACCCCGAATTTCAAANNKAATTTCGATCTTGCAGA |
| SEQ ID NO: 255 | CCCGAATTTCAAAAGTNNKTTCGATCTTGCAGAAGA |
| SEQ ID NO: 256 | CGAATTTCAAAAGTAATNNKGATCTTGCAGAAGACGCC |
| SEQ ID NO: 257 | ATTTCAAAAGTAATTTCNNKCTTGCAGAAGACGCCAA |
| SEQ ID NO: 258 | TCAAAAGTAATTTCGATNNKGCAGAAGACGCCAAATT |
| SEQ ID NO: 259 | CAAAAGTAATTTCGATCTTNNKGAAGACGCCAAATTACAACT |
| SEQ ID NO: 260 | GTAATTTCGATCTTGCANNKGACGCCAAATTACAACT |
| SEQ ID NO: 261 | ATTTCGATCTTGCAGAANNKGCCAAATTACAACTGTC |
| SEQ ID NO: 262 | TCGATCTTGCAGAAGACNNKAAATTACAACTGTCGAA |
| SEQ ID NO: 263 | TTGCAGAAGACGCCNNKTTACAACTGTCGAA |
| SEQ ID NO: 264 | AGAAGACGCCAAANNKCAACTGTCGAAGGA |
| SEQ ID NO: 265 | CAGAAGACGCCAAATTANNKCTGTCGAAGGATACTTA |
| SEQ ID NO: 266 | AGAAGACGCCAAATTACAANNKTCGAAGGATACTTATGATG |
| SEQ ID NO: 267 | GACGCCAAATTACAACTGNNKAAGGATACTTATGATGACG |
| SEQ ID NO: 268 | GCCAAATTACAACTGTCGNNKGATACTTATGATGACGAT |
| SEQ ID NO: 269 | AAATTACAACTGTCGAAGNNKACTTATGATGACGATCTCG |
| SEQ ID NO: 270 | TACAACTGTCGAAGGATNNKTATGATGACGATCTCGA |
| SEQ ID NO: 271 | AACTGTCGAAGGATACTNNKGATGACGATCTCGATAA |
| SEQ ID NO: 272 | CTGTCGAAGGATACTTATNNKGACGATCTCGATAATCTGT |
| SEQ ID NO: 273 | TGTCGAAGGATACTTATGATNNKGATCTCGATAATCTGTTAGC |
| SEQ ID NO: 274 | AAGGATACTTATGATGACNNKCTCGATAATCTGTTAGCGC |
| SEQ ID NO: 275 | GGATACTTATGATGACGATNNKGATAATCTGTTAGCGCAGA |
| SEQ ID NO: 276 | CTTATGATGACGATCTCNNKAATCTGTTAGCGCAGAT |
| SEQ ID NO: 277 | GATGACGATCTCGATNNKCTGTTAGCGCAGATT |
| SEQ ID NO: 278 | TGACGATCTCGATAATNNKTTAGCGCAGATTGGTG |
| SEQ ID NO: 279 | ATCTCGATAATCTGNNKGCGCAGATTGGTGAC |
| SEQ ID NO: 280 | GATCTCGATAATCTGTTANNKCAGATTGGTGACCAATACG |
| SEQ ID NO: 281 | CGATAATCTGTTAGCGNNKATTGGTGACCAATACG |
| SEQ ID NO: 282 | TCTGTTAGCGCAGNNKGGTGACCAATACG |
| SEQ ID NO: 283 | GTTAGCGCAGATTNNKGACCAATACGCCG |
| SEQ ID NO: 284 | GCGCAGATTGGTNNKCAATACGCCGATC |
| SEQ ID NO: 285 | GCAGATTGGTGACNNKTACGCCGATCTTTT |
| SEQ ID NO: 286 | CAGATTGGTGACCAANNKGCCGATCTTTTTCTG |
| SEQ ID NO: 287 | ATTGGTGACCAATACNNKGATCTTTTTCTGGCG |
| SEQ ID NO: 288 | GACCAATACGCCNNKCTTTTTCTGGCGG |
| SEQ ID NO: 289 | CAATACGCCGATNNKTTTCTGGCGGCTA |
| SEQ ID NO: 290 | AATACGCCGATCTTNNKCTGGCGGCTAAAAAT |
| SEQ ID NO: 291 | TACGCCGATCTTTTTNNKGCGGCTAAAAATCTG |
| SEQ ID NO: 292 | GCCGATCTTTTTCTGNNKGCTAAAAATCTGAGCG |
| SEQ ID NO: 293 | GATCTTTTTCTGGCGNNKAAAAATCTGAGCGACG |
| SEQ ID NO: 294 | TTTCTGGCGGCTNNKAATCTGAGCGAC |
| SEQ ID NO: 295 | TGGCGGCTAAANNKCTGAGCGACGC |
| SEQ ID NO: 296 | GCGGCTAAAAATNNKAGCGACGCCATCT |
| SEQ ID NO: 297 | GGCTAAAAATCTGNNKGACGCCATCTTGCT |
| SEQ ID NO: 298 | CTAAAAATCTGAGCNNKGCCATCTTGCTTTCG |
| SEQ ID NO: 299 | GCTAAAAATCTGAGCGACNNKATCTTGCTTTCGGATATT |
| SEQ ID NO: 300 | TGAGCGACGCCNNKTTGCTTTCGGA |
| SEQ ID NO: 301 | GAGCGACGCCATCNNKCTTTCGGATATTCT |
| SEQ ID NO: 302 | CGACGCCATCTTGNNKTCGGATATTCTCC |
| SEQ ID NO: 303 | CGCCATCTTGCTTNNKGATATTCTCCGCG |
| SEQ ID NO: 304 | CCATCTTGCTITCGNNKATTCTCCGCGTTAAC |
| SEQ ID NO: 305 | CTTGCTTTCGGATNNKCTCCGCGTTAACAC |
| SEQ ID NO: 306 | TGCTTTCGGATATTNNKCGCGTTAACACCGA |
| SEQ ID NO: 307 | GCTTTCGGATATTCTCNNKGTTAACACCGAAATCAC |
| SEQ ID NO: 308 | CGGATATTCTCCGCNNKAACACCGAAATCAC |
| SEQ ID NO: 309 | GATATTCTCCGCGTTNNKACCGAAATCACGAAAG |
| SEQ ID NO: 310 | TTCTCCGCGTTAACNNKGAAATCACGAAAGC |
| SEQ ID NO: 311 | CGCGTTAACACCNNKATCACGAAAGCG |
| SEQ ID NO: 312 | GTTAACACCGAANNKACGAAAGCGCCTC |
| SEQ ID NO: 313 | GTTAACACCGAAATCNNKAAAGCGCCTCTTAGTG |
| SEQ ID NO: 314 | ACCGAAATCACGNNKGCGCCTCTTAGT |
| SEQ ID NO: 315 | CCGAAATCACGAAANNKCCTCTTAGTGCCAG |
| SEQ ID NO: 316 | ATCACGAAAGCGNNKCTTAGTGCCAGC |
| SEQ ID NO: 317 | CGAAAGCGCCTNNKAGTGCCAGCATG |
| SEQ ID NO: 318 | GAAAGCGCCTCTTNNKGCCAGCATGATTAA |
| SEQ ID NO: 319 | CGAAAGCGCCTCTTAGTNNKAGCATGATTAAACGTTA |
| SEQ ID NO: 320 | GCGCCTCTTAGTGCCNNKATGATTAAACGTTATG |
| SEQ ID NO: 321 | GCCTCTTAGTGCCAGCNNKATTAAACGTTATGATGA |
| SEQ ID NO: 322 | CTCTTAGTGCCAGCATGNNKAAACGTTATGATGAACA |
| SEQ ID NO: 323 | TAGTGCCAGCATGATTNNKCGTTATGATGAACACC |
| SEQ ID NO: 324 | TGCCAGCATGATTAAANNKTATGATGAACACCACCA |
| SEQ ID NO: 325 | AGCATGATTAAACGTNNKGATGAACACCACCAG |
| SEQ ID NO: 326 | CATGATTAAACGTTATNNKGAACACCACCAGGACCT |
| SEQ ID NO: 327 | GATTAAACGTTATGATNNKCACCACCAGGACCTGAC |
| SEQ ID NO: 328 | TAAACGTTATGATGAANNKCACCAGGACCTGACCT |
| SEQ ID NO: 329 | CGTTATGATGAACACNNKCAGGACCTGACCTTAC |
| SEQ ID NO: 330 | TTATGATGAACACCACNNKGACCTGACCTTACTCAA |
| SEQ ID NO: 331 | GATGAACACCACCAGNNKCTGACCTTACTCAAAG |
| SEQ ID NO: 332 | AACACCACCAGGACNNKACCTTACTCAAAGC |
| SEQ ID NO: 333 | CCACCAGGACCTGNNKTTACTCAAAGCGTT |
| SEQ ID NO: 334 | CAGGACCTGACCNNKCTCAAAGCGTTG |
| SEQ ID NO: 335 | GGACCTGACCTTANNKAAAGCGTTGGTTCG |
| SEQ ID NO: 336 | CTGACCTTACTCNNKGCGTTGGTTCGC |
| SEQ ID NO: 337 | TGACCTTACTCAAANNKTTGGTTCGCCAGCAA |
| SEQ ID NO: 338 | CTTACTCAAAGCGNNKGTTCGCCAGCAAC |
| SEQ ID NO: 339 | TCAAAGCGTTGNNKCGCCAGCAACT |
| SEQ ID NO: 340 | AAAGCGTTGGTTNNKCAGCAACTGCCAG |
| SEQ ID NO: 341 | CGTTGGTTCGCNNKCAACTGCCAGAG |
| SEQ ID NO: 342 | TGGTTCGCCAGNNKCTGCCAGAGAA |
| SEQ ID NO: 343 | GGTTCGCCAGCAANNKCCAGAGAAGTACA |
| SEQ ID NO: 344 | TTCGCCAGCAACTGNNKGAGAAGTACAAAGAA |
| SEQ ID NO: 345 | GCCAGCAACTGCCANNKAAGTACAAAGAAATC |
| SEQ ID NO: 346 | CCAGCAACTGCCAGAGNNKTACAAAGAAATCTTCTT |
| SEQ ID NO: 347 | AGCAACTGCCAGAGAAGNNKAAAGAAATCTTCTTTGA |
| SEQ ID NO: 348 | AACTGCCAGAGAAGTACNNKGAAATCTTCTTTGATCA |
| SEQ ID NO: 349 | CTGCCAGAGAAGTACAAANNKATCTTCTTTGATCAGTCA |
| SEQ ID NO: 350 | GCCAGAGAAGTACAAAGAANNKTTCTTTGATCAGTCAAAGA |
| SEQ ID NO: 351 | CAGAGAAGTACAAAGAAATCNNKTTTGATCAGTCAAAGAATGGT |
| SEQ ID NO: 352 | GAGAAGTACAAAGAAATCTTCNNKGATCAGTCAAAGAATGGTTAT |
| SEQ ID NO: 353 | AGTACAAAGAAATCTTCTTTNNKCAGTCAAAGAATGGTTATGCC |
| SEQ ID NO: 354 | CAAAGAAATCTTCTTTGATNNKTCAAAGAATGGTTATGCCGG |
| SEQ ID NO: 355 | AAATCTTCTTTGATCAGNNKAAGAATGGTTATGCCGGC |
| SEQ ID NO: 356 | CTTCTTTGATCAGTCANNKAATGGTTATGCCGGCTA |
| SEQ ID NO: 357 | CTTTGATCAGTCAAAGNNKGGTTATGCCGGCTATAT |
| SEQ ID NO: 358 | TTTGATCAGTCAAAGAATNNKTATGCCGGCTATATTGACG |
| SEQ ID NO: 359 | CAGTCAAAGAATGGTNNKGCCGGCTATATTGAC |
| SEQ ID NO: 360 | GTCAAAGAATGGTTATNNKGGCTATATTGACGGGGG |
| SEQ ID NO: 361 | AAAGAATGGTTATGCCNNKTATATTGACGGGGGTG |
| SEQ ID NO: 362 | GTTATGCCGGCNNKATTGACGGGGG |
| SEQ ID NO: 363 | ATGCCGGCTATNNKGACGGGGGTGC |
| SEQ ID NO: 364 | CCGGCTATATTNNKGGGGGTGCAAGC |
| SEQ ID NO: 365 | CGGCTATATTGACNNKGGTGCAAGCCAAG |
| SEQ ID NO: 366 | CTATATTGACGGGNNKGCAAGCCAAGAGGA |
| SEQ ID NO: 367 | CTATATTGACGGGGGTNNKAGCCAAGAGGAATTCTA |
| SEQ ID NO: 368 | GACGGGGGTGCANNKCAAGAGGAATTCT |
| SEQ ID NO: 369 | GGGGGTGCAAGCNNKGAGGAATTCTACA |
| SEQ ID NO: 370 | GGGGGTGCAAGCCAANNKGAATTCTACAAATTTA |
| SEQ ID NO: 371 | GGGTGCAAGCCAAGAGNNKTTCTACAAATTTATCAA |
| SEQ ID NO: 372 | GTGCAAGCCAAGAGGAANNKTACAAATTTATCAAGCC |
| SEQ ID NO: 373 | GCAAGCCAAGAGGAATTCNNKAAATTTATCAAGCCTATT |
| SEQ ID NO: 374 | AGCCAAGAGGAATTCTACNNKTTTATCAAGCCTATTCTGG |
| SEQ ID NO: 375 | CCAAGAGGAATTCTACAAANNKATCAAGCCTATTCTGGAGA |
| SEQ ID NO: 376 | CAAGAGGAATTCTACAAATTTNNKAAGCCTATTCTGGAGAAAATGG |
| SEQ ID NO: 377 | GAGGAATTCTACAAATTTATCNNKCCTATTCTGGAGAAAATGGATG |
| SEQ ID NO: 378 | GAATTCTACAAATTTATCAAGNNKATTCTGGAGAAAATGGATGGC |
| SEQ ID NO: 379 | ACAAATTTATCAAGCCTNNKCTGGAGAAAATGGATGGC |
| SEQ ID NO: 380 | AAATTTATCAAGCCTATTNNKGAGAAAATGGATGGCACCG |
| SEQ ID NO: 381 | TATCAAGCCTATTCTGNNKAAAATGGATGGCACCG |
| SEQ ID NO: 382 | GCCTATTCTGGAGNNKATGGATGGCACCG |
| SEQ ID NO: 383 | CCTATTCTGGAGAAANNKGATGGCACCGAAGAG |
| SEQ ID NO: 384 | CTATTCTGGAGAAAATGNNKGGCACCGAAGAGTTATT |
| SEQ ID NO: 385 | TTCTGGAGAAAATGGATNNKACCGAAGAGTTATTGGTG |
| SEQ ID NO: 386 | GGAGAAAATGGATGGCNNKGAAGAGTTATTGGTGAA |
| SEQ ID NO: 387 | AAAATGGATGGCACCNNKGAGTTATTGGTGAAG |
| SEQ ID NO: 388 | AAATGGATGGCACCGAANNKTTATTGGTGAAGCTTAA |
| SEQ ID NO: 389 | ATGGCACCGAAGAGNNKTTGGTGAAGCTTAA |
| SEQ ID NO: 390 | GCACCGAAGAGTTANNKGTGAAGCTTAACCGT |
| SEQ ID NO: 391 | CACCGAAGAGTTATTGNNKAAGCTTAACCGTGAAGA |
| SEQ ID NO: 392 | GAAGAGTTATTGGTGNNKCTTAACCGTGAAGACC |
| SEQ ID NO: 393 | GAGTTATTGGTGAAGNNKAACCGTGAAGACCTCC |
| SEQ ID NO: 394 | TTATTGGTGAAGCTTNNKCGTGAAGACCTCCTG |
| SEQ ID NO: 395 | GGTGAAGCTTAACNNKGAAGACCTCCTGC |
| SEQ ID NO: 396 | AAGCTTAACCGTNNKGACCTCCTGCGG |
| SEQ ID NO: 397 | GCTTAACCGTGAANNKCTCCTGCGGAAAC |
| SEQ ID NO: 398 | AACCGTGAAGACNNKCTGCGGAAACAG |
| SEQ ID NO: 399 | GTGAAGACCTCNNKCGGAAACAGCGC |
| SEQ ID NO: 400 | TGAAGACCTCCTGNNKAAACAGCGCACATT |
| SEQ ID NO: 401 | ACCTCCTGCGGNNKCAGCGCACATT |
| SEQ ID NO: 402 | CTCCTGCGGAAANNKCGCACATTCGATA |
| SEQ ID NO: 403 | CTCCTGCGGAAACAGNNKACATTCGATAATGGTT |
| SEQ ID NO: 404 | GCGGAAACAGCGCNNKTTCGATAATGGTT |
| SEQ ID NO: 405 | GGAAACAGCGCACANNKGATAATGGTTCGATC |
| SEQ ID NO: 406 | ACAGCGCACATTCNNKAATGGTTCGATCC |
| SEQ ID NO: 407 | CGCACATTCGATNNKGGTTCGATCCCAC |
| SEQ ID NO: 408 | GCACATTCGATAATNNKTCGATCCCACACCAA |
| SEQ ID NO: 409 | CACATTCGATAATGGTNNKATCCCACACCAAATCCA |
| SEQ ID NO: 410 | TTCGATAATGGTTCGNNKCCACACCAAATCCAT |
| SEQ ID NO: 411 | CGATAATGGTTCGATCNNKCACCAAATCCATTTGG |
| SEQ ID NO: 412 | ATGGTTCGATCCCANNKCAAATCCATTTGGG |
| SEQ ID NO: 413 | TTCGATCCCACACNNKATCCATTTGGGGG |
| SEQ ID NO: 414 | GATCCCACACCAANNKCATTTGGGGGAGTT |
| SEQ ID NO: 415 | CCCACACCAAATCNNKTTGGGGGAGTTACA |
| SEQ ID NO: 416 | ACACCAAATCCATNNKGGGGAGTTACACG |
| SEQ ID NO: 417 | CACACCAAATCCATTTGNNKGAGTTACACGCTATTTT |
| SEQ ID NO: 418 | CAAATCCATTTGGGGNNKTTACACGCTATTTTGC |
| SEQ ID NO: 419 | CATTTGGGGGAGNNKCACGCTATTTTGC |
| SEQ ID NO: 420 | ATTTGGGGGAGTTANNKGCTATTTTGCGTCG |
| SEQ ID NO: 421 | GGGGAGTTACACNNKATTTTGCGTCGCC |
| SEQ ID NO: 422 | AGTTACACGCTNNKTTGCGTCGCCAG |
| SEQ ID NO: 423 | GTTACACGCTATTNNKCGTCGCCAGGAAGA |
| SEQ ID NO: 424 | TACACGCTATTTTGNNKCGCCAGGAAGACTT |
| SEQ ID NO: 425 | CACGCTATTTTGCGTNNKCAGGAAGACTTTTACC |
| SEQ ID NO: 426 | CTATTTTGCGTCGCNNKGAAGACTTTTACCCT |
| SEQ ID NO: 427 | TTGCGTCGCCAGNNKGACTTTTACCCTT |
| SEQ ID NO: 428 | GCGTCGCCAGGAANNKTTTTACCCTTTCC |
| SEQ ID NO: 429 | TCGCCAGGAAGACNNKTACCCTTTCCTGAA |
| SEQ ID NO: 430 | GCCAGGAAGACTTTNNKCCTTTCCTGAAGGAT |
| SEQ ID NO: 431 | CCAGGAAGACTTTTACNNKTTCCTGAAGGATAACCG |
| SEQ ID NO: 432 | GAAGACTTTTACCCTNNKCTGAAGGATAACCGGG |
| SEQ ID NO: 433 | AAGACTITTACCCTTTCNNKAAGGATAACCGGGAGAA |
| SEQ ID NO: 434 | CTTTTACCCTTTCCTGNNKGATAACCGGGAGAAAAT |
| SEQ ID NO: 435 | TTACCCTTTCCTGAAGNNKAACCGGGAGAAAATTG |
| SEQ ID NO: 436 | CCTTTCCTGAAGGATNNKCGGGAGAAAATTGAGA |
| SEQ ID NO: 437 | CCTTTCCTGAAGGATAACNNKGAGAAAATTGAGAAGATCC |
| SEQ ID NO: 438 | TCCTGAAGGATAACCGGNNKAAAATTGAGAAGATCCT |
| SEQ ID NO: 439 | TGAAGGATAACCGGGAGNNKATTGAGAAGATCCTTAC |
| SEQ ID NO: 440 | GGATAACCGGGAGAAANNKGAGAAGATCCTTACCTT |
| SEQ ID NO: 441 | GGATAACCGGGAGAAAATTNNKAAGATCCTTACCTTTCGTA |
| SEQ ID NO: 442 | ACCGGGAGAAAATTGAGNNKATCCTTACCTTTCGTAT |
| SEQ ID NO: 443 | GGGAGAAAATTGAGAAGNNKCTTACCTTTCGTATTCCG |
| SEQ ID NO: 444 | GGAGAAAATTGAGAAGATCNNKACCTTTCGTATTCCGTATT |
| SEQ ID NO: 445 | AGAAAATTGAGAAGATCCTTNNKTTTCGTATTCCGTATTACGT |
| SEQ ID NO: 446 | TTGAGAAGATCCTTACCNNKCGTATTCCGTATTACGT |
| SEQ ID NO: 447 | GAGAAGATCCTTACCTTTNNKATTCCGTATTACGTAGGCC |
| SEQ ID NO: 448 | ATCCTTACCTTTCGTNNKCCGTATTACGTAGGC |
| SEQ ID NO: 449 | TCCTTACCTTTCGTATTNNKTATTACGTAGGCCCCTT |
| SEQ ID NO: 450 | CTTTCGTATTCCGNNKTACGTAGGCCCCTT |
| SEQ ID NO: 451 | TTTCGTATTCCGTATNNKGTAGGCCCCTTAGCA |
| SEQ ID NO: 452 | GTATTCCGTATTACNNKGGCCCCTTAGCACG |
| SEQ ID NO: 453 | TCCGTATTACGTANNKCCCTTAGCACGGG |
| SEQ ID NO: 454 | CGTATTACGTAGGCNNKTTAGCACGGGGTAAT |
| SEQ ID NO: 455 | ACGTAGGCCCCNNKGCACGGGGTAA |
| SEQ ID NO: 456 | GTAGGCCCCTTANNKCGGGGTAATAGCC |
| SEQ ID NO: 457 | TAGGCCCCTTAGCANNKGGTAATAGCCGTTT |
| SEQ ID NO: 458 | CCCTTAGCACGGNNKAATAGCCGTTTCG |
| SEQ ID NO: 459 | TAGCACGGGGTNNKAGCCGTTTCGC |
| SEQ ID NO: 460 | CACGGGGTAATNNKCGTTTCGCGTGG |
| SEQ ID NO: 461 | CGGGGTAATAGCNNKTTCGCGTGGATGA |
| SEQ ID NO: 462 | GGTAATAGCCGTNNKGCGTGGATGACAC |
| SEQ ID NO: 463 | GGTAATAGCCGTTTCNNKTGGATGACACGGAAG |
| SEQ ID NO: 464 | GCCGTTTCGCGNNKATGACACGGAA |
| SEQ ID NO: 465 | GTTTCGCGTGGNNKACACGGAAGTCG |
| SEQ ID NO: 466 | TCGCGTGGATGNNKCGGAAGTCGGA |
| SEQ ID NO: 467 | CGCGTGGATGACANNKAAGTCGGAAGAGA |
| SEQ ID NO: 468 | TGGATGACACGGNNKTCGGAAGAGACG |
| SEQ ID NO: 469 | GGATGACACGGAAGNNKGAAGAGACGATCAC |
| SEQ ID NO: 470 | ACACGGAAGTCGNNKGAGACGATCACC |
| SEQ ID NO: 471 | GGAAGTCGGAANNKACGATCACCCCG |
| SEQ ID NO: 472 | AAGTCGGAAGAGNNKATCACCCCGTGGA |
| SEQ ID NO: 473 | CGGAAGAGACGNNKACCCCGTGGAA |
| SEQ ID NO: 474 | GGAAGAGACGATCNNKCCGTGGAACTTCG |
| SEQ ID NO: 475 | GAAGAGACGATCACCNNKTGGAACTTCGAAGAG |
| SEQ ID NO: 476 | GACGATCACCCCGNNKAACTTCGAAGAGG |
| SEQ ID NO: 477 | GATCACCCCGTGGNNKTTCGAAGAGGTAG |
| SEQ ID NO: 478 | ACCCCGTGGAACNNKGAAGAGGTAGTC |
| SEQ ID NO: 479 | CCGTGGAACTTCNNKGAGGTAGTCGACA |
| SEQ ID NO: 480 | CGTGGAACTTCGAANNKGTAGTCGACAAGGG |
| SEQ ID NO: 481 | AACTTCGAAGAGNNKGTCGACAAGGGCG |
| SEQ ID NO: 482 | CTTCGAAGAGGTANNKGACAAGGGCGCAT |
| SEQ ID NO: 483 | GAAGAGGTAGTCNNKAAGGGCGCATCAG |
| SEQ ID NO: 484 | AGGTAGTCGACNNKGGCGCATCAGC |
| SEQ ID NO: 485 | GTAGTCGACAAGNNKGCATCAGCGCAG |
| SEQ ID NO: 486 | TCGACAAGGGCNNKTCAGCGCAGTC |
| SEQ ID NO: 487 | ACAAGGGCGCANNKGCGCAGTCTTT |
| SEQ ID NO: 488 | GACAAGGGCGCATCANNKCAGTCTTTTATTGAAC |
| SEQ ID NO: 489 | GGGCGCATCAGCGNNKTCTTTTATTGAAC |
| SEQ ID NO: 490 | GCGCATCAGCGCAGNNKTTTATTGAACGTAT |
| SEQ ID NO: 491 | CATCAGCGCAGTCTNNKATTGAACGTATGACG |
| SEQ ID NO: 492 | CAGCGCAGTCTTTTNNKGAACGTATGACGAAT |
| SEQ ID NO: 493 | AGCGCAGTCTTTTATTNNKCGTATGACGAATTTCGA |
| SEQ ID NO: 494 | AGCGCAGTCTTTTATTGAANNKATGACGAATTTCGATAAAA |
| SEQ ID NO: 495 | CGCAGTCTTTTATTGAACGTNNKACGAATTTCGATAAAAACTT |
| SEQ ID NO: 496 | AGTCTTTTATTGAACGTATGNNKAATTTCGATAAAAACTTGCCC |
| SEQ ID NO: 497 | TTATTGAACGTATGACGNNKTTCGATAAAAACTTGCCC |
| SEQ ID NO: 498 | ATTGAACGTATGACGAATNNKGATAAAAACTTGCCCAATG |
| SEQ ID NO: 499 | AACGTATGACGAATTTCNNKAAAAACTTGCCCAATGA |
| SEQ ID NO: 500 | GTATGACGAATTTCGATNNKAACTTGCCCAATGAGAAG |
| SEQ ID NO: 501 | TGACGAATTTCGATAAANNKTTGCCCAATGAGAAGGT |
| SEQ ID NO: 502 | GAATTTCGATAAAAACNNKCCCAATGAGAAGGTGCT |
| SEQ ID NO: 503 | ATTTCGATAAAAACTTGNNKAATGAGAAGGTGCTTCCG |
| SEQ ID NO: 504 | ATAAAAACTTGCCCNNKGAGAAGGTGCTTCC |
| SEQ ID NO: 505 | TAAAAACTTGCCCAATNNKAAGGTGCTTCCGAAAC |
| SEQ ID NO: 506 | CTTGCCCAATGAGNNKGTGCTTCCGAAAC |
| SEQ ID NO: 507 | TGCCCAATGAGAAGNNKCTTCCGAAACATTC |
| SEQ ID NO: 508 | CCAATGAGAAGGTGNNKCCGAAACATTCCTTG |
| SEQ ID NO: 509 | GCCCAATGAGAAGGTGCTTNNKAAACATTCCTTGTTATATG |
| SEQ ID NO: 510 | TGAGAAGGTGCTTCCGNNKCATTCCTTGTTATATG |
| SEQ ID NO: 511 | ATGAGAAGGTGCTTCCGAAANNKTCCTTGTTATATGAATATTT |
| SEQ ID NO: 512 | GAGAAGGTGCTTCCGAAACATNNKTTGTTATATGAATATTTTACAG |
| SEQ ID NO: 513 | AGGTGCTTCCGAAACATTCCNNKTTATATGAATATTTTACAGTT |
| SEQ ID NO: 514 | GTGCTTCCGAAACATTCCTTGNNKTATGAATATTTTACAGTTTAC |
| SEQ ID NO: 515 | CTTCCGAAACATTCCTTGTTANNKGAATATTTTACAGTTTACAACG |
| SEQ ID NO: 516 | CCGAAACATTCCTTGTTATATNNKTATTTTACAGTTTACAACGAGC |
| SEQ ID NO: 517 | AAACATTCCTTGTTATATGAANNKTTTACAGTTTACAACGAGCTGA |
| SEQ ID NO: 518 | ATTCCTTGTTATATGAATATNNKACAGTTTACAACGAGCTGACC |
| SEQ ID NO: 519 | CCTTGTTATATGAATATTTTNNKGTTTACAACGAGCTGACCAAG |
| SEQ ID NO: 520 | TTGTTATATGAATATTTTACANNKTACAACGAGCTGACCAAGGTT |
| SEQ ID NO: 521 | TGTTATATGAATATTTTACAGTTNNKAACGAGCTGACCAAGGTTAAATAC |
| SEQ ID NO: 522 | TATGAATATTTTACAGTTTACNNKGAGCTGACCAAGGTTAAATACG |
| SEQ ID NO: 523 | GAATATTTTACAGTTTACAACNNKCTGACCAAGGTTAAATACGTGA |
| SEQ ID NO: 524 | TTTACAGTTTACAACGAGNNKACCAAGGTTAAATACGTGA |
| SEQ ID NO: 525 | CAGTTTACAACGAGCTGNNKAAGGTTAAATACGTGAC |
| SEQ ID NO: 526 | TACAACGAGCTGACCNNKGTTAAATACGTGACG |
| SEQ ID NO: 527 | CGAGCTGACCAAGNNKAAATACGTGACGGA |
| SEQ ID NO: 528 | GCTGACCAAGGTTNNKTACGTGACGGAAG |
| SEQ ID NO: 529 | CTGACCAAGGTTAAANNKGTGACGGAAGGAATG |
| SEQ ID NO: 530 | CCAAGGTTAAATACNNKACGGAAGGAATGCGC |
| SEQ ID NO: 531 | AAGGTTAAATACGTGNNKGAAGGAATGCGCAAG |
| SEQ ID NO: 532 | TAAATACGTGACGNNKGGAATGCGCAAGC |
| SEQ ID NO: 533 | ACGTGACGGAANNKATGCGCAAGCC |
| SEQ ID NO: 534 | TGACGGAAGGANNKCGCAAGCCCGC |
| SEQ ID NO: 535 | GACGGAAGGAATGNNKAAGCCCGCTTTTC |
| SEQ ID NO: 536 | GAAGGAATGCGCNNKCCCGCTTTTCTTA |
| SEQ ID NO: 537 | AGGAATGCGCAAGNNKGCTTTTCTTAGCG |
| SEQ ID NO: 538 | TGCGCAAGCCCNNKTTTCTTAGCGG |
| SEQ ID NO: 539 | GCAAGCCCGCTNNKCTTAGCGGTGA |
| SEQ ID NO: 540 | AGCCCGCTTTTNNKAGCGGTGAGCA |
| SEQ ID NO: 541 | AGCCCGCTTTTCTTNNKGGTGAGCAAAAAAA |
| SEQ ID NO: 542 | CCGCTTTTCTTAGCNNKGAGCAAAAAAAGGC |
| SEQ ID NO: 543 | GCTTTTCTTAGCGGTNNKCAAAAAAAGGCGATC |
| SEQ ID NO: 544 | TTCTTAGCGGTGAGNNKAAAAAGGCGATCGT |
| SEQ ID NO: 545 | GCGGTGAGCAANNKAAGGCGATCGT |
| SEQ ID NO: 546 | GGTGAGCAAAAANNKGCGATCGTCGACC |
| SEQ ID NO: 547 | GGTGAGCAAAAAAAGNNKATCGTCGACCTGTTAT |
| SEQ ID NO: 548 | GAGCAAAAAAAGGCGNNKGTCGACCTGTTATTC |
| SEQ ID NO: 549 | AGCAAAAAAAGGCGATCNNKGACCTGTTATTCAAAAC |
| SEQ ID NO: 550 | AAAAAAAGGCGATCGTCNNKCTGTTATTCAAAACGAA |
| SEQ ID NO: 551 | AAAGGCGATCGTCGACNNKTTATTCAAAACGAATC |
| SEQ ID NO: 552 | CGATCGTCGACCTGNNKTTCAAAACGAATCG |
| SEQ ID NO: 553 | GATCGTCGACCTGTTANNKAAAACGAATCGTAAGGT |
| SEQ ID NO: 554 | GTCGACCTGTTATTCNNKACGAATCGTAAGGTGA |
| SEQ ID NO: 555 | TCGACCTGTTATTCAAANNKAATCGTAAGGTGACTGT |
| SEQ ID NO: 556 | ACCTGTTATTCAAAACGNNKCGTAAGGTGACTGTAAA |
| SEQ ID NO: 557 | CCTGTTATTCAAAACGAATNNKAAGGTGACTGTAAAGCAAC |
| SEQ ID NO: 558 | TATTCAAAACGAATCGTNNKGTGACTGTAAAGCAACTC |
| SEQ ID NO: 559 | TTCAAAACGAATCGTAAGNNKACTGTAAAGCAACTCAAAG |
| SEQ ID NO: 560 | AAACGAATCGTAAGGTGNNKGTAAAGCAACTCAAAGA |
| SEQ ID NO: 561 | CGAATCGTAAGGTGACTNNKAAGCAACTCAAAGAAGA |
| SEQ ID NO: 562 | GAATCGTAAGGTGACTGTANNKCAACTCAAAGAAGATTACTT |
| SEQ ID NO: 563 | CGTAAGGTGACTGTAAAGNNKCTCAAAGAAGATTACTTCA |
| SEQ ID NO: 564 | CGTAAGGTGACTGTAAAGCAANNKAAAGAAGATTACTTCAAAAAG |
| SEQ ID NO: 565 | GGTGACTGTAAAGCAACTCNNKGAAGATTACTTCAAAAAGA |
| SEQ ID NO: 566 | GTGACTGTAAAGCAACTCAAANNKGATTACTTCAAAAAGATTGAG |
| SEQ ID NO: 567 | CTGTAAAGCAACTCAAAGAANNKTACTTCAAAAAGATTGAGTGC |
| SEQ ID NO: 568 | AAGCAACTCAAAGAAGATNNKTTCAAAAAGATTGAGTGCT |
| SEQ ID NO: 569 | GCAACTCAAAGAAGATTACNNKAAAAAGATTGAGTGCTTCG |
| SEQ ID NO: 570 | CTCAAAGAAGATTACTTCNNKAAGATTGAGTGCTTCGACA |
| SEQ ID NO: 571 | AAGAAGATTACTTCAAANNKATTGAGTGCTTCGACAGC |
| SEQ ID NO: 572 | GATTACTTCAAAAAGNNKGAGTGCTTCGACAGCG |
| SEQ ID NO: 573 | TTACTTCAAAAAGATTNNKTGCTTCGACAGCGTCG |
| SEQ ID NO: 574 | CTTCAAAAAGATTGAGNNKTTCGACAGCGTCGAAAT |
| SEQ ID NO: 575 | AAAAAGATTGAGTGCNNKGACAGCGTCGAAATC |
| SEQ ID NO: 576 | AAGATTGAGTGCTTCNNKAGCGTCGAAATCTCT |
| SEQ ID NO: 577 | TTGAGTGCTTCGACNNKGTCGAAATCTCTGG |
| SEQ ID NO: 578 | TGCTTCGACAGCNNKGAAATCTCTGGG |
| SEQ ID NO: 579 | CTTCGACAGCGTCNNKATCTCTGGGGTAG |
| SEQ ID NO: 580 | GACAGCGTCGAANNKTCTGGGGTAGAGG |
| SEQ ID NO: 581 | CAGCGTCGAAATCNNKGGGGTAGAGGATC |
| SEQ ID NO: 582 | AGCGTCGAAATCTCTNNKGTAGAGGATCGGTTTA |
| SEQ ID NO: 583 | GTCGAAATCTCTGGGNNKGAGGATCGGTTTAAC |
| SEQ ID NO: 584 | GAAATCTCTGGGGTANNKGATCGGTTTAACGCAA |
| SEQ ID NO: 585 | CTCTGGGGTAGAGNNKCGGTTTAACGCAA |
| SEQ ID NO: 586 | CTCTGGGGTAGAGGATNNKTTTAACGCAAGTTTAGG |
| SEQ ID NO: 587 | GGGTAGAGGATCGGNNKAACGCAAGTTTAGG |
| SEQ ID NO: 588 | GGTAGAGGATCGGTTTNNKGCAAGTTTAGGTACCTA |
| SEQ ID NO: 589 | GTAGAGGATCGGTTTAACNNKAGTTTAGGTACCTACCAT |
| SEQ ID NO: 590 | GGATCGGTTTAACGCANNKTTAGGTACCTACCATG |
| SEQ ID NO: 591 | GGTTTAACGCAAGTNNKGGTACCTACCATGAC |
| SEQ ID NO: 592 | GTTTAACGCAAGTTTANNKACCTACCATGACCTGC |
| SEQ ID NO: 593 | TTAACGCAAGTTTAGGTNNKTACCATGACCTGCTTAA |
| SEQ ID NO: 594 | CGCAAGTTTAGGTACCNNKCATGACCTGCTTAAAAT |
| SEQ ID NO: 595 | GCAAGTTTAGGTACCTACNNKGACCTGCTTAAAATCATT |
| SEQ ID NO: 596 | GCAAGTTTAGGTACCTACCATNNKCTGCTTAAAATCATTAAGGAT |
| SEQ ID NO: 597 | AAGTTTAGGTACCTACCATGACNNKCTTAAAATCATTAAGGATAAAGA |
| SEQ ID NO: 598 | AGGTACCTACCATGACCTGNNKAAAATCATTAAGGATAAAGA |
| SEQ ID NO: 599 | ACCTACCATGACCTGCTTNNKATCATTAAGGATAAAGACT |
| SEQ ID NO: 600 | ACCTACCATGACCTGCTTAAANNKATTAAGGATAAAGACTTCTTA |
| SEQ ID NO: 601 | ACCATGACCTGCTTAAAATCNNKAAGGATAAAGACTTCTTAGA |
| SEQ ID NO: 602 | CCATGACCTGCTTAAAATCATTNNKGATAAAGACTTCTTAGATAATGA |
| SEQ ID NO: 603 | CATGACCTGCTTAAAATCATTAAGNNKAAAGACTTCTTAGATAATGAAGAG |
| SEQ ID NO: 604 | ACCTGCTTAAAATCATTAAGGATNNKGACTTCTTAGATAATGAAGAGAA |
| SEQ ID NO: 605 | CTGCTTAAAATCATTAAGGATAAANNKTTCTTAGATAATGAAGAGAACGAA |
| SEQ ID NO: 606 | CTTAAAATCATTAAGGATAAAGACNNKTTAGATAATGAAGAGAACGAAGAT |
| SEQ ID NO: 607 | AAAATCATTAAGGATAAAGACTTCNNKGATAATGAAGAGAACGAAGATATT |
| SEQ ID NO: 608 | ATCATTAAGGATAAAGACTTCTTANNKAATGAAGAGAACGAAGATATTCTC |
| SEQ ID NO: 609 | AAGGATAAAGACTTCTTAGATNNKGAAGAGAACGAAGATATTCTCG |
| SEQ ID NO: 610 | GGATAAAGACTTCTTAGATAATNNKGAGAACGAAGATATTCTCGAGG |
| SEQ ID NO: 611 | AAAGACTTCTTAGATAATGAANNKAACGAAGATATTCTCGAGGACA |
| SEQ ID NO: 612 | GACTTCTTAGATAATGAAGAGNNKGAAGATATTCTCGAGGACATC |
| SEQ ID NO: 613 | CTTAGATAATGAAGAGAACNNKGATATTCTCGAGGACATCGT |
| SEQ ID NO: 614 | ATAATGAAGAGAACGAANNKATTCTCGAGGACATCGTC |
| SEQ ID NO: 615 | GAAGAGAACGAAGATNNKCTCGAGGACATCGTCT |
| SEQ ID NO: 616 | AAGAGAACGAAGATATTNNKGAGGACATCGTCTTGACG |
| SEQ ID NO: 617 | GAACGAAGATATTCTCNNKGACATCGTCTTGACGTT |
| SEQ ID NO: 618 | ACGAAGATATTCTCGAGNNKATCGTCTTGACGTTAAC |
| SEQ ID NO: 619 | AAGATATTCTCGAGGACNNKGTCTTGACGTTAACCTT |
| SEQ ID NO: 620 | AGATATTCTCGAGGACATCNNKTTGACGTTAACCTTATTTGA |
| SEQ ID NO: 621 | TCTCGAGGACATCGTCNNKACGTTAACCTTATTTGA |
| SEQ ID NO: 622 | TCGAGGACATCGTCTTGNNKTTAACCTTATTTGAGGA |
| SEQ ID NO: 623 | GGACATCGTCTTGACGNNKACCTTATTTGAGGATC |
| SEQ ID NO: 624 | ACATCGTCTTGACGTTANNKTTATTTGAGGATCGTGAA |
| SEQ ID NO: 625 | CGTCTTGACGTTAACCNNKTTTGAGGATCGTGAAAT |
| SEQ ID NO: 626 | GTCTTGACGTTAACCTTANNKGAGGATCGTGAAATGATT |
| SEQ ID NO: 627 | CTTGACGTTAACCTTATTTNNKGATCGTGAAATGATTGAGG |
| SEQ ID NO: 628 | ACGTTAACCTTATTTGAGNNKCGTGAAATGATTGAGGAA |
| SEQ ID NO: 629 | TTAACCTTATTTGAGGATNNKGAAATGATTGAGGAACGCC |
| SEQ ID NO: 630 | CTTATTTGAGGATCGTNNKATGATTGAGGAACGCCT |
| SEQ ID NO: 631 | TTGAGGATCGTGAANNKATTGAGGAACGCCTC |
| SEQ ID NO: 632 | GGATCGTGAAATGNNKGAGGAACGCCTCAA |
| SEQ ID NO: 633 | AGGATCGTGAAATGATTNNKGAACGCCTCAAAACTTA |
| SEQ ID NO: 634 | TCGTGAAATGATTGAGNNKCGCCTCAAAACTTATG |
| SEQ ID NO: 635 | GTGAAATGATTGAGGAANNKCTCAAAACTTATGCCCAC |
| SEQ ID NO: 636 | ATGATTGAGGAACGCNNKAAAACTTATGCCCAC |
| SEQ ID NO: 637 | GAGGAACGCCTCNNKACTTATGCCCAC |
| SEQ ID NO: 638 | GAACGCCTCAAANNKTATGCCCACCTGT |
| SEQ ID NO: 639 | CGCCTCAAAACTNNKGCCCACCTGTTC |
| SEQ ID NO: 640 | CGCCTCAAAACTTATNNKCACCTGTTCGACGATA |
| SEQ ID NO: 641 | CTCAAAACTTATGCCNNKCTGTTCGACGATAAGG |
| SEQ ID NO: 642 | CAAAACTTATGCCCACNNKTTCGACGATAAGGTGAT |
| SEQ ID NO: 643 | CTTATGCCCACCTGNNKGACGATAAGGTGATG |
| SEQ ID NO: 644 | TATGCCCACCTGTTCNNKGATAAGGTGATGAAG |
| SEQ ID NO: 645 | CCACCTGTTCGACNNKAAGGTGATGAAGC |
| SEQ ID NO: 646 | CCTGTTCGACGATNNKGTGATGAAGCAGC |
| SEQ ID NO: 647 | CTGTTCGACGATAAGNNKATGAAGCAGCTGAAAC |
| SEQ ID NO: 648 | CGACGATAAGGTGNNKAAGCAGCTGAAACG |
| SEQ ID NO: 649 | CGATAAGGTGATGNNKCAGCTGAAACGTCG |
| SEQ ID NO: 650 | TAAGGTGATGAAGNNKCTGAAACGTCGGC |
| SEQ ID NO: 651 | TGATGAAGCAGNNKAAACGTCGGCGC |
| SEQ ID NO: 652 | TGAAGCAGCTGNNKCGTCGGCGCTA |
| SEQ ID NO: 653 | AGCAGCTGAAANNKCGGCGCTACAC |
| SEQ ID NO: 654 | CAGCTGAAACGTNNKCGCTACACAGGAT |
| SEQ ID NO: 655 | CTGAAACGTCGGNNKTACACAGGATGGG |
| SEQ ID NO: 656 | AACGTCGGCGCNNKACAGGATGGGG |
| SEQ ID NO: 657 | GTCGGCGCTACNNKGGATGGGGCCG |
| SEQ ID NO: 658 | GGCGCTACACANNKTGGGGCCGCTT |
| SEQ ID NO: 659 | GCTACACAGGANNKGGCCGCTTGAGT |
| SEQ ID NO: 660 | ACACAGGATGGNNKCGCTTGAGTCG |
| SEQ ID NO: 661 | ACAGGATGGGGCNNKTTGAGTCGCAAA |
| SEQ ID NO: 662 | GATGGGGCCGCNNKAGTCGCAAACT |
| SEQ ID NO: 663 | GGGGCCGCTTGNNKCGCAAACTTAT |
| SEQ ID NO: 664 | GGGGCCGCTTGAGTNNKAAACTTATTAACGGA |
| SEQ ID NO: 665 | CCGCTTGAGTCGCNNKCTTATTAACGGAAT |
| SEQ ID NO: 666 | GCTTGAGTCGCAAANNKATTAACGGAATCCGT |
| SEQ ID NO: 667 | TGAGTCGCAAACTTNNKAACGGAATCCGTGA |
| SEQ ID NO: 668 | AGTCGCAAACTTATTNNKGGAATCCGTGACAAG |
| SEQ ID NO: 669 | GTCGCAAACTTATTAACNNKATCCGTGACAAGCAATC |
| SEQ ID NO: 670 | CAAACTTATTAACGGANNKCGTGACAAGCAATCCG |
| SEQ ID NO: 671 | CTTATTAACGGAATCNNKGACAAGCAATCCGGC |
| SEQ ID NO: 672 | TATTAACGGAATCCGTNNKAAGCAATCCGGCAAAA |
| SEQ ID NO: 673 | GGAATCCGTGACNNKCAATCCGGCAAAA |
| SEQ ID NO: 674 | ATCCGTGACAAGNNKTCCGGCAAAACG |
| SEQ ID NO: 675 | CCGTGACAAGCAANNKGGCAAAACGATTCT |
| SEQ ID NO: 676 | CGTGACAAGCAATCCNNKAAAACGATTCTGGATT |
| SEQ ID NO: 677 | CAAGCAATCCGGCNNKACGATTCTGGATTT |
| SEQ ID NO: 678 | CAAGCAATCCGGCAAANNKATTCTGGATTTCTTGAA |
| SEQ ID NO: 679 | AATCCGGCAAAACGNNKCTGGATTTCTTGAAG |
| SEQ ID NO: 680 | TCCGGCAAAACGATTNNKGATTTCTTGAAGTCG |
| SEQ ID NO: 681 | GGCAAAACGATTCTGNNKTTCTTGAAGTCGGAC |
| SEQ ID NO: 682 | AAAACGATTCTGGATNNKTTGAAGTCGGACGGAT |
| SEQ ID NO: 683 | ACGATTCTGGATITCNNKAAGTCGGACGGATTT |
| SEQ ID NO: 684 | GATTCTGGATTTCTTGNNKTCGGACGGATTTGCTAA |
| SEQ ID NO: 685 | TTCTGGATTTCTTGAAGNNKGACGGATTTGCTAATCG |
| SEQ ID NO: 686 | GATTTCTTGAAGTCGNNKGGATTTGCTAATCGCA |
| SEQ ID NO: 687 | TTTCTTGAAGTCGGACNNKTTTGCTAATCGCAACT |
| SEQ ID NO: 688 | GAAGTCGGACGGANNKGCTAATCGCAACTT |
| SEQ ID NO: 689 | AAGTCGGACGGATTTNNKAATCGCAACTTCATG |
| SEQ ID NO: 690 | GACGGATTTGCTNNKCGCAACTTCATGC |
| SEQ ID NO: 691 | GGACGGATTTGCTAATNNKAACTTCATGCAGTTGAT |
| SEQ ID NO: 692 | GGATTTGCTAATCGCNNKTTCATGCAGTTGATCC |
| SEQ ID NO: 693 | ATTTGCTAATCGCAACNNKATGCAGTTGATCCATG |
| SEQ ID NO: 694 | GCTAATCGCAACTTCNNKCAGTTGATCCATGATG |
| SEQ ID NO: 695 | CTAATCGCAACTTCATGNNKTTGATCCATGATGACTCC |
| SEQ ID NO: 696 | GCAACTTCATGCAGNNKATCCATGATGACTCC |
| SEQ ID NO: 697 | ACTTCATGCAGTTGNNKCATGATGACTCCCT |
| SEQ ID NO: 698 | TCATGCAGTTGATCNNKGATGACTCCCTGAC |
| SEQ ID NO: 699 | CATGCAGTTGATCCATNNKGACTCCCTGACTTTTAA |
| SEQ ID NO: 700 | TGCAGTTGATCCATGATNNKTCCCTGACTTTTAAAGA |
| SEQ ID NO: 701 | CAGTTGATCCATGATGACNNKCTGACTTTTAAAGAGGAT |
| SEQ ID NO: 702 | AGTTGATCCATGATGACTCCNNKACTITTAAAGAGGATATTCA |
| SEQ ID NO: 703 | TGATCCATGATGACTCCCTGNNKTTTAAAGAGGATATTCAAAA |
| SEQ ID NO: 704 | CATGATGACTCCCTGACTNNKAAAGAGGATATTCAAAAGG |
| SEQ ID NO: 705 | ATGACTCCCTGACTTTTNNKGAGGATATTCAAAAGGC |
| SEQ ID NO: 706 | ACTCCCTGACTTTTAAANNKGATATTCAAAAGGCGCA |
| SEQ ID NO: 707 | CCTGACTTTTAAAGAGNNKATTCAAAAGGCGCAGG |
| SEQ ID NO: 708 | TGACTTTTAAAGAGGATNNKCAAAAGGCGCAGGTTAG |
| SEQ ID NO: 709 | CTTTTAAAGAGGATATTNNKAAGGCGCAGGTTAGTGGT |
| SEQ ID NO: 710 | TAAAGAGGATATTCAANNKGCGCAGGTTAGTGGTCA |
| SEQ ID NO: 711 | AAGAGGATATTCAAAAGNNKCAGGTTAGTGGTCAAGG |
| SEQ ID NO: 712 | GATATTCAAAAGGCGNNKGTTAGTGGTCAAGGC |
| SEQ ID NO: 713 | CAAAAGGCGCAGNNKAGTGGTCAAGGC |
| SEQ ID NO: 714 | AGGCGCAGGTTNNKGGTCAAGGCGA |
| SEQ ID NO: 715 | CGCAGGTTAGTNNKCAAGGCGACAGC |
| SEQ ID NO: 716 | CAGGTTAGTGGTNNKGGCGACAGCTTAC |
| SEQ ID NO: 717 | CAGGTTAGTGGTCAANNKGACAGCTTACACGAAC |
| SEQ ID NO: 718 | TTAGTGGTCAAGGCNNKAGCTTACACGAACAC |
| SEQ ID NO: 719 | TGGTCAAGGCGACNNKTTACACGAACACAT |
| SEQ ID NO: 720 | CAAGGCGACAGCNNKCACGAACACATC |
| SEQ ID NO: 721 | AGGCGACAGCTTANNKGAACACATCGCAA |
| SEQ ID NO: 722 | GCGACAGCTTACACNNKCACATCGCAAATTT |
| SEQ ID NO: 723 | GACAGCTTACACGAANNKATCGCAAATTTGGCT |
| SEQ ID NO: 724 | CTTACACGAACACNNKGCAAATTTGGCTGG |
| SEQ ID NO: 725 | TTACACGAACACATCNNKAATTTGGCTGGTTCG |
| SEQ ID NO: 726 | GAACACATCGCANNKTTGGCTGGTTCG |
| SEQ ID NO: 727 | ACATCGCAAATNNKGCTGGTTCGCC |
| SEQ ID NO: 728 | TCGCAAATTTGNNKGGTTCGCCGGC |
| SEQ ID NO: 729 | CAAATTTGGCTNNKTCGCCGGCCATT |
| SEQ ID NO: 730 | CAAATTTGGCTGGTNNKCCGGCCATTAAAAAG |
| SEQ ID NO: 731 | ATTTGGCTGGTTCGNNKGCCATTAAAAAGGG |
| SEQ ID NO: 732 | GCTGGTTCGCCGNNKATTAAAAAGGGGA |
| SEQ ID NO: 733 | GTTCGCCGGCCNNKAAAAAGGGGATC |
| SEQ ID NO: 734 | CGCCGGCCATTNNKAAGGGGATCCT |
| SEQ ID NO: 735 | CCGGCCATTAAANNKGGGATCCTCCAGA |
| SEQ ID NO: 736 | CGGCCATTAAAAAGNNKATCCTCCAGACCGT |
| SEQ ID NO: 737 | CCATTAAAAAGGGGNNKCTCCAGACCGTGAA |
| SEQ ID NO: 738 | CATTAAAAAGGGGATCNNKCAGACCGTGAAAGTTGT |
| SEQ ID NO: 739 | TAAAAAGGGGATCCTCNNKACCGTGAAAGTTGTAGA |
| SEQ ID NO: 740 | AAGGGGATCCTCCAGNNKGTGAAAGTTGTAGAT |
| SEQ ID NO: 741 | GGGATCCTCCAGACCNNKAAAGTTGTAGATGAG |
| SEQ ID NO: 742 | CCTCCAGACCGTGNNKGTTGTAGATGAGC |
| SEQ ID NO: 743 | CTCCAGACCGTGAAANNKGTAGATGAGCTTGTTA |
| SEQ ID NO: 744 | CAGACCGTGAAAGTTNNKGATGAGCTTGTTAAGG |
| SEQ ID NO: 745 | GACCGTGAAAGTTGTANNKGAGCTTGTTAAGGTCAT |
| SEQ ID NO: 746 | CCGTGAAAGTTGTAGATNNKCTTGTTAAGGTCATGGG |
| SEQ ID NO: 747 | GAAAGTTGTAGATGAGNNKGTTAAGGTCATGGGTCG |
| SEQ ID NO: 748 | GTTGTAGATGAGCTTNNKAAGGTCATGGGTCGTC |
| SEQ ID NO: 749 | GTAGATGAGCTTGTTNNKGTCATGGGTCGTCATA |
| SEQ ID NO: 750 | AGATGAGCTTGTTAAGNNKATGGGTCGTCATAAGC |
| SEQ ID NO: 751 | AGCTTGTTAAGGTCNNKGGTCGTCATAAGCC |
| SEQ ID NO: 752 | TTGTTAAGGTCATGNNKCGTCATAAGCCCGAA |
| SEQ ID NO: 753 | GTTAAGGTCATGGGTNNKCATAAGCCCGAAAACA |
| SEQ ID NO: 754 | GTCATGGGTCGTNNKAAGCCCGAAAACA |
| SEQ ID NO: 755 | CATGGGTCGTCATNNKCCCGAAAACATCG |
| SEQ ID NO: 756 | CATGGGTCGTCATAAGNNKGAAAACATCGTGATTGA |
| SEQ ID NO: 757 | GTCGTCATAAGCCCNNKAACATCGTGATTGAA |
| SEQ ID NO: 758 | CGTCATAAGCCCGAANNKATCGTGATTGAAATGG |
| SEQ ID NO: 759 | ATAAGCCCGAAAACNNKGTGATTGAAATGGCG |
| SEQ ID NO: 760 | GCCCGAAAACATCNNKATTGAAATGGCGC |
| SEQ ID NO: 761 | GAAAACATCGTGNNKGAAATGGCGCGG |
| SEQ ID NO: 762 | AAACATCGTGATTNNKATGGCGCGGGAGAA |
| SEQ ID NO: 763 | CATCGTGATTGAANNKGCGCGGGAGAATC |
| SEQ ID NO: 764 | ATCGTGATTGAAATGNNKCGGGAGAATCAAACG |
| SEQ ID NO: 765 | GTGATTGAAATGGCGNNKGAGAATCAAACGACC |
| SEQ ID NO: 766 | GAAATGGCGCGGNNKAATCAAACGACC |
| SEQ ID NO: 767 | TGGCGCGGGAGNNKCAAACGACCCA |
| SEQ ID NO: 768 | CGCGGGAGAATNNKACGACCCAGAAA |
| SEQ ID NO: 769 | GCGGGAGAATCAANNKACCCAGAAAGGACA |
| SEQ ID NO: 770 | CGGGAGAATCAAACGNNKCAGAAAGGACAAAAG |
| SEQ ID NO: 771 | GGGAGAATCAAACGACCNNKAAAGGACAAAAGAATAGC |
| SEQ ID NO: 772 | AATCAAACGACCCAGNNKGGACAAAAGAATAGCC |
| SEQ ID NO: 773 | CAAACGACCCAGAAANNKCAAAAGAATAGCCGTG |
| SEQ ID NO: 774 | CGACCCAGAAAGGANNKAAGAATAGCCGTGAA |
| SEQ ID NO: 775 | CCCAGAAAGGACAANNKAATAGCCGTGAACG |
| SEQ ID NO: 776 | AGAAAGGACAAAAGNNKAGCCGTGAACGGAT |
| SEQ ID NO: 777 | GAAAGGACAAAAGAATNNKCGTGAACGGATGAAGC |
| SEQ ID NO: 778 | GACAAAAGAATAGCNNKGAACGGATGAAGCGG |
| SEQ ID NO: 779 | AAAAGAATAGCCGTNNKCGGATGAAGCGGAT |
| SEQ ID NO: 780 | AGAATAGCCGTGAANNKATGAAGCGGATCGAG |
| SEQ ID NO: 781 | GCCGTGAACGGNNKAAGCGGATCGA |
| SEQ ID NO: 782 | GTGAACGGATGNNKCGGATCGAGGAA |
| SEQ ID NO: 783 | GTGAACGGATGAAGNNKATCGAGGAAGGCATT |
| SEQ ID NO: 784 | CGGATGAAGCGGNNKGAGGAAGGCATTA |
| SEQ ID NO: 785 | GGATGAAGCGGATCNNKGAAGGCATTAAAGAG |
| SEQ ID NO: 786 | GAAGCGGATCGAGNNKGGCATTAAAGAGCT |
| SEQ ID NO: 787 | AGCGGATCGAGGAANNKATTAAAGAGCTGGG |
| SEQ ID NO: 788 | ATCGAGGAAGGCNNKAAAGAGCTGGGG |
| SEQ ID NO: 789 | GAGGAAGGCATTNNKGAGCTGGGGTCTC |
| SEQ ID NO: 790 | GAGGAAGGCATTAAANNKCTGGGGTCTCAAATCT |
| SEQ ID NO: 791 | GGAAGGCATTAAAGAGNNKGGGTCTCAAATCTTGAA |
| SEQ ID NO: 792 | AAGGCATTAAAGAGCTGNNKTCTCAAATCTTGAAGGA |
| SEQ ID NO: 793 | CATTAAAGAGCTGGGGNNKCAAATCTTGAAGGAAC |
| SEQ ID NO: 794 | TAAAGAGCTGGGGTCTNNKATCTTGAAGGAACACC |
| SEQ ID NO: 795 | GCTGGGGTCTCAANNKTTGAAGGAACACC |
| SEQ ID NO: 796 | GGGGTCTCAAATCNNKAAGGAACACCCTGT |
| SEQ ID NO: 797 | GGTCTCAAATCTTGNNKGAACACCCTGTGGAG |
| SEQ ID NO: 798 | TCTCAAATCTTGAAGNNKCACCCTGTGGAGAAC |
| SEQ ID NO: 799 | TCAAATCTTGAAGGAANNKCCTGTGGAGAACACTC |
| SEQ ID NO: 800 | ATCTTGAAGGAACACNNKGTGGAGAACACTCAG |
| SEQ ID NO: 801 | GAAGGAACACCCTNNKGAGAACACTCAGCT |
| SEQ ID NO: 802 | GAACACCCTGTGNNKAACACTCAGCTCC |
| SEQ ID NO: 803 | CACCCTGTGGAGNNKACTCAGCTCCAAA |
| SEQ ID NO: 804 | CCCTGTGGAGAACNNKCAGCTCCAAAATGA |
| SEQ ID NO: 805 | CCCTGTGGAGAACACTNNKCTCCAAAATGAAAAACT |
| SEQ ID NO: 806 | CCTGTGGAGAACACTCAGNNKCAAAATGAAAAACTTTAC |
| SEQ ID NO: 807 | TGGAGAACACTCAGCTCNNKAATGAAAAACTTTACCT |
| SEQ ID NO: 808 | AGAACACTCAGCTCCAANNKGAAAAACTTTACCTGTAC |
| SEQ ID NO: 809 | AGAACACTCAGCTCCAAAATNNKAAACTTTACCTGTACTATTTG |
| SEQ ID NO: 810 | ACTCAGCTCCAAAATGAANNKCTTTACCTGTACTATTTGC |
| SEQ ID NO: 811 | TCAGCTCCAAAATGAAAAANNKTACCTGTACTATTTGCAGA |
| SEQ ID NO: 812 | GCTCCAAAATGAAAAACTTNNKCTGTACTATTTGCAGAACG |
| SEQ ID NO: 813 | CCAAAATGAAAAACTTTACNNKTACTATTTGCAGAACGGACG |
| SEQ ID NO: 814 | ATGAAAAACTTTACCTGNNKTATTTGCAGAACGGACG |
| SEQ ID NO: 815 | AACTTTACCTGTACNNKTTGCAGAACGGACG |
| SEQ ID NO: 816 | AACTITACCTGTACTATNNKCAGAACGGACGCGATAT |
| SEQ ID NO: 817 | TTTACCTGTACTATTTGNNKAACGGACGCGATATGTAC |
| SEQ ID NO: 818 | CTGTACTATTTGCAGNNKGGACGCGATATGTACG |
| SEQ ID NO: 819 | TACTATTTGCAGAACNNKCGCGATATGTACGTGG |
| SEQ ID NO: 820 | CTATTTGCAGAACGGANNKGATATGTACGTGGACC |
| SEQ ID NO: 821 | CAGAACGGACGCNNKATGTACGTGGAC |
| SEQ ID NO: 822 | AACGGACGCGATNNKTACGTGGACCAA |
| SEQ ID NO: 823 | GGACGCGATATGNNKGTGGACCAAGAGT |
| SEQ ID NO: 824 | GACGCGATATGTACNNKGACCAAGAGTTGGAT |
| SEQ ID NO: 825 | GACGCGATATGTACGTGNNKCAAGAGTTGGATATTAA |
| SEQ ID NO: 826 | GCGATATGTACGTGGACNNKGAGTTGGATATTAATCG |
| SEQ ID NO: 827 | ATATGTACGTGGACCAANNKTTGGATATTAATCGGCTG |
| SEQ ID NO: 828 | TACGTGGACCAAGAGNNKGATATTAATCGGCTGA |
| SEQ ID NO: 829 | GTGGACCAAGAGTTGNNKATTAATCGGCTGAGT |
| SEQ ID NO: 830 | GACCAAGAGTTGGATNNKAATCGGCTGAGTGAC |
| SEQ ID NO: 831 | CAAGAGTTGGATATTNNKCGGCTGAGTGACTACG |
| SEQ ID NO: 832 | CAAGAGTTGGATATTAATNNKCTGAGTGACTACGACGTTG |
| SEQ ID NO: 833 | AGTTGGATATTAATCGGNNKAGTGACTACGACGTTGA |
| SEQ ID NO: 834 | GGATATTAATCGGCTGNNKGACTACGACGTTGATG |
| SEQ ID NO: 835 | TTAATCGGCTGAGTNNKTACGACGTTGATGC |
| SEQ ID NO: 836 | GGCTGAGTGACNNKGACGTTGATGCG |
| SEQ ID NO: 837 | GCTGAGTGACTACNNKGTTGATGCGATCG |
| SEQ ID NO: 838 | GAGTGACTACGACNNKGATGCGATCGTCC |
| SEQ ID NO: 839 | ACTACGACGTTNNKGCGATCGTCCC |
| SEQ ID NO: 840 | CTACGACGTTGATNNKATCGTCCCGCAGAG |
| SEQ ID NO: 841 | ACGTTGATGCGNNKGTCCCGCAGAG |
| SEQ ID NO: 842 | GTTGATGCGATCNNKCCGCAGAGCTTC |
| SEQ ID NO: 843 | GATGCGATCGTCNNKCAGAGCTTCCTCA |
| SEQ ID NO: 844 | CGATCGTCCCGNNKAGCTTCCTCAAA |
| SEQ ID NO: 845 | ATCGTCCCGCAGNNKTTCCTCAAAGAC |
| SEQ ID NO: 846 | GTCCCGCAGAGCNNKCTCAAAGACGAT |
| SEQ ID NO: 847 | GTCCCGCAGAGCTTCNNKAAAGACGATTCTATT |
| SEQ ID NO: 848 | CGCAGAGCTTCCTCNNKGACGATTCTATTGA |
| SEQ ID NO: 849 | CCGCAGAGCTTCCTCAAANNKGATTCTATTGACAATAAG |
| SEQ ID NO: 850 | GCAGAGCTTCCTCAAAGACNNKTCTATTGACAATAAGGTAC |
| SEQ ID NO: 851 | AGCTTCCTCAAAGACGATNNKATTGACAATAAGGTACTGA |
| SEQ ID NO: 852 | TCCTCAAAGACGATTCTNNKGACAATAAGGTACTGACG |
| SEQ ID NO: 853 | TCAAAGACGATTCTATTNNKAATAAGGTACTGACGCGC |
| SEQ ID NO: 854 | ACGATTCTATTGACNNKAAGGTACTGACGCGC |
| SEQ ID NO: 855 | GATTCTATTGACAATNNKGTACTGACGCGCTCTG |
| SEQ ID NO: 856 | ATTCTATTGACAATAAGNNKCTGACGCGCTCTGATAA |
| SEQ ID NO: 857 | TCTATTGACAATAAGGTANNKACGCGCTCTGATAAAAACC |
| SEQ ID NO: 858 | TTGACAATAAGGTACTGNNKCGCTCTGATAAAAACCG |
| SEQ ID NO: 859 | ACAATAAGGTACTGACGNNKTCTGATAAAAACCGTGG |
| SEQ ID NO: 860 | GGTACTGACGCGCNNKGATAAAAACCGTG |
| SEQ ID NO: 861 | TACTGACGCGCTCTNNKAAAAACCGTGGTAAG |
| SEQ ID NO: 862 | GACGCGCTCTGATNNKAACCGTGGTAAGT |
| SEQ ID NO: 863 | GCGCTCTGATAAANNKCGTGGTAAGTCGGA |
| SEQ ID NO: 864 | CGCTCTGATAAAAACNNKGGTAAGTCGGACAAC |
| SEQ ID NO: 865 | TCTGATAAAAACCGTNNKAAGTCGGACAACGTG |
| SEQ ID NO: 866 | TAAAAACCGTGGTNNKTCGGACAACGTGC |
| SEQ ID NO: 867 | ACCGTGGTAAGNNKGACAACGTGCC |
| SEQ ID NO: 868 | GTGGTAAGTCGNNKAACGTGCCCTCC |
| SEQ ID NO: 869 | GTAAGTCGGACNNKGTGCCCTCCGAA |
| SEQ ID NO: 870 | AAGTCGGACAACNNKCCCTCCGAAGAG |
| SEQ ID NO: 871 | TCGGACAACGTGNNKTCCGAAGAGGTTG |
| SEQ ID NO: 872 | GACAACGTGCCCNNKGAAGAGGTTGTG |
| SEQ ID NO: 873 | AACGTGCCCTCCNNKGAGGTTGTGAAA |
| SEQ ID NO: 874 | ACGTGCCCTCCGAANNKGTTGTGAAAAAGAT |
| SEQ ID NO: 875 | GTGCCCTCCGAAGAGNNKGTGAAAAAGATGAAA |
| SEQ ID NO: 876 | CGTGCCCTCCGAAGAGGTTNNKAAAAAGATGAAAAATTATT |
| SEQ ID NO: 877 | CCCTCCGAAGAGGTTGTGNNKAAGATGAAAAATTATTGG |
| SEQ ID NO: 878 | CCGAAGAGGTTGTGAAANNKATGAAAAATTATTGGCGC |
| SEQ ID NO: 879 | AAGAGGTTGTGAAAAAGNNKAAAAATTATTGGCGCCA |
| SEQ ID NO: 880 | GTTGTGAAAAAGATGNNKAATTATTGGCGCCAGC |
| SEQ ID NO: 881 | TTGTGAAAAAGATGAAANNKTATTGGCGCCAGCTTTT |
| SEQ ID NO: 882 | TGAAAAAGATGAAAAATNNKTGGCGCCAGCTTTTAAAC |
| SEQ ID NO: 883 | AAAAGATGAAAAATTATNNKCGCCAGCTTTTAAACGCG |
| SEQ ID NO: 884 | AAGATGAAAAATTATTGGNNKCAGCTTTTAAACGCGAAGC |
| SEQ ID NO: 885 | GAAAAATTATTGGCGCNNKCTTTTAAACGCGAAGC |
| SEQ ID NO: 886 | TATTGGCGCCAGNNKTTAAACGCGAAGC |
| SEQ ID NO: 887 | GGCGCCAGCTTNNKAACGCGAAGCT |
| SEQ ID NO: 888 | CGCCAGCTTTTANNKGCGAAGCTGATCA |
| SEQ ID NO: 889 | CGCCAGCTTTTAAACNNKAAGCTGATCACACAA |
| SEQ ID NO: 890 | GCTTTTAAACGCGNNKCTGATCACACAACG |
| SEQ ID NO: 891 | AGCTTTTAAACGCGAAGNNKATCACACAACGTAAATT |
| SEQ ID NO: 892 | TTTAAACGCGAAGCTGNNKACACAACGTAAATTCG |
| SEQ ID NO: 893 | TAAACGCGAAGCTGATCNNKCAACGTAAATTCGATAA |
| SEQ ID NO: 894 | CGCGAAGCTGATCACANNKCGTAAATTCGATAATTT |
| SEQ ID NO: 895 | GCGAAGCTGATCACACAANNKAAATTCGATAATTTGACC |
| SEQ ID NO: 896 | GCTGATCACACAACGTNNKTTCGATAATTTGACCAA |
| SEQ ID NO: 897 | TGATCACACAACGTAAANNKGATAATTTGACCAAGGCT |
| SEQ ID NO: 898 | CACACAACGTAAATTCNNKAATTTGACCAAGGCTGA |
| SEQ ID NO: 899 | ACAACGTAAATTCGATNNKTTGACCAAGGCTGAAC |
| SEQ ID NO: 900 | ACGTAAATTCGATAATNNKACCAAGGCTGAACGGG |
| SEQ ID NO: 901 | TAAATTCGATAATTTGNNKAAGGCTGAACGGGGTG |
| SEQ ID NO: 902 | CGATAATTTGACCNNKGCTGAACGGGGTG |
| SEQ ID NO: 903 | AATTTGACCAAGNNKGAACGGGGTGGC |
| SEQ ID NO: 904 | TGACCAAGGCTNNKCGGGGTGGCCT |
| SEQ ID NO: 905 | CCAAGGCTGAANNKGGTGGCCTGAG |
| SEQ ID NO: 906 | AGGCTGAACGGNNKGGCCTGAGCGA |
| SEQ ID NO: 907 | GCTGAACGGGGTNNKCTGAGCGAGTTA |
| SEQ ID NO: 908 | GAACGGGGTGGCNNKAGCGAGTTAGATA |
| SEQ ID NO: 909 | CGGGGTGGCCTGNNKGAGTTAGATAAGG |
| SEQ ID NO: 910 | GGGTGGCCTGAGCNNKTTAGATAAGGCAG |
| SEQ ID NO: 911 | GCCTGAGCGAGNNKGATAAGGCAGGA |
| SEQ ID NO: 912 | GGCCTGAGCGAGTTANNKAAGGCAGGATTTATTA |
| SEQ ID NO: 913 | CCTGAGCGAGTTAGATNNKGCAGGATTTATTAAACG |
| SEQ ID NO: 914 | TGAGCGAGTTAGATAAGNNKGGATTTATTAAACGCCAG |
| SEQ ID NO: 915 | AGCGAGTTAGATAAGGCANNKTTTATTAAACGCCAGTTA |
| SEQ ID NO: 916 | AGTTAGATAAGGCAGGANNKATTAAACGCCAGTTAGTG |
| SEQ ID NO: 917 | TAGATAAGGCAGGATTINNKAAACGCCAGTTAGTGGA |
| SEQ ID NO: 918 | TAAGGCAGGATTTATTNNKCGCCAGTTAGTGGAGA |
| SEQ ID NO: 919 | AGGCAGGATTTATTAAANNKCAGTTAGTGGAGACTCGT |
| SEQ ID NO: 920 | CAGGATTTATTAAACGCNNKTTAGTGGAGACTCGTCA |
| SEQ ID NO: 921 | GATTTATTAAACGCCAGNNKGTGGAGACTCGTCAAAT |
| SEQ ID NO: 922 | TTATTAAACGCCAGTTANNKGAGACTCGTCAAATCACC |
| SEQ ID NO: 923 | AACGCCAGTTAGTGNNKACTCGTCAAATCAC |
| SEQ ID NO: 924 | CCAGTTAGTGGAGNNKCGTCAAATCACCAA |
| SEQ ID NO: 925 | CCAGTTAGTGGAGACTNNKCAAATCACCAAACATGT |
| SEQ ID NO: 926 | GTTAGTGGAGACTCGTNNKATCACCAAACATGTCG |
| SEQ ID NO: 927 | GGAGACTCGTCAANNKACCAAACATGTCGC |
| SEQ ID NO: 928 | GACTCGTCAAATCNNKAAACATGTCGCGCA |
| SEQ ID NO: 929 | GTCAAATCACCNNKCATGTCGCGCAG |
| SEQ ID NO: 930 | GTCAAATCACCAAANNKGTCGCGCAGATTTTG |
| SEQ ID NO: 931 | CAAATCACCAAACATNNKGCGCAGATTTTGGAC |
| SEQ ID NO: 932 | ATCACCAAACATGTCNNKCAGATTTTGGACAGC |
| SEQ ID NO: 933 | CAAACATGTCGCGNNKATTTTGGACAGCC |
| SEQ ID NO: 934 | ATGTCGCGCAGNNKTTGGACAGCCG |
| SEQ ID NO: 935 | TCGCGCAGATTNNKGACAGCCGGAT |
| SEQ ID NO: 936 | GCGCAGATTITGNNKAGCCGGATGAACA |
| SEQ ID NO: 937 | GCAGATTTTGGACNNKCGGATGAACACCAA |
| SEQ ID NO: 938 | CAGATTTTGGACAGCNNKATGAACACCAAGTACG |
| SEQ ID NO: 939 | TTGGACAGCCGGNNKAACACCAAGTAC |
| SEQ ID NO: 940 | GACAGCCGGATGNNKACCAAGTACGATG |
| SEQ ID NO: 941 | GACAGCCGGATGAACNNKAAGTACGATGAAAATG |
| SEQ ID NO: 942 | AGCCGGATGAACACCNNKTACGATGAAAATGAC |
| SEQ ID NO: 943 | CCGGATGAACACCAAGNNKGATGAAAATGACAAAC |
| SEQ ID NO: 944 | GGATGAACACCAAGTACNNKGAAAATGACAAACTGATC |
| SEQ ID NO: 945 | TGAACACCAAGTACGATNNKAATGACAAACTGATCCG |
| SEQ ID NO: 946 | ACCAAGTACGATGAANNKGACAAACTGATCCGTG |
| SEQ ID NO: 947 | CCAAGTACGATGAAAATNNKAAACTGATCCGTGAGGT |
| SEQ ID NO: 948 | TACGATGAAAATGACNNKCTGATCCGTGAGGTG |
| SEQ ID NO: 949 | CGATGAAAATGACAAANNKATCCGTGAGGTGAAAGT |
| SEQ ID NO: 950 | GAAAATGACAAACTGNNKCGTGAGGTGAAAGTCA |
| SEQ ID NO: 951 | TGAAAATGACAAACTGATCNNKGAGGTGAAAGTCATTACTC |
| SEQ ID NO: 952 | ATGACAAACTGATCCGTNNKGTGAAAGTCATTACTCT |
| SEQ ID NO: 953 | ACAAACTGATCCGTGAGNNKAAAGTCATTACTCTGAAG |
| SEQ ID NO: 954 | CTGATCCGTGAGGTGNNKGTCATTACTCTGAAG |
| SEQ ID NO: 955 | GATCCGTGAGGTGAAANNKATTACTCTGAAGTCCAA |
| SEQ ID NO: 956 | CGTGAGGTGAAAGTCNNKACTCTGAAGTCCAAAT |
| SEQ ID NO: 957 | CGTGAGGTGAAAGTCATTNNKCTGAAGTCCAAATTAGTT |
| SEQ ID NO: 958 | CGTGAGGTGAAAGTCATTACTNNKAAGTCCAAATTAGTTAGTGAT |
| SEQ ID NO: 959 | AGGTGAAAGTCATTACTCTGNNKTCCAAATTAGTTAGTGATTTC |
| SEQ ID NO: 960 | TGAAAGTCATTACTCTGAAGNNKAAATTAGTTAGTGATTTCCGG |
| SEQ ID NO: 961 | GTCATTACTCTGAAGTCCNNKTTAGTTAGTGATTTCCGGA |
| SEQ ID NO: 962 | TTACTCTGAAGTCCAAANNKGTTAGTGATTTCCGGAAG |
| SEQ ID NO: 963 | CTCTGAAGTCCAAATTANNKAGTGATTTCCGGAAGGA |
| SEQ ID NO: 964 | TGAAGTCCAAATTAGTTNNKGATTTCCGGAAGGACTT |
| SEQ ID NO: 965 | AAGTCCAAATTAGTTAGTNNKTTCCGGAAGGACTTTCAA |
| SEQ ID NO: 966 | GTCCAAATTAGTTAGTGATNNKCGGAAGGACTTTCAATTCT |
| SEQ ID NO: 967 | GTCCAAATTAGTTAGTGATTTCNNKAAGGACTTTCAATTCTACAAAGT |
| SEQ ID NO: 968 | AATTAGTTAGTGATTTCCGGNNKGACTTTCAATTCTACAAAGT |
| SEQ ID NO: 969 | AGTTAGTGATTTCCGGAAGNNKTTTCAATTCTACAAAGTCC |
| SEQ ID NO: 970 | TGATTTCCGGAAGGACNNKCAATTCTACAAAGTCC |
| SEQ ID NO: 971 | TTTCCGGAAGGACTTTNNKTTCTACAAAGTCCGTG |
| SEQ ID NO: 972 | CCGGAAGGACTTTCAANNKTACAAAGTCCGTGAAAT |
| SEQ ID NO: 973 | CGGAAGGACTTTCAATTCNNKAAAGTCCGTGAAATTAAT |
| SEQ ID NO: 974 | GGAAGGACTTTCAATTCTACNNKGTCCGTGAAATTAATAACTA |
| SEQ ID NO: 975 | GGAAGGACTTTCAATTCTACAAANNKCGTGAAATTAATAACTATCATCA |
| SEQ ID NO: 976 | GGACTTTCAATTCTACAAAGTCNNKGAAATTAATAACTATCATCACGC |
| SEQ ID NO: 977 | TTTCAATTCTACAAAGTCCGTNNKATTAATAACTATCATCACGCA |
| SEQ ID NO: 978 | TCTACAAAGTCCGTGAANNKAATAACTATCATCACGCA |
| SEQ ID NO: 979 | ACAAAGTCCGTGAAATTNNKAACTATCATCACGCACA |
| SEQ ID NO: 980 | AAGTCCGTGAAATTAATNNKTATCATCACGCACATGAC |
| SEQ ID NO: 981 | CCGTGAAATTAATAACNNKCATCACGCACATGACG |
| SEQ ID NO: 982 | GTGAAATTAATAACTATNNKCACGCACATGACGCGTAC |
| SEQ ID NO: 983 | GAAATTAATAACTATCATNNKGCACATGACGCGTACCTG |
| SEQ ID NO: 984 | ATTAATAACTATCATCACNNKCATGACGCGTACCTGAATG |
| SEQ ID NO: 985 | CTATCATCACGCANNKGACGCGTACCTGAA |
| SEQ ID NO: 986 | TCATCACGCACATNNKGCGTACCTGAATG |
| SEQ ID NO: 987 | ATCACGCACATGACNNKTACCTGAATGCAGT |
| SEQ ID NO: 988 | CACATGACGCGNNKCTGAATGCAGTG |
| SEQ ID NO: 989 | ACATGACGCGTACNNKAATGCAGTGGTTG |
| SEQ ID NO: 990 | ACGCGTACCTGNNKGCAGTGGTTGG |
| SEQ ID NO: 991 | GCGTACCTGAATNNKGTGGTTGGGACC |
| SEQ ID NO: 992 | TACCTGAATGCANNKGTTGGGACCGCC |
| SEQ ID NO: 993 | TGAATGCAGTGNNKGGGACCGCCCT |
| SEQ ID NO: 994 | TGAATGCAGTGGTTNNKACCGCCCTTATCAAG |
| SEQ ID NO: 995 | GCAGTGGTTGGGNNKGCCCTTATCAAGA |
| SEQ ID NO: 996 | TGCAGTGGTTGGGACCNNKCTTATCAAGAAATATC |
| SEQ ID NO: 997 | TGGTTGGGACCGCCNNKATCAAGAAATATCC |
| SEQ ID NO: 998 | GTTGGGACCGCCCTTNNKAAGAAATATCCTAAGC |
| SEQ ID NO: 999 | GGGACCGCCCTTATCNNKAAATATCCTAAGCTG |
| SEQ ID NO: 1000 | ACCGCCCTTATCAAGNNKTATCCTAAGCTGGAG |
| SEQ ID NO: 1001 | GCCCTTATCAAGAAANNKCCTAAGCTGGAGTCG |
| SEQ ID NO: 1002 | GCCCTTATCAAGAAATATNNKAAGCTGGAGTCGGAGTTT |
| SEQ ID NO: 1003 | CTTATCAAGAAATATCCTNNKCTGGAGTCGGAGTTTGTC |
| SEQ ID NO: 1004 | TATCAAGAAATATCCTAAGNNKGAGTCGGAGTTTGTCTATGG |
| SEQ ID NO: 1005 | AGAAATATCCTAAGCTGNNKTCGGAGTTTGTCTATGGC |
| SEQ ID NO: 1006 | ATATCCTAAGCTGGAGNNKGAGTTTGTCTATGGCG |
| SEQ ID NO: 1007 | CTAAGCTGGAGTCGNNKTTTGTCTATGGCGAC |
| SEQ ID NO: 1008 | CTGGAGTCGGAGNNKGTCTATGGCGAC |
| SEQ ID NO: 1009 | CTGGAGTCGGAGTTTNNKTATGGCGACTATAAGG |
| SEQ ID NO: 1010 | AGTCGGAGTTTGTCNNKGGCGACTATAAGGTA |
| SEQ ID NO: 1011 | GGAGTCGGAGTTTGTCTATNNKGACTATAAGGTATACGATG |
| SEQ ID NO: 1012 | TCGGAGTTTGTCTATGGCNNKTATAAGGTATACGATGTT |
| SEQ ID NO: 1013 | GTTTGTCTATGGCGACNNKAAGGTATACGATGTTC |
| SEQ ID NO: 1014 | TGTCTATGGCGACTATNNKGTATACGATGTTCGCA |
| SEQ ID NO: 1015 | TCTATGGCGACTATAAGNNKTACGATGTTCGCAAAAT |
| SEQ ID NO: 1016 | ATGGCGACTATAAGGTANNKGATGTTCGCAAAATGAT |
| SEQ ID NO: 1017 | GCGACTATAAGGTATACNNKGTTCGCAAAATGATTGC |
| SEQ ID NO: 1018 | CGACTATAAGGTATACGATNNKCGCAAAATGATTGCGAAAT |
| SEQ ID NO: 1019 | GACTATAAGGTATACGATGTTNNKAAAATGATTGCGAAATCTGAGC |
| SEQ ID NO: 1020 | GGTATACGATGTTCGCNNKATGATTGCGAAATCTG |
| SEQ ID NO: 1021 | ATACGATGTTCGCAAANNKATTGCGAAATCTGAGC |
| SEQ ID NO: 1022 | ATGTTCGCAAAATGNNKGCGAAATCTGAGCAG |
| SEQ ID NO: 1023 | GATGTTCGCAAAATGATTNNKAAATCTGAGCAGGAGATC |
| SEQ ID NO: 1024 | GCAAAATGATTGCGNNKTCTGAGCAGGAGATC |
| SEQ ID NO: 1025 | AAAATGATTGCGAAANNKGAGCAGGAGATCGGTA |
| SEQ ID NO: 1026 | AATGATTGCGAAATCTNNKCAGGAGATCGGTAAGG |
| SEQ ID NO: 1027 | TTGCGAAATCTGAGNNKGAGATCGGTAAGGC |
| SEQ ID NO: 1028 | CGAAATCTGAGCAGNNKATCGGTAAGGCAAC |
| SEQ ID NO: 1029 | TCTGAGCAGGAGNNKGGTAAGGCAACC |
| SEQ ID NO: 1030 | AGCAGGAGATCNNKAAGGCAACCGCA |
| SEQ ID NO: 1031 | CAGGAGATCGGTNNKGCAACCGCAAAAT |
| SEQ ID NO: 1032 | GCAGGAGATCGGTAAGNNKACCGCAAAATATTTCTT |
| SEQ ID NO: 1033 | CAGGAGATCGGTAAGGCANNKGCAAAATATTTCTTTTAC |
| SEQ ID NO: 1034 | GGAGATCGGTAAGGCAACCNNKAAATATTTCTTTTACTCAA |
| SEQ ID NO: 1035 | TCGGTAAGGCAACCGCANNKTATTTCTTTTACTCAAA |
| SEQ ID NO: 1036 | GGTAAGGCAACCGCAAAANNKTTCTTTTACTCAAACATT |
| SEQ ID NO: 1037 | GGTAAGGCAACCGCAAAATATNNKTTTTACTCAAACATTATGAAT |
| SEQ ID NO: 1038 | AGGCAACCGCAAAATATTTCNNKTACTCAAACATTATGAATTT |
| SEQ ID NO: 1039 | GGCAACCGCAAAATATTTCTTTNNKTCAAACATTATGAATTTCTTTA |
| SEQ ID NO: 1040 | GCAACCGCAAAATATTTCTTTTACNNKAACATTATGAATTTCTTTAAGACA |
| SEQ ID NO: 1041 | ACCGCAAAATATTTCTTTTACTCANNKATTATGAATTTCTTTAAGACAGAA |
| SEQ ID NO: 1042 | GCAAAATATTTCTTTTACTCAAACNNKATGAATTTCTTTAAGACAGAAATC |
| SEQ ID NO: 1043 | AAATATTTCTTTTACTCAAACATTNNKAATTTCTTTAAGACAGAAATCACT |
| SEQ ID NO: 1044 | TATTTCTTTTACTCAAACATTATGNNKTTCTTTAAGACAGAAATCACTCTG |
| SEQ ID NO: 1045 | CTTTTACTCAAACATTATGAATNNKTTTAAGACAGAAATCACTCTGGC |
| SEQ ID NO: 1046 | CTCAAACATTATGAATTTCNNKAAGACAGAAATCACTCTGGC |
| SEQ ID NO: 1047 | CAAACATTATGAATTTCTTTNNKACAGAAATCACTCTGGCCAA |
| SEQ ID NO: 1048 | CATTATGAATTTCTTTAAGNNKGAAATCACTCTGGCCAACG |
| SEQ ID NO: 1049 | GAATTTCTTTAAGACANNKATCACTCTGGCCAACGG |
| SEQ ID NO: 1050 | TTCTTTAAGACAGAANNKACTCTGGCCAACGGG |
| SEQ ID NO: 1051 | TAAGACAGAAATCNNKCTGGCCAACGGGG |
| SEQ ID NO: 1052 | GACAGAAATCACTNNKGCCAACGGGGAGAT |
| SEQ ID NO: 1053 | CAGAAATCACTCTGNNKAACGGGGAGATTCG |
| SEQ ID NO: 1054 | TCACTCTGGCCNNKGGGGAGATTCG |
| SEQ ID NO: 1055 | CACTCTGGCCAACNNKGAGATTCGCAAAC |
| SEQ ID NO: 1056 | TGGCCAACGGGNNKATTCGCAAACG |
| SEQ ID NO: 1057 | CCAACGGGGAGNNKCGCAAACGTCC |
| SEQ ID NO: 1058 | CCAACGGGGAGATTNNKAAACGTCCGTTGAT |
| SEQ ID NO: 1059 | GGGAGATTCGCNNKCGTCCGTTGATC |
| SEQ ID NO: 1060 | GGGAGATTCGCAAANNKCCGTTGATCGAAAC |
| SEQ ID NO: 1061 | GAGATTCGCAAACGTNNKTTGATCGAAACAAACG |
| SEQ ID NO: 1062 | TCGCAAACGTCCGNNKATCGAAACAAACG |
| SEQ ID NO: 1063 | AAACGTCCGTTGNNKGAAACAAACGGC |
| SEQ ID NO: 1064 | CGTCCGTTGATCNNKACAAACGGCGAG |
| SEQ ID NO: 1065 | CCGTTGATCGAANNKAACGGCGAGACTG |
| SEQ ID NO: 1066 | TTGATCGAAACANNKGGCGAGACTGGC |
| SEQ ID NO: 1067 | TTGATCGAAACAAACNNKGAGACTGGCGAAATTG |
| SEQ ID NO: 1068 | CGAAACAAACGGCNNKACTGGCGAAATTG |
| SEQ ID NO: 1069 | AACAAACGGCGAGNNKGGCGAAATTGTTT |
| SEQ ID NO: 1070 | CAAACGGCGAGACTNNKGAAATTGTTTGGGA |
| SEQ ID NO: 1071 | GGCGAGACTGGCNNKATTGTTTGGGAC |
| SEQ ID NO: 1072 | CGAGACTGGCGAANNKGTTTGGGACAAAG |
| SEQ ID NO: 1073 | GACTGGCGAAATTNNKTGGGACAAAGGGC |
| SEQ ID NO: 1074 | GGCGAAATTGTTNNKGACAAAGGGCGTG |
| SEQ ID NO: 1075 | GCGAAATTGTTTGGNNKAAAGGGCGTGATTTC |
| SEQ ID NO: 1076 | AATTGTTTGGGACNNKGGGCGTGATTTCG |
| SEQ ID NO: 1077 | TGTTTGGGACAAANNKCGTGATTTCGCGAC |
| SEQ ID NO: 1078 | GGGACAAAGGGNNKGATTTCGCGACG |
| SEQ ID NO: 1079 | ACAAAGGGCGTNNKTTCGCGACGGT |
| SEQ ID NO: 1080 | AAGGGCGTGATNNKGCGACGGTGCG |
| SEQ ID NO: 1081 | GGCGTGATTTCNNKACGGTGCGCAA |
| SEQ ID NO: 1082 | GTGATTTCGCGNNKGTGCGCAAGGTA |
| SEQ ID NO: 1083 | ATTTCGCGACGNNKCGCAAGGTACTG |
| SEQ ID NO: 1084 | TCGCGACGGTGNNKAAGGTACTGAGC |
| SEQ ID NO: 1085 | CGACGGTGCGCNNKGTACTGAGCAT |
| SEQ ID NO: 1086 | CGGTGCGCAAGNNKCTGAGCATGCC |
| SEQ ID NO: 1087 | GTGCGCAAGGTANNKAGCATGCCTCAA |
| SEQ ID NO: 1088 | CGCAAGGTACTGNNKATGCCTCAAGTCA |
| SEQ ID NO: 1089 | CGCAAGGTACTGAGCNNKCCTCAAGTCAATATT |
| SEQ ID NO: 1090 | GCAAGGTACTGAGCATGNNKCAAGTCAATATTGTTAAG |
| SEQ ID NO: 1091 | AGGTACTGAGCATGCCTNNKGTCAATATTGTTAAGAAA |
| SEQ ID NO: 1092 | GTACTGAGCATGCCTCAANNKAATATTGTTAAGAAAACCG |
| SEQ ID NO: 1093 | GAGCATGCCTCAAGTCNNKATTGTTAAGAAAACCG |
| SEQ ID NO: 1094 | CATGCCTCAAGTCAATNNKGTTAAGAAAACCGAAGT |
| SEQ ID NO: 1095 | TGCCTCAAGTCAATATTNNKAAGAAAACCGAAGTGCA |
| SEQ ID NO: 1096 | CTCAAGTCAATATTGTTNNKAAAACCGAAGTGCAGACG |
| SEQ ID NO: 1097 | AGTCAATATTGTTAAGNNKACCGAAGTGCAGACGG |
| SEQ ID NO: 1098 | CAATATTGTTAAGAAANNKGAAGTGCAGACGGGCG |
| SEQ ID NO: 1099 | GTTAAGAAAACCNNKGTGCAGACGGGCG |
| SEQ ID NO: 1100 | AGAAAACCGAANNKCAGACGGGCGG |
| SEQ ID NO: 1101 | AAACCGAAGTGNNKACGGGCGGGTT |
| SEQ ID NO: 1102 | CCGAAGTGCAGNNKGGCGGGTTTTC |
| SEQ ID NO: 1103 | GAAGTGCAGACGNNKGGGTTTTCCAAGG |
| SEQ ID NO: 1104 | AGTGCAGACGGGCNNKTTTTCCAAGGAAA |
| SEQ ID NO: 1105 | AGACGGGCGGGNNKTCCAAGGAAAG |
| SEQ ID NO: 1106 | ACGGGCGGGTTTNNKAAGGAAAGCATCT |
| SEQ ID NO: 1107 | GGGCGGGTTTTCCNNKGAAAGCATCTTAC |
| SEQ ID NO: 1108 | CGGGTTTTCCAAGNNKAGCATCTTACCCAA |
| SEQ ID NO: 1109 | CGGGTTTTCCAAGGAANNKATCTTACCCAAACGTA |
| SEQ ID NO: 1110 | GGTTTTCCAAGGAAAGCNNKTTACCCAAACGTAATTC |
| SEQ ID NO: 1111 | TCCAAGGAAAGCATCNNKCCCAAACGTAATTCAG |
| SEQ ID NO: 1112 | GGTTTTCCAAGGAAAGCATCTTANNKAAACGTAATTCAGATAAACTTAT |
| SEQ ID NO: 1113 | CAAGGAAAGCATCTTACCCNNKCGTAATTCAGATAAACTTA |
| SEQ ID NO: 1114 | AGGAAAGCATCTTACCCAAANNKAATTCAGATAAACTTATTGCA |
| SEQ ID NO: 1115 | AGCATCTTACCCAAACGTNNKTCAGATAAACTTATTGCA |
| SEQ ID NO: 1116 | ATCTTACCCAAACGTAATNNKGATAAACTTATTGCACGC |
| SEQ ID NO: 1117 | TACCCAAACGTAATTCANNKAAACTTATTGCACGCAA |
| SEQ ID NO: 1118 | CCCAAACGTAATTCAGATNNKCTTATTGCACGCAAAAAG |
| SEQ ID NO: 1119 | AAACGTAATTCAGATAAANNKATTGCACGCAAAAAGGACT |
| SEQ ID NO: 1120 | GTAATTCAGATAAACTTNNKGCACGCAAAAAGGACTGG |
| SEQ ID NO: 1121 | TAATTCAGATAAACTTATTNNKCGCAAAAAGGACTGGGATCC |
| SEQ ID NO: 1122 | CAGATAAACTTATTGCANNKAAAAAGGACTGGGATCCG |
| SEQ ID NO: 1123 | ACTTATTGCACGCNNKAAGGACTGGGATCC |
| SEQ ID NO: 1124 | TTATTGCACGCAAANNKGACTGGGATCCGAA |
| SEQ ID NO: 1125 | TTGCACGCAAAAAGNNKTGGGATCCGAAAAA |
| SEQ ID NO: 1126 | GCACGCAAAAAGGACNNKGATCCGAAAAAGTAT |
| SEQ ID NO: 1127 | GCAAAAAGGACTGGNNKCCGAAAAAGTATGGA |
| SEQ ID NO: 1128 | GCAAAAAGGACTGGGATNNKAAAAAGTATGGAGGCTT |
| SEQ ID NO: 1129 | GACTGGGATCCGNNKAAGTATGGAGGCT |
| SEQ ID NO: 1130 | ACTGGGATCCGAAANNKTATGGAGGCTTCGA |
| SEQ ID NO: 1131 | GGATCCGAAAAAGNNKGGAGGCTTCGACA |
| SEQ ID NO: 1132 | GATCCGAAAAAGTATNNKGGCTTCGACAGTCCAA |
| SEQ ID NO: 1133 | CCGAAAAAGTATGGANNKTTCGACAGTCCAACC |
| SEQ ID NO: 1134 | AAAGTATGGAGGCNNKGACAGTCCAACCG |
| SEQ ID NO: 1135 | GTATGGAGGCTTCNNKAGTCCAACCGTAGC |
| SEQ ID NO: 1136 | GAGGCTTCGACNNKCCAACCGTAGC |
| SEQ ID NO: 1137 | AGGCTTCGACAGTNNKACCGTAGCCTACT |
| SEQ ID NO: 1138 | CTTCGACAGTCCANNKGTAGCCTACTCTGT |
| SEQ ID NO: 1139 | CGACAGTCCAACCNNKGCCTACTCTGTTC |
| SEQ ID NO: 1140 | GACAGTCCAACCGTANNKTACTCTGTTCTCGTTG |
| SEQ ID NO: 1141 | TCCAACCGTAGCCNNKTCTGTTCTCGTTG |
| SEQ ID NO: 1142 | AACCGTAGCCTACNNKGTTCTCGTTGTAGC |
| SEQ ID NO: 1143 | CGTAGCCTACTCTNNKCTCGTTGTAGCGAA |
| SEQ ID NO: 1144 | CGTAGCCTACTCTGTTNNKGTTGTAGCGAAAGTAG |
| SEQ ID NO: 1145 | TAGCCTACTCTGTTCTCNNKGTAGCGAAAGTAGAAAA |
| SEQ ID NO: 1146 | CTACTCTGTTCTCGTTNNKGCGAAAGTAGAAAAGGG |
| SEQ ID NO: 1147 | CCTACTCTGTTCTCGTTGTANNKAAAGTAGAAAAGGGTAAATC |
| SEQ ID NO: 1148 | CTGTTCTCGTTGTAGCGNNKGTAGAAAAGGGTAAATC |
| SEQ ID NO: 1149 | TTCTCGTTGTAGCGAAANNKGAAAAGGGTAAATCCAA |
| SEQ ID NO: 1150 | TCGTTGTAGCGAAAGTANNKAAGGGTAAATCCAAGAA |
| SEQ ID NO: 1151 | GTTGTAGCGAAAGTAGAANNKGGTAAATCCAAGAAACTGA |
| SEQ ID NO: 1152 | TTGTAGCGAAAGTAGAAAAGNNKAAATCCAAGAAACTGAAATCT |
| SEQ ID NO: 1153 | GCGAAAGTAGAAAAGGGTNNKTCCAAGAAACTGAAATCT |
| SEQ ID NO: 1154 | CGAAAGTAGAAAAGGGTAAANNKAAGAAACTGAAATCTGTCAAG |
| SEQ ID NO: 1155 | AGTAGAAAAGGGTAAATCCNNKAAACTGAAATCTGTCAAGG |
| SEQ ID NO: 1156 | AAAAGGGTAAATCCAAGNNKCTGAAATCTGTCAAGGA |
| SEQ ID NO: 1157 | AAGGGTAAATCCAAGAAANNKAAATCTGTCAAGGAGTTGC |
| SEQ ID NO: 1158 | GTAAATCCAAGAAACTGNNKTCTGTCAAGGAGTTGCT |
| SEQ ID NO: 1159 | ATCCAAGAAACTGAAANNKGTCAAGGAGTTGCTTG |
| SEQ ID NO: 1160 | TCCAAGAAACTGAAATCTNNKAAGGAGTTGCTTGGAATC |
| SEQ ID NO: 1161 | GAAACTGAAATCTGTCNNKGAGTTGCTTGGAATCAC |
| SEQ ID NO: 1162 | AACTGAAATCTGTCAAGNNKTTGCTTGGAATCACCAT |
| SEQ ID NO: 1163 | TGAAATCTGTCAAGGAGNNKCTTGGAATCACCATTAT |
| SEQ ID NO: 1164 | AATCTGTCAAGGAGTTGNNKGGAATCACCATTATGGA |
| SEQ ID NO: 1165 | TGTCAAGGAGTTGCTTNNKATCACCATTATGGAGC |
| SEQ ID NO: 1166 | AGGAGTTGCTTGGANNKACCATTATGGAGCG |
| SEQ ID NO: 1167 | GAGTTGCTTGGAATCNNKATTATGGAGCGTAGCT |
| SEQ ID NO: 1168 | CTTGGAATCACCNNKATGGAGCGTAGCT |
| SEQ ID NO: 1169 | TTGGAATCACCATTNNKGAGCGTAGCTCCTTC |
| SEQ ID NO: 1170 | GGAATCACCATTATGNNKCGTAGCTCCTTCGAG |
| SEQ ID NO: 1171 | AATCACCATTATGGAGNNKAGCTCCTTCGAGAAGAA |
| SEQ ID NO: 1172 | ACCATTATGGAGCGTNNKTCCTTCGAGAAGAAC |
| SEQ ID NO: 1173 | TTATGGAGCGTAGCNNKTTCGAGAAGAACCC |
| SEQ ID NO: 1174 | GAGCGTAGCTCCNNKGAGAAGAACCCG |
| SEQ ID NO: 1175 | GCGTAGCTCCTTCNNKAAGAACCCGATTGA |
| SEQ ID NO: 1176 | GTAGCTCCTTCGAGNNKAACCCGATTGACTTT |
| SEQ ID NO: 1177 | GCTCCTTCGAGAAGNNKCCGATTGACTTTCT |
| SEQ ID NO: 1178 | CTCCTTCGAGAAGAACNNKATTGACTTTCTGGAAGC |
| SEQ ID NO: 1179 | CGAGAAGAACCCGNNKGACTTTCTGGAAG |
| SEQ ID NO: 1180 | GAGAAGAACCCGATTNNKTTTCTGGAAGCCAAAG |
| SEQ ID NO: 1181 | GAACCCGATTGACNNKCTGGAAGCCAAAG |
| SEQ ID NO: 1182 | AGAACCCGATTGACTTTNNKGAAGCCAAAGGATATAA |
| SEQ ID NO: 1183 | CCCGATTGACTTTCTGNNKGCCAAAGGATATAAAGA |
| SEQ ID NO: 1184 | CCGATTGACTTTCTGGAANNKAAAGGATATAAAGAGGTCA |
| SEQ ID NO: 1185 | TTGACTTTCTGGAAGCCNNKGGATATAAAGAGGTCAA |
| SEQ ID NO: 1186 | TGACTTTCTGGAAGCCAAANNKTATAAAGAGGTCAAGAAAG |
| SEQ ID NO: 1187 | TCTGGAAGCCAAAGGANNKAAAGAGGTCAAGAAAG |
| SEQ ID NO: 1188 | TGGAAGCCAAAGGATATNNKGAGGTCAAGAAAGATCT |
| SEQ ID NO: 1189 | CTGGAAGCCAAAGGATATAAANNKGTCAAGAAAGATCTTATCATT |
| SEQ ID NO: 1190 | GGAAGCCAAAGGATATAAAGAGNNKAAGAAAGATCTTATCATTAAGC |
| SEQ ID NO: 1191 | GCCAAAGGATATAAAGAGGTCNNKAAAGATCTTATCATTAAGCTG |
| SEQ ID NO: 1192 | AGGATATAAAGAGGTCAAGNNKGATCTTATCATTAAGCTGCC |
| SEQ ID NO: 1193 | GGATATAAAGAGGTCAAGAAANNKCTTATCATTAAGCTGCCTAAG |
| SEQ ID NO: 1194 | GATATAAAGAGGTCAAGAAAGATNNKATCATTAAGCTGCCTAAGTATTCA |
| SEQ ID NO: 1195 | AAGAGGTCAAGAAAGATCTTNNKATTAAGCTGCCTAAGTATTCA |
| SEQ ID NO: 1196 | GGTCAAGAAAGATCTTATCNNKAAGCTGCCTAAGTATTCAC |
| SEQ ID NO: 1197 | GTCAAGAAAGATCTTATCATTNNKCTGCCTAAGTATTCACTCTTC |
| SEQ ID NO: 1198 | AAGAAAGATCTTATCATTAAGNNKCCTAAGTATTCACTCTTCGAGC |
| SEQ ID NO: 1199 | AGATCTTATCATTAAGCTGNNKAAGTATTCACTCTTCGAGCT |
| SEQ ID NO: 1200 | TTATCATTAAGCTGCCTNNKTATTCACTCTTCGAGCTG |
| SEQ ID NO: 1201 | ATTAAGCTGCCTAAGNNKTCACTCTTCGAGCTG |
| SEQ ID NO: 1202 | TTAAGCTGCCTAAGTATNNKCTCTTCGAGCTGGAAAA |
| SEQ ID NO: 1203 | GCTGCCTAAGTATTCANNKTTCGAGCTGGAAAATG |
| SEQ ID NO: 1204 | GCCTAAGTATTCACTCNNKGAGCTGGAAAATGGTC |
| SEQ ID NO: 1205 | CCTAAGTATTCACTCTTCNNKCTGGAAAATGGTCGTAAAC |
| SEQ ID NO: 1206 | AGTATTCACTCTTCGAGNNKGAAAATGGTCGTAAACG |
| SEQ ID NO: 1207 | CACTCTTCGAGCTGNNKAATGGTCGTAAACG |
| SEQ ID NO: 1208 | TTCGAGCTGGAANNKGGTCGTAAACGCA |
| SEQ ID NO: 1209 | CGAGCTGGAAAATNNKCGTAAACGCATGCT |
| SEQ ID NO: 1210 | GCTGGAAAATGGTNNKAAACGCATGCTCG |
| SEQ ID NO: 1211 | GAAAATGGTCGTNNKCGCATGCTCGCTT |
| SEQ ID NO: 1212 | AAAATGGTCGTAAANNKATGCTCGCTTCTGCC |
| SEQ ID NO: 1213 | GTCGTAAACGCNNKCTCGCTTCTGCC |
| SEQ ID NO: 1214 | GTAAACGCATGNNKGCTTCTGCCGG |
| SEQ ID NO: 1215 | AACGCATGCTCNNKTCTGCCGGCGA |
| SEQ ID NO: 1216 | GCATGCTCGCTNNKGCCGGCGAGTT |
| SEQ ID NO: 1217 | TGCTCGCTTCTNNKGGCGAGTTGCA |
| SEQ ID NO: 1218 | CTCGCTTCTGCCNNKGAGTTGCAGAAG |
| SEQ ID NO: 1219 | CTTCTGCCGGCNNKTTGCAGAAGGG |
| SEQ ID NO: 1220 | CTGCCGGCGAGNNKCAGAAGGGCAA |
| SEQ ID NO: 1221 | CCGGCGAGTTGNNKAAGGGCAATGAA |
| SEQ ID NO: 1222 | CGGCGAGTTGCAGNNKGGCAATGAATTAG |
| SEQ ID NO: 1223 | GGCGAGTTGCAGAAGNNKAATGAATTAGCACTTC |
| SEQ ID NO: 1224 | GTTGCAGAAGGGCNNKGAATTAGCACTTCC |
| SEQ ID NO: 1225 | TTGCAGAAGGGCAATNNKTTAGCACTTCCATCA |
| SEQ ID NO: 1226 | CAGAAGGGCAATGAANNKGCACTTCCATCAAAG |
| SEQ ID NO: 1227 | AGAAGGGCAATGAATTANNKCTTCCATCAAAGTACGT |
| SEQ ID NO: 1228 | AGGGCAATGAATTAGCANNKCCATCAAAGTACGTTAA |
| SEQ ID NO: 1229 | GGCAATGAATTAGCACTTNNKTCAAAGTACGTTAACTTCC |
| SEQ ID NO: 1230 | AATGAATTAGCACTTCCANNKAAGTACGTTAACTTCCTGT |
| SEQ ID NO: 1231 | ATGAATTAGCACTTCCATCANNKTACGTTAACTTCCTGTATTTG |
| SEQ ID NO: 1232 | TTAGCACTTCCATCAAAGNNKGTTAACTTCCTGTATTTGG |
| SEQ ID NO: 1233 | CACTTCCATCAAAGTACNNKAACTTCCTGTATTTGGC |
| SEQ ID NO: 1234 | TCCATCAAAGTACGTTNNKTTCCTGTATTTGGCCAG |
| SEQ ID NO: 1235 | TCAAAGTACGTTAACNNKCTGTATTTGGCCAGCC |
| SEQ ID NO: 1236 | CAAAGTACGTTAACTTCNNKTATTTGGCCAGCCATTAC |
| SEQ ID NO: 1237 | TACGTTAACTTCCTGNNKTTGGCCAGCCATTAC |
| SEQ ID NO: 1238 | GTTAACTTCCTGTATNNKGCCAGCCATTACGAGA |
| SEQ ID NO: 1239 | GTTAACTTCCTGTATTTGNNKAGCCATTACGAGAAACTGA |
| SEQ ID NO: 1240 | CTTCCTGTATTTGGCCNNKCATTACGAGAAACTGAA |
| SEQ ID NO: 1241 | CTGTATTTGGCCAGCNNKTACGAGAAACTGAAG |
| SEQ ID NO: 1242 | TTTGGCCAGCCATNNKGAGAAACTGAAGG |
| SEQ ID NO: 1243 | GCCAGCCATTACNNKAAACTGAAGGGGT |
| SEQ ID NO: 1244 | CAGCCATTACGAGNNKCTGAAGGGGTCTC |
| SEQ ID NO: 1245 | GCCATTACGAGAAANNKAAGGGGTCTCCAGAG |
| SEQ ID NO: 1246 | ATTACGAGAAACTGNNKGGGTCTCCAGAGGAC |
| SEQ ID NO: 1247 | TACGAGAAACTGAAGNNKTCTCCAGAGGACAAC |
| SEQ ID NO: 1248 | AAACTGAAGGGGNNKCCAGAGGACAACG |
| SEQ ID NO: 1249 | ACTGAAGGGGTCTNNKGAGGACAACGAAC |
| SEQ ID NO: 1250 | GAAGGGGTCTCCANNKGACAACGAACAGAA |
| SEQ ID NO: 1251 | AGGGGTCTCCAGAGNNKAACGAACAGAAACA |
| SEQ ID NO: 1252 | GGGTCTCCAGAGGACNNKGAACAGAAACAATTAT |
| SEQ ID NO: 1253 | GGTCTCCAGAGGACAACNNKCAGAAACAATTATTTGT |
| SEQ ID NO: 1254 | TCTCCAGAGGACAACGAANNKAAACAATTATTTGTAGAGC |
| SEQ ID NO: 1255 | CAGAGGACAACGAACAGNNKCAATTATTTGTAGAGCA |
| SEQ ID NO: 1256 | AGGACAACGAACAGAAANNKTTATTTGTAGAGCAGCA |
| SEQ ID NO: 1257 | CAACGAACAGAAACAANNKTTTGTAGAGCAGCACAA |
| SEQ ID NO: 1258 | CGAACAGAAACAATTANNKGTAGAGCAGCACAAGC |
| SEQ ID NO: 1259 | CGAACAGAAACAATTATTTNNKGAGCAGCACAAGCATTATC |
| SEQ ID NO: 1260 | GAACAGAAACAATTATTTGTANNKCAGCACAAGCATTATCTTGAT |
| SEQ ID NO: 1261 | CAGAAACAATTATTTGTAGAGNNKCACAAGCATTATCTTGATGAAA |
| SEQ ID NO: 1262 | AGAAACAATTATTTGTAGAGCAGNNKAAGCATTATCTTGATGAAATCAT |
| SEQ ID NO: 1263 | CAATTATTTGTAGAGCAGCACNNKCATTATCTTGATGAAATCATTG |
| SEQ ID NO: 1264 | ATTTGTAGAGCAGCACAAGNNKTATCTTGATGAAATCATTGA |
| SEQ ID NO: 1265 | TAGAGCAGCACAAGCATNNKCTTGATGAAATCATTGA |
| SEQ ID NO: 1266 | GCAGCACAAGCATTATNNKGATGAAATCATTGAGCA |
| SEQ ID NO: 1267 | GCAGCACAAGCATTATCTTNNKGAAATCATTGAGCAAATTT |
| SEQ ID NO: 1268 | GCACAAGCATTATCTTGATNNKATCATTGAGCAAATTTCCG |
| SEQ ID NO: 1269 | ACAAGCATTATCTTGATGAANNKATTGAGCAAATTTCCGAATT |
| SEQ ID NO: 1270 | GCATTATCTTGATGAAATCNNKGAGCAAATTTCCGAATTCA |
| SEQ ID NO: 1271 | GCATTATCTTGATGAAATCATTNNKCAAATTTCCGAATTCAGTAAACG |
| SEQ ID NO: 1272 | ATCTTGATGAAATCATTGAGNNKATTTCCGAATTCAGTAAACGC |
| SEQ ID NO: 1273 | ATGAAATCATTGAGCAANNKTCCGAATTCAGTAAACGC |
| SEQ ID NO: 1274 | TGAAATCATTGAGCAAATTNNKGAATTCAGTAAACGCGTAA |
| SEQ ID NO: 1275 | TCATTGAGCAAATTTCCNNKTTCAGTAAACGCGTAATC |
| SEQ ID NO: 1276 | TGAGCAAATTTCCGAANNKAGTAAACGCGTAATCC |
| SEQ ID NO: 1277 | CAAATTTCCGAATTCNNKAAACGCGTAATCCTGG |
| SEQ ID NO: 1278 | TTCCGAATTCAGTNNKCGCGTAATCCTGG |
| SEQ ID NO: 1279 | TTCCGAATTCAGTAAANNKGTAATCCTGGCCGATG |
| SEQ ID NO: 1280 | ATTCAGTAAACGCNNKATCCTGGCCGATG |
| SEQ ID NO: 1281 | GTAAACGCGTANNKCTGGCCGATGCA |
| SEQ ID NO: 1282 | TAAACGCGTAATCNNKGCCGATGCAAACC |
| SEQ ID NO: 1283 | CGCGTAATCCTGNNKGATGCAAACCTCG |
| SEQ ID NO: 1284 | GTAATCCTGGCCNNKGCAAACCTCGAC |
| SEQ ID NO: 1285 | TAATCCTGGCCGATNNKAACCTCGACAAGGT |
| SEQ ID NO: 1286 | TGGCCGATGCANNKCTCGACAAGGT |
| SEQ ID NO: 1287 | CCGATGCAAACNNKGACAAGGTGCTG |
| SEQ ID NO: 1288 | GATGCAAACCTCNNKAAGGTGCTGAGCG |
| SEQ ID NO: 1289 | CAAACCTCGACNNKGTGCTGAGCGC |
| SEQ ID NO: 1290 | AAACCTCGACAAGNNKCTGAGCGCTTACA |
| SEQ ID NO: 1291 | AACCTCGACAAGGTGNNKAGCGCTTACAATAAG |
| SEQ ID NO: 1292 | TCGACAAGGTGCTGNNKGCTTACAATAAGCAT |
| SEQ ID NO: 1293 | ACAAGGTGCTGAGCNNKTACAATAAGCATCG |
| SEQ ID NO: 1294 | GTGCTGAGCGCTNNKAATAAGCATCGC |
| SEQ ID NO: 1295 | CTGAGCGCTTACNNKAAGCATCGCGAC |
| SEQ ID NO: 1296 | GAGCGCTTACAATNNKCATCGCGACAAAC |
| SEQ ID NO: 1297 | GCGCTTACAATAAGNNKCGCGACAAACCTATC |
| SEQ ID NO: 1298 | CGCTTACAATAAGCATNNKGACAAACCTATCCGTGA |
| SEQ ID NO: 1299 | TACAATAAGCATCGCNNKAAACCTATCCGTGAGC |
| SEQ ID NO: 1300 | AAGCATCGCGACNNKCCTATCCGTGAG |
| SEQ ID NO: 1301 | CATCGCGACAAANNKATCCGTGAGCAGG |
| SEQ ID NO: 1302 | GCGACAAACCTNNKCGTGAGCAGGC |
| SEQ ID NO: 1303 | CGACAAACCTATCNNKGAGCAGGCTGAAAA |
| SEQ ID NO: 1304 | CGACAAACCTATCCGTNNKCAGGCTGAAAATATCAT |
| SEQ ID NO: 1305 | ACAAACCTATCCGTGAGNNKGCTGAAAATATCATTCA |
| SEQ ID NO: 1306 | ACCTATCCGTGAGCAGNNKGAAAATATCATTCACCT |
| SEQ ID NO: 1307 | TATCCGTGAGCAGGCTNNKAATATCATTCACCTGTT |
| SEQ ID NO: 1308 | GTGAGCAGGCTGAANNKATCATTCACCTGTTC |
| SEQ ID NO: 1309 | TGAGCAGGCTGAAAATNNKATTCACCTGTTCACATT |
| SEQ ID NO: 1310 | AGCAGGCTGAAAATATCNNKCACCTGTTCACATTAAC |
| SEQ ID NO: 1311 | CAGGCTGAAAATATCATTNNKCTGTTCACATTAACGAACC |
| SEQ ID NO: 1312 | GCTGAAAATATCATTCACNNKTTCACATTAACGAACCTGG |
| SEQ ID NO: 1313 | AAAATATCATTCACCTGNNKACATTAACGAACCTGGGC |
| SEQ ID NO: 1314 | ATCATTCACCTGTTCNNKTTAACGAACCTGGGC |
| SEQ ID NO: 1315 | CACCTGTTCACANNKACGAACCTGGGC |
| SEQ ID NO: 1316 | CCTGTTCACATTANNKAACCTGGGCGCTC |
| SEQ ID NO: 1317 | TCACATTAACGNNKCTGGGCGCTCC |
| SEQ ID NO: 1318 | CATTAACGAACNNKGGCGCTCCGGC |
| SEQ ID NO: 1319 | TAACGAACCTGNNKGCTCCGGCCGC |
| SEQ ID NO: 1320 | CGAACCTGGGCNNKCCGGCCGCTTT |
| SEQ ID NO: 1321 | ACCTGGGCGCTNNKGCCGCTTTTAA |
| SEQ ID NO: 1322 | CCTGGGCGCTCCGNNKGCTTTTAAATATTT |
| SEQ ID NO: 1323 | GGGCGCTCCGGCCNNKTTTAAATATTTCG |
| SEQ ID NO: 1324 | GCTCCGGCCGCTNNKAAATATTTCGACA |
| SEQ ID NO: 1325 | CCGGCCGCTTTTNNKTATTTCGACACGA |
| SEQ ID NO: 1326 | CGGCCGCTTTTAAANNKTTCGACACGACAAT |
| SEQ ID NO: 1327 | GCCGCTITTAAATATNNKGACACGACAATCGACC |
| SEQ ID NO: 1328 | CCGCTTTTAAATATTTCNNKACGACAATCGACCGTAA |
| SEQ ID NO: 1329 | CTTTTAAATATTTCGACNNKACAATCGACCGTAAGCGC |
| SEQ ID NO: 1330 | AATATTTCGACACGNNKATCGACCGTAAGCG |
| SEQ ID NO: 1331 | TCGACACGACANNKGACCGTAAGCG |
| SEQ ID NO: 1332 | TCGACACGACAATCNNKCGTAAGCGCTATAC |
| SEQ ID NO: 1333 | CACGACAATCGACNNKAAGCGCTATACCAG |
| SEQ ID NO: 1334 | CGACAATCGACCGTNNKCGCTATACCAGTAC |
| SEQ ID NO: 1335 | GACAATCGACCGTAAGNNKTATACCAGTACGAAAGA |
| SEQ ID NO: 1336 | GACCGTAAGCGCNNKACCAGTACGAAAG |
| SEQ ID NO: 1337 | GACCGTAAGCGCTATNNKAGTACGAAAGAAGTGT |
| SEQ ID NO: 1338 | CGTAAGCGCTATACCNNKACGAAAGAAGTGTTG |
| SEQ ID NO: 1339 | TAAGCGCTATACCAGTNNKAAAGAAGTGTTGGATGC |
| SEQ ID NO: 1340 | GCTATACCAGTACGNNKGAAGTGTTGGATGC |
| SEQ ID NO: 1341 | TATACCAGTACGAAANNKGTGTTGGATGCGACC |
| SEQ ID NO: 1342 | CCAGTACGAAAGAANNKTTGGATGCGACCCTT |
| SEQ ID NO: 1343 | CAGTACGAAAGAAGTGNNKGATGCGACCCTTATTC |
| SEQ ID NO: 1344 | CGAAAGAAGTGTTGNNKGCGACCCTTATTCAC |
| SEQ ID NO: 1345 | CGAAAGAAGTGTTGGATNNKACCCTTATTCACCAGTC |
| SEQ ID NO: 1346 | AAGTGTTGGATGCGNNKCTTATTCACCAGTCA |
| SEQ ID NO: 1347 | TGTTGGATGCGACCNNKATTCACCAGTCAAT |
| SEQ ID NO: 1348 | TGGATGCGACCCTTNNKCACCAGTCAATTAC |
| SEQ ID NO: 1349 | GGATGCGACCCTTATTNNKCAGTCAATTACAGGATT |
| SEQ ID NO: 1350 | GGATGCGACCCTTATTCACNNKTCAATTACAGGATTATATG |
| SEQ ID NO: 1351 | GCGACCCTTATTCACCAGNNKATTACAGGATTATATGAGA |
| SEQ ID NO: 1352 | ACCCTTATTCACCAGTCANNKACAGGATTATATGAGACC |
| SEQ ID NO: 1353 | CCTTATTCACCAGTCAATTNNKGGATTATATGAGACCCGTA |
| SEQ ID NO: 1354 | TATTCACCAGTCAATTACANNKTTATATGAGACCCGTATCGA |
| SEQ ID NO: 1355 | CCAGTCAATTACAGGANNKTATGAGACCCGTATCGA |
| SEQ ID NO: 1356 | GTCAATTACAGGATTANNKGAGACCCGTATCGACCT |
| SEQ ID NO: 1357 | TCAATTACAGGATTATATNNKACCCGTATCGACCTTAGCC |
| SEQ ID NO: 1358 | TTACAGGATTATATGAGNNKCGTATCGACCTTAGCCA |
| SEQ ID NO: 1359 | ACAGGATTATATGAGACCNNKATCGACCTTAGCCAATTA |
| SEQ ID NO: 1360 | GATTATATGAGACCCGTNNKGACCTTAGCCAATTAGGT |
| SEQ ID NO: 1361 | TATATGAGACCCGTATCNNKCTTAGCCAATTAGGTGG |
| SEQ ID NO: 1362 | GACCCGTATCGACNNKAGCCAATTAGGTGG |
| SEQ ID NO: 1363 | CCGTATCGACCTTNNKCAATTAGGTGGGGA |
| SEQ ID NO: 1364 | GTATCGACCTTAGCNNKTTAGGTGGGGATAGC |
| SEQ ID NO: 1365 | GACCTTAGCCAANNKGGTGGGGATAGCG |
| SEQ ID NO: 1366 | CCTTAGCCAATTANNKGGGGATAGCGGTGG |
| SEQ ID NO: 1367 | CTTAGCCAATTAGGTNNKGATAGCGGTGGTAGC |
| SEQ ID NO: 1368 | CAATTAGGTGGGNNKAGCGGTGGTAGCA |
| SEQ ID NO: 1369 | TTAGGTGGGGATNNKGGTGGTAGCAGC |
| SEQ ID NO: 1370 | GTGGGGATAGCNNKGGTAGCAGCGG |
| SEQ ID NO: 1371 | GGGATAGCGGTNNKAGCAGCGGTGG |
| SEQ ID NO: 1372 | GATAGCGGTGGTNNKAGCGGTGGTTCAA |
| SEQ ID NO: 1373 | GCGGTGGTAGCNNKGGTGGTTCAAG |
| SEQ ID NO: 1374 | GTGGTAGCAGCNNKGGTTCAAGCGG |
| SEQ ID NO: 1375 | GTAGCAGCGGTNNKTCAAGCGGTAGC |
| SEQ ID NO: 1376 | GCAGCGGTGGTNNKAGCGGTAGCGA |
| SEQ ID NO: 1377 | AGCGGTGGTTCANNKGGTAGCGAAACA |
| SEQ ID NO: 1378 | GTGGTTCAAGCNNKAGCGAAACACCG |
| SEQ ID NO: 1379 | GTTCAAGCGGTNNKGAAACACCGGGT |
| SEQ ID NO: 1380 | CAAGCGGTAGCNNKACACCGGGTAC |
| SEQ ID NO: 1381 | GCGGTAGCGAANNKCCGGGTACAAG |
| SEQ ID NO: 1382 | CGGTAGCGAAACANNKGGTACAAGCGAAAG |
| SEQ ID NO: 1383 | AGCGAAACACCGNNKACAAGCGAAAGC |
| SEQ ID NO: 1384 | AAACACCGGGTNNKAGCGAAAGCGC |
| SEQ ID NO: 1385 | CACCGGGTACANNKGAAAGCGCAACA |
| SEQ ID NO: 1386 | CGGGTACAAGCNNKAGCGCAACACC |
| SEQ ID NO: 1387 | GGTACAAGCGAANNKGCAACACCGGAAA |
| SEQ ID NO: 1388 | ACAAGCGAAAGCNNKACACCGGAAAGC |
| SEQ ID NO: 1389 | GCGAAAGCGCANNKCCGGAAAGCAG |
| SEQ ID NO: 1390 | GAAAGCGCAACANNKGAAAGCAGTGGTG |
| SEQ ID NO: 1391 | GCGCAACACCGNNKAGCAGTGGTGG |
| SEQ ID NO: 1392 | GCAACACCGGAANNKAGTGGTGGTAGC |
| SEQ ID NO: 1393 | CACCGGAAAGCNNKGGTGGTAGCTC |
| SEQ ID NO: 1394 | CCGGAAAGCAGTNNKGGTAGCTCAGGT |
| SEQ ID NO: 1395 | GGAAAGCAGTGGTNNKAGCTCAGGTGGTAG |
| SEQ ID NO: 1396 | AAAGCAGTGGTGGTNNKTCAGGTGGTAGTAG |
| SEQ ID NO: 1397 | AGTGGTGGTAGCNNKGGTGGTAGTAGCA |
| SEQ ID NO: 1398 | TGGTGGTAGCTCANNKGGTAGTAGCACCC |
| SEQ ID NO: 1399 | GTGGTAGCTCAGGTNNKAGTAGCACCCTGAAT |
| SEQ ID NO: 1400 | GTAGCTCAGGTGGTNNKAGCACCCTGAATATT |

TABLE 2

| | | Fold- | Fold- | Fold- | Fold- | Fold- |
|---|---|---|---|---|---|---|
| | | Change | Change | Change | Change | Change |
| Library | Mutation | R1 | R2 | R3 | R4 | R5 |
| 1 | D2K | 4.6 | 6.4 | 9.1 | 7.2 | 11.5 |
| 1 | D2T | 4.0 | 5.0 | 4.3 | 11.8 | 19.5 |
| 1 | D2T | 3.0 | 4.1 | 6.1 | 5.7 | 10.0 |
| 1 | D2S | 3.3 | 3.3 | 2.1 | 4.3 | 3.7 |
| 1 | D2I | 2.1 | 3.0 | 4.3 | 8.5 | 5.7 |
| 1 | D2Q | 3.3 | 2.4 | 0.5 | 0.7 | 1.4 |
| 1 | D2P | 2.8 | 3.0 | 2.9 | 9.4 | 6.5 |
| 1 | D2R | 3.0 | 3.7 | 5.8 | 3.1 | 4.5 |
| 1 | D2E | 2.8 | 2.8 | 2.7 | 2.5 | 3.2 |
| 1 | D2D | 1.9 | 1.9 | 2.9 | 1.3 | 1.3 |
| 1 | D2A | 3.8 | 5.2 | 7.5 | 6.3 | 9.2 |
| 1 | D2A | 2.9 | 3.2 | 2.2 | 1.4 | 1.5 |
| 1 | D2G | 1.7 | 1.0 | 0.9 | 1.4 | 1.5 |
| 1 | D2G | 3.4 | 4.7 | 6.2 | 6.0 | 11.8 |
| 1 | D2V | 3.1 | 2.9 | 2.2 | 2.0 | 3.5 |
| 1 | D2V | 3.1 | 3.9 | 3.8 | 8.0 | 10.0 |
| 1 | D2S | 3.1 | 4.4 | 5.7 | 3.8 | 2.8 |
| 1 | D2S | 5.7 | 8.7 | 8.9 | 7.2 | 4.4 |
| 1 | D2W | 1.8 | 2.0 | 1.8 | 5.7 | 5.9 |
| 1 | K3K | 3.0 | 1.6 | 0.8 | 1.3 | 2.4 |
| 1 | K3S | 4.2 | 5.0 | 7.6 | 4.6 | 2.1 |
| 1 | K3I | 1.6 | 1.2 | 1.3 | 1.5 | 1.3 |
| 1 | K3A | 2.6 | 2.1 | 0.3 | 3.0 | 1.0 |
| 1 | K3G | 3.3 | 5.2 | 6.2 | 7.7 | 7.9 |
| 1 | K3G | 1.7 | 2.2 | 1.6 | 1.4 | 1.0 |
| 1 | K3C | 2.2 | 2.4 | 1.8 | 0.9 | 1.2 |
| 1 | K3L | 2.3 | 1.6 | 2.2 | 5.7 | 5.3 |
| 1 | K4T | 2.3 | 2.7 | 3.4 | 20.3 | 10.5 |
| 1 | K4R | 2.1 | 2.3 | 1.9 | 2.2 | 2.2 |
| 1 | K4R | 3.0 | 3.2 | 7.6 | 9.2 | 2.6 |
| 1 | K4E | 1.8 | 1.7 | 1.9 | 1.4 | 1.0 |
| 1 | K4A | 1.0 | 1.3 | 0.7 | 2.0 | 1.9 |
| 1 | Y5Y | 1.7 | 1.5 | 1.7 | 2.1 | 2.6 |
| 1 | S6T | 2.6 | 3.2 | 2.2 | 2.8 | 2.6 |
| 1 | S6T | 3.4 | 5.4 | 6.5 | 5.5 | 7.5 |
| 1 | S6R | 1.0 | 1.5 | 1.1 | 2.4 | 1.6 |
| 1 | S6A | 1.3 | 1.7 | 0.5 | 1.2 | 1.1 |
| 1 | S6S | 2.1 | 2.7 | 4.5 | 13.6 | 5.7 |
| 1 | G8G | 2.4 | 2.4 | 5.4 | 3.3 | 5.3 |
| 1 | G8G | 3.6 | 4.4 | 4.7 | 6.8 | 8.4 |
| 1 | I11I | 1.3 | 1.5 | 4.1 | 0.2 | 1.5 |
| 1 | N14G | 0.8 | 0.5 | 0.3 | 1.9 | 2.5 |
| 1 | N14V | 2.7 | 1.9 | 2.3 | 2.7 | 2.8 |
| 1 | S15T | 1.8 | 0.8 | 1.2 | 7.5 | 3.8 |
| 1 | S15S | 3.1 | 3.9 | 4.2 | 3.1 | 7.8 |
| 1 | S15S | 2.5 | 2.3 | 1.8 | 2.6 | 2.2 |
| 1 | V16I | 1.8 | 2.4 | 2.7 | 35.1 | 26.4 |
| 1 | A19A | 2.9 | 3.4 | 4.8 | 8.6 | 13.8 |
| 1 | I21I | 2.9 | 3.7 | 2.7 | 3.7 | 2.1 |
| 1 | T22T | 2.4 | 3.1 | 3.0 | 1.7 | 1.3 |
| 1 | T22L | 1.5 | 1.8 | 7.1 | 6.2 | 4.5 |
| 1 | T22D | 3.6 | 4.4 | 2.1 | 2.3 | 1.9 |
| 1 | T22V | 2.9 | 3.9 | 7.8 | 11.8 | 8.2 |
| 1 | T22S | 1.6 | 2.1 | 0.8 | 1.7 | 1.4 |
| 1 | T22S | 1.4 | 1.2 | 2.0 | 5.2 | 2.8 |
| 1 | D23K | 5.9 | 12.3 | 22.6 | 26.5 | 32.8 |
| 1 | D23T | 1.1 | 1.8 | 2.3 | 4.9 | 6.7 |
| 1 | D23R | 2.7 | 3.8 | 5.8 | 6.5 | 6.0 |
| 1 | D23S | 4.3 | 4.3 | 4.1 | 10.4 | 14.4 |
| 1 | D23Q | 3.5 | 3.9 | 3.2 | 5.0 | 3.7 |
| 1 | D23H | 2.0 | 2.1 | 2.2 | 3.1 | 4.3 |
| 1 | D23R | 3.2 | 5.3 | 5.8 | 8.9 | 17.4 |
| 1 | D23R | 4.3 | 6.2 | 7.6 | 3.4 | 9.9 |
| 1 | D23E | 1.7 | 2.2 | 3.5 | 3.9 | 2.9 |
| 1 | D23D | 1.0 | 1.5 | 3.9 | 1.5 | 3.5 |
| 1 | D23A | 1.5 | 2.8 | 3.8 | 3.6 | 3.0 |
| 1 | D23G | 2.6 | 3.4 | 4.1 | 4.6 | 6.3 |
| 1 | D23S | 2.5 | 2.8 | 3.0 | 0.3 | 1.0 |
| 1 | E24K | 0.7 | 0.8 | 5.4 | 5.1 | 4.7 |
| 1 | E24Q | 2.8 | 2.0 | 4.9 | 2.7 | 1.2 |
| 1 | Y25F | 2.4 | 2.0 | 1.2 | 1.3 | 1.1 |
| 1 | K26I | 1.9 | 0.9 | 0.4 | 1.3 | 3.8 |
| 1 | V27I | 0.2 | 0.4 | 1.6 | 4.0 | 7.5 |
| 1 | V27V | 2.0 | 2.2 | 2.0 | 2.8 | 1.8 |
| 1 | P28P | 2.9 | 2.3 | 2.9 | 1.7 | 1.0 |
| 1 | S29S | 2.8 | 3.2 | 10.9 | 4.0 | 17.0 |
| 1 | S29M | 4.5 | 7.4 | 6.1 | 12.3 | 79.6 |
| 1 | S29H | 3.6 | 3.6 | 13.7 | 55.2 | 69.1 |
| 1 | S29L | 4.0 | 8.4 | 22.3 | 28.6 | 38.4 |
| 1 | S29E | 5.2 | 7.7 | 11.3 | 5.4 | 2.6 |
| 1 | S29A | 3.4 | 5.7 | 3.7 | 7.9 | 6.8 |
| 1 | S29A | 3.1 | 3.4 | 8.1 | 11.8 | 26.4 |
| 1 | S29V | 3.6 | 2.6 | 2.7 | 23.7 | 19.7 |
| 1 | S29Y | 3.1 | 4.0 | 2.6 | 17.6 | 22.4 |
| 1 | S29S | 3.5 | 4.5 | 1.9 | 4.6 | 5.8 |
| 1 | K30R | 3.9 | 8.0 | 11.3 | 19.6 | 11.1 |
| 1 | K30M | 2.3 | 3.0 | 1.4 | 10.2 | 42.6 |
| 1 | K30L | 1.8 | 4.8 | 3.0 | 1.6 | 1.6 |
| 1 | K30A | 3.0 | 0.6 | 2.0 | 9.7 | 1.1 |
| 1 | K30V | 3.7 | 3.2 | 1.4 | 6.5 | 2.9 |
| 1 | K30S | 5.9 | 8.9 | 7.2 | 9.9 | 8.0 |
| 1 | K30L | 1.1 | 0.6 | 1.3 | 4.3 | 3.2 |
| 1 | K31T | 5.7 | 7.3 | 17.4 | 16.9 | 23.7 |
| 1 | K31S | 5.4 | 8.1 | 4.2 | 9.1 | 13.1 |
| 1 | K31M | 1.4 | 1.0 | 1.0 | 3.6 | 8.7 |
| 1 | K31H | 4.6 | 8.8 | 21.9 | 24.3 | 13.4 |
| 1 | K31Y | 11.3 | 17.3 | 25.2 | 88.6 | 67.1 |
| 1 | K31F | 2.8 | 2.8 | 3.5 | 20.4 | 25.5 |
| 1 | F32Y | 2.3 | 1.1 | 5.2 | 5.8 | 7.3 |
| 1 | K33N | 2.8 | 4.0 | 3.9 | 5.2 | 9.5 |
| 1 | V34V | 2.9 | 2.6 | 3.3 | 7.8 | 15.2 |
| 1 | V34S | 3.0 | 0.4 | 3.6 | 4.3 | 3.5 |
| 1 | L35L | 1.3 | 1.8 | 14.3 | 22.7 | 20.3 |
| 1 | L35W | 3.2 | 3.6 | 5.5 | 10.7 | 2.4 |
| 1 | G36G | 2.3 | 3.3 | 2.0 | 4.6 | 6.5 |
| 1 | G36G | 2.4 | 3.5 | 3.7 | 3.8 | 2.7 |
| 1 | N37N | 3.2 | 3.2 | 2.0 | 3.1 | 2.4 |
| 1 | N37H | 2.0 | 2.5 | 2.3 | 3.5 | 1.2 |
| 1 | T38R | 1.6 | 1.5 | 5.1 | 2.4 | 1.2 |
| 1 | T38D | 1.7 | 2.7 | 7.0 | 1.8 | 1.1 |
| 1 | T38G | 6.4 | 7.7 | 4.5 | 3.5 | 3.2 |
| 1 | T38S | 2.0 | 1.1 | 5.4 | 2.6 | 3.9 |
| 1 | T38F | 1.2 | 1.2 | 1.8 | 0.7 | 1.3 |
| 1 | D39K | 2.1 | 2.6 | 0.8 | 3.2 | 3.5 |
| 1 | D39T | 3.7 | 5.3 | 4.9 | 3.0 | 1.9 |
| 1 | D39E | 2.5 | 3.2 | 3.1 | 3.0 | 5.7 |
| 1 | D39A | 2.2 | 2.6 | 2.1 | 0.9 | 2.0 |
| 1 | D39A | 1.4 | 2.0 | 1.5 | 2.1 | 2.0 |
| 1 | D39G | 3.5 | 3.4 | 2.7 | 4.0 | 3.5 |
| 1 | D39V | 2.4 | 1.9 | 1.7 | 2.9 | 1.2 |
| 1 | D39S | 4.9 | 5.4 | 3.3 | 5.7 | 1.8 |
| 1 | R40S | 1.6 | 3.0 | 2.5 | 29.1 | 59.7 |
| 1 | R40M | 2.1 | 4.6 | 6.2 | 15.2 | 39.3 |
| 1 | R40I | 0.5 | 1.3 | 4.3 | 12.3 | 28.7 |
| 1 | R40Q | 1.9 | 3.6 | 1.8 | 3.6 | 6.1 |
| 1 | R40P | 4.0 | 7.2 | 7.9 | 1.4 | 12.8 |
| 1 | R40R | 2.8 | 3.2 | 3.3 | 3.0 | 2.5 |
| 1 | R40L | 3.5 | 6.7 | 6.3 | 18.5 | 35.8 |
| 1 | R40L | 4.3 | 9.1 | 17.4 | 14.4 | 38.4 |
| 1 | R40E | 0.8 | 0.8 | 0.9 | 2.7 | 2.3 |
| 1 | R40A | 2.6 | 3.5 | 2.9 | 4.3 | 8.5 |
| 1 | R40A | 2.7 | 5.2 | 5.7 | 25.5 | 40.1 |
| 1 | R40G | 1.7 | 2.7 | 5.8 | 2.3 | 1.8 |
| 1 | R40V | 1.7 | 2.6 | 2.8 | 7.5 | 15.3 |
| 1 | R40S | 1.6 | 3.3 | 5.1 | 45.0 | 37.4 |
| 1 | R40C | 3.0 | 3.0 | 6.6 | 16.8 | 42.5 |
| 1 | R40L | 1.8 | 3.1 | 2.0 | 3.9 | 5.3 |
| 1 | R40F | 5.5 | 13.6 | 11.7 | 30.1 | 30.2 |
| 1 | H41N | 2.1 | 2.5 | 5.4 | 13.1 | 33.4 |
| 1 | H41S | 1.9 | 3.2 | 7.0 | 2.2 | 6.0 |
| 1 | H41P | 2.6 | 4.4 | 6.1 | 8.2 | 5.0 |
| 1 | H41L | 4.3 | 6.0 | 7.7 | 12.4 | 9.4 |
| 1 | H41L | 2.5 | 4.9 | 2.3 | 3.5 | 3.6 |
| 1 | H41E | 2.8 | 5.1 | 11.7 | 23.5 | 33.4 |
| 1 | H41A | 4.5 | 4.4 | 14.3 | 5.6 | 12.0 |
| 1 | H41G | 2.7 | 4.3 | 4.8 | 7.7 | 13.3 |
| 1 | H41G | 4.1 | 5.0 | 15.1 | 19.6 | 13.0 |
| 1 | H41V | 2.8 | 2.5 | 18.4 | 15.8 | 20.5 |
| 1 | H41S | 2.7 | 1.1 | 15.2 | 13.1 | 8.8 |
| 1 | H41C | 2.7 | 3.4 | 2.8 | 5.5 | 9.3 |
| 1 | H41L | 4.9 | 7.3 | 16.0 | 17.1 | 12.5 |
| 1 | S42S | 2.9 | 3.5 | 4.1 | 6.8 | 2.9 |
| 1 | S42M | 3.0 | 4.1 | 4.1 | 25.3 | 64.0 |
| 1 | S42P | 1.2 | 1.7 | 1.1 | 9.0 | 5.4 |
| 1 | S42L | 1.5 | 2.3 | 2.2 | 3.2 | 2.9 |
| 1 | S42E | 6.8 | 12.4 | 18.0 | 25.1 | 46.7 |
| 1 | S42D | 1.4 | 1.4 | 1.7 | 6.0 | 7.4 |
| 1 | S42A | 4.9 | 8.3 | 19.0 | 41.1 | 36.8 |
| 1 | S42V | 4.0 | 7.1 | 8.8 | 10.0 | 10.5 |
| 1 | S42V | 4.5 | 6.4 | 10.2 | 3.0 | 4.0 |
| 1 | S42S | 2.7 | 3.3 | 2.8 | 3.1 | 3.9 |
| 1 | S42S | 3.5 | 4.8 | 9.4 | 3.5 | 1.2 |
| 1 | I43D | 4.0 | 4.3 | 51.7 | 4.9 | 6.8 |
| 1 | I43C | 2.2 | 2.1 | 2.0 | 5.5 | 2.1 |
| 1 | K44N | 2.2 | 3.3 | 2.6 | 1.5 | 2.6 |
| 1 | K44T | 4.3 | 6.5 | 6.8 | 8.4 | 13.4 |
| 1 | K44R | 3.6 | 6.8 | 12.2 | 6.9 | 1.8 |
| 1 | K44D | 2.7 | 5.7 | 37.0 | 173.5 | 121.1 |
| 1 | K44A | 1.7 | 1.0 | 0.2 | 0.5 | 1.8 |
| 1 | K44G | 2.0 | 1.6 | 0.9 | 5.9 | 3.7 |
| 1 | K44V | 4.7 | 5.9 | 12.0 | 17.4 | 13.1 |
| 1 | K44S | 2.6 | 2.5 | 5.4 | 19.9 | 12.9 |
| 1 | K44W | 1.5 | 1.4 | 0.5 | 5.5 | 3.6 |
| 1 | K44C | 5.0 | 4.3 | 8.1 | 2.9 | 4.5 |
| 1 | K44L | 1.3 | 2.6 | 25.4 | 34.1 | 4.5 |
| 1 | K45N | 1.8 | 1.2 | 9.9 | 2.0 | 1.3 |
| 1 | K45R | 5.7 | 9.5 | 14.2 | 11.2 | 10.4 |
| 1 | K45STOP | 1.3 | 1.8 | 1.6 | 1.6 | 2.0 |
| 1 | N46T | 7.2 | 9.1 | 24.4 | 13.1 | 43.0 |
| 1 | N46E | 4.5 | 7.7 | 8.7 | 15.2 | 6.9 |
| 1 | L47L | 1.5 | 1.5 | 1.6 | 1.5 | 1.1 |
| 1 | L47V | 1.4 | 1.7 | 1.7 | 3.2 | 1.4 |
| 1 | L47STOP | 0.9 | 0.9 | 0.9 | 0.9 | 1.0 |
| 1 | I48T | 0.7 | 1.2 | 1.2 | 1.4 | 1.3 |
| 1 | I48S | 1.9 | 2.7 | 2.2 | 0.6 | 1.3 |
| 1 | I48F | 3.1 | 2.4 | 1.9 | 1.9 | 1.5 |
| 1 | G49R | 0.9 | 1.1 | 0.9 | 0.9 | 1.5 |
| 1 | G49G | 3.2 | 3.8 | 6.8 | 3.8 | 3.3 |
| 1 | A50S | 2.1 | 0.5 | 0.7 | 0.9 | 1.2 |
| 1 | A50A | 3.3 | 5.8 | 2.7 | 7.9 | 7.5 |
| 1 | A50V | 1.9 | 3.7 | 6.3 | 6.5 | 12.0 |
| 1 | L51T | 3.5 | 2.6 | 3.3 | 2.2 | 1.4 |
| 1 | L51Q | 1.1 | 1.3 | 1.4 | 1.2 | 1.6 |
| 1 | L51V | 3.1 | 4.3 | 1.8 | 5.0 | 11.0 |
| 1 | L52V | 1.6 | 1.4 | 0.5 | 1.3 | 1.9 |
| 1 | F53Y | 1.6 | 1.6 | 2.2 | 4.2 | 1.4 |
| 1 | S55K | 2.3 | 2.0 | 3.9 | 10.7 | 1.2 |
| 1 | S55R | 4.0 | 4.4 | 5.7 | 3.9 | 3.9 |
| 1 | S55A | 6.7 | 8.3 | 4.6 | 18.0 | 21.7 |
| 1 | S55S | 1.9 | 3.0 | 1.3 | 1.8 | 1.0 |
| 1 | G56N | 4.5 | 1.4 | 0.0 | 23.8 | 2.3 |
| 1 | G56R | 1.0 | 1.5 | 4.1 | 4.6 | 1.4 |
| 1 | G56A | 8.3 | 2.9 | 4.7 | 6.1 | 10.4 |
| 1 | E57K | 6.2 | 8.2 | 17.7 | 25.8 | 45.3 |
| 1 | E57N | 3.4 | 6.4 | 17.7 | 35.7 | 19.2 |
| 1 | E57D | 3.3 | 3.4 | 6.6 | 11.0 | 19.6 |
| 1 | E57G | 2.0 | 1.8 | 0.1 | 2.1 | 5.8 |
| 1 | E57V | 1.3 | 1.9 | 0.7 | 1.8 | 3.1 |
| 1 | T58T | 2.5 | 2.7 | 7.0 | 7.4 | 11.4 |
| 1 | A59P | 3.5 | 0.7 | 2.7 | 7.1 | 3.3 |
| 1 | A59A | 6.8 | 8.3 | 10.3 | 12.6 | 18.1 |
| 1 | A59A | 1.8 | 1.1 | 2.1 | 2.4 | 2.5 |
| 1 | E60I | 4.4 | 4.0 | 9.4 | 8.1 | 13.6 |
| 1 | E60W | 1.0 | 1.2 | 0.8 | 4.3 | 4.6 |
| 1 | A61N | 4.9 | 3.1 | 0.3 | 1.2 | 1.0 |
| 1 | A61M | 2.4 | 3.9 | 4.0 | 25.2 | 15.9 |
| 1 | A61E | 2.0 | 2.3 | 2.2 | 6.9 | 10.8 |
| 1 | A61A | 3.0 | 4.6 | 3.7 | 1.5 | 1.7 |
| 1 | A61V | 4.3 | 5.6 | 1.6 | 1.8 | 1.0 |
| 1 | A61S | 4.5 | 4.4 | 2.9 | 8.1 | 2.7 |
| 1 | T62S | 5.2 | 6.5 | 2.4 | 2.3 | 6.9 |
| 1 | T62S | 8.5 | 9.8 | 1.5 | 1.4 | 1.3 |
| 1 | R63R | 2.1 | 1.4 | 6.7 | 0.1 | 4.6 |
| 1 | L64P | 2.0 | 1.8 | 1.8 | 1.5 | 2.4 |
| 1 | L64V | 2.4 | 1.8 | 1.3 | 4.4 | 6.4 |
| 1 | K65K | 0.8 | 0.8 | 1.2 | 1.1 | 1.0 |
| 1 | K65L | 3.5 | 2.1 | 7.5 | 14.3 | 9.4 |
| 1 | K65A | 2.8 | 2.8 | 11.9 | 7.4 | 17.1 |
| 1 | T67T | 3.0 | 2.6 | 2.8 | 5.0 | 6.5 |
| 1 | T67S | 6.0 | 9.1 | 11.2 | 6.1 | 10.5 |
| 1 | A68A | 3.6 | 6.7 | 6.7 | 33.7 | 57.1 |
| 1 | R69R | 1.2 | 2.3 | 2.3 | 3.1 | 3.5 |
| 1 | R69R | 1.3 | 2.1 | 2.1 | 2.7 | 3.5 |
| 1 | Y72Y | 1.1 | 1.3 | 5.2 | 3.0 | 3.0 |
| 1 | R75R | 2.5 | 4.3 | 1.4 | 29.2 | 35.4 |
| 1 | N77Y | 1.1 | 1.4 | 0.9 | 1.2 | 1.2 |
| 1 | R78R | 1.6 | 2.3 | 1.0 | 3.7 | 2.6 |
| 1 | I79T | 1.6 | 2.2 | 2.7 | 6.5 | 1.6 |
| 1 | I79R | 0.6 | 0.7 | 1.6 | 1.1 | 1.7 |
| 1 | I79L | 0.5 | 0.7 | 4.0 | 4.0 | 3.5 |
| 1 | I79G | 1.2 | 1.0 | 0.7 | 1.4 | 1.2 |
| 1 | C80K | 1.3 | 0.3 | 0.1 | 0.6 | 1.0 |
| 1 | C80Q | 2.0 | 3.0 | 5.3 | 3.9 | 4.8 |
| 1 | C80R | 2.0 | 2.6 | 4.9 | 8.4 | 23.9 |
| 1 | Q83T | 1.5 | 0.9 | 1.3 | 2.5 | 4.1 |
| 1 | Q83V | 0.9 | 0.4 | 0.8 | 0.9 | 1.1 |
| 1 | E84N | 2.2 | 2.1 | 3.0 | 0.9 | 1.1 |
| 1 | E84E | 1.5 | 1.7 | 6.4 | 6.6 | 4.9 |
| 1 | E84A | 1.3 | 1.6 | 2.4 | 6.5 | 9.8 |
| 1 | E84G | 1.0 | 0.8 | 1.1 | 1.4 | 2.3 |
| 1 | E84S | 1.1 | 1.0 | 1.5 | 5.6 | 8.4 |
| 1 | I85L | 0.7 | 1.2 | 1.2 | 3.4 | 7.6 |
| 1 | I85L | 1.4 | 1.2 | 1.4 | 2.3 | 2.5 |
| 1 | S87R | 1.3 | 1.5 | 2.0 | 1.8 | 2.6 |
| 1 | S87S | 1.1 | 1.0 | 1.9 | 2.1 | 2.2 |
| 1 | S87R | 1.5 | 2.0 | 2.7 | 4.4 | 1.0 |
| 1 | S87S | 1.5 | 2.3 | 2.3 | 2.6 | 4.6 |
| 1 | S87C | 0.6 | 0.6 | 0.6 | 0.9 | 1.4 |
| 1 | N88N | 0.5 | 0.9 | 1.5 | 1.1 | 1.1 |
| 1 | N88R | 1.4 | 1.1 | 4.3 | 32.1 | 26.2 |
| 1 | N88R | 0.8 | 1.5 | 0.6 | 2.0 | 1.3 |
| 1 | N88E | 1.2 | 2.1 | 2.7 | 1.4 | 2.6 |
| 1 | N88S | 0.3 | 0.4 | 0.8 | 1.0 | 1.7 |
| 1 | N88C | 2.1 | 2.8 | 3.5 | 2.7 | 1.2 |
| 1 | M90G | 0.3 | 0.2 | 0.5 | 3.5 | 2.8 |
| 1 | M90L | 0.8 | 0.9 | 1.2 | 0.7 | 1.0 |
| 1 | A91K | 1.5 | 1.4 | 0.5 | 0.5 | 3.1 |
| 1 | A91N | 0.6 | 1.2 | 0.5 | 2.4 | 8.7 |
| 1 | A91I | 2.0 | 1.4 | 3.1 | 4.2 | 7.9 |
| 1 | A91Q | 0.9 | 0.6 | 1.7 | 1.5 | 2.5 |
| 1 | A91L | 1.4 | 1.7 | 9.7 | 15.8 | 14.5 |
| 1 | A91D | 1.2 | 1.5 | 1.0 | 4.7 | 3.3 |
| 1 | A91A | 1.3 | 1.4 | 2.0 | 3.0 | 4.7 |
| 1 | A91G | 1.0 | 1.0 | 1.5 | 2.1 | 5.4 |
| 1 | A91S | 0.7 | 0.9 | 0.5 | 2.6 | 3.2 |
| 1 | K92H | 0.9 | 1.3 | 0.8 | 1.7 | 1.9 |
| 1 | K92R | 0.8 | 0.8 | 0.6 | 1.1 | 1.4 |
| 1 | V93F | 0.5 | 0.4 | 0.6 | 2.0 | 4.4 |
| 1 | D94G | 0.7 | 0.8 | 0.3 | 0.5 | 1.1 |
| 1 | D95Q | 0.5 | 0.5 | 0.4 | 0.6 | 1.2 |
| 1 | D95P | 1.2 | 0.6 | 0.7 | 2.1 | 3.6 |
| 1 | D95L | 1.0 | 0.7 | 0.5 | 2.2 | 2.0 |
| 1 | D95E | 1.0 | 1.3 | 1.6 | 3.6 | 9.6 |
| 1 | D95A | 0.9 | 0.7 | 0.7 | 2.4 | 2.1 |
| 1 | D95V | 0.8 | 1.0 | 1.3 | 3.8 | 5.3 |
| 1 | D95Y | 1.4 | 1.6 | 1.5 | 0.9 | 1.4 |
| 1 | D95S | 1.1 | 0.8 | 0.9 | 2.1 | 2.1 |
| 1 | S96K | 1.7 | 2.3 | 2.4 | 2.1 | 1.7 |
| 1 | S96M | 1.3 | 1.9 | 1.9 | 3.0 | 6.4 |
| 1 | S96E | 1.4 | 1.7 | 2.0 | 3.7 | 3.5 |
| 1 | S96A | 1.0 | 0.9 | 3.2 | 3.2 | 3.1 |
| 1 | S96V | 1.2 | 1.1 | 1.1 | 1.2 | 1.6 |
| 1 | S96C | 1.0 | 1.0 | 0.2 | 1.2 | 2.4 |
| 1 | F97S | 1.1 | 0.8 | 1.2 | 2.3 | 1.6 |
| 1 | F97C | 0.9 | 0.8 | 0.7 | 0.8 | 1.1 |
| 1 | F98I | 0.5 | 0.4 | 1.6 | 1.6 | 1.1 |
| 1 | F98S | 0.6 | 0.4 | 1.7 | 2.3 | 1.1 |
| 1 | H99T | 0.8 | 1.9 | 2.4 | 5.0 | 8.1 |
| 1 | H99Q | 1.7 | 1.4 | 1.1 | 0.9 | 1.0 |
| 1 | H99V | 0.4 | 0.8 | 0.3 | 2.8 | 2.5 |
| 1 | H99L | 1.8 | 3.3 | 7.3 | 46.6 | 25.6 |
| 1 | H99F | 0.3 | 0.4 | 9.8 | 2.0 | 4.0 |
| 1 | R100K | 1.0 | 0.6 | 2.1 | 2.4 | 2.6 |
| 1 | R100R | 1.1 | 1.3 | 1.4 | 4.9 | 4.5 |
| 1 | R100R | 1.2 | 1.8 | 4.3 | 5.0 | 2.6 |
| 1 | R100R | 1.1 | 1.0 | 0.4 | 1.0 | 1.8 |
| 2 | L101N | 0.8 | 1.0 | 1.1 | 0.7 | 2.0 |
| 2 | L101T | 2.1 | 2.4 | 2.2 | 3.8 | 9.6 |
| 2 | L101S | 1.0 | 1.5 | 2.2 | 1.4 | 8.4 |
| 2 | E102K | 1.3 | 2.9 | 2.7 | 1.9 | 1.6 |
| 2 | E102T | 1.0 | 1.4 | 1.8 | 1.1 | 8.6 |
| 2 | E102V | 0.8 | 1.1 | 1.2 | 1.5 | 5.0 |
| 2 | E102S | 1.0 | 2.3 | 1.3 | 2.9 | 5.7 |
| 2 | E103Q | 2.2 | 4.0 | 5.8 | 15.7 | 32.7 |
| 2 | E103E | 1.1 | 1.6 | 1.5 | 1.4 | 2.0 |
| 2 | S104S | 1.4 | 2.1 | 2.1 | 1.4 | 1.8 |
| 2 | S104Q | 0.9 | 1.3 | 0.9 | 3.0 | 4.3 |
| 2 | S104G | 1.5 | 2.1 | 2.3 | 3.8 | 3.2 |
| 2 | F105I | 1.1 | 0.9 | 0.8 | 1.5 | 1.1 |
| 2 | F105C | 0.6 | 0.9 | 0.8 | 1.1 | 1.0 |
| 2 | L106N | 0.8 | 0.7 | 0.7 | 1.6 | 4.2 |
| 2 | L106W | 1.8 | 2.1 | 3.3 | 6.4 | 6.4 |
| 2 | E108R | 1.6 | 0.2 | 0.2 | 1.6 | 3.0 |
| 2 | E108Q | 1.5 | 1.1 | 1.3 | 2.8 | 2.1 |
| 2 | E108P | 1.4 | 2.2 | 1.7 | 10.4 | 11.6 |
| 2 | E108R | 1.9 | 2.0 | 2.3 | 6.1 | 4.1 |
| 2 | E108G | 1.4 | 1.2 | 1.6 | 1.8 | 4.1 |
| 2 | E109T | 1.7 | 0.7 | 1.0 | 2.8 | 2.1 |
| 2 | E109L | 0.6 | 0.4 | 1.0 | 3.2 | 9.6 |
| 2 | E109G | 1.6 | 2.3 | 3.3 | 2.3 | 1.2 |
| 2 | E109V | 1.3 | 0.6 | 0.8 | 3.8 | 2.7 |
| 2 | K111K | 1.6 | 2.0 | 2.9 | 1.5 | 1.2 |
| 2 | K112R | 1.1 | 1.1 | 1.1 | 1.0 | 1.1 |
| 2 | K112Q | 1.4 | 1.3 | 1.4 | 1.1 | 1.3 |
| 2 | H113R | 1.9 | 4.2 | 5.2 | 20.3 | 42.7 |
| 2 | H113R | 2.3 | 2.4 | 2.6 | 3.0 | 1.1 |
| 2 | H113Y | 1.5 | 2.0 | 1.6 | 2.1 | 12.5 |
| 2 | E114K | 2.1 | 1.9 | 1.4 | 2.2 | 7.2 |
| 2 | E114Q | 1.1 | 1.7 | 1.8 | 0.7 | 1.3 |
| 2 | E114R | 1.9 | 3.5 | 2.3 | 2.3 | 2.9 |
| 2 | E114V | 1.2 | 1.3 | 1.4 | 2.7 | 1.8 |
| 2 | R115R | 1.6 | 1.1 | 1.5 | 2.1 | 2.5 |
| 2 | H116Y | 1.1 | 1.5 | 1.8 | 4.0 | 2.6 |
| 2 | P117T | 1.4 | 1.1 | 1.3 | 2.1 | 2.7 |
| 2 | P117T | 1.8 | 1.6 | 2.2 | 7.0 | 2.4 |
| 2 | P117S | 1.2 | 2.0 | 2.1 | 7.3 | 9.6 |
| 2 | P117M | 1.1 | 0.8 | 0.9 | 1.6 | 6.1 |
| 2 | P117Q | 0.7 | 1.1 | 1.3 | 3.5 | 1.9 |
| 2 | P117P | 1.1 | 1.2 | 1.5 | 2.7 | 1.2 |
| 2 | P117G | 0.5 | 0.5 | 0.6 | 0.9 | 1.3 |
| 2 | P117L | 0.7 | 1.5 | 1.6 | 0.7 | 5.9 |
| 2 | I118I | 1.8 | 2.0 | 1.5 | 1.6 | 2.5 |
| 2 | I118A | 1.4 | 1.7 | 1.2 | 0.8 | 1.9 |
| 2 | I118Y | 1.7 | 2.9 | 2.9 | 4.0 | 35.5 |
| 2 | I118C | 1.3 | 1.9 | 1.9 | 2.6 | 2.9 |
| 2 | F119L | 1.7 | 0.3 | 0.3 | 2.8 | 4.5 |
| 2 | F119S | 2.6 | 3.3 | 3.5 | 4.7 | 3.5 |
| 2 | G120R | 1.6 | 1.1 | 0.7 | 2.4 | 19.9 |
| 2 | G120G | 0.8 | 0.6 | 0.7 | 1.1 | 1.1 |
| 2 | N121R | 1.4 | 2.6 | 1.6 | 2.2 | 4.4 |
| 2 | N121A | 1.2 | 0.8 | 0.7 | 3.1 | 31.4 |
| 2 | I122S | 1.4 | 1.9 | 2.3 | 2.4 | 1.7 |
| 2 | I122M | 2.2 | 2.3 | 2.7 | 1.3 | 2.1 |
| 2 | I122Q | 3.2 | 7.2 | 8.3 | 8.2 | 11.5 |
| 2 | I122P | 1.2 | 8.4 | 7.5 | 7.1 | 20.8 |
| 2 | I122P | 1.0 | 1.8 | 0.8 | 4.2 | 6.7 |
| 2 | I122E | 1.5 | 0.7 | 1.2 | 2.9 | 15.5 |
| 2 | I122D | 1.5 | 5.4 | 7.2 | 9.4 | 7.2 |
| 2 | I122G | 1.5 | 2.0 | 3.4 | 4.1 | 1.8 |
| 2 | V123T | 0.8 | 1.7 | 3.6 | 14.6 | 7.9 |
| 2 | V123P | 2.0 | 2.9 | 3.8 | 0.1 | 13.8 |
| 2 | V123R | 3.0 | 2.5 | 1.8 | 6.0 | 9.9 |
| 2 | V123G | 1.2 | 0.8 | 1.1 | 1.9 | 1.0 |
| 2 | D124E | 1.3 | 1.3 | 1.2 | 0.4 | 1.1 |
| 2 | D124G | 1.7 | 1.5 | 1.4 | 0.3 | 1.3 |
| 2 | E125E | 1.4 | 1.8 | 2.2 | 2.8 | 4.0 |
| 2 | V126R | 1.3 | 2.0 | 1.9 | 1.0 | 7.9 |
| 2 | V126R | 1.6 | 2.6 | 2.8 | 3.9 | 11.9 |
| 2 | V126L | 1.7 | 3.1 | 4.9 | 3.0 | 2.2 |
| 2 | V126V | 1.0 | 1.2 | 1.5 | 0.9 | 1.5 |
| 2 | V126L | 1.4 | 2.0 | 2.4 | 3.4 | 1.6 |
| 2 | A127M | 2.3 | 4.6 | 4.4 | 0.8 | 1.2 |
| 2 | Y128C | 0.6 | 0.6 | 0.8 | 1.6 | 1.4 |
| 2 | H129Q | 0.9 | 0.7 | 0.6 | 1.4 | 2.0 |
| 2 | H129C | 0.7 | 0.5 | 0.6 | 1.1 | 5.5 |
| 2 | E130Q | 5.9 | 6.5 | 8.2 | 15.0 | 1.5 |
| 2 | E130V | 0.7 | 0.5 | 0.6 | 2.9 | 6.2 |
| 2 | E130V | 1.6 | 1.2 | 0.7 | 1.3 | 1.7 |
| 2 | Y132V | 0.8 | 0.6 | 0.9 | 0.7 | 3.4 |
| 2 | Y132Y | 2.1 | 1.4 | 0.8 | 2.0 | 3.7 |
| 2 | P133P | 0.8 | 1.4 | 1.2 | 0.7 | 1.5 |
| 2 | L138L | 3.5 | 4.0 | 3.3 | 9.0 | 11.8 |
| 2 | L138L | 0.7 | 0.7 | 0.8 | 1.8 | 1.6 |
| 2 | K140R | 4.2 | 4.2 | 6.1 | 4.2 | 12.5 |
| 2 | K140R | 6.8 | 6.3 | 8.7 | 6.8 | 17.4 |
| 2 | L142L | 1.3 | 0.8 | 1.4 | 3.3 | 2.2 |
| 2 | L142V | 0.2 | 0.1 | 0.1 | 0.6 | 1.0 |
| 2 | S145G | 1.7 | 1.5 | 1.7 | 3.8 | 3.8 |
| 2 | S145G | 1.3 | 1.7 | 2.2 | 3.8 | 3.3 |
| 2 | S145V | 1.3 | 1.7 | 1.7 | 1.5 | 1.4 |
| 2 | T146T | 1.2 | 1.3 | 1.0 | 1.4 | 1.1 |
| 2 | T146I | 2.1 | 1.1 | 1.6 | 0.8 | 1.2 |
| 2 | T146P | 1.5 | 2.4 | 4.3 | 8.3 | 18.1 |
| 2 | T146A | 1.6 | 1.3 | 1.4 | 1.1 | 1.2 |
| 2 | D147G | 1.8 | 1.9 | 1.6 | 0.7 | 2.0 |
| 2 | R152R | 1.8 | 1.9 | 2.0 | 1.8 | 7.0 |
| 2 | L153H | 1.2 | 1.4 | 1.4 | 1.2 | 3.3 |
| 2 | L153R | 0.9 | 0.9 | 1.0 | 1.0 | 1.0 |
| 2 | L153V | 0.4 | 0.4 | 0.3 | 0.3 | 1.7 |
| 2 | I154V | 0.8 | 1.3 | 1.1 | 1.0 | 1.2 |
| 2 | L158M | 1.3 | 1.3 | 0.9 | 2.4 | 1.5 |
| 2 | M161I | 1.1 | 2.0 | 2.3 | 2.9 | 16.1 |
| 2 | M161A | 3.9 | 3.7 | 3.1 | 5.3 | 2.1 |
| 2 | M161G | 0.6 | 0.6 | 0.8 | 1.9 | 1.3 |
| 2 | M161V | 0.3 | 0.3 | 0.2 | 1.6 | 2.7 |
| 2 | M161C | 0.9 | 0.9 | 1.2 | 2.0 | 1.6 |
| 2 | I162V | 0.4 | 0.2 | 0.1 | 0.3 | 5.9 |
| 2 | F164A | 8.8 | 9.1 | 5.1 | 0.3 | 15.7 |
| 2 | F164Y | 0.9 | 1.3 | 2.0 | 2.6 | 5.7 |
| 2 | R165R | 0.9 | 1.0 | 1.9 | 0.9 | 2.1 |
| 2 | G166G | 0.8 | 1.1 | 1.2 | 2.2 | 2.0 |
| 2 | F168F | 1.6 | 2.3 | 2.6 | 2.2 | 1.5 |
| 2 | L169R | 2.2 | 3.3 | 3.4 | 4.1 | 15.4 |
| 2 | L169L | 1.2 | 1.3 | 1.1 | 1.7 | 1.6 |
| 2 | D173D | 1.9 | 2.5 | 2.2 | 3.4 | 6.7 |
| 2 | L174G | 1.8 | 1.9 | 1.0 | 0.3 | 6.1 |
| 2 | N175S | 1.6 | 4.3 | 7.2 | 8.6 | 3.8 |
| 2 | N175L | 8.2 | 12.3 | 7.2 | 16.0 | 14.4 |
| 2 | N175C | 4.4 | 5.7 | 8.5 | 22.0 | 6.3 |
| 2 | D177S | 1.3 | 1.2 | 1.2 | 0.6 | 2.3 |
| 2 | D177C | 1.8 | 2.7 | 2.4 | 7.3 | 8.4 |
| 2 | N178F | 1.9 | 2.4 | 3.5 | 5.5 | 6.1 |
| 2 | S179G | 0.6 | 0.8 | 1.1 | 2.8 | 5.1 |
| 2 | S179S | 1.4 | 2.4 | 2.7 | 2.4 | 10.6 |
| 2 | S179C | 0.9 | 1.1 | 1.1 | 1.4 | 1.1 |
| 2 | D180H | 2.9 | 8.4 | 13.7 | 15.7 | 30.3 |
| 2 | V181P | 3.9 | 8.0 | 8.1 | 20.1 | 64.9 |
| 2 | V181C | 1.0 | 1.1 | 1.0 | 2.1 | 1.4 |
| 2 | D182A | 0.8 | 1.7 | 2.6 | 5.7 | 4.4 |
| 2 | D182G | 1.3 | 1.5 | 2.2 | 4.5 | 1.1 |
| 2 | D182G | 1.4 | 1.2 | 1.6 | 1.5 | 3.0 |
| 2 | D182S | 1.4 | 1.7 | 2.1 | 6.5 | 33.5 |
| 2 | D182C | 4.1 | 4.0 | 3.9 | 8.5 | 5.6 |
| 2 | K183T | 6.7 | 8.0 | 9.2 | 27.5 | 13.1 |
| 2 | K183L | 2.3 | 1.9 | 2.0 | 3.0 | 7.6 |
| 2 | K183V | 7.7 | 3.5 | 9.6 | 13.4 | 14.6 |
| 2 | L184STOP | 1.0 | 0.9 | 1.1 | 1.2 | 1.0 |
| 2 | F185T | 16.4 | 19.4 | 17.1 | 42.2 | 28.6 |
| 2 | F185A | 0.1 | 0.5 | 1.1 | 1.3 | 3.6 |
| 2 | F185V | 1.8 | 2.4 | 2.4 | 1.9 | 2.3 |
| 2 | F185Y | 1.3 | 1.3 | 1.4 | 1.4 | 1.2 |
| 2 | Q187K | 1.6 | 3.7 | 3.9 | 9.4 | 8.1 |
| 2 | Q187V | 2.5 | 4.6 | 4.9 | 8.9 | 6.4 |
| 2 | L188T | 2.2 | 3.8 | 1.0 | 8.2 | 15.3 |
| 2 | L188V | 2.7 | 1.7 | 1.4 | 4.7 | 11.0 |
| 2 | V189R | 4.7 | 8.0 | 5.0 | 14.7 | 101.3 |
| 2 | Q190H | 1.4 | 1.2 | 1.4 | 1.2 | 1.1 |
| 2 | T191S | 0.8 | 0.5 | 0.4 | 0.4 | 1.5 |
| 2 | T191D | 1.8 | 5.4 | 5.8 | 6.1 | 7.9 |
| 2 | T191S | 1.6 | 2.7 | 3.2 | 1.2 | 1.1 |
| 2 | T191L | 0.6 | 1.4 | 2.3 | 2.1 | 10.6 |
| 2 | Y192S | 4.8 | 5.0 | 5.0 | 11.1 | 12.7 |
| 2 | Y192V | 2.4 | 4.1 | 5.9 | 1.4 | 2.0 |
| 2 | N193S | 1.6 | 2.7 | 2.9 | 4.4 | 2.3 |
| 2 | N193M | 1.7 | 4.0 | 4.4 | 7.6 | 68.9 |
| 2 | N193Q | 2.6 | 7.7 | 7.7 | 6.1 | 4.8 |
| 2 | N193E | 1.7 | 3.5 | 5.3 | 5.3 | 13.6 |
| 2 | N193D | 1.4 | 1.7 | 2.9 | 6.8 | 9.0 |
| 2 | Q194P | 1.6 | 3.6 | 5.5 | 5.8 | 2.0 |
| 2 | Q194R | 2.8 | 2.4 | 2.1 | 2.8 | 9.4 |
| 2 | Q194D | 1.8 | 2.5 | 1.8 | 1.7 | 3.7 |
| 2 | Q194G | 3.8 | 2.0 | 2.1 | 3.7 | 1.3 |
| 2 | L195Q | 1.7 | 1.3 | 1.9 | 3.4 | 25.4 |
| 2 | F196E | 4.7 | 8.1 | 6.5 | 9.1 | 10.5 |
| 2 | E198D | 1.3 | 1.2 | 1.2 | 1.7 | 1.0 |
| 2 | E198A | 4.5 | 3.8 | 4.2 | 0.1 | 14.2 |
| 2 | N199T | 0.4 | 0.5 | 0.9 | 3.8 | 15.7 |
| 2 | N199Q | 1.7 | 1.3 | 1.1 | 6.3 | 27.0 |
| 2 | N199V | 1.9 | 2.1 | 2.1 | 8.2 | 19.4 |
| 2 | N199F | 1.8 | 2.5 | 2.0 | 15.2 | 2.4 |
| 2 | P200M | 1.1 | 2.2 | 1.8 | 3.0 | 23.4 |
| 2 | P200P | 1.1 | 1.2 | 1.2 | 1.2 | 1.1 |
| 2 | P200E | 0.9 | 1.6 | 1.4 | 0.1 | 3.3 |
| 2 | P200S | 2.1 | 3.5 | 3.4 | 7.4 | 2.4 |
| 3 | I201T | 0.8 | 0.8 | 0.8 | 1.3 | 2.6 |
| 3 | I201I | 0.9 | 0.9 | 1.0 | 1.0 | 0.5 |
| 3 | I201Q | 1.4 | 1.7 | 1.4 | 2.0 | 2.2 |
| 3 | I201R | 0.8 | 0.4 | 0.5 | 0.5 | 1.1 |
| 3 | I201L | 1.0 | 0.8 | 0.9 | 1.1 | 1.0 |
| 3 | I201V | 1.1 | 1.0 | 1.1 | 1.3 | 1.4 |
| 3 | N202K | 1.0 | 0.9 | 0.8 | 0.6 | 1.2 |
| 3 | N202M | 1.2 | 1.1 | 1.1 | 1.1 | 1.0 |
| 3 | N202I | 1.2 | 1.4 | 1.5 | 1.7 | 3.9 |
| 3 | N202H | 1.2 | 1.6 | 1.7 | 2.1 | 2.9 |
| 3 | N202R | 2.0 | 1.8 | 1.5 | 2.2 | 7.0 |
| 3 | N202E | 1.0 | 0.8 | 1.0 | 1.2 | 1.2 |
| 3 | N202D | 0.8 | 0.7 | 0.7 | 1.0 | 2.0 |
| 3 | N202G | 1.6 | 1.2 | 1.3 | 2.0 | 1.8 |
| 3 | N202G | 0.8 | 0.7 | 0.8 | 0.5 | 1.1 |
| 3 | N202V | 1.1 | 1.1 | 1.1 | 0.7 | 2.0 |
| 3 | N202L | 1.2 | 0.8 | 0.9 | 1.2 | 1.8 |
| 3 | N202F | 1.4 | 1.2 | 1.1 | 1.9 | 2.3 |
| 3 | A203I | 1.5 | 2.0 | 1.9 | 1.5 | 1.0 |
| 3 | A203P | 1.1 | 1.9 | 1.8 | 3.1 | 4.2 |
| 3 | A203L | 1.4 | 2.0 | 1.4 | 1.4 | 5.9 |
| 3 | A203E | 1.7 | 1.3 | 1.3 | 2.2 | 2.0 |
| 3 | A203D | 0.9 | 0.8 | 1.0 | 0.7 | 0.5 |
| 3 | A203Y | 0.8 | 0.9 | 0.8 | 2.1 | 9.1 |
| 3 | A203S | 1.3 | 0.7 | 1.0 | 1.7 | 5.8 |
| 3 | A203C | 0.9 | 1.6 | 2.3 | 1.5 | 2.8 |
| 3 | A203L | 1.1 | 0.8 | 1.2 | 0.9 | 1.0 |
| 3 | S204K | 0.8 | 0.8 | 0.9 | 1.1 | 2.4 |
| 3 | S204T | 1.3 | 2.2 | 1.8 | 2.3 | 2.5 |
| 3 | S204M | 0.9 | 0.8 | 0.9 | 0.6 | 1.8 |
| 3 | S204Q | 0.6 | 0.4 | 0.6 | 0.7 | 2.6 |
| 3 | S204R | 1.1 | 0.8 | 0.8 | 0.9 | 3.7 |
| 3 | S204E | 1.2 | 1.0 | 0.9 | 1.2 | 1.0 |
| 3 | S204G | 0.8 | 1.3 | 1.2 | 1.5 | 4.2 |
| 3 | S204V | 1.1 | 0.9 | 0.9 | 2.3 | 1.4 |
| 3 | S204Y | 1.2 | 1.3 | 1.4 | 1.8 | 1.9 |
| 3 | S204W | 1.2 | 0.8 | 1.0 | 1.2 | 1.0 |
| 3 | S204C | 0.9 | 0.9 | 0.9 | 1.3 | 5.2 |
| 3 | S204L | 1.0 | 1.4 | 1.5 | 2.1 | 2.4 |
| 3 | G205N | 0.3 | 1.0 | 1.2 | 0.9 | 2.4 |
| 3 | G205T | 2.6 | 3.2 | 3.8 | 3.9 | 5.7 |
| 3 | G205Q | 1.5 | 2.1 | 1.6 | 1.7 | 4.0 |
| 3 | G205G | 1.1 | 0.9 | 1.1 | 1.3 | 0.5 |
| 3 | G205Y | 1.1 | 1.3 | 2.2 | 1.4 | 1.6 |
| 3 | G205L | 1.0 | 1.1 | 0.9 | 0.4 | 2.0 |
| 3 | V206T | 0.5 | 1.0 | 1.8 | 1.4 | 4.5 |
| 3 | V206R | 1.0 | 1.0 | 1.1 | 1.4 | 1.3 |
| 3 | V206S | 0.8 | 1.1 | 1.0 | 0.7 | 4.2 |
| 3 | V206M | 0.7 | 0.4 | 0.6 | 0.7 | 2.2 |
| 3 | V206H | 0.7 | 1.4 | 1.7 | 2.0 | 14.6 |
| 3 | V206L | 1.1 | 1.4 | 1.6 | 1.5 | 1.2 |
| 3 | V206E | 1.4 | 1.6 | 2.2 | 2.7 | 3.0 |
| 3 | V206A | 1.0 | 1.4 | 1.3 | 1.3 | 1.4 |
| 3 | V206A | 1.2 | 1.5 | 1.1 | 2.0 | 1.8 |
| 3 | V206G | 1.1 | 0.9 | 1.0 | 1.0 | 0.5 |
| 3 | V206Y | 0.8 | 0.6 | 0.4 | 0.8 | 1.7 |
| 3 | V206W | 1.0 | 1.4 | 1.8 | 2.9 | 3.1 |
| 3 | V206C | 1.1 | 1.7 | 1.6 | 1.5 | 3.4 |
| 3 | V206L | 1.2 | 1.2 | 1.3 | 1.9 | 4.6 |
| 3 | V206F | 1.6 | 1.7 | 1.1 | 1.8 | 0.5 |
| 3 | D207T | 1.7 | 2.2 | 1.9 | 2.7 | 1.5 |
| 3 | D207R | 1.0 | 0.7 | 0.7 | 1.0 | 0.5 |
| 3 | D207E | 1.0 | 1.1 | 1.1 | 1.6 | 2.3 |
| 3 | D207A | 1.2 | 1.0 | 1.1 | 1.4 | 1.8 |
| 3 | D207G | 1.0 | 0.9 | 0.9 | 1.1 | 1.2 |
| 3 | D207S | 1.3 | 1.3 | 1.4 | 2.4 | 2.0 |
| 3 | A208I | 1.4 | 1.7 | 1.5 | 0.7 | 1.0 |
| 3 | A208Q | 1.1 | 1.6 | 1.5 | 0.8 | 1.1 |
| 3 | A208P | 1.5 | 1.0 | 1.3 | 0.9 | 1.4 |
| 3 | A208L | 0.7 | 0.6 | 0.5 | 0.5 | 1.8 |
| 3 | A208L | 1.5 | 2.3 | 2.8 | 1.1 | 1.2 |
| 3 | A208E | 1.0 | 1.0 | 1.2 | 1.5 | 2.0 |
| 3 | A208A | 0.6 | 0.7 | 0.7 | 0.7 | 0.5 |
| 3 | A208G | 1.1 | 1.0 | 1.1 | 1.0 | 1.5 |
| 3 | A208G | 1.3 | 0.8 | 1.0 | 1.0 | 0.5 |
| 3 | A208V | 1.2 | 1.3 | 1.4 | 1.8 | 1.1 |
| 3 | A208V | 1.0 | 1.3 | 1.4 | 1.2 | 1.6 |
| 3 | A208S | 1.8 | 1.7 | 2.2 | 1.2 | 1.7 |
| 3 | A208S | 1.5 | 2.0 | 2.1 | 3.0 | 2.3 |
| 3 | A208W | 1.4 | 1.5 | 2.0 | 2.7 | 3.4 |
| 3 | A208L | 1.6 | 1.3 | 1.2 | 1.5 | 1.7 |
| 3 | K209T | 0.8 | 1.0 | 1.1 | 1.3 | 1.3 |
| 3 | K209M | 0.7 | 0.8 | 0.3 | 1.0 | 1.5 |
| 3 | K209I | 1.5 | 2.2 | 2.5 | 1.9 | 3.1 |
| 3 | K209Q | 1.6 | 0.8 | 0.5 | 0.5 | 1.3 |
| 3 | K209S | 1.7 | 1.7 | 2.1 | 2.8 | 2.6 |
| 3 | A210K | 0.8 | 0.9 | 1.0 | 1.2 | 3.6 |
| 3 | A210S | 1.4 | 1.8 | 1.9 | 1.1 | 0.5 |
| 3 | A210M | 1.2 | 1.0 | 1.0 | 1.2 | 0.5 |
| 3 | A210Q | 0.7 | 1.3 | 1.3 | 0.4 | 2.5 |
| 3 | A210P | 1.0 | 0.8 | 0.9 | 2.6 | 1.1 |
| 3 | A210P | 0.3 | 1.0 | 0.8 | 1.1 | 1.1 |
| 3 | A210L | 0.9 | 1.3 | 1.2 | 2.0 | 3.0 |
| 3 | A210E | 1.1 | 1.3 | 1.5 | 2.0 | 1.7 |
| 3 | A210D | 1.1 | 1.6 | 1.6 | 1.4 | 2.6 |
| 3 | A210A | 1.1 | 0.9 | 0.7 | 0.7 | 1.0 |
| 3 | A210A | 1.5 | 1.4 | 1.3 | 1.5 | 2.9 |
| 3 | A210G | 1.2 | 1.5 | 1.6 | 1.4 | 1.7 |
| 3 | A210V | 0.8 | 0.9 | 0.9 | 0.7 | 1.0 |
| 3 | A210S | 0.9 | 0.8 | 1.4 | 1.8 | 1.3 |
| 3 | A210W | 0.8 | 1.0 | 1.0 | 1.1 | 1.2 |
| 3 | A210L | 1.8 | 1.6 | 1.3 | 1.7 | 5.5 |
| 3 | A210F | 1.0 | 1.6 | 1.4 | 1.2 | 1.5 |
| 3 | I211N | 1.1 | 1.1 | 1.2 | 1.3 | 1.0 |
| 3 | I211S | 1.0 | 1.0 | 1.1 | 1.4 | 2.6 |
| 3 | I211H | 0.7 | 1.3 | 0.9 | 0.8 | 1.0 |
| 3 | I211R | 1.2 | 1.2 | 1.2 | 1.7 | 1.5 |
| 3 | I211L | 1.6 | 1.7 | 1.9 | 2.5 | 1.0 |
| 3 | I211D | 0.9 | 1.1 | 1.4 | 1.0 | 1.2 |
| 3 | I211A | 1.2 | 1.6 | 2.3 | 1.9 | 2.9 |
| 3 | I211G | 1.1 | 1.5 | 1.5 | 1.8 | 1.5 |
| 3 | I211Y | 1.3 | 1.4 | 1.6 | 2.4 | 3.0 |
| 3 | I211S | 1.0 | 0.3 | 0.4 | 0.6 | 1.8 |
| 3 | I211C | 1.3 | 1.2 | 1.0 | 1.6 | 1.1 |
| 3 | A210K | 0.8 | 1.2 | 1.1 | 1.8 | 4.8 |
| 3 | A210M | 1.4 | 1.7 | 1.0 | 1.5 | 1.0 |
| 3 | A210L | 1.1 | 1.9 | 2.3 | 3.8 | 4.4 |
| 3 | A210E | 1.0 | 0.7 | 0.9 | 1.6 | 3.8 |
| 3 | A210D | 1.6 | 2.8 | 3.5 | 2.8 | 4.4 |
| 3 | A210A | 1.5 | 1.8 | 1.7 | 2.6 | 3.0 |
| 3 | A210A | 1.7 | 1.6 | 1.5 | 1.7 | 3.2 |
| 3 | A210G | 1.5 | 1.8 | 1.8 | 1.7 | 1.1 |
| 3 | A210G | 1.6 | 2.5 | 2.6 | 2.1 | 2.9 |
| 3 | A210S | 1.2 | 1.1 | 1.5 | 2.3 | 1.3 |
| 3 | A210S | 1.7 | 1.0 | 1.6 | 4.7 | 2.4 |
| 3 | A210W | 1.0 | 1.2 | 1.1 | 1.8 | 1.5 |
| 3 | A210L | 1.9 | 1.8 | 1.2 | 2.5 | 9.1 |
| 3 | A210F | 1.5 | 1.9 | 2.4 | 2.5 | 1.5 |
| 3 | I211N | 1.3 | 1.2 | 1.4 | 2.4 | 1.6 |
| 3 | I211S | 1.5 | 1.8 | 2.0 | 1.8 | 1.7 |
| 3 | I211M | 1.1 | 1.1 | 1.4 | 1.7 | 3.0 |
| 3 | I211R | 1.1 | 1.0 | 1.3 | 1.5 | 1.5 |
| 3 | I211L | 1.2 | 2.2 | 3.7 | 4.0 | 1.1 |
| 3 | I211E | 2.2 | 3.2 | 3.6 | 2.4 | 1.2 |
| 3 | I211A | 2.5 | 3.1 | 4.6 | 4.8 | 7.7 |
| 3 | I211G | 1.8 | 2.2 | 2.2 | 3.8 | 2.8 |
| 3 | I211V | 1.4 | 1.1 | 1.2 | 1.1 | 1.2 |
| 3 | I211S | 2.3 | 0.5 | 0.9 | 1.8 | 6.1 |
| 3 | I211C | 1.1 | 0.6 | 0.5 | 0.7 | 1.5 |
| 3 | L212S | 1.7 | 1.3 | 1.2 | 3.7 | 4.7 |
| 3 | L212L | 1.5 | 2.5 | 2.8 | 3.6 | 2.3 |
| 3 | L212F | 1.4 | 1.7 | 2.3 | 2.4 | 2.0 |
| 3 | S213T | 0.7 | 0.8 | 1.1 | 1.0 | 1.5 |
| 3 | S213M | 0.9 | 3.1 | 3.1 | 1.4 | 1.2 |
| 3 | S213H | 3.3 | 6.9 | 4.3 | 5.3 | 10.6 |
| 3 | S213D | 2.1 | 2.9 | 2.8 | 9.6 | 26.2 |
| 3 | S213G | 1.1 | 1.6 | 1.2 | 0.8 | 1.4 |
| 3 | A214L | 1.1 | 2.1 | 4.1 | 9.6 | 6.7 |
| 3 | R215N | 1.1 | 0.7 | 1.7 | 2.0 | 1.6 |
| 3 | R215R | 1.7 | 2.1 | 3.3 | 3.1 | 1.2 |
| 3 | R215S | 2.2 | 4.6 | 4.9 | 4.0 | 7.9 |
| 3 | R215M | 3.2 | 4.5 | 9.2 | 16.0 | 10.4 |
| 3 | R215I | 2.2 | 0.9 | 0.6 | 2.9 | 6.0 |
| 3 | R215Q | 1.6 | 1.1 | 1.5 | 3.3 | 5.1 |
| 3 | R215L | 0.8 | 0.7 | 1.3 | 1.8 | 1.5 |
| 3 | R215L | 3.3 | 5.3 | 10.1 | 16.8 | 7.9 |
| 3 | R215E | 1.4 | 2.5 | 2.6 | 3.9 | 3.7 |
| 3 | R215D | 1.5 | 3.4 | 1.5 | 2.8 | 1.8 |
| 3 | R215A | 2.1 | 2.4 | 3.2 | 1.5 | 2.3 |
| 3 | R215V | 1.7 | 2.3 | 2.6 | 3.0 | 1.2 |
| 3 | R215Y | 1.8 | 1.6 | 1.9 | 0.7 | 2.3 |
| 3 | R215S | 1.3 | 2.3 | 3.1 | 1.6 | 1.7 |
| 3 | R215W | 1.5 | 1.1 | 1.2 | 1.6 | 1.7 |
| 3 | R215C | 1.7 | 4.1 | 5.5 | 7.9 | 3.4 |
| 3 | R215L | 1.7 | 3.3 | 7.1 | 12.7 | 10.5 |
| 3 | L216T | 1.8 | 3.3 | 1.9 | 5.8 | 9.5 |
| 3 | L216R | 0.5 | 1.4 | 1.8 | 2.9 | 13.3 |
| 3 | L216S | 2.8 | 3.4 | 3.7 | 5.2 | 6.7 |
| 3 | L216P | 1.2 | 0.4 | 0.3 | 0.4 | 1.0 |
| 3 | L216D | 3.0 | 3.0 | 4.7 | 4.7 | 1.6 |
| 3 | L216V | 0.4 | 0.5 | 0.5 | 0.8 | 1.0 |
| 3 | L216W | 1.0 | 2.3 | 2.6 | 2.3 | 5.6 |
| 3 | L216L | 1.4 | 1.5 | 3.0 | 4.7 | 4.9 |
| 3 | L216F | 1.2 | 1.3 | 1.7 | 2.2 | 1.1 |
| 3 | S217T | 1.9 | 1.0 | 1.7 | 3.4 | 2.0 |
| 3 | S217R | 1.0 | 1.0 | 0.4 | 0.7 | 1.8 |
| 3 | S217Q | 2.1 | 3.7 | 3.7 | 6.1 | 11.3 |
| 3 | S217H | 4.9 | 5.0 | 5.8 | 17.0 | 4.2 |
| 3 | S217P | 1.7 | 2.9 | 4.0 | 3.5 | 6.5 |
| 3 | S217A | 2.9 | 3.6 | 3.9 | 4.1 | 4.1 |
| 3 | S217G | 1.5 | 1.8 | 2.1 | 1.1 | 1.2 |
| 3 | S217S | 3.2 | 3.5 | 5.7 | 4.9 | 2.4 |
| 3 | K218N | 1.9 | 1.0 | 1.3 | 1.4 | 2.7 |
| 3 | K218R | 1.2 | 0.7 | 0.8 | 1.4 | 2.2 |
| 3 | S219I | 0.8 | 0.7 | 1.6 | 2.6 | 4.4 |
| 3 | S219D | 0.4 | 0.4 | 0.2 | 1.3 | 1.8 |
| 3 | S219G | 1.3 | 1.0 | 0.9 | 2.2 | 36.9 |
| 3 | S219V | 2.1 | 2.0 | 1.5 | 2.9 | 2.6 |
| 3 | S219S | 2.3 | 3.1 | 3.4 | 2.0 | 2.9 |
| 3 | S219L | 1.7 | 1.1 | 1.1 | 6.6 | 5.4 |
| 3 | R220K | 1.7 | 1.3 | 1.0 | 1.1 | 7.2 |
| 3 | R220N | 2.3 | 3.1 | 4.2 | 3.4 | 4.4 |
| 3 | R220T | 0.8 | 2.1 | 2.9 | 5.7 | 3.2 |
| 3 | R220M | 1.5 | 3.8 | 5.3 | 5.6 | 7.8 |
| 3 | R220I | 1.1 | 2.6 | 2.7 | 4.1 | 15.4 |
| 3 | R220Q | 2.6 | 2.9 | 4.1 | 5.4 | 6.1 |
| 3 | R220H | 1.9 | 2.0 | 2.9 | 2.4 | 3.0 |
| 3 | R220R | 1.9 | 2.3 | 3.2 | 4.1 | 2.7 |
| 3 | R220E | 2.1 | 3.2 | 4.1 | 6.5 | 12.6 |
| 3 | R220D | 2.4 | 1.4 | 2.1 | 3.6 | 1.7 |
| 3 | R220A | 0.9 | 2.2 | 2.3 | 7.0 | 7.6 |
| 3 | R220G | 1.8 | 1.3 | 1.3 | 2.5 | 8.9 |
| 3 | R220G | 2.5 | 2.8 | 3.3 | 4.3 | 3.5 |
| 3 | R220V | 1.5 | 2.3 | 2.8 | 2.6 | 2.0 |
| 3 | R220V | 1.6 | 2.8 | 4.1 | 7.1 | 2.0 |
| 3 | R220C | 2.9 | 2.6 | 3.0 | 2.6 | 6.2 |
| 3 | R221K | 0.6 | 0.8 | 1.0 | 1.1 | 6.3 |
| 3 | R221P | 4.4 | 5.8 | 4.1 | 5.8 | 8.5 |
| 3 | R221A | 1.2 | 1.6 | 2.3 | 4.3 | 1.9 |
| 3 | R221V | 2.0 | 2.1 | 1.3 | 3.7 | 6.0 |
| 3 | L222K | 0.4 | 0.2 | 0.6 | 0.9 | 1.9 |
| 3 | L222S | 0.7 | 2.3 | 2.8 | 11.3 | 2.5 |
| 3 | L222H | 6.1 | 12.8 | 11.5 | 15.4 | 13.6 |
| 3 | L222A | 1.2 | 2.5 | 3.2 | 1.8 | 1.5 |
| 3 | L222V | 0.9 | 0.7 | 1.1 | 0.6 | 1.3 |
| 3 | L222F | 1.7 | 2.9 | 4.0 | 9.6 | 4.8 |
| 3 | E223K | 1.0 | 1.5 | 0.9 | 2.2 | 6.7 |
| 3 | E223R | 1.6 | 3.4 | 2.2 | 4.6 | 5.5 |
| 3 | E223I | 1.6 | 4.0 | 2.5 | 7.2 | 3.0 |
| 3 | E223P | 0.8 | 2.5 | 1.6 | 12.0 | 2.0 |
| 3 | E223E | 1.2 | 1.0 | 1.1 | 1.2 | 1.5 |
| 3 | E223G | 1.5 | 2.6 | 3.8 | 4.5 | 3.5 |
| 3 | E223G | 0.9 | 1.3 | 1.0 | 1.2 | 2.2 |
| 3 | E223W | 1.5 | 2.2 | 1.3 | 3.0 | 1.4 |
| 3 | E223C | 1.1 | 0.6 | 1.1 | 3.3 | 11.5 |
| 3 | N224N | 1.3 | 1.1 | 1.5 | 1.3 | 15.4 |
| 3 | N224T | 1.5 | 1.0 | 1.0 | 1.3 | 1.3 |
| 3 | N224R | 1.9 | 1.7 | 1.0 | 0.2 | 1.2 |
| 3 | N224S | 1.0 | 2.1 | 1.5 | 3.1 | 5.0 |
| 3 | N224M | 0.5 | 0.6 | 0.4 | 0.2 | 2.3 |
| 3 | N224R | 2.9 | 2.8 | 2.8 | 4.3 | 26.8 |
| 3 | N224E | 0.9 | 1.3 | 0.9 | 4.0 | 2.4 |
| 3 | N224G | 1.7 | 1.9 | 2.3 | 2.0 | 1.2 |
| 3 | N224W | 3.4 | 7.7 | 7.5 | 8.7 | 10.2 |
| 3 | N224C | 0.9 | 2.3 | 1.3 | 3.2 | 1.7 |
| 3 | L225Q | 1.0 | 1.3 | 1.6 | 0.2 | 3.8 |
| 3 | I226T | 1.2 | 1.2 | 0.9 | 2.9 | 22.6 |
| 3 | I226S | 4.4 | 12.4 | 13.6 | 8.4 | 23.1 |
| 3 | I226E | 3.2 | 0.7 | 0.4 | 1.3 | 1.8 |
| 3 | A227K | 1.5 | 1.9 | 1.9 | 0.8 | 1.4 |
| 3 | A227M | 1.7 | 1.0 | 1.0 | 1.7 | 4.7 |
| 3 | A227P | 1.5 | 3.6 | 3.5 | 1.5 | 5.7 |
| 3 | A227D | 2.0 | 2.1 | 2.1 | 10.2 | 13.4 |
| 3 | A227A | 2.4 | 3.3 | 2.6 | 2.0 | 4.5 |
| 3 | A227C | 1.0 | 3.9 | 5.8 | 17.4 | 7.7 |
| 3 | Q228T | 2.0 | 3.6 | 3.5 | 1.3 | 8.1 |
| 3 | Q228R | 2.1 | 1.4 | 1.6 | 2.5 | 16.7 |
| 3 | Q228M | 2.0 | 2.0 | 1.0 | 1.2 | 7.7 |
| 3 | Q228I | 1.8 | 3.1 | 4.0 | 3.8 | 3.7 |
| 3 | Q228R | 1.1 | 0.3 | 0.4 | 0.2 | 1.0 |
| 3 | Q228L | 2.4 | 2.5 | 2.2 | 1.8 | 1.4 |
| 3 | Q228L | 0.7 | 1.7 | 1.1 | 1.2 | 2.1 |
| 3 | Q228S | 0.6 | 0.7 | 1.2 | 1.7 | 4.2 |
| 3 | Q228L | 0.7 | 0.8 | 0.8 | 1.1 | 1.5 |
| 3 | L229N | 0.2 | 1.1 | 1.1 | 1.4 | 1.4 |
| 3 | L229Q | 1.3 | 2.4 | 2.5 | 4.0 | 20.8 |
| 3 | L229H | 3.2 | 6.8 | 9.2 | 5.0 | 2.4 |
| 3 | L229L | 1.1 | 1.4 | 1.7 | 1.3 | 1.8 |
| 3 | L229V | 2.2 | 2.8 | 3.8 | 3.4 | 3.7 |
| 3 | P230R | 7.4 | 6.4 | 6.3 | 7.3 | 9.5 |
| 3 | P230R | 2.7 | 4.7 | 11.1 | 27.4 | 75.4 |
| 3 | P230L | 1.7 | 4.8 | 5.4 | 7.7 | 34.5 |
| 3 | P230L | 2.3 | 2.4 | 2.2 | 1.0 | 2.3 |
| 3 | P230D | 0.6 | 0.4 | 0.3 | 0.3 | 2.3 |
| 3 | P230W | 2.7 | 4.8 | 5.2 | 11.9 | 8.2 |
| 3 | P230C | 4.3 | 12.2 | 16.6 | 11.7 | 26.8 |
| 3 | G231K | 2.6 | 5.1 | 7.9 | 9.9 | 4.6 |
| 3 | G231R | 0.8 | 1.5 | 0.8 | 0.9 | 1.7 |
| 3 | G231Q | 0.1 | 0.2 | 0.4 | 0.6 | 5.7 |
| 3 | G231H | 4.0 | 4.8 | 4.4 | 1.9 | 2.7 |
| 3 | G231A | 0.8 | 1.5 | 1.7 | 0.7 | 1.6 |
| 3 | E232M | 1.2 | 1.4 | 1.5 | 0.8 | 5.3 |
| 3 | E232Q | 0.7 | 1.1 | 2.1 | 6.9 | 2.3 |
| 3 | E232R | 1.9 | 3.3 | 3.7 | 4.2 | 1.4 |
| 3 | E232L | 2.6 | 1.2 | 1.9 | 3.1 | 2.1 |
| 3 | E232G | 1.3 | 2.4 | 1.6 | 2.3 | 5.1 |
| 3 | E232V | 1.4 | 1.3 | 1.6 | 1.2 | 15.0 |
| 3 | E232V | 0.9 | 1.5 | 1.8 | 3.9 | 2.2 |
| 3 | E232W | 2.3 | 3.3 | 4.1 | 5.2 | 5.4 |
| 3 | K233T | 1.3 | 1.8 | 2.4 | 2.6 | 4.1 |
| 3 | K234R | 1.9 | 2.2 | 1.5 | 1.4 | 1.1 |
| 3 | N235T | 2.0 | 2.4 | 3.6 | 4.4 | 1.3 |
| 3 | N235R | 1.9 | 1.3 | 1.5 | 2.5 | 1.9 |
| 3 | N235S | 1.2 | 2.8 | 2.2 | 2.0 | 1.8 |
| 3 | N235I | 1.6 | 1.7 | 1.8 | 3.9 | 1.3 |
| 3 | N235Q | 0.9 | 1.2 | 1.2 | 2.2 | 2.5 |
| 3 | N235H | 1.4 | 1.2 | 1.6 | 0.5 | 1.1 |
| 3 | N235P | 0.0 | 0.2 | 0.1 | 1.0 | 1.2 |
| 3 | N235G | 1.8 | 3.4 | 3.1 | 1.9 | 3.3 |
| 3 | G236T | 1.8 | 2.2 | 1.5 | 3.8 | 8.5 |
| 3 | G236S | 1.0 | 1.7 | 1.8 | 3.6 | 4.1 |
| 3 | G236H | 1.5 | 1.9 | 1.1 | 0.3 | 1.7 |
| 3 | G236R | 1.8 | 3.9 | 4.5 | 5.7 | 2.3 |
| 3 | G236E | 1.0 | 1.6 | 1.9 | 3.5 | 8.4 |
| 3 | G236A | 0.4 | 0.8 | 0.6 | 0.7 | 1.1 |
| 3 | G236A | 0.6 | 1.3 | 1.5 | 2.3 | 5.3 |
| 3 | G236G | 1.7 | 1.7 | 1.6 | 1.8 | 3.3 |
| 3 | G236S | 2.4 | 3.9 | 3.3 | 4.1 | 1.1 |
| 3 | G236C | 1.4 | 1.3 | 1.9 | 1.7 | 1.7 |
| 3 | G236F | 1.7 | 2.9 | 2.8 | 9.1 | 11.0 |
| 3 | L237T | 2.0 | 7.2 | 6.5 | 17.2 | 24.4 |
| 3 | L237M | 2.7 | 1.4 | 1.7 | 4.6 | 7.4 |
| 3 | L237R | 2.3 | 4.3 | 3.9 | 3.9 | 11.9 |
| 3 | L237R | 0.8 | 1.4 | 4.6 | 4.1 | 8.8 |
| 3 | L237L | 0.6 | 0.8 | 1.0 | 1.4 | 1.9 |
| 3 | L237G | 1.2 | 1.4 | 1.0 | 1.3 | 5.4 |
| 3 | L237G | 2.5 | 2.3 | 3.9 | 15.2 | 18.7 |
| 3 | L237V | 1.4 | 1.4 | 2.5 | 2.7 | 1.6 |
| 3 | L237V | 2.1 | 0.3 | 0.5 | 0.4 | 1.1 |
| 3 | F238S | 2.2 | 3.0 | 1.9 | 1.1 | 3.6 |
| 3 | F238I | 1.0 | 1.3 | 2.2 | 2.2 | 2.9 |
| 3 | F238P | 4.1 | 5.6 | 6.2 | 9.3 | 14.4 |
| 3 | F238P | 5.7 | 7.8 | 10.7 | 34.5 | 29.1 |
| 3 | F238L | 1.1 | 0.5 | 0.6 | 1.0 | 1.9 |
| 3 | F238V | 0.9 | 1.0 | 1.2 | 1.3 | 1.7 |
| 3 | G239R | 0.9 | 0.9 | 0.7 | 1.1 | 1.5 |
| 3 | G239R | 2.2 | 0.3 | 0.6 | 0.6 | 1.7 |
| 3 | G239L | 1.0 | 0.2 | 0.4 | 2.6 | 3.1 |
| 3 | G239C | 1.5 | 6.7 | 5.8 | 9.7 | 5.0 |
| 3 | N240K | 1.3 | 2.5 | 2.3 | 4.9 | 3.7 |
| 3 | N240T | 1.8 | 2.0 | 2.2 | 1.9 | 3.7 |
| 3 | N240S | 0.9 | 2.6 | 3.5 | 8.5 | 6.9 |
| 3 | N240R | 2.7 | 4.1 | 4.9 | 5.8 | 2.2 |
| 3 | N240L | 1.7 | 0.5 | 0.4 | 1.5 | 8.1 |
| 3 | N240A | 1.6 | 1.0 | 0.2 | 0.4 | 1.7 |
| 3 | N240A | 2.8 | 3.1 | 2.6 | 5.0 | 4.1 |
| 3 | N240V | 0.9 | 0.3 | 0.3 | 1.7 | 2.9 |
| 3 | L241I | 0.2 | 0.0 | 0.2 | 0.2 | 1.1 |
| 3 | L241V | 1.1 | 1.4 | 2.0 | 2.3 | 11.7 |
| 3 | L241F | 1.6 | 0.7 | 0.5 | 0.8 | 1.0 |
| 3 | I242T | 0.7 | 1.9 | 1.6 | 1.9 | 1.4 |
| 3 | I242L | 1.3 | 0.8 | 1.0 | 2.1 | 4.9 |
| 3 | I242V | 1.8 | 4.4 | 2.6 | 4.6 | 2.0 |
| 3 | A243R | 1.5 | 1.6 | 1.5 | 1.2 | 3.9 |
| 3 | A243S | 2.1 | 0.6 | 0.4 | 0.5 | 3.2 |
| 3 | A243A | 1.3 | 2.5 | 2.7 | 2.2 | 1.4 |
| 3 | A243G | 2.4 | 4.2 | 5.7 | 10.0 | 11.0 |
| 3 | A243L | 2.3 | 2.6 | 2.7 | 0.4 | 2.5 |
| 3 | L244L | 2.7 | 3.6 | 3.6 | 0.8 | 1.6 |
| 3 | L244V | 1.5 | 1.6 | 1.8 | 3.9 | 1.0 |
| 3 | L244V | 1.0 | 3.9 | 3.9 | 3.8 | 5.9 |
| 3 | S245T | 0.6 | 0.2 | 0.2 | 0.7 | 1.2 |
| 3 | S245S | 2.3 | 3.3 | 2.8 | 1.2 | 1.7 |
| 3 | S245A | 2.1 | 3.8 | 3.8 | 3.0 | 2.5 |
| 3 | S245F | 2.5 | 4.8 | 5.6 | 4.6 | 9.6 |
| 3 | L246S | 1.4 | 3.8 | 3.2 | 3.4 | 7.7 |
| 3 | L246L | 0.4 | 0.4 | 0.3 | 0.9 | 1.7 |
| 3 | L246G | 1.4 | 0.8 | 1.0 | 0.7 | 1.2 |
| 3 | L246L | 1.1 | 1.1 | 1.6 | 4.1 | 2.3 |
| 3 | L246F | 0.9 | 1.0 | 1.3 | 2.7 | 1.3 |
| 3 | G247S | 0.4 | 0.2 | 0.2 | 0.9 | 2.7 |
| 3 | G247E | 0.6 | 1.4 | 1.2 | 1.3 | 1.0 |
| 3 | G247D | 0.8 | 0.9 | 1.6 | 2.4 | 2.2 |
| 3 | G247G | 1.2 | 1.1 | 1.1 | 2.1 | 3.7 |
| 3 | G247G | 0.7 | 1.2 | 0.8 | 0.5 | 1.2 |
| 3 | G247S | 1.5 | 0.8 | 0.5 | 0.7 | 1.3 |
| 3 | G247L | 1.3 | 0.5 | 0.5 | 1.4 | 1.4 |
| 3 | L248R | 3.2 | 3.4 | 4.1 | 4.6 | 5.4 |
| 3 | L248L | 1.6 | 3.1 | 4.1 | 6.3 | 11.5 |
| 3 | T249T | 1.6 | 2.3 | 2.6 | 3.7 | 5.9 |
| 3 | T249H | 1.7 | 2.6 | 4.2 | 3.7 | 1.8 |
| 3 | T249E | 0.2 | 0.6 | 0.5 | 1.6 | 1.8 |
| 3 | T249A | 1.1 | 1.3 | 3.0 | 1.2 | 1.4 |
| 3 | T249V | 5.0 | 5.8 | 11.5 | 13.0 | 1.6 |
| 3 | T249Y | 0.5 | 0.4 | 0.3 | 2.2 | 4.4 |
| 3 | T249S | 2.1 | 3.1 | 3.0 | 1.4 | 2.5 |
| 3 | P250H | 0.5 | 0.7 | 1.2 | 1.4 | 5.2 |
| 3 | P250L | 2.5 | 4.2 | 4.6 | 2.9 | 6.7 |
| 3 | P250G | 1.9 | 2.1 | 3.3 | 5.9 | 10.9 |
| 3 | N251Q | 1.9 | 1.6 | 3.1 | 4.1 | 1.1 |
| 3 | N251S | 2.0 | 2.7 | 3.9 | 4.9 | 2.1 |
| 3 | F252I | 0.7 | 1.4 | 1.8 | 2.3 | 2.9 |
| 3 | F252V | 0.7 | 1.1 | 1.1 | 1.3 | 1.1 |
| 3 | K253S | 1.1 | 1.2 | 0.9 | 1.4 | 1.1 |
| 3 | S254N | 0.5 | 0.6 | 0.8 | 2.0 | 1.6 |
| 3 | S254Q | 1.3 | 3.9 | 3.8 | 3.2 | 1.1 |
| 3 | S254R | 0.6 | 1.6 | 1.9 | 6.2 | 11.2 |
| 3 | S254R | 3.1 | 2.0 | 1.9 | 3.5 | 9.3 |
| 3 | S254G | 1.9 | 2.1 | 1.4 | 2.3 | 4.9 |
| 3 | S254G | 1.8 | 0.7 | 0.9 | 1.0 | 1.6 |
| 3 | N255T | 1.0 | 0.9 | 0.4 | 0.4 | 7.4 |
| 3 | N255R | 1.9 | 3.0 | 2.6 | 2.3 | 2.8 |
| 3 | N255Q | 6.6 | 7.7 | 6.5 | 8.4 | 20.9 |
| 3 | N255A | 1.5 | 3.7 | 2.0 | 3.7 | 2.6 |
| 3 | N255V | 1.3 | 1.0 | 0.5 | 1.7 | 1.6 |
| 3 | N255Y | 1.5 | 1.9 | 1.3 | 0.7 | 2.9 |
| 3 | N255S | 2.2 | 6.9 | 4.4 | 11.9 | 6.9 |
| 3 | N255S | 1.2 | 0.5 | 0.4 | 1.1 | 1.0 |
| 3 | N255W | 2.7 | 2.1 | 1.7 | 2.5 | 1.8 |
| 3 | F256L | 1.4 | 1.6 | 1.5 | 0.9 | 1.0 |
| 3 | D257G | 1.1 | 0.7 | 1.2 | 1.0 | 2.8 |
| 3 | L258K | 0.5 | 0.6 | 1.1 | 1.7 | 2.2 |
| 3 | L258T | 0.7 | 0.5 | 0.6 | 2.1 | 4.6 |
| 3 | L258T | 2.3 | 2.3 | 3.0 | 8.4 | 3.5 |
| 3 | L258M | 3.4 | 7.7 | 6.7 | 10.7 | 5.6 |
| 3 | L258Q | 4.0 | 4.6 | 4.0 | 0.9 | 6.7 |
| 3 | A259K | 4.0 | 1.5 | 3.0 | 4.2 | 7.5 |
| 3 | A259T | 2.6 | 2.1 | 5.4 | 7.4 | 15.1 |
| 3 | A259P | 0.0 | 0.0 | 0.2 | 0.1 | 1.7 |
| 3 | A259R | 2.5 | 3.6 | 5.7 | 10.8 | 6.1 |
| 3 | A259A | 1.9 | 2.0 | 2.3 | 4.7 | 1.7 |
| 3 | A259A | 2.0 | 6.7 | 7.5 | 6.2 | 55.1 |
| 3 | A259S | 3.1 | 4.5 | 8.5 | 10.9 | 28.8 |
| 3 | A259F | 3.7 | 8.3 | 11.3 | 0.2 | 106.4 |
| 3 | E260T | 0.6 | 5.5 | 3.9 | 14.7 | 1.4 |
| 3 | E260R | 3.2 | 3.3 | 5.2 | 10.0 | 11.8 |
| 3 | E260I | 2.4 | 3.1 | 6.5 | 3.5 | 11.0 |
| 3 | E260Q | 1.6 | 1.6 | 0.8 | 0.1 | 1.3 |
| 3 | E260D | 0.5 | 1.3 | 1.4 | 1.6 | 2.2 |
| 3 | E260G | 2.3 | 2.4 | 4.1 | 1.2 | 3.3 |
| 3 | E260Y | 1.4 | 0.8 | 1.2 | 0.1 | 2.2 |
| 3 | E260L | 6.3 | 3.0 | 7.9 | 8.0 | 34.7 |
| 3 | D261D | 0.6 | 0.6 | 1.0 | 1.9 | 1.3 |
| 3 | D261C | 5.2 | 4.7 | 6.5 | 2.6 | 8.2 |
| 3 | A262K | 2.7 | 0.9 | 1.3 | 3.4 | 4.5 |
| 3 | A262R | 1.7 | 1.0 | 4.1 | 15.8 | 24.3 |
| 3 | A262A | 1.5 | 1.6 | 1.8 | 4.7 | 1.7 |
| 3 | A262A | 1.2 | 2.8 | 3.2 | 7.8 | 5.0 |
| 3 | A262G | 1.9 | 1.1 | 1.6 | 6.8 | 5.4 |
| 3 | A262Y | 0.4 | 1.4 | 1.3 | 3.3 | 2.0 |
| 3 | A262S | 0.8 | 1.2 | 2.2 | 0.4 | 7.6 |
| 3 | K263K | 0.6 | 0.2 | 0.3 | 0.8 | 2.1 |
| 3 | L264M | 1.1 | 2.9 | 1.4 | 1.2 | 4.2 |
| 3 | L264L | 2.1 | 3.2 | 4.0 | 1.2 | 1.7 |
| 3 | L264V | 1.8 | 1.0 | 1.0 | 0.6 | 1.0 |
| 3 | Q265T | 1.3 | 4.5 | 3.8 | 0.1 | 10.3 |
| 3 | Q265I | 0.9 | 1.3 | 2.6 | 2.5 | 1.9 |
| 3 | Q265A | 4.4 | 2.7 | 4.7 | 18.7 | 6.4 |
| 3 | L266I | 1.0 | 1.6 | 1.7 | 0.5 | 1.6 |
| 3 | L266H | 1.1 | 1.3 | 2.6 | 3.5 | 32.7 |
| 3 | L266S | 0.6 | 1.0 | 0.4 | 4.5 | 6.0 |
| 3 | L266L | 0.9 | 0.8 | 1.1 | 1.1 | 2.4 |
| 3 | S267I | 1.0 | 0.5 | 0.6 | 1.3 | 1.2 |
| 3 | S267L | 2.3 | 2.6 | 3.6 | 1.3 | 1.7 |
| 3 | K268T | 0.2 | 0.3 | 0.3 | 1.8 | 7.7 |
| 3 | K268L | 3.3 | 7.4 | 6.5 | 7.4 | 19.2 |
| 3 | K268L | 1.7 | 1.2 | 1.2 | 4.1 | 95.2 |
| 3 | K268V | 1.4 | 2.2 | 1.1 | 1.6 | 11.9 |
| 3 | K268C | 1.4 | 3.6 | 4.0 | 1.8 | 23.3 |
| 3 | K268L | 2.2 | 2.7 | 3.0 | 2.4 | 1.3 |
| 3 | K268F | 3.8 | 5.1 | 7.1 | 1.8 | 7.0 |
| 3 | D269G | 2.0 | 1.2 | 0.8 | 5.4 | 10.5 |
| 3 | T270N | 0.8 | 0.9 | 0.5 | 2.1 | 1.4 |
| 3 | T270P | 1.1 | 1.8 | 1.0 | 1.7 | 1.3 |
| 3 | T270D | 2.9 | 4.6 | 6.0 | 21.0 | 100.9 |
| 3 | Y271H | 1.2 | 0.5 | 0.6 | 0.5 | 1.1 |
| 3 | Y271C | 0.4 | 0.5 | 0.4 | 0.5 | 1.6 |
| 3 | D272S | 1.4 | 1.8 | 2.7 | 8.7 | 4.9 |
| 3 | D272W | 1.4 | 4.3 | 3.0 | 3.0 | 1.2 |
| 3 | D273S | 0.7 | 1.8 | 1.4 | 1.8 | 2.0 |
| 3 | D273A | 0.9 | 2.2 | 1.3 | 0.1 | 6.4 |
| 3 | D273G | 1.5 | 0.9 | 1.2 | 2.0 | 2.8 |
| 3 | D273V | 1.0 | 0.9 | 1.4 | 1.7 | 1.1 |
| 3 | D273C | 2.0 | 2.5 | 1.2 | 1.1 | 1.2 |
| 3 | D274L | 0.7 | 0.5 | 0.7 | 0.8 | 2.9 |
| 3 | D274E | 1.9 | 1.0 | 1.5 | 0.6 | 1.6 |
| 3 | D274V | 1.5 | 0.9 | 1.0 | 3.8 | 3.0 |
| 3 | D274Y | 1.3 | 1.2 | 1.8 | 0.9 | 1.1 |
| 3 | L275R | 1.0 | 4.0 | 6.4 | 9.3 | 2.3 |
| 3 | L275L | 2.8 | 3.4 | 3.3 | 4.2 | 1.4 |
| 3 | L275A | 1.3 | 5.0 | 5.2 | 1.5 | 1.0 |
| 3 | D276H | 1.7 | 1.3 | 0.1 | 0.4 | 1.0 |
| 3 | D276V | 1.0 | 1.1 | 1.3 | 2.4 | 3.5 |
| 3 | N277P | 2.1 | 2.8 | 1.9 | 11.8 | 7.8 |
| 3 | N277R | 2.7 | 1.8 | 2.0 | 0.4 | 1.0 |
| 3 | N277A | 3.7 | 4.0 | 1.0 | 5.3 | 11.4 |
| 3 | N277Y | 1.9 | 2.5 | 2.6 | 3.0 | 14.8 |
| 3 | N277W | 1.3 | 1.0 | 0.8 | 1.4 | 3.8 |
| 3 | N277F | 1.7 | 1.0 | 0.9 | 1.4 | 2.2 |
| 3 | L278L | 1.4 | 1.8 | 3.0 | 3.4 | 3.0 |
| 3 | L278F | 1.5 | 1.7 | 2.1 | 5.9 | 3.1 |
| 3 | L279M | 2.7 | 1.5 | 3.0 | 1.0 | 9.3 |
| 3 | L279F | 0.6 | 0.5 | 0.7 | 0.6 | 4.7 |
| 3 | A280T | 0.6 | 0.5 | 0.8 | 0.4 | 1.1 |
| 3 | A280S | 0.3 | 0.4 | 0.5 | 0.1 | 2.6 |
| 3 | A280M | 0.2 | 0.7 | 1.1 | 6.0 | 2.0 |
| 3 | A280P | 2.4 | 5.3 | 5.3 | 7.2 | 1.1 |
| 3 | A280Y | 0.5 | 0.1 | 0.6 | 0.7 | 1.7 |
| 3 | Q281S | 1.4 | 2.6 | 8.1 | 16.6 | 46.5 |
| 3 | Q281M | 3.6 | 0.9 | 1.9 | 2.2 | 3.8 |
| 3 | Q281I | 0.3 | 2.8 | 2.5 | 5.7 | 6.2 |
| 3 | Q281R | 1.0 | 0.5 | 0.6 | 1.1 | 1.6 |
| 3 | Q281L | 1.2 | 2.0 | 4.3 | 8.2 | 70.2 |
| 3 | I282T | 2.0 | 5.1 | 6.8 | 2.2 | 1.2 |
| 3 | I282A | 4.7 | 14.2 | 19.1 | 29.4 | 67.5 |
| 3 | I282G | 2.3 | 2.4 | 2.7 | 3.9 | 26.4 |
| 3 | I282F | 0.9 | 1.0 | 1.2 | 1.2 | 4.8 |
| 3 | G283R | 0.9 | 2.1 | 1.4 | 3.9 | 7.6 |
| 3 | G283Q | 3.0 | 2.3 | 2.7 | 4.6 | 2.5 |
| 3 | G283P | 1.8 | 2.8 | 2.9 | 2.0 | 3.4 |
| 3 | G283Y | 6.9 | 7.9 | 6.8 | 4.3 | 1.0 |
| 3 | G283S | 5.0 | 1.1 | 2.3 | 2.8 | 5.5 |
| 3 | G283F | 2.0 | 1.2 | 3.2 | 10.8 | 8.3 |
| 3 | D284T | 0.3 | 0.8 | 1.0 | 1.2 | 3.7 |
| 3 | D284W | 1.4 | 1.1 | 0.8 | 1.6 | 1.0 |
| 3 | D284F | 0.7 | 2.0 | 1.7 | 2.2 | 1.1 |
| 3 | G283P | 1.9 | 1.7 | 1.6 | 1.1 | 1.7 |
| 3 | G283Y | 0.9 | 0.5 | 0.3 | 1.5 | 3.1 |
| 3 | G283S | 2.1 | 2.3 | 1.8 | 1.8 | 2.9 |
| 3 | G283F | 1.1 | 1.0 | 0.7 | 2.1 | 2.2 |
| 3 | Q285K | 0.8 | 0.3 | 0.4 | 0.4 | 1.3 |
| 3 | Q285M | 0.7 | 0.9 | 1.8 | 4.1 | 1.4 |
| 3 | Q285I | 1.5 | 2.0 | 1.4 | 3.2 | 4.4 |
| 3 | Q285Q | 1.1 | 1.4 | 1.3 | 1.5 | 7.4 |
| 3 | Q285H | 1.6 | 2.0 | 2.9 | 4.8 | 1.7 |
| 3 | Q285R | 1.2 | 1.7 | 1.9 | 4.8 | 1.6 |
| 3 | Q285L | 1.0 | 0.2 | 0.2 | 0.9 | 1.7 |
| 3 | Q285V | 0.9 | 1.3 | 1.1 | 2.0 | 1.0 |
| 3 | Q285Y | 0.8 | 2.3 | 3.2 | 3.3 | 4.4 |
| 3 | Q285W | 1.7 | 1.5 | 2.1 | 3.4 | 6.0 |
| 3 | Q285L | 1.2 | 1.2 | 1.5 | 4.0 | 2.7 |
| 3 | Y286G | 0.8 | 2.2 | 2.0 | 3.4 | 6.6 |
| 3 | Y286W | 0.7 | 0.8 | 0.7 | 1.2 | 1.7 |
| 3 | Y286F | 0.8 | 1.9 | 1.8 | 0.5 | 1.2 |
| 3 | A287R | 0.6 | 0.5 | 0.7 | 0.8 | 22.3 |
| 3 | A287D | 0.4 | 0.7 | 0.9 | 1.4 | 11.9 |
| 3 | D288S | 0.5 | 0.4 | 0.3 | 2.1 | 1.7 |
| 3 | L289T | 0.9 | 0.8 | 1.2 | 2.7 | 2.0 |
| 3 | L289H | 0.8 | 0.7 | 1.2 | 1.3 | 1.5 |
| 3 | L289P | 0.5 | 0.4 | 0.5 | 0.5 | 5.0 |
| 3 | L289V | 1.4 | 1.9 | 2.6 | 3.7 | 1.1 |
| 3 | L289V | 1.4 | 0.7 | 0.8 | 2.0 | 1.1 |
| 3 | L289S | 1.0 | 0.8 | 1.3 | 1.8 | 13.2 |
| 3 | F290V | 0.4 | 0.3 | 0.3 | 0.8 | 1.5 |
| 3 | F290L | 1.0 | 0.6 | 0.7 | 0.8 | 2.4 |
| 3 | L291W | 1.3 | 2.1 | 2.6 | 2.5 | 5.0 |
| 3 | L291L | 0.7 | 0.7 | 0.8 | 4.3 | 3.6 |
| 3 | L291F | 1.0 | 1.5 | 1.7 | 1.1 | 3.5 |
| 3 | A292T | 1.1 | 1.5 | 2.3 | 2.3 | 3.2 |
| 3 | A292R | 1.1 | 1.8 | 2.3 | 1.9 | 1.4 |
| 3 | A292M | 0.7 | 1.1 | 0.9 | 1.5 | 1.7 |
| 3 | A292Q | 1.9 | 0.9 | 1.0 | 1.2 | 1.8 |
| 3 | A292W | 1.1 | 0.8 | 0.7 | 0.5 | 1.1 |
| 3 | A292L | 1.5 | 1.7 | 1.2 | 1.0 | 1.3 |
| 3 | A292F | 1.1 | 0.5 | 0.7 | 0.8 | 1.5 |
| 3 | A293S | 0.7 | 0.8 | 1.3 | 2.6 | 1.5 |
| 3 | A293L | 1.0 | 1.7 | 1.3 | 3.7 | 5.8 |
| 3 | A293D | 0.6 | 0.9 | 0.8 | 1.0 | 1.0 |
| 3 | A293A | 1.9 | 1.1 | 1.2 | 1.2 | 1.1 |
| 3 | A293G | 1.3 | 1.6 | 1.5 | 1.5 | 2.8 |
| 3 | A293V | 1.5 | 1.4 | 1.4 | 1.0 | 1.6 |
| 3 | A293S | 0.7 | 0.9 | 0.9 | 2.5 | 1.2 |
| 3 | K294K | 1.5 | 1.7 | 2.0 | 1.8 | 3.1 |
| 3 | K294W | 0.8 | 1.3 | 0.9 | 1.0 | 4.8 |
| 3 | N295K | 1.4 | 1.4 | 1.7 | 2.1 | 1.5 |
| 3 | N295T | 0.9 | 1.3 | 1.7 | 1.6 | 1.4 |
| 3 | N295R | 1.1 | 1.5 | 2.0 | 1.8 | 1.5 |
| 3 | N295S | 1.4 | 2.6 | 2.3 | 1.9 | 1.9 |
| 3 | N295M | 0.9 | 0.9 | 1.3 | 2.3 | 4.7 |
| 3 | N295I | 0.9 | 1.4 | 1.7 | 2.6 | 3.8 |
| 3 | N295H | 0.9 | 0.9 | 1.1 | 1.9 | 1.1 |
| 3 | N295P | 1.0 | 1.8 | 1.6 | 1.2 | 2.5 |
| 3 | N295R | 1.0 | 1.5 | 1.9 | 3.2 | 1.2 |
| 3 | N295V | 0.5 | 0.6 | 0.6 | 2.9 | 1.4 |
| 3 | N295Y | 1.0 | 1.0 | 1.1 | 2.0 | 1.2 |
| 3 | N295S | 0.5 | 0.8 | 1.3 | 2.1 | 7.7 |
| 3 | N295S | 0.7 | 0.5 | 1.3 | 1.2 | 1.6 |
| 3 | N295W | 0.7 | 0.8 | 0.6 | 1.1 | 1.2 |
| 3 | N295C | 1.4 | 0.9 | 0.7 | 0.5 | 1.5 |
| 3 | N295F | 1.1 | 0.8 | 1.0 | 2.0 | 1.1 |
| 3 | L296H | 1.0 | 0.6 | 0.4 | 1.4 | 1.1 |
| 3 | L296L | 0.9 | 1.8 | 2.0 | 4.7 | 4.8 |
| 3 | L296Y | 0.4 | 0.4 | 0.4 | 1.3 | 3.9 |
| 3 | S297M | 1.0 | 0.5 | 0.6 | 2.0 | 1.0 |
| 3 | S297H | 0.6 | 0.7 | 1.6 | 1.8 | 25.5 |
| 3 | S297A | 0.6 | 1.0 | 1.4 | 2.4 | 5.7 |
| 3 | S297A | 0.9 | 0.8 | 0.9 | 1.5 | 1.5 |
| 3 | D298L | 0.9 | 1.4 | 1.5 | 4.3 | 9.2 |
| 3 | D298V | 1.6 | 1.3 | 0.7 | 2.9 | 3.0 |
| 3 | A299T | 1.0 | 1.4 | 2.4 | 6.4 | 9.7 |
| 3 | A299H | 1.4 | 0.5 | 1.3 | 1.5 | 4.5 |
| 3 | A299W | 0.6 | 1.7 | 1.6 | 2.8 | 3.4 |
| 3 | I300H | 1.0 | 1.0 | 1.1 | 1.1 | 2.0 |
| 3 | I300L | 0.4 | 0.2 | 0.5 | 0.9 | 1.7 |
| 3 | I300Y | 0.4 | 0.8 | 1.3 | 1.4 | 2.2 |
| 3 | I300L | 1.6 | 1.8 | 2.2 | 2.4 | 1.8 |
| 4 | L301K | 1.1 | 1.1 | 3.2 | 4.1 | 3.0 |
| 4 | L301T | 1.6 | 2.9 | 5.0 | 9.8 | 22.5 |
| 4 | L301M | 1.5 | 2.1 | 2.9 | 5.1 | 3.6 |
| 4 | L301I | 1.2 | 2.3 | 5.3 | 6.6 | 9.0 |
| 4 | L301Q | 2.0 | 1.6 | 0.8 | 1.5 | 7.6 |
| 4 | L301R | 1.1 | 3.1 | 2.5 | 1.8 | 2.4 |
| 4 | L301L | 1.2 | 1.7 | 3.0 | 3.8 | 9.4 |
| 4 | L301A | 1.0 | 1.0 | 2.8 | 2.8 | 2.7 |
| 4 | L301G | 1.5 | 2.2 | 2.4 | 3.3 | 3.9 |
| 4 | L301G | 2.0 | 2.5 | 5.0 | 5.3 | 5.7 |
| 4 | L301V | 1.3 | 1.6 | 4.5 | 4.8 | 5.5 |
| 4 | L301V | 1.1 | 0.1 | 1.6 | 2.2 | 4.3 |
| 4 | L301C | 1.4 | 4.1 | 5.3 | 6.6 | 6.2 |
| 4 | L301F | 1.2 | 1.0 | 1.3 | 1.7 | 1.7 |
| 4 | L302R | 1.0 | 0.3 | 0.8 | 1.4 | 2.9 |
| 4 | L302L | 1.4 | 2.7 | 1.6 | 1.2 | 2.0 |
| 4 | L302L | 0.9 | 1.1 | 3.7 | 3.9 | 6.8 |
| 4 | S303K | 1.3 | 2.9 | 5.0 | 2.8 | 2.0 |
| 4 | S303Q | 2.2 | 1.5 | 4.6 | 3.8 | 8.6 |
| 4 | S303E | 1.9 | 1.7 | 2.0 | 1.6 | 2.8 |
| 4 | S303A | 1.0 | 2.6 | 1.6 | 4.5 | 2.3 |
| 4 | S303A | 1.5 | 1.4 | 1.7 | 2.2 | 1.6 |
| 4 | S303S | 1.2 | 2.5 | 3.8 | 4.4 | 3.0 |
| 4 | S303L | 1.4 | 2.7 | 3.9 | 6.1 | 8.4 |
| 4 | D304T | 2.2 | 7.7 | 11.4 | 8.1 | 15.1 |
| 4 | D304I | 1.8 | 5.8 | 9.0 | 12.2 | 8.3 |
| 4 | D304L | 1.7 | 4.4 | 10.4 | 9.5 | 6.6 |
| 4 | D304W | 1.4 | 2.5 | 5.4 | 4.7 | 6.2 |
| 4 | L306L | 1.3 | 2.9 | 1.2 | 1.4 | 1.5 |
| 4 | R307A | 2.3 | 4.5 | 11.2 | 15.1 | 16.8 |
| 4 | R307V | 1.4 | 2.0 | 4.3 | 6.0 | 2.2 |
| 4 | R307C | 1.4 | 1.9 | 9.1 | 13.8 | 15.2 |
| 4 | V308R | 0.6 | 3.5 | 9.6 | 4.0 | 4.8 |
| 4 | V308S | 1.1 | 2.5 | 14.5 | 13.7 | 10.9 |
| 4 | V308L | 1.9 | 3.8 | 11.2 | 13.2 | 4.8 |
| 4 | V308E | 1.5 | 3.1 | 8.1 | 3.8 | 5.0 |
| 4 | V308A | 1.9 | 2.2 | 1.5 | 3.5 | 6.1 |
| 4 | V308A | 1.1 | 1.7 | 0.4 | 0.9 | 2.5 |
| 4 | V308G | 1.2 | 1.6 | 0.8 | 0.6 | 1.2 |
| 4 | V308C | 1.1 | 4.0 | 8.2 | 5.5 | 1.8 |
| 4 | N309R | 3.0 | 3.3 | 6.3 | 8.5 | 10.5 |
| 4 | T310S | 2.8 | 5.5 | 10.3 | 15.7 | 16.5 |
| 4 | T310P | 2.8 | 3.4 | 0.2 | 2.0 | 4.5 |
| 4 | T310L | 4.8 | 9.6 | 13.2 | 14.8 | 21.6 |
| 4 | T310A | 2.1 | 3.4 | 7.3 | 8.9 | 2.1 |
| 4 | T310G | 0.9 | 3.1 | 11.8 | 10.4 | 6.0 |
| 4 | T310W | 2.6 | 4.6 | 7.8 | 6.0 | 3.5 |
| 4 | E311E | 1.4 | 2.0 | 1.5 | 2.1 | 1.8 |
| 4 | E311A | 2.1 | 7.1 | 10.2 | 7.6 | 2.3 |
| 4 | E311G | 1.6 | 2.4 | 8.8 | 6.9 | 13.0 |
| 4 | E311C | 3.2 | 7.8 | 15.1 | 10.2 | 13.5 |
| 4 | I312R | 0.9 | 0.9 | 0.6 | 1.2 | 1.9 |
| 4 | I312S | 1.4 | 4.8 | 9.2 | 3.5 | 4.1 |
| 4 | I312E | 1.8 | 1.0 | 2.2 | 2.7 | 2.2 |
| 4 | I312A | 3.7 | 22.9 | 46.2 | 29.1 | 47.2 |
| 4 | I312S | 2.3 | 0.7 | 4.8 | 3.6 | 3.1 |
| 4 | I312C | 3.0 | 7.9 | 7.7 | 13.1 | 9.0 |
| 4 | T313E | 0.8 | 4.4 | 14.7 | 18.9 | 27.2 |
| 4 | T313C | 0.8 | 0.8 | 1.1 | 2.6 | 2.3 |
| 4 | T313F | 3.1 | 9.6 | 10.0 | 18.6 | 1.8 |
| 4 | K314Q | 3.1 | 5.8 | 8.9 | 4.7 | 3.1 |
| 4 | K314G | 1.1 | 1.2 | 2.9 | 6.6 | 6.1 |
| 4 | A315C | 5.2 | 4.9 | 7.1 | 7.2 | 4.0 |
| 4 | P316S | 3.3 | 6.4 | 0.3 | 1.3 | 1.2 |
| 4 | P316D | 0.6 | 2.2 | 0.5 | 1.3 | 4.1 |
| 4 | S318S | 1.3 | 4.9 | 13.4 | 8.3 | 4.2 |
| 4 | A319T | 1.2 | 4.1 | 0.7 | 0.4 | 1.3 |
| 4 | A319M | 1.2 | 6.2 | 14.2 | 13.6 | 8.2 |
| 4 | A319I | 1.3 | 5.5 | 6.0 | 1.7 | 1.4 |
| 4 | A319H | 1.0 | 0.8 | 3.3 | 5.4 | 3.1 |
| 4 | A319L | 2.4 | 0.1 | 1.2 | 1.1 | 2.3 |
| 4 | A319G | 0.8 | 1.6 | 2.8 | 1.1 | 3.9 |
| 4 | A319S | 2.1 | 6.5 | 12.7 | 13.4 | 5.6 |
| 4 | A319F | 3.1 | 3.1 | 3.2 | 4.8 | 3.1 |
| 4 | S320N | 1.9 | 3.4 | 4.0 | 3.0 | 1.6 |
| 4 | S320S | 1.6 | 2.3 | 6.3 | 6.3 | 6.1 |
| 4 | S320A | 1.5 | 3.8 | 1.0 | 1.4 | 1.5 |
| 4 | M321Y | 2.6 | 0.7 | 0.2 | 1.2 | 4.7 |
| 4 | I322T | 2.0 | 5.3 | 15.2 | 31.6 | 43.2 |
| 4 | I322S | 2.6 | 8.2 | 12.1 | 18.6 | 27.0 |
| 4 | I322M | 1.5 | 3.3 | 5.0 | 5.7 | 5.9 |
| 4 | I322R | 3.8 | 6.9 | 22.0 | 32.5 | 34.0 |
| 4 | I322E | 2.0 | 6.9 | 9.0 | 10.7 | 6.4 |
| 4 | I322G | 1.7 | 0.6 | 0.5 | 1.9 | 2.0 |
| 4 | K323K | 1.2 | 4.0 | 3.9 | 4.4 | 2.2 |
| 4 | K323R | 1.3 | 3.8 | 7.6 | 19.1 | 8.1 |
| 4 | K323V | 1.5 | 0.1 | 0.5 | 1.6 | 2.3 |
| 4 | K323V | 2.0 | 1.2 | 3.4 | 6.3 | 9.8 |
| 4 | K323S | 1.1 | 3.5 | 5.4 | 7.0 | 6.9 |
| 4 | R324R | 1.4 | 2.2 | 3.5 | 3.1 | 1.9 |
| 4 | R324G | 1.0 | 0.7 | 1.8 | 0.4 | 1.1 |
| 4 | Y325S | 1.5 | 1.7 | 1.8 | 1.7 | 1.8 |
| 4 | Y325C | 0.9 | 1.1 | 1.0 | 1.2 | 1.3 |
| 4 | Y325F | 1.4 | 1.7 | 1.2 | 1.6 | 1.6 |
| 4 | D326Q | 0.9 | 2.9 | 6.0 | 1.6 | 1.4 |
| 4 | D326H | 2.8 | 3.4 | 1.6 | 1.8 | 2.3 |
| 4 | D326E | 1.6 | 1.8 | 2.5 | 2.1 | 2.0 |
| 4 | D326A | 1.4 | 3.6 | 3.3 | 2.3 | 2.0 |
| 4 | D326G | 1.1 | 2.7 | 4.8 | 1.3 | 2.2 |
| 4 | E327E | 1.0 | 1.2 | 1.6 | 2.2 | 1.5 |
| 4 | H329M | 0.2 | 1.8 | 0.2 | 1.1 | 1.3 |
| 4 | H329R | 1.0 | 5.6 | 3.0 | 11.3 | 2.4 |
| 4 | H329S | 2.8 | 12.6 | 12.5 | 9.4 | 11.6 |
| 4 | Q330H | 2.7 | 4.5 | 1.1 | 1.2 | 1.4 |
| 4 | Q330E | 0.6 | 0.5 | 0.9 | 1.5 | 2.0 |
| 4 | Q330G | 1.6 | 4.9 | 1.2 | 1.9 | 3.9 |
| 4 | Q330S | 1.4 | 5.7 | 8.3 | 24.6 | 13.8 |
| 4 | Q330L | 2.0 | 1.8 | 4.0 | 6.6 | 6.4 |
| 4 | L332I | 1.1 | 4.6 | 16.2 | 23.2 | 18.2 |
| 4 | L332L | 1.4 | 2.9 | 3.6 | 3.6 | 3.3 |
| 4 | T333M | 1.3 | 0.8 | 8.1 | 15.9 | 9.7 |
| 4 | T333R | 0.9 | 0.8 | 1.3 | 1.1 | 1.0 |
| 4 | T333S | 1.0 | 1.2 | 1.3 | 2.3 | 5.7 |
| 4 | T333W | 1.4 | 4.0 | 7.6 | 14.2 | 14.3 |
| 4 | T333L | 1.7 | 5.5 | 20.9 | 16.3 | 8.1 |
| 4 | T333F | 2.0 | 5.1 | 8.0 | 9.5 | 7.4 |
| 4 | L334S | 3.0 | 13.2 | 18.2 | 19.3 | 10.1 |
| 4 | L335V | 1.4 | 2.0 | 7.7 | 12.6 | 6.9 |
| 4 | K336R | 2.1 | 5.8 | 4.6 | 1.4 | 1.0 |
| 4 | A337T | 1.2 | 0.6 | 0.8 | 3.2 | 1.2 |
| 4 | A337I | 2.2 | 7.7 | 19.8 | 36.5 | 23.8 |
| 4 | A337G | 2.2 | 4.8 | 8.5 | 1.7 | 1.3 |
| 4 | L338V | 0.7 | 1.2 | 2.5 | 1.2 | 1.6 |
| 4 | L338C | 2.1 | 0.8 | 1.3 | 3.1 | 1.8 |
| 4 | V339A | 1.7 | 2.9 | 0.5 | 2.5 | 2.6 |
| 4 | V339V | 0.9 | 6.6 | 3.7 | 2.9 | 1.3 |
| 4 | R340K | 0.7 | 3.6 | 8.0 | 8.5 | 10.2 |
| 4 | R340R | 0.6 | 4.4 | 2.7 | 7.3 | 1.4 |
| 4 | R340S | 0.3 | 1.1 | 5.0 | 5.1 | 3.8 |
| 4 | Q341M | 0.8 | 4.3 | 11.4 | 10.5 | 6.8 |
| 4 | Q341I | 0.8 | 3.9 | 7.4 | 13.2 | 1.6 |
| 4 | Q341V | 2.2 | 8.6 | 26.3 | 15.1 | 16.5 |
| 4 | L343R | 2.3 | 4.9 | 2.1 | 2.6 | 1.0 |
| 4 | L343R | 1.3 | 0.8 | 1.0 | 1.4 | 1.8 |
| 4 | L343L | 2.1 | 3.6 | 2.9 | 1.8 | 1.5 |
| 4 | L343Y | 0.8 | 11.1 | 24.6 | 34.8 | 30.1 |
| 4 | L343S | 2.1 | 6.9 | 5.0 | 3.5 | 1.8 |
| 4 | L343C | 1.1 | 0.8 | 3.0 | 7.0 | 5.5 |
| 4 | L343L | 1.2 | 9.6 | 2.6 | 1.4 | 2.8 |
| 4 | L343F | 2.1 | 2.4 | 3.2 | 3.1 | 1.4 |
| 4 | P344T | 4.4 | 4.2 | 31.6 | 25.0 | 12.3 |
| 4 | E345K | 1.0 | 3.6 | 1.4 | 2.0 | 3.5 |
| 4 | E345Y | 2.9 | 7.9 | 24.7 | 18.8 | 14.6 |
| 4 | E345W | 1.4 | 0.5 | 0.4 | 0.7 | 1.0 |
| 4 | K346R | 4.0 | 7.9 | 17.8 | 20.5 | 36.1 |
| 4 | Y347S | 0.7 | 0.0 | 11.8 | 21.8 | 20.0 |
| 4 | K348R | 2.1 | 15.2 | 44.4 | 48.2 | 27.1 |
| 4 | K348G | 1.2 | 16.5 | 8.8 | 17.4 | 11.1 |
| 4 | E349E | 0.9 | 3.6 | 5.3 | 8.0 | 2.2 |
| 4 | F351C | 0.5 | 2.7 | 3.5 | 3.1 | 1.3 |
| 4 | F352M | 1.8 | 1.3 | 6.3 | 5.3 | 1.3 |
| 4 | F352E | 0.4 | 2.0 | 11.3 | 5.5 | 4.9 |
| 4 | F352Y | 1.1 | 7.4 | 4.2 | 5.5 | 11.8 |
| 4 | F352S | 2.9 | 2.2 | 5.6 | 4.0 | 1.2 |
| 4 | D353V | 1.2 | 0.2 | 1.0 | 1.1 | 2.1 |
| 4 | D353F | 0.7 | 2.1 | 4.3 | 2.2 | 1.2 |
| 4 | F352E | 0.3 | 1.1 | 7.4 | 3.4 | 3.2 |
| 4 | F352Y | 0.6 | 3.7 | 3.2 | 3.6 | 7.5 |
| 4 | F352S | 2.0 | 3.7 | 9.9 | 6.5 | 2.3 |
| 4 | D353V | 1.0 | 0.2 | 2.1 | 1.9 | 3.7 |
| 4 | Q354K | 1.2 | 3.9 | 4.2 | 4.5 | 5.6 |
| 4 | Q354T | 3.5 | 6.0 | 3.2 | 16.6 | 13.1 |
| 4 | Q354S | 1.0 | 1.1 | 0.0 | 1.4 | 1.6 |
| 4 | S355K | 1.1 | 2.7 | 3.2 | 5.6 | 2.5 |
| 4 | S355S | 0.5 | 3.0 | 2.4 | 2.3 | 2.0 |
| 4 | S355H | 1.5 | 2.5 | 11.2 | 8.1 | 10.8 |
| 4 | S355E | 1.2 | 1.7 | 1.9 | 3.8 | 2.8 |
| 4 | S355A | 1.1 | 2.8 | 3.0 | 1.9 | 1.1 |
| 4 | S355S | 1.4 | 4.9 | 9.6 | 9.6 | 2.7 |
| 4 | N357S | 1.1 | 3.8 | 3.6 | 8.0 | 11.2 |
| 4 | N357A | 2.0 | 8.8 | 7.6 | 9.4 | 55.4 |
| 4 | N357G | 0.7 | 2.0 | 4.0 | 7.3 | 15.3 |
| 4 | N357V | 2.3 | 6.8 | 16.9 | 13.1 | 13.9 |
| 4 | N357Y | 1.3 | 1.4 | 1.3 | 1.3 | 1.5 |
| 4 | Y359F | 1.1 | 0.3 | 1.5 | 1.4 | 1.5 |
| 4 | A360G | 0.9 | 1.8 | 7.2 | 8.3 | 5.9 |
| 4 | Y362L | 3.6 | 1.1 | 5.8 | 8.7 | 10.8 |
| 4 | I363R | 0.7 | 2.6 | 4.6 | 2.3 | 1.6 |
| 4 | I363V | 0.9 | 1.7 | 2.1 | 2.3 | 3.3 |
| 4 | D364N | 1.1 | 3.5 | 4.1 | 13.4 | 28.7 |
| 4 | D364T | 0.9 | 7.7 | 18.5 | 27.9 | 20.6 |
| 4 | D364I | 1.2 | 0.0 | 1.0 | 1.6 | 1.7 |
| 4 | D364Q | 1.5 | 3.5 | 0.9 | 1.8 | 1.8 |
| 4 | D364D | 1.1 | 0.6 | 2.1 | 2.3 | 4.4 |
| 4 | D364G | 1.8 | 2.5 | 4.1 | 3.9 | 5.6 |
| 4 | D364S | 2.7 | 12.3 | 35.9 | 62.0 | 104.4 |
| 4 | G366H | 4.5 | 9.3 | 14.0 | 11.1 | 2.7 |
| 4 | G366R | 1.1 | 4.1 | 12.1 | 4.5 | 5.6 |
| 4 | A367Q | 1.7 | 8.9 | 9.8 | 15.1 | 11.8 |
| 4 | S368T | 1.6 | 4.6 | 2.6 | 0.1 | 4.0 |
| 4 | S368R | 1.0 | 0.7 | 0.8 | 1.9 | 2.8 |
| 4 | S368E | 0.5 | 0.6 | 4.1 | 2.1 | 2.2 |
| 4 | S368G | 0.9 | 1.6 | 3.5 | 4.1 | 3.2 |
| 4 | S368S | 1.3 | 1.5 | 10.3 | 7.5 | 5.6 |
| 4 | S368S | 1.3 | 2.9 | 3.8 | 3.7 | 5.1 |
| 4 | Q369I | 1.0 | 1.7 | 1.3 | 2.4 | 1.7 |
| 4 | E370D | 1.9 | 6.7 | 4.9 | 9.2 | 7.0 |
| 4 | E370A | 1.4 | 1.2 | 5.1 | 1.3 | 1.3 |
| 4 | E371H | 1.6 | 4.7 | 8.3 | 3.8 | 3.7 |
| 4 | E371F | 1.9 | 2.7 | 6.8 | 6.7 | 1.6 |
| 4 | F372Y | 1.3 | 1.8 | 5.5 | 2.9 | 2.6 |
| 4 | Y373G | 1.3 | 2.2 | 5.7 | 7.0 | 4.9 |
| 4 | K374M | 1.9 | 2.9 | 4.6 | 3.2 | 3.2 |
| 4 | K374R | 2.5 | 13.6 | 25.8 | 23.5 | 31.4 |
| 4 | K374G | 1.3 | 3.0 | 0.2 | 2.2 | 5.0 |
| 4 | K374S | 2.1 | 9.7 | 4.1 | 2.7 | 9.2 |
| 4 | F375S | 2.4 | 5.7 | 28.5 | 16.0 | 4.1 |
| 4 | F375I | 1.1 | 0.9 | 0.9 | 1.0 | 1.2 |
| 4 | F375A | 2.4 | 5.8 | 7.9 | 4.3 | 2.7 |
| 4 | F375Y | 2.0 | 1.1 | 1.0 | 1.0 | 1.3 |
| 4 | I376T | 1.6 | 3.7 | 17.5 | 19.3 | 45.2 |
| 4 | I376L | 2.5 | 8.6 | 11.5 | 8.4 | 14.6 |
| 4 | K377T | 1.5 | 2.6 | 1.1 | 1.0 | 1.5 |
| 4 | K377Q | 4.5 | 4.0 | 19.0 | 24.8 | 21.4 |
| 4 | P378P | 1.2 | 1.2 | 1.2 | 1.3 | 1.3 |
| 4 | P378R | 0.6 | 0.2 | 1.5 | 1.3 | 1.1 |
| 4 | P378A | 0.8 | 1.6 | 8.0 | 5.7 | 4.9 |
| 4 | P378S | 1.5 | 1.2 | 6.5 | 5.2 | 4.3 |
| 4 | I379M | 0.9 | 1.6 | 4.8 | 6.4 | 4.6 |
| 4 | I379V | 2.2 | 3.2 | 2.8 | 3.7 | 3.5 |
| 4 | I379S | 0.6 | 4.5 | 10.7 | 7.7 | 3.1 |
| 4 | I379F | 1.4 | 3.2 | 3.4 | 3.0 | 2.9 |
| 4 | E381Q | 1.4 | 7.0 | 21.2 | 13.8 | 13.4 |
| 4 | K382S | 0.6 | 4.8 | 23.3 | 30.6 | 38.4 |
| 4 | K382M | 1.9 | 5.9 | 12.4 | 13.4 | 6.9 |
| 4 | K382A | 2.3 | 3.9 | 1.2 | 7.9 | 28.4 |
| 4 | K382G | 1.7 | 4.8 | 15.4 | 6.9 | 2.9 |
| 4 | D384E | 1.0 | 3.4 | 12.6 | 21.0 | 27.2 |
| 4 | D384G | 2.0 | 2.2 | 6.9 | 5.4 | 7.2 |
| 4 | G385G | 1.9 | 3.1 | 0.3 | 1.9 | 4.4 |
| 4 | T386K | 1.4 | 4.2 | 7.1 | 6.5 | 5.1 |
| 4 | T386T | 1.0 | 1.5 | 3.0 | 3.0 | 2.4 |
| 4 | E387E | 0.3 | 1.0 | 0.9 | 0.9 | 1.0 |
| 4 | E388N | 0.3 | 3.0 | 4.6 | 10.6 | 9.8 |
| 4 | E388T | 1.9 | 5.0 | 7.3 | 13.5 | 11.0 |
| 4 | E388P | 2.4 | 12.5 | 20.9 | 25.8 | 47.9 |
| 4 | E388D | 1.1 | 2.6 | 8.0 | 6.5 | 9.2 |
| 4 | E388F | 1.3 | 3.3 | 9.7 | 3.7 | 1.7 |
| 4 | L390M | 1.6 | 1.5 | 8.3 | 8.4 | 6.6 |
| 4 | L390F | 1.2 | 2.9 | 4.1 | 2.3 | 1.5 |
| 4 | V391D | 0.7 | 3.1 | 14.4 | 3.0 | 1.5 |
| 4 | V391S | 3.5 | 6.2 | 6.0 | 5.4 | 9.0 |
| 4 | K392R | 2.6 | 5.8 | 7.8 | 7.7 | 4.8 |
| 4 | L393L | 1.1 | 1.0 | 1.1 | 1.1 | 1.1 |
| 4 | R395I | 1.1 | 3.9 | 10.7 | 2.6 | 4.0 |
| 4 | R395C | 0.8 | 2.2 | 4.0 | 2.4 | 4.8 |
| 4 | E396D | 1.8 | 3.5 | 4.9 | 7.5 | 5.0 |
| 4 | D397D | 1.0 | 1.9 | 5.8 | 7.2 | 4.0 |
| 4 | D397C | 0.6 | 1.2 | 6.0 | 4.6 | 1.1 |
| 4 | L398V | 2.4 | 7.3 | 13.2 | 8.9 | 4.5 |
| 4 | R400L | 2.4 | 3.1 | 1.5 | 2.4 | 4.2 |
| 5 | Q402L | 1.1 | 0.5 | 1.1 | 1.3 | 1.8 |
| 5 | R403R | 1.2 | 1.1 | 0.7 | 0.6 | 1.4 |
| 5 | R403R | 1.8 | 2.1 | 2.2 | 2.8 | 2.4 |
| 5 | T404T | 0.9 | 1.1 | 1.0 | 2.1 | 5.0 |
| 5 | T404S | 1.2 | 1.2 | 2.2 | 2.1 | 1.5 |
| 5 | T404A | 1.4 | 1.8 | 2.8 | 2.2 | 2.0 |
| 5 | T404V | 1.5 | 1.7 | 2.1 | 1.6 | 2.3 |
| 5 | T404V | 2.1 | 1.6 | 1.9 | 2.2 | 2.0 |
| 5 | T404S | 1.1 | 2.6 | 2.8 | 1.3 | 1.8 |
| 5 | F405I | 3.8 | 1.5 | 0.7 | 1.0 | 1.6 |
| 5 | F405P | 11.2 | 6.6 | 6.1 | 4.2 | 3.1 |
| 5 | F405G | 1.8 | 0.4 | 1.6 | 2.1 | 1.0 |
| 5 | F405S | 3.8 | 4.6 | 2.6 | 2.8 | 2.7 |
| 5 | F405F | 1.9 | 4.0 | 3.6 | 5.0 | 3.5 |
| 5 | D406R | 1.4 | 0.9 | 1.3 | 1.2 | 1.2 |
| 5 | G408T | 2.4 | 6.1 | 5.5 | 2.4 | 2.4 |
| 5 | G408R | 1.2 | 2.0 | 0.7 | 1.1 | 1.1 |
| 5 | G408E | 2.1 | 2.0 | 1.4 | 1.4 | 1.5 |
| 5 | G408A | 2.2 | 1.9 | 2.6 | 1.4 | 1.2 |
| 5 | G408G | 1.7 | 2.3 | 2.5 | 2.1 | 2.1 |
| 5 | G408C | 1.2 | 2.0 | 1.1 | 1.5 | 1.1 |
| 5 | S409T | 1.9 | 1.5 | 1.1 | 0.9 | 1.1 |
| 5 | S409S | 0.6 | 1.0 | 0.5 | 1.1 | 3.0 |
| 5 | S409L | 7.9 | 6.1 | 1.8 | 3.3 | 1.7 |
| 5 | S409V | 2.0 | 2.3 | 1.2 | 2.2 | 1.9 |
| 5 | S409STOP | 0.9 | 0.4 | 0.4 | 0.4 | 1.1 |
| 5 | S409C | 4.8 | 5.7 | 9.7 | 7.1 | 7.2 |
| 5 | I410I | 2.2 | 0.9 | 2.3 | 2.9 | 1.9 |
| 5 | P411P | 1.2 | 1.2 | 1.3 | 1.1 | 1.0 |
| 5 | Q413Q | 1.1 | 1.2 | 1.1 | 1.3 | 1.2 |
| 5 | Q413H | 1.0 | 1.1 | 1.0 | 1.0 | 1.0 |
| 5 | I414I | 1.3 | 1.1 | 1.1 | 1.3 | 1.0 |
| 5 | I414V | 1.8 | 1.1 | 0.7 | 0.5 | 1.4 |
| 5 | H415Q | 1.0 | 1.3 | 0.5 | 0.9 | 1.0 |
| 5 | L416R | 1.0 | 1.2 | 1.9 | 1.2 | 1.9 |
| 5 | L416L | 0.9 | 1.0 | 0.9 | 1.0 | 1.0 |
| 5 | G417M | 2.1 | 1.5 | 2.2 | 5.4 | 7.9 |
| 5 | G417I | 1.4 | 1.7 | 2.9 | 3.7 | 5.2 |
| 5 | G417L | 0.7 | 2.3 | 4.3 | 5.5 | 4.0 |
| 5 | G417E | 0.7 | 0.7 | 1.0 | 1.3 | 1.3 |
| 5 | G417S | 0.5 | 3.0 | 5.5 | 3.2 | 2.5 |
| 5 | E418V | 1.1 | 1.1 | 0.9 | 0.9 | 1.0 |
| 5 | E418STOP | 1.0 | 0.4 | 0.6 | 0.6 | 1.1 |
| 5 | L419L | 0.6 | 0.8 | 1.8 | 0.7 | 1.6 |
| 5 | L419A | 0.9 | 0.7 | 1.9 | 2.0 | 1.0 |
| 5 | H420N | 2.9 | 1.8 | 2.6 | 4.5 | 8.1 |
| 5 | H420Q | 1.5 | 3.3 | 4.2 | 6.6 | 8.2 |
| 5 | H420R | 1.3 | 2.6 | 4.9 | 6.1 | 7.9 |
| 5 | H420D | 2.3 | 4.4 | 2.9 | 5.3 | 2.9 |
| 5 | H420C | 2.4 | 0.2 | 1.4 | 1.6 | 1.0 |
| 5 | A421T | 1.6 | 2.2 | 1.3 | 2.1 | 2.1 |
| 5 | A421E | 1.3 | 1.2 | 2.3 | 3.8 | 4.8 |
| 5 | A421G | 1.1 | 1.4 | 1.7 | 1.4 | 1.8 |
| 5 | A421S | 0.8 | 1.4 | 2.1 | 4.4 | 5.5 |
| 5 | A421S | 1.1 | 1.2 | 1.7 | 1.5 | 1.0 |
| 5 | A421W | 1.8 | 2.5 | 1.0 | 2.1 | 2.1 |
| 5 | A421C | 2.4 | 1.7 | 2.4 | 2.9 | 2.9 |
| 5 | A421F | 1.0 | 0.3 | 2.7 | 1.6 | 1.5 |
| 5 | I422M | 0.5 | 1.0 | 0.9 | 1.0 | 1.0 |
| 5 | R424K | 4.9 | 8.7 | 8.1 | 10.0 | 11.6 |
| 5 | R424N | 0.8 | 2.3 | 1.4 | 4.5 | 5.1 |
| 5 | R424S | 1.5 | 1.7 | 1.5 | 1.2 | 1.1 |
| 5 | R424D | 0.1 | 0.9 | 2.5 | 2.5 | 4.2 |
| 5 | R424A | 1.0 | 1.4 | 2.0 | 5.1 | 1.2 |
| 5 | R424G | 1.8 | 1.7 | 1.1 | 1.8 | 2.6 |
| 5 | R424S | 1.5 | 3.0 | 3.0 | 2.0 | 3.3 |
| 5 | R424S | 4.4 | 2.4 | 0.9 | 0.5 | 1.2 |
| 5 | R424W | 0.5 | 1.1 | 1.3 | 1.1 | 1.1 |
| 5 | R425R | 1.8 | 1.7 | 1.2 | 1.1 | 1.2 |
| 5 | R425M | 1.4 | 1.3 | 1.8 | 1.4 | 1.9 |
| 5 | R425H | 1.6 | 2.6 | 0.6 | 0.9 | 1.3 |
| 5 | R425R | 0.7 | 1.3 | 1.0 | 1.0 | 1.2 |
| 5 | R425A | 3.0 | 3.3 | 4.2 | 4.1 | 3.6 |
| 5 | R425A | 1.6 | 3.2 | 3.3 | 3.4 | 3.5 |
| 5 | R425G | 1.9 | 1.6 | 2.4 | 1.5 | 1.2 |
| 5 | R425S | 1.2 | 1.6 | 1.1 | 2.4 | 2.3 |
| 5 | Q426H | 0.9 | 0.4 | 0.3 | 0.4 | 1.2 |
| 5 | E427S | 1.0 | 1.3 | 1.7 | 2.7 | 3.8 |
| 5 | E427Q | 1.6 | 2.4 | 2.2 | 3.8 | 3.6 |
| 5 | E427L | 0.2 | 0.4 | 0.7 | 1.3 | 1.1 |
| 5 | E427E | 1.4 | 1.6 | 1.8 | 1.6 | 1.9 |
| 5 | E427A | 0.9 | 1.8 | 2.3 | 4.9 | 13.1 |
| 5 | E427A | 0.5 | 0.4 | 0.8 | 1.6 | 1.3 |
| 5 | E427W | 0.9 | 0.5 | 0.7 | 0.9 | 1.0 |
| 5 | D428K | 1.5 | 0.9 | 1.0 | 1.7 | 2.3 |
| 5 | D428N | 2.1 | 2.6 | 1.2 | 2.1 | 1.3 |
| 5 | D428T | 0.6 | 1.4 | 0.8 | 1.4 | 1.6 |
| 5 | D428T | 0.5 | 0.6 | 0.4 | 0.6 | 1.1 |
| 5 | D428R | 1.2 | 1.5 | 1.5 | 1.9 | 2.0 |
| 5 | D428M | 1.6 | 1.9 | 1.8 | 2.2 | 1.5 |
| 5 | D428H | 2.2 | 1.1 | 2.2 | 5.2 | 8.4 |
| 5 | D428P | 1.4 | 2.0 | 1.9 | 1.8 | 3.2 |
| 5 | D428R | 1.7 | 2.1 | 3.0 | 4.2 | 3.6 |
| 5 | D428L | 1.4 | 1.8 | 0.7 | 1.4 | 2.0 |
| 5 | D428A | 0.9 | 1.1 | 1.6 | 1.6 | 2.3 |
| 5 | D428G | 1.3 | 1.3 | 1.3 | 1.3 | 1.3 |
| 5 | D428G | 1.6 | 0.9 | 0.7 | 0.8 | 1.0 |
| 5 | D428V | 0.8 | 2.0 | 2.3 | 1.7 | 1.4 |
| 5 | D428S | 1.5 | 2.2 | 2.4 | 1.8 | 2.7 |
| 5 | D428S | 1.6 | 2.4 | 2.8 | 5.0 | 4.9 |
| 5 | D428C | 1.0 | 1.2 | 1.4 | 2.6 | 1.6 |
| 5 | F429Y | 1.7 | 2.4 | 3.0 | 2.6 | 2.2 |
| 5 | Y430Y | 1.5 | 1.3 | 0.8 | 0.8 | 2.0 |
| 5 | Y430W | 1.5 | 4.5 | 3.6 | 4.8 | 5.1 |
| 5 | P431T | 1.0 | 0.9 | 0.7 | 0.5 | 1.8 |
| 5 | P431H | 1.6 | 0.3 | 0.2 | 0.3 | 2.0 |
| 5 | K434R | 1.0 | 1.0 | 1.0 | 1.1 | 1.5 |
| 5 | K434R | 0.7 | 0.3 | 2.0 | 3.5 | 3.3 |
| 5 | K434E | 0.7 | 1.0 | 0.9 | 1.3 | 1.7 |
| 5 | K434G | 1.4 | 1.6 | 1.2 | 1.7 | 1.9 |
| 5 | K434S | 0.8 | 0.8 | 1.9 | 2.9 | 3.5 |
| 5 | D435N | 2.3 | 1.6 | 1.0 | 1.0 | 1.3 |
| 5 | D435L | 0.3 | 4.0 | 5.2 | 5.7 | 4.7 |
| 5 | D435F | 5.5 | 2.4 | 6.4 | 6.4 | 7.4 |
| 5 | N436K | 1.1 | 1.0 | 1.1 | 1.0 | 1.1 |
| 5 | N436N | 3.1 | 3.6 | 3.1 | 1.1 | 1.1 |
| 5 | N436D | 0.7 | 2.0 | 3.3 | 1.3 | 1.0 |
| 5 | R437K | 0.2 | 0.8 | 4.3 | 1.5 | 1.7 |
| 5 | R437S | 1.1 | 0.7 | 1.3 | 1.1 | 2.5 |
| 5 | R437R | 2.9 | 4.6 | 3.3 | 2.4 | 1.8 |
| 5 | R437L | 1.9 | 0.6 | 0.5 | 0.5 | 3.8 |
| 5 | R437A | 1.8 | 4.4 | 2.1 | 4.3 | 3.6 |
| 5 | R437S | 1.4 | 1.4 | 0.7 | 2.7 | 6.5 |
| 5 | R437F | 1.1 | 1.7 | 1.4 | 3.3 | 3.0 |
| 5 | E438R | 0.8 | 2.2 | 2.2 | 1.7 | 1.1 |
| 5 | E438Q | 2.5 | 0.3 | 0.5 | 1.3 | 3.7 |
| 5 | K439N | 1.4 | 1.6 | 1.4 | 1.4 | 1.3 |
| 5 | K439R | 0.7 | 2.7 | 3.3 | 1.3 | 2.0 |
| 5 | I440L | 2.5 | 2.7 | 1.3 | 1.0 | 1.1 |
| 5 | E441D | 0.6 | 0.5 | 1.0 | 1.4 | 1.4 |
| 5 | E441A | 1.4 | 2.2 | 2.4 | 2.2 | 1.7 |
| 5 | K442R | 1.3 | 0.8 | 1.1 | 2.0 | 1.9 |
| 5 | K442R | 1.4 | 1.4 | 2.0 | 2.0 | 1.4 |
| 5 | L444I | 0.7 | 0.9 | 1.5 | 1.3 | 1.4 |
| 5 | L444L | 2.2 | 1.4 | 2.6 | 4.3 | 2.9 |
| 5 | T445Q | 2.0 | 2.9 | 9.0 | 4.1 | 2.6 |
| 5 | F446Y | 1.1 | 0.9 | 1.1 | 1.0 | 1.0 |
| 5 | F446S | 0.9 | 0.8 | 0.9 | 0.8 | 1.0 |
| 5 | R447R | 1.6 | 1.6 | 0.9 | 0.9 | 1.1 |
| 5 | R447A | 1.5 | 0.4 | 1.2 | 1.4 | 1.0 |
| 5 | P449P | 1.7 | 1.2 | 0.5 | 0.8 | 2.0 |
| 5 | Y450H | 1.3 | 1.5 | 1.3 | 1.6 | 1.2 |
| 5 | Y450A | 1.5 | 1.8 | 3.6 | 11.0 | 13.9 |
| 5 | Y450S | 1.3 | 4.3 | 3.2 | 1.5 | 2.8 |
| 5 | Y450W | 0.7 | 1.8 | 2.3 | 3.7 | 1.2 |
| 5 | Y450C | 2.1 | 2.3 | 2.7 | 1.6 | 1.5 |
| 5 | Y451STOP | 1.0 | 0.9 | 0.9 | 0.9 | 1.2 |
| 5 | V452V | 1.6 | 2.2 | 2.1 | 1.8 | 1.1 |
| 5 | G453G | 1.2 | 1.3 | 1.3 | 1.3 | 1.6 |
| 5 | L455L | 0.4 | 1.2 | 0.9 | 1.3 | 1.0 |
| 5 | L455L | 2.6 | 5.6 | 6.0 | 8.7 | 10.8 |
| 5 | A456A | 0.8 | 0.8 | 0.9 | 1.2 | 1.2 |
| 5 | A456S | 2.5 | 2.3 | 1.8 | 1.7 | 1.4 |
| 5 | R457K | 2.1 | 1.8 | 0.9 | 5.6 | 1.8 |
| 5 | R457N | 2.5 | 3.7 | 5.1 | 1.4 | 1.0 |
| 5 | R457S | 4.2 | 3.3 | 8.1 | 10.9 | 15.2 |
| 5 | R457M | 9.3 | 14.4 | 18.2 | 9.2 | 5.4 |
| 5 | R457R | 2.7 | 3.8 | 2.0 | 1.3 | 5.8 |
| 5 | R457L | 2.1 | 0.8 | 0.3 | 0.4 | 5.9 |
| 5 | R457A | 4.2 | 4.1 | 4.7 | 5.6 | 7.5 |
| 5 | R457V | 1.6 | 3.0 | 3.9 | 4.5 | 8.0 |
| 5 | R457S | 0.6 | 3.6 | 5.9 | 5.8 | 9.3 |
| 5 | G458R | 2.2 | 3.9 | 3.1 | 2.7 | 1.2 |
| 5 | G458E | 0.7 | 0.4 | 0.6 | 0.8 | 1.0 |
| 5 | G458G | 1.5 | 1.4 | 1.5 | 1.4 | 1.9 |
| 5 | G458V | 1.0 | 0.4 | 0.9 | 1.0 | 1.2 |
| 5 | G458S | 6.9 | 7.8 | 4.5 | 2.2 | 1.5 |
| 5 | N459T | 0.9 | 1.2 | 2.2 | 1.4 | 2.3 |
| 5 | N459S | 1.4 | 1.1 | 1.2 | 1.3 | 1.0 |
| 5 | N459M | 1.0 | 1.9 | 1.6 | 0.7 | 1.0 |
| 5 | N459P | 1.4 | 2.3 | 3.6 | 6.6 | 3.6 |
| 5 | N459A | 2.5 | 5.0 | 5.2 | 5.1 | 10.4 |
| 5 | N459G | 0.8 | 1.4 | 2.9 | 3.7 | 5.0 |
| 5 | N459Y | 0.9 | 1.0 | 1.7 | 1.6 | 1.3 |
| 5 | N459W | 1.3 | 1.3 | 2.1 | 1.7 | 2.1 |
| 5 | R461S | 2.2 | 0.6 | 1.0 | 0.3 | 1.4 |
| 5 | F462H | 2.2 | 2.9 | 3.7 | 4.6 | 2.4 |
| 5 | F462Y | 1.6 | 1.9 | 1.7 | 1.5 | 1.5 |
| 5 | A463S | 0.8 | 0.4 | 1.1 | 1.3 | 2.4 |
| 5 | A463S | 1.1 | 1.3 | 0.8 | 1.0 | 1.9 |
| 5 | M465L | 1.6 | 1.0 | 1.2 | 1.7 | 1.3 |
| 5 | T466K | 1.4 | 1.9 | 1.8 | 2.0 | 1.1 |
| 5 | T466T | 0.9 | 0.8 | 1.1 | 1.9 | 2.3 |
| 5 | T466T | 1.1 | 1.1 | 1.1 | 1.1 | 1.3 |
| 5 | T466H | 1.4 | 1.5 | 2.2 | 2.1 | 3.1 |
| 5 | T466E | 0.7 | 2.5 | 4.8 | 3.3 | 2.4 |
| 5 | T466A | 1.4 | 1.9 | 2.8 | 1.5 | 1.4 |
| 5 | T466V | 1.1 | 0.4 | 0.6 | 1.0 | 1.2 |
| 5 | T466S | 3.3 | 6.1 | 5.7 | 2.8 | 2.3 |
| 5 | K468R | 0.9 | 1.1 | 0.7 | 0.8 | 1.0 |
| 5 | K468M | 1.3 | 1.3 | 1.3 | 1.3 | 1.3 |
| 5 | K468STOP | 0.9 | 1.0 | 1.1 | 1.1 | 1.3 |
| 5 | S469K | 2.2 | 2.4 | 1.9 | 1.7 | 1.2 |
| 5 | S469T | 0.5 | 0.7 | 1.2 | 1.0 | 1.3 |
| 5 | S469L | 1.3 | 1.7 | 1.1 | 0.9 | 1.1 |
| 5 | S469S | 1.0 | 1.6 | 1.5 | 1.0 | 1.1 |
| 5 | E470R | 0.6 | 1.2 | 1.8 | 1.8 | 1.6 |
| 5 | E470D | 1.5 | 1.7 | 1.6 | 2.0 | 1.9 |
| 5 | E470A | 0.7 | 1.2 | 2.5 | 1.2 | 1.0 |
| 5 | E470A | 2.1 | 3.4 | 1.3 | 2.1 | 1.4 |
| 5 | E470G | 0.9 | 1.0 | 1.1 | 1.4 | 1.2 |
| 5 | E470S | 2.1 | 2.5 | 1.8 | 3.6 | 3.5 |
| 5 | E471A | 1.5 | 1.1 | 3.0 | 2.4 | 1.2 |
| 5 | E471V | 0.7 | 0.9 | 0.9 | 0.9 | 1.2 |
| 5 | T472T | 1.0 | 1.2 | 1.4 | 1.6 | 1.6 |
| 5 | T472R | 1.5 | 3.2 | 4.0 | 3.6 | 4.6 |
| 5 | T472S | 0.9 | 4.7 | 4.8 | 6.2 | 5.0 |
| 5 | T472R | 2.1 | 1.4 | 1.9 | 1.1 | 1.3 |
| 5 | T472R | 0.4 | 1.6 | 0.8 | 1.9 | 2.9 |
| 5 | T472A | 1.7 | 2.2 | 2.5 | 4.4 | 5.2 |
| 5 | T472G | 0.8 | 1.0 | 1.1 | 1.6 | 1.3 |
| 5 | T472S | 0.9 | 1.0 | 1.0 | 1.2 | 1.2 |
| 5 | T472S | 1.5 | 2.2 | 3.5 | 3.8 | 10.4 |
| 5 | I473I | 0.9 | 1.0 | 0.9 | 0.7 | 1.0 |
| 5 | I473V | 1.1 | 0.6 | 1.1 | 1.1 | 1.3 |
| 5 | T474K | 1.3 | 2.3 | 3.0 | 3.8 | 1.6 |
| 5 | T474T | 1.1 | 1.8 | 0.7 | 1.9 | 3.3 |
| 5 | T474R | 6.9 | 5.5 | 10.6 | 5.9 | 4.9 |
| 5 | T474R | 3.2 | 2.9 | 1.4 | 1.0 | 1.7 |
| 5 | P475T | 0.5 | 0.3 | 0.3 | 0.3 | 1.8 |
| 5 | P475Q | 0.7 | 0.3 | 0.2 | 0.2 | 1.7 |
| 5 | P475P | 1.3 | 1.5 | 2.5 | 1.5 | 1.8 |
| 5 | E479E | 1.5 | 2.5 | 2.4 | 2.3 | 1.9 |
| 5 | E479D | 1.2 | 1.5 | 1.3 | 1.4 | 1.6 |
| 5 | E480Q | 1.8 | 3.7 | 6.0 | 8.8 | 6.4 |
| 5 | E480R | 0.7 | 1.8 | 1.8 | 0.8 | 2.4 |
| 5 | E480L | 0.7 | 2.6 | 3.4 | 3.9 | 2.9 |
| 5 | V481V | 1.3 | 1.1 | 1.2 | 1.9 | 2.0 |
| 5 | V481C | 2.6 | 3.6 | 1.9 | 0.8 | 1.4 |
| 5 | V482V | 1.1 | 1.1 | 1.1 | 1.1 | 1.1 |
| 5 | V482V | 1.2 | 1.1 | 1.1 | 1.2 | 1.3 |
| 5 | D483D | 2.0 | 2.9 | 2.6 | 3.8 | 3.1 |
| 5 | K484R | 1.4 | 1.7 | 1.6 | 1.3 | 1.5 |
| 5 | K484R | 0.7 | 1.4 | 2.4 | 3.6 | 3.9 |
| 5 | K484L | 3.6 | 9.5 | 11.6 | 12.0 | 14.5 |
| 5 | K484V | 1.4 | 2.3 | 2.7 | 5.3 | 3.4 |
| 5 | G485K | 2.0 | 3.0 | 1.2 | 1.7 | 4.0 |
| 5 | G485H | 1.7 | 1.4 | 4.4 | 5.5 | 2.6 |
| 5 | G485P | 4.9 | 12.5 | 17.5 | 9.3 | 12.0 |
| 5 | G485D | 1.0 | 2.2 | 1.7 | 2.1 | 2.4 |
| 5 | G485A | 1.7 | 1.8 | 1.9 | 1.3 | 2.0 |
| 5 | G485G | 1.8 | 1.3 | 1.7 | 1.6 | 1.7 |
| 5 | G485Y | 3.9 | 5.7 | 10.3 | 9.0 | 6.5 |
| 5 | G485S | 8.4 | 7.4 | 11.2 | 7.9 | 4.0 |
| 5 | A486K | 1.9 | 3.3 | 2.4 | 3.6 | 3.8 |
| 5 | A486R | 1.5 | 1.8 | 3.2 | 3.0 | 2.5 |
| 5 | A486M | 1.0 | 0.8 | 0.6 | 2.1 | 2.5 |
| 5 | A486Q | 1.2 | 1.4 | 0.8 | 2.1 | 1.5 |
| 5 | A486R | 1.1 | 0.8 | 1.1 | 1.8 | 1.1 |
| 5 | A486A | 1.0 | 1.0 | 1.2 | 1.2 | 1.1 |
| 5 | A486V | 1.1 | 1.0 | 0.8 | 1.5 | 1.3 |
| 5 | S487S | 1.1 | 0.9 | 1.3 | 0.7 | 1.5 |
| 5 | A488S | 4.9 | 2.0 | 0.6 | 1.4 | 1.0 |
| 5 | A488A | 1.0 | 0.9 | 0.9 | 0.6 | 1.1 |
| 5 | Q489R | 0.5 | 0.3 | 0.6 | 0.7 | 1.3 |
| 5 | Q489L | 1.1 | 1.3 | 1.0 | 1.1 | 1.0 |
| 5 | Q489L | 10.7 | 4.0 | 8.3 | 4.5 | 4.1 |
| 5 | Q489A | 2.0 | 1.6 | 1.1 | 1.0 | 1.1 |
| 5 | Q489S | 1.1 | 1.1 | 1.0 | 1.5 | 1.2 |
| 5 | Q489L | 2.5 | 3.2 | 3.0 | 4.5 | 3.3 |
| 5 | Q489F | 4.0 | 1.1 | 1.3 | 3.0 | 3.3 |
| 5 | S490S | 1.7 | 1.2 | 0.4 | 0.8 | 1.6 |
| 5 | S490R | 2.0 | 2.3 | 1.9 | 1.9 | 1.8 |
| 5 | S490L | 1.0 | 1.3 | 2.8 | 4.0 | 1.3 |
| 5 | S490E | 1.3 | 0.9 | 1.4 | 1.9 | 1.5 |
| 5 | S490G | 0.6 | 0.4 | 0.2 | 0.7 | 1.1 |
| 5 | S490G | 0.9 | 1.2 | 1.1 | 1.2 | 1.3 |
| 5 | S490S | 1.3 | 1.1 | 1.0 | 1.3 | 1.2 |
| 5 | F491Y | 2.4 | 2.9 | 3.1 | 3.1 | 3.0 |
| 5 | A488S | 5.1 | 2.2 | 0.5 | 1.1 | 1.0 |
| 5 | Q489R | 1.0 | 1.1 | 1.0 | 1.1 | 1.0 |
| 5 | Q489R | 0.7 | 0.4 | 0.7 | 0.7 | 2.2 |
| 5 | Q489L | 1.7 | 1.5 | 2.2 | 1.1 | 1.2 |
| 5 | Q489A | 2.1 | 1.5 | 1.3 | 1.1 | 1.1 |
| 5 | Q489S | 0.9 | 0.9 | 0.9 | 1.4 | 1.0 |
| 5 | Q489L | 2.4 | 3.1 | 2.9 | 4.1 | 3.3 |
| 5 | Q489F | 2.4 | 0.8 | 0.8 | 1.9 | 2.5 |
| 5 | S490S | 1.9 | 0.9 | 0.3 | 0.7 | 1.7 |
| 5 | S490R | 1.7 | 1.7 | 1.7 | 1.8 | 1.9 |
| 5 | S490L | 1.2 | 1.4 | 3.0 | 3.8 | 1.6 |
| 5 | S490E | 1.1 | 0.8 | 1.6 | 1.8 | 1.5 |
| 5 | S490G | 0.5 | 0.4 | 0.3 | 0.5 | 1.1 |
| 5 | S490G | 0.8 | 1.1 | 0.9 | 0.9 | 1.2 |
| 5 | S490S | 1.3 | 1.1 | 1.1 | 1.3 | 1.2 |
| 5 | F491Y | 2.1 | 2.3 | 2.8 | 2.3 | 2.9 |
| 5 | F491W | 1.0 | 2.8 | 1.5 | 0.8 | 1.0 |
| 5 | I492T | 0.9 | 1.7 | 1.7 | 1.4 | 1.1 |
| 5 | I492R | 0.9 | 1.7 | 0.8 | 1.2 | 1.3 |
| 5 | I492L | 1.0 | 1.6 | 2.9 | 3.4 | 2.1 |
| 5 | I492A | 1.4 | 3.5 | 2.5 | 5.4 | 3.0 |
| 5 | I492G | 1.5 | 1.3 | 1.0 | 1.2 | 1.3 |
| 5 | I492V | 2.0 | 2.9 | 4.2 | 3.2 | 3.8 |
| 5 | I492S | 1.2 | 1.4 | 1.3 | 0.6 | 2.4 |
| 5 | E493M | 1.8 | 5.2 | 7.7 | 5.9 | 5.7 |
| 5 | E493E | 1.3 | 1.5 | 1.7 | 1.4 | 1.0 |
| 5 | E493G | 1.7 | 1.9 | 2.8 | 2.2 | 1.8 |
| 5 | E493G | 6.7 | 6.5 | 6.2 | 2.5 | 3.4 |
| 5 | E493L | 4.0 | 0.1 | 2.2 | 5.2 | 1.1 |
| 5 | R494H | 1.3 | 2.1 | 1.4 | 1.4 | 1.2 |
| 5 | M495H | 2.7 | 1.1 | 0.3 | 1.1 | 1.8 |
| 5 | M495L | 1.2 | 1.0 | 1.2 | 1.0 | 1.1 |
| 5 | M495E | 0.1 | 0.5 | 1.8 | 2.6 | 1.0 |
| 5 | M495A | 0.7 | 0.8 | 2.5 | 2.1 | 1.7 |
| 5 | M495V | 2.6 | 2.3 | 2.8 | 3.5 | 2.2 |
| 5 | M495S | 0.5 | 2.6 | 1.9 | 0.6 | 1.3 |
| 5 | M495S | 1.1 | 2.7 | 2.4 | 4.8 | 3.7 |
| 5 | M495W | 0.6 | 0.6 | 0.4 | 0.7 | 1.0 |
| 5 | T496R | 2.0 | 3.7 | 2.7 | 1.7 | 1.9 |
| 5 | T496M | 1.8 | 2.2 | 2.3 | 3.6 | 4.1 |
| 5 | T496R | 3.2 | 2.4 | 1.6 | 2.2 | 3.3 |
| 5 | T496L | 1.3 | 0.5 | 2.4 | 1.8 | 2.2 |
| 5 | T496A | 1.9 | 1.6 | 1.9 | 1.6 | 1.9 |
| 5 | T496A | 2.2 | 2.2 | 0.3 | 1.5 | 3.1 |
| 5 | T496G | 0.5 | 0.2 | 0.2 | 0.4 | 1.1 |
| 5 | T496S | 0.9 | 1.1 | 1.0 | 1.0 | 1.0 |
| 5 | T496L | 3.3 | 4.5 | 4.2 | 5.6 | 4.3 |
| 5 | T496F | 2.9 | 2.1 | 3.7 | 9.9 | 7.5 |
| 5 | N497K | 1.4 | 1.7 | 1.9 | 2.2 | 1.8 |
| 5 | N497T | 1.4 | 2.4 | 1.5 | 2.5 | 2.9 |
| 5 | N497R | 1.0 | 1.4 | 0.9 | 1.6 | 1.4 |
| 5 | N497M | 1.8 | 2.6 | 3.3 | 3.0 | 2.5 |
| 5 | N497I | 1.1 | 1.1 | 1.3 | 0.6 | 1.0 |
| 5 | N497Q | 1.6 | 1.3 | 2.2 | 2.8 | 2.8 |
| 5 | N497E | 0.7 | 1.2 | 1.5 | 1.5 | 1.5 |
| 5 | N497D | 1.1 | 1.0 | 1.1 | 1.0 | 1.4 |
| 5 | N497S | 1.4 | 2.2 | 2.5 | 1.7 | 2.6 |
| 5 | N497S | 0.8 | 0.4 | 1.1 | 1.5 | 1.0 |
| 5 | N497W | 1.2 | 0.8 | 0.3 | 1.6 | 2.0 |
| 5 | N497F | 0.8 | 1.1 | 1.0 | 1.2 | 1.0 |
| 5 | F498K | 0.3 | 2.8 | 3.1 | 6.3 | 11.1 |
| 5 | F498P | 0.1 | 1.2 | 5.3 | 3.8 | 5.2 |
| 5 | F498P | 0.3 | 0.7 | 7.0 | 9.1 | 1.7 |
| 5 | F498G | 1.3 | 2.5 | 1.8 | 1.1 | 2.9 |
| 5 | F498V | 2.3 | 2.7 | 4.1 | 2.8 | 3.8 |
| 5 | F498C | 0.6 | 2.3 | 3.0 | 1.8 | 2.0 |
| 5 | K500Q | 2.4 | 2.0 | 3.4 | 9.2 | 9.5 |
| 5 | K500C | 4.2 | 3.8 | 6.1 | 4.4 | 2.8 |
| 5 | K500F | 5.0 | 2.9 | 2.9 | 1.8 | 2.6 |
| 6 | N501T | 1.7 | 2.3 | 1.8 | 3.5 | 2.3 |
| 6 | N501R | 1.9 | 1.7 | 2.2 | 2.8 | 2.1 |
| 6 | N501I | 1.2 | 0.9 | 3.2 | 4.3 | 3.6 |
| 6 | N501Q | 1.2 | 1.1 | 3.2 | 3.0 | 1.2 |
| 6 | N501L | 2.7 | 5.1 | 5.8 | 8.4 | 10.3 |
| 6 | N501A | 0.7 | 1.8 | 1.3 | 2.9 | 5.7 |
| 6 | N501V | 1.2 | 1.2 | 3.4 | 5.5 | 5.4 |
| 6 | N501V | 1.0 | 2.0 | 1.9 | 2.0 | 1.3 |
| 6 | N501S | 2.3 | 3.7 | 2.9 | 3.6 | 3.9 |
| 6 | N501W | 1.4 | 1.5 | 5.1 | 8.6 | 7.8 |
| 6 | N501C | 1.2 | 2.1 | 2.8 | 2.4 | 2.5 |
| 6 | L502M | 2.1 | 2.3 | 3.0 | 1.9 | 1.7 |
| 6 | L502I | 2.8 | 1.5 | 3.9 | 3.1 | 2.5 |
| 6 | L502L | 1.5 | 2.0 | 0.2 | 4.0 | 2.3 |
| 6 | L502V | 1.3 | 1.2 | 1.2 | 1.3 | 1.8 |
| 6 | L502W | 1.1 | 1.2 | 3.6 | 3.0 | 3.0 |
| 6 | P503R | 0.3 | 1.6 | 0.6 | 0.6 | 1.5 |
| 6 | P503Q | 0.7 | 1.4 | 12.2 | 5.3 | 5.5 |
| 6 | P503P | 2.2 | 1.9 | 2.4 | 4.0 | 2.4 |
| 6 | P503L | 1.4 | 2.5 | 0.9 | 1.3 | 1.1 |
| 6 | P503Y | 1.2 | 2.0 | 6.5 | 6.8 | 9.0 |
| 6 | N504R | 1.8 | 2.0 | 4.1 | 3.9 | 4.5 |
| 6 | N504S | 1.7 | 3.2 | 5.3 | 7.8 | 9.6 |
| 6 | N504Q | 2.6 | 1.8 | 4.4 | 1.4 | 1.4 |
| 6 | N504E | 1.6 | 2.9 | 3.1 | 3.2 | 4.0 |
| 6 | N504A | 1.7 | 3.2 | 4.9 | 6.4 | 8.1 |
| 6 | N504G | 1.2 | 2.4 | 3.1 | 1.9 | 1.6 |
| 6 | N504S | 2.3 | 4.5 | 7.6 | 5.4 | 6.1 |
| 6 | N504C | 2.1 | 3.3 | 4.5 | 8.1 | 8.5 |
| 6 | E505C | 1.4 | 1.0 | 6.9 | 1.1 | 1.3 |
| 6 | K506I | 1.9 | 1.8 | 1.6 | 0.9 | 1.2 |
| 6 | K506R | 2.5 | 1.3 | 3.4 | 4.5 | 4.9 |
| 6 | K506R | 2.0 | 3.8 | 2.3 | 2.0 | 1.3 |
| 6 | K506G | 0.8 | 0.6 | 1.7 | 1.3 | 2.1 |
| 6 | K506C | 0.9 | 0.5 | 0.7 | 1.0 | 1.7 |
| 6 | V507T | 1.2 | 1.7 | 1.9 | 1.2 | 2.4 |
| 6 | V507R | 1.3 | 1.2 | 1.6 | 1.6 | 1.1 |
| 6 | V507A | 1.4 | 1.2 | 0.8 | 1.1 | 1.0 |
| 6 | V507V | 1.1 | 1.7 | 1.3 | 2.6 | 2.1 |
| 6 | V507C | 1.5 | 2.2 | 2.0 | 4.9 | 4.1 |
| 6 | L508L | 2.7 | 3.6 | 5.4 | 8.8 | 10.6 |
| 6 | P509S | 1.7 | 1.1 | 2.5 | 2.9 | 7.7 |
| 6 | P509P | 1.5 | 2.4 | 2.0 | 5.5 | 3.1 |
| 6 | K510N | 1.4 | 2.5 | 1.6 | 6.0 | 7.8 |
| 6 | K510R | 0.4 | 1.2 | 1.8 | 5.9 | 8.3 |
| 6 | K510L | 1.9 | 4.5 | 10.5 | 12.1 | 13.4 |
| 6 | K510E | 0.8 | 2.3 | 0.2 | 1.9 | 1.1 |
| 6 | K510G | 2.8 | 2.3 | 2.9 | 2.7 | 2.7 |
| 6 | K510S | 1.9 | 4.1 | 3.2 | 3.4 | 3.9 |
| 6 | H511N | 1.5 | 1.8 | 0.6 | 1.9 | 1.7 |
| 6 | H511R | 0.9 | 1.0 | 1.2 | 1.0 | 1.1 |
| 6 | H511Q | 1.2 | 1.6 | 2.7 | 2.4 | 3.6 |
| 6 | H511P | 1.2 | 1.2 | 1.7 | 2.8 | 4.2 |
| 6 | H511R | 1.7 | 1.5 | 1.9 | 0.9 | 1.6 |
| 6 | H511A | 1.2 | 1.1 | 4.6 | 6.9 | 6.3 |
| 6 | H511S | 0.8 | 2.2 | 7.0 | 2.5 | 1.9 |
| 6 | S512S | 0.7 | 1.2 | 5.8 | 16.3 | 16.2 |
| 6 | L513N | 2.0 | 3.1 | 3.1 | 2.9 | 3.7 |
| 6 | L513T | 2.2 | 1.1 | 1.4 | 0.4 | 1.2 |
| 6 | L513Q | 1.2 | 0.5 | 4.1 | 4.4 | 4.3 |
| 6 | L513H | 1.0 | 0.7 | 3.3 | 3.8 | 4.8 |
| 6 | L513P | 1.7 | 3.2 | 3.1 | 3.1 | 5.2 |
| 6 | L513A | 1.6 | 1.2 | 1.4 | 1.7 | 2.4 |
| 6 | L513V | 1.4 | 1.3 | 2.7 | 3.0 | 3.1 |
| 6 | L513C | 1.9 | 1.6 | 6.2 | 8.9 | 6.9 |
| 6 | L514T | 1.1 | 0.3 | 4.4 | 7.2 | 9.1 |
| 6 | L514S | 0.8 | 0.1 | 1.6 | 8.6 | 12.4 |
| 6 | L514L | 1.6 | 2.7 | 5.0 | 1.9 | 2.9 |
| 6 | L514V | 1.8 | 2.2 | 1.7 | 2.4 | 2.7 |
| 6 | L514V | 1.5 | 1.8 | 0.3 | 2.0 | 1.3 |
| 6 | Y515N | 1.1 | 1.0 | 11.2 | 16.2 | 18.5 |
| 6 | Y515R | 1.4 | 0.9 | 2.2 | 2.5 | 1.3 |
| 6 | Y515M | 0.9 | 2.3 | 4.1 | 4.7 | 7.5 |
| 6 | Y515H | 1.0 | 0.5 | 11.4 | 4.2 | 3.3 |
| 6 | Y515V | 2.7 | 3.2 | 6.5 | 7.9 | 3.1 |
| 6 | E516E | 1.6 | 1.4 | 2.4 | 5.1 | 4.0 |
| 6 | E516A | 0.8 | 2.5 | 10.3 | 1.7 | 1.8 |
| 6 | Y517N | 2.6 | 3.3 | 1.8 | 2.4 | 1.5 |
| 6 | Y517L | 1.7 | 21.6 | 58.6 | 51.9 | 81.7 |
| 6 | Y517S | 0.6 | 0.1 | 1.6 | 4.5 | 1.7 |
| 6 | Y517W | 1.1 | 0.9 | 1.5 | 1.6 | 1.1 |
| 6 | Y517C | 1.6 | 3.1 | 6.2 | 7.3 | 7.9 |
| 6 | Y517F | 1.5 | 1.3 | 1.7 | 1.4 | 2.9 |
| 6 | T519N | 3.7 | 7.0 | 39.5 | 26.9 | 37.4 |
| 6 | T519L | 2.3 | 0.0 | 1.9 | 2.8 | 1.1 |
| 6 | V520I | 2.5 | 3.0 | 4.2 | 2.4 | 1.3 |
| 6 | V520V | 1.3 | 0.3 | 1.8 | 4.0 | 5.8 |
| 6 | V520C | 2.3 | 5.3 | 13.2 | 29.5 | 20.7 |
| 6 | Y521Y | 1.3 | 1.5 | 1.7 | 2.0 | 1.4 |
| 6 | N522I | 1.6 | 0.1 | 20.6 | 0.3 | 1.3 |
| 6 | N522G | 1.5 | 1.6 | 4.7 | 4.6 | 4.2 |
| 6 | E523I | 1.1 | 4.2 | 49.4 | 11.9 | 9.5 |
| 6 | L524L | 1.4 | 1.2 | 3.4 | 7.6 | 5.5 |
| 6 | T525S | 0.9 | 1.8 | 1.8 | 2.0 | 3.2 |
| 6 | T525C | 2.4 | 3.6 | 4.4 | 5.2 | 5.8 |
| 6 | K526S | 1.6 | 1.9 | 2.9 | 7.2 | 8.9 |
| 6 | K526F | 1.1 | 1.7 | 0.7 | 1.9 | 2.0 |
| 6 | V527I | 2.8 | 3.7 | 3.4 | 2.4 | 3.6 |
| 6 | V527C | 1.6 | 2.1 | 3.5 | 3.3 | 4.3 |
| 6 | K528M | 1.4 | 2.5 | 8.2 | 7.1 | 7.2 |
| 6 | K528Q | 0.7 | 0.3 | 1.8 | 4.5 | 3.2 |
| 6 | K528L | 1.1 | 6.0 | 9.9 | 26.8 | 36.0 |
| 6 | K528E | 4.7 | 4.8 | 22.8 | 7.5 | 6.8 |
| 6 | K528A | 3.6 | 8.8 | 14.4 | 14.7 | 9.3 |
| 6 | K528F | 1.9 | 4.2 | 11.1 | 6.3 | 8.7 |
| 6 | Y529R | 1.9 | 1.5 | 7.1 | 11.4 | 7.5 |
| 6 | V530T | 0.7 | 2.4 | 4.3 | 9.4 | 9.7 |
| 6 | V530M | 1.1 | 1.7 | 4.5 | 4.3 | 5.5 |
| 6 | V530R | 2.4 | 4.7 | 20.1 | 14.7 | 13.7 |
| 6 | V530L | 3.1 | 5.3 | 23.0 | 10.4 | 8.1 |
| 6 | V530D | 1.9 | 2.6 | 6.4 | 3.1 | 2.1 |
| 6 | V530V | 0.9 | 0.9 | 2.6 | 3.5 | 4.3 |
| 6 | V530S | 3.0 | 4.3 | 2.3 | 5.9 | 11.8 |
| 6 | V530L | 2.2 | 1.8 | 5.0 | 5.4 | 5.7 |
| 6 | T531M | 1.8 | 1.3 | 1.2 | 1.3 | 1.0 |
| 6 | T531Q | 3.3 | 1.6 | 13.2 | 14.8 | 18.4 |
| 6 | T531G | 2.7 | 3.7 | 41.8 | 53.5 | 37.1 |
| 6 | T531S | 2.6 | 10.7 | 18.8 | 16.2 | 16.7 |
| 6 | E532K | 2.4 | 16.6 | 14.1 | 16.7 | 12.0 |
| 6 | E532E | 2.5 | 2.6 | 4.8 | 5.4 | 10.8 |
| 6 | E532V | 1.4 | 2.5 | 2.3 | 1.4 | 2.1 |
| 6 | E532W | 1.3 | 2.1 | 5.8 | 9.6 | 4.3 |
| 6 | G533G | 0.9 | 0.9 | 3.2 | 2.6 | 3.4 |
| 6 | M534K | 1.3 | 0.4 | 1.2 | 0.2 | 1.4 |
| 6 | M534T | 2.2 | 2.2 | 3.9 | 2.5 | 1.2 |
| 6 | M534R | 1.0 | 0.9 | 1.9 | 3.3 | 2.8 |
| 6 | M534G | 12.0 | 10.0 | 11.1 | 29.8 | 13.8 |
| 6 | M534V | 1.1 | 0.5 | 2.1 | 2.3 | 1.7 |
| 6 | M534C | 1.1 | 1.9 | 3.1 | 3.1 | 2.5 |
| 6 | R535R | 1.6 | 3.0 | 6.1 | 4.5 | 3.6 |
| 6 | R535R | 1.5 | 3.0 | 4.8 | 6.1 | 8.1 |
| 6 | R535R | 1.8 | 0.6 | 0.4 | 3.8 | 4.0 |
| 6 | R535L | 3.3 | 7.0 | 4.8 | 18.8 | 28.0 |
| 6 | R535D | 5.6 | 0.6 | 17.9 | 29.8 | 28.4 |
| 6 | R535A | 1.1 | 3.4 | 6.8 | 3.2 | 1.1 |
| 6 | R535G | 1.6 | 2.9 | 0.2 | 5.2 | 3.6 |
| 6 | R535G | 1.4 | 2.8 | 5.7 | 3.1 | 2.4 |
| 6 | R535W | 0.9 | 0.9 | 2.2 | 0.8 | 1.0 |
| 6 | K536N | 1.6 | 1.8 | 8.4 | 16.7 | 15.8 |
| 6 | K536S | 1.8 | 3.9 | 3.4 | 2.1 | 1.9 |
| 6 | K536M | 2.2 | 0.7 | 2.6 | 6.2 | 4.5 |
| 6 | K536R | 1.5 | 3.1 | 6.7 | 10.6 | 12.3 |
| 6 | K536L | 4.2 | 3.0 | 53.6 | 15.7 | 8.6 |
| 6 | K536E | 1.1 | 1.1 | 4.2 | 1.5 | 1.0 |
| 6 | K536A | 2.0 | 0.9 | 1.3 | 1.4 | 1.2 |
| 6 | K536G | 1.5 | 2.1 | 5.7 | 6.7 | 8.0 |
| 6 | P537R | 3.4 | 3.6 | 18.4 | 8.9 | 10.8 |
| 6 | P537P | 2.9 | 5.1 | 8.2 | 2.3 | 3.5 |
| 6 | P537A | 3.8 | 2.3 | 6.7 | 13.2 | 5.8 |
| 6 | P537V | 5.1 | 7.7 | 16.8 | 17.9 | 15.4 |
| 6 | A538D | 3.7 | 9.0 | 8.1 | 0.2 | 2.7 |
| 6 | A538G | 2.0 | 2.6 | 8.2 | 27.9 | 21.9 |
| 6 | A538V | 1.0 | 3.4 | 10.3 | 6.1 | 2.1 |
| 6 | A538V | 1.3 | 1.2 | 0.4 | 1.7 | 1.3 |
| 6 | A538S | 0.7 | 2.6 | 13.9 | 11.2 | 15.1 |
| 6 | F539P | 6.4 | 22.0 | 30.7 | 18.1 | 21.8 |
| 6 | F539V | 2.5 | 4.2 | 5.2 | 8.4 | 8.1 |
| 6 | F539S | 1.2 | 2.4 | 5.1 | 3.3 | 3.5 |
| 6 | F539C | 2.6 | 2.8 | 4.0 | 4.6 | 3.7 |
| 6 | F539L | 1.4 | 1.6 | 1.3 | 2.2 | 1.5 |
| 6 | L540M | 0.7 | 1.9 | 11.8 | 3.9 | 5.2 |
| 6 | L540A | 0.6 | 0.1 | 19.3 | 1.9 | 1.8 |
| 6 | S541K | 1.8 | 1.4 | 2.3 | 2.5 | 1.0 |
| 6 | S541N | 2.1 | 1.0 | 1.3 | 3.1 | 2.1 |
| 6 | S541T | 1.2 | 0.3 | 10.2 | 3.4 | 5.0 |
| 6 | S541Q | 1.1 | 1.0 | 1.4 | 3.2 | 1.0 |
| 6 | S541R | 0.8 | 2.4 | 0.1 | 5.6 | 4.1 |
| 6 | S541G | 3.1 | 5.8 | 6.0 | 6.2 | 6.5 |
| 6 | G542R | 2.2 | 3.3 | 1.1 | 2.7 | 1.8 |
| 6 | G542M | 1.0 | 0.7 | 4.4 | 4.6 | 4.6 |
| 6 | G542P | 2.8 | 3.8 | 9.3 | 14.1 | 6.6 |
| 6 | G542E | 1.5 | 3.4 | 3.7 | 2.4 | 4.2 |
| 6 | G542G | 1.5 | 1.7 | 1.7 | 2.5 | 1.7 |
| 6 | G542S | 2.2 | 1.2 | 0.0 | 1.5 | 1.4 |
| 6 | G542C | 1.3 | 1.6 | 2.5 | 2.8 | 2.7 |
| 6 | G542L | 1.7 | 1.2 | 0.6 | 1.1 | 1.2 |
| 6 | G542F | 1.3 | 3.8 | 10.7 | 8.1 | 9.9 |
| 6 | E543R | 1.0 | 2.6 | 2.8 | 7.6 | 5.7 |
| 6 | E543Q | 1.0 | 2.8 | 3.9 | 8.2 | 2.9 |
| 6 | E543H | 1.2 | 5.7 | 33.4 | 60.4 | 56.4 |
| 6 | E543A | 0.7 | 0.9 | 4.4 | 4.6 | 4.0 |
| 6 | E543V | 1.5 | 0.9 | 1.7 | 1.0 | 1.8 |
| 6 | E543C | 0.9 | 4.1 | 3.0 | 16.8 | 13.3 |
| 6 | E543F | 1.6 | 2.8 | 5.4 | 8.4 | 6.4 |
| 6 | Q544L | 1.8 | 1.3 | 4.8 | 8.3 | 10.1 |
| 6 | K546N | 1.4 | 1.3 | 5.0 | 5.6 | 4.7 |
| 6 | K546T | 1.2 | 2.6 | 25.8 | 32.6 | 37.5 |
| 6 | K546R | 1.8 | 1.5 | 2.8 | 1.4 | 1.9 |
| 6 | K546Q | 2.1 | 6.2 | 10.5 | 14.0 | 15.2 |
| 6 | K546L | 3.5 | 2.1 | 0.9 | 2.9 | 1.3 |
| 6 | K546V | 4.2 | 9.6 | 0.6 | 5.7 | 1.7 |
| 6 | A547S | 2.1 | 4.7 | 9.6 | 6.3 | 4.7 |
| 6 | A547M | 0.8 | 0.0 | 1.8 | 1.6 | 2.1 |
| 6 | A547R | 2.1 | 2.1 | 5.0 | 9.8 | 8.1 |
| 6 | A547R | 1.2 | 1.1 | 3.2 | 5.3 | 4.1 |
| 6 | A547L | 2.3 | 4.3 | 8.6 | 19.7 | 23.2 |
| 6 | A547E | 1.1 | 2.5 | 2.0 | 2.4 | 3.2 |
| 6 | A547G | 1.3 | 2.6 | 1.0 | 0.8 | 1.5 |
| 6 | A547V | 1.8 | 2.9 | 2.3 | 4.6 | 3.8 |
| 6 | A547V | 0.6 | 1.2 | 2.5 | 2.0 | 1.2 |
| 6 | A547C | 1.6 | 2.8 | 4.1 | 10.7 | 10.1 |
| 6 | A547L | 1.2 | 2.5 | 2.1 | 5.1 | 1.9 |
| 6 | A547F | 1.2 | 2.7 | 8.1 | 3.0 | 3.4 |
| 6 | I548L | 2.2 | 0.8 | 13.5 | 6.6 | 2.8 |
| 6 | I548A | 0.9 | 0.9 | 4.2 | 6.0 | 4.5 |
| 6 | I548S | 0.6 | 0.0 | 8.6 | 2.1 | 2.7 |
| 6 | V549R | 1.4 | 4.1 | 14.3 | 31.9 | 17.6 |
| 6 | V549Q | 2.1 | 5.0 | 7.0 | 4.3 | 3.4 |
| 6 | D550K | 6.2 | 15.0 | 28.6 | 19.2 | 22.4 |
| 6 | D550E | 1.7 | 1.1 | 5.1 | 2.9 | 2.5 |
| 6 | D550V | 1.0 | 2.0 | 3.1 | 4.4 | 2.2 |
| 6 | L551N | 1.7 | 3.7 | 3.1 | 10.5 | 12.0 |
| 6 | L551T | 3.3 | 2.3 | 3.1 | 1.2 | 1.9 |
| 6 | L551R | 0.8 | 1.4 | 1.3 | 1.6 | 1.2 |
| 6 | L551A | 1.4 | 4.6 | 14.4 | 2.9 | 4.0 |
| 6 | L551V | 1.0 | 1.1 | 2.8 | 2.2 | 1.9 |
| 6 | L551V | 0.6 | 1.6 | 3.6 | 2.5 | 4.1 |
| 6 | L551Y | 0.7 | 0.6 | 9.8 | 8.3 | 12.8 |
| 6 | L551F | 1.9 | 1.8 | 10.2 | 4.4 | 3.8 |
| 6 | L552I | 2.8 | 5.7 | 5.8 | 10.3 | 15.3 |
| 6 | L552G | 0.6 | 0.6 | 2.2 | 1.6 | 2.1 |
| 6 | L552F | 2.1 | 2.9 | 7.1 | 16.8 | 17.6 |
| 6 | K554L | 2.1 | 6.3 | 6.0 | 3.1 | 3.1 |
| 6 | K554S | 0.5 | 2.3 | 6.4 | 4.2 | 4.5 |
| 6 | T555T | 1.7 | 3.4 | 1.0 | 0.6 | 1.1 |
| 6 | T555I | 1.3 | 1.2 | 5.1 | 12.8 | 16.4 |
| 6 | T555Q | 1.3 | 1.2 | 1.9 | 2.1 | 1.6 |
| 6 | T555P | 1.1 | 1.0 | 0.9 | 0.8 | 1.0 |
| 6 | T555R | 1.0 | 0.9 | 2.8 | 6.5 | 5.8 |
| 6 | T555V | 1.0 | 0.5 | 0.7 | 2.5 | 1.0 |
| 6 | T555S | 1.8 | 2.3 | 2.9 | 8.5 | 11.1 |
| 6 | T555L | 1.5 | 2.8 | 6.7 | 4.6 | 3.6 |
| 6 | N556K | 1.3 | 2.2 | 2.0 | 2.0 | 2.4 |
| 6 | N556T | 1.7 | 2.4 | 2.1 | 1.5 | 2.3 |
| 6 | N556S | 3.2 | 4.4 | 3.6 | 4.1 | 3.3 |
| 6 | N556I | 1.6 | 2.0 | 6.4 | 8.0 | 7.0 |
| 6 | N556H | 0.8 | 1.2 | 0.6 | 0.6 | 1.0 |
| 6 | N556L | 4.0 | 16.9 | 30.8 | 61.4 | 72.8 |
| 6 | R557P | 1.3 | 2.6 | 4.8 | 10.8 | 6.3 |
| 6 | R557E | 2.7 | 4.0 | 2.2 | 2.5 | 1.1 |
| 6 | R557G | 1.5 | 3.1 | 3.4 | 10.8 | 7.6 |
| 6 | R557G | 3.5 | 2.3 | 3.2 | 8.3 | 7.5 |
| 6 | R557V | 1.4 | 2.0 | 7.6 | 8.3 | 8.7 |
| 6 | R557S | 0.6 | 1.8 | 6.1 | 8.3 | 9.8 |
| 6 | R557W | 1.0 | 1.1 | 6.4 | 5.6 | 5.1 |
| 6 | R557L | 1.5 | 3.1 | 0.1 | 4.0 | 2.8 |
| 6 | K558I | 2.2 | 4.7 | 20.3 | 33.6 | 26.5 |
| 6 | K558D | 3.9 | 1.3 | 2.2 | 3.5 | 1.1 |
| 6 | K558S | 2.9 | 2.7 | 3.9 | 6.0 | 1.9 |
| 6 | K558S | 6.7 | 7.4 | 30.4 | 8.8 | 8.5 |
| 6 | K558L | 2.4 | 0.8 | 0.4 | 1.8 | 1.2 |
| 6 | R557K | 0.3 | 1.4 | 5.5 | 5.9 | 5.1 |
| 6 | R557P | 1.1 | 1.3 | 1.1 | 2.9 | 1.0 |
| 6 | R557E | 1.3 | 2.7 | 3.6 | 4.5 | 3.8 |
| 6 | R557G | 0.8 | 1.4 | 1.4 | 4.5 | 2.9 |
| 6 | R557G | 1.7 | 1.2 | 1.2 | 3.2 | 2.7 |
| 6 | R557V | 0.7 | 1.2 | 2.6 | 2.6 | 2.3 |
| 6 | R557S | 0.2 | 0.8 | 2.4 | 3.1 | 3.3 |
| 6 | R557W | 0.7 | 0.4 | 2.6 | 2.1 | 1.8 |
| 6 | R557L | 0.7 | 1.3 | 0.0 | 1.9 | 1.2 |
| 6 | K558I | 1.1 | 3.2 | 7.4 | 12.8 | 9.6 |
| 6 | K558G | 1.4 | 2.7 | 4.7 | 2.5 | 1.6 |
| 6 | K558G | 1.5 | 0.6 | 4.0 | 2.5 | 2.4 |
| 6 | K558S | 2.1 | 3.2 | 9.8 | 2.7 | 2.8 |
| 6 | K558L | 1.5 | 0.7 | 0.4 | 1.3 | 1.5 |
| 6 | T560A | 0.8 | 1.5 | 0.5 | 2.9 | 2.5 |
| 6 | V561T | 1.3 | 2.9 | 12.4 | 4.4 | 4.4 |
| 6 | V561T | 0.7 | 1.7 | 2.3 | 2.8 | 1.7 |
| 6 | V561H | 2.3 | 2.3 | 4.9 | 9.0 | 9.3 |
| 6 | V561V | 0.9 | 1.8 | 3.9 | 2.4 | 1.2 |
| 6 | K562T | 0.9 | 0.7 | 2.3 | 5.1 | 3.9 |
| 6 | K562S | 2.6 | 4.8 | 13.0 | 15.7 | 12.0 |
| 6 | K562P | 1.7 | 2.1 | 0.9 | 0.7 | 1.2 |
| 6 | K562L | 1.4 | 1.3 | 0.1 | 2.8 | 2.3 |
| 6 | K562L | 1.5 | 1.1 | 7.7 | 11.1 | 5.9 |
| 6 | K562G | 1.2 | 0.8 | 3.2 | 4.5 | 4.1 |
| 6 | K562L | 1.4 | 1.1 | 0.1 | 1.1 | 1.0 |
| 6 | Q563E | 1.0 | 2.2 | 1.1 | 3.6 | 3.4 |
| 6 | L564V | 0.9 | 2.8 | 9.9 | 4.2 | 1.9 |
| 6 | K565R | 0.9 | 1.0 | 4.0 | 1.2 | 2.1 |
| 6 | K565A | 1.6 | 2.5 | 9.9 | 10.8 | 10.7 |
| 6 | K565S | 2.0 | 2.8 | 9.3 | 16.3 | 19.9 |
| 6 | E566Q | 3.0 | 2.5 | 0.1 | 2.3 | 4.0 |
| 6 | E566G | 1.2 | 1.3 | 3.7 | 2.6 | 3.2 |
| 6 | E566W | 0.8 | 1.6 | 3.5 | 3.6 | 3.2 |
| 6 | D567R | 0.7 | 0.9 | 7.0 | 5.7 | 3.4 |
| 6 | D567V | 1.6 | 4.7 | 11.8 | 22.2 | 19.2 |
| 6 | I572Q | 5.2 | 38.5 | 36.4 | 33.9 | 43.1 |
| 6 | I572R | 3.4 | 10.2 | 40.4 | 61.8 | 95.6 |
| 6 | I572F | 1.1 | 1.1 | 1.2 | 1.1 | 1.1 |
| 6 | C574G | 0.8 | 3.1 | 4.9 | 2.9 | 2.4 |
| 6 | F575E | 0.9 | 1.2 | 4.4 | 1.8 | 1.0 |
| 6 | F575L | 1.2 | 1.9 | 3.6 | 4.8 | 4.1 |
| 6 | D576I | 1.5 | 1.9 | 6.5 | 2.6 | 2.4 |
| 6 | D576L | 1.3 | 3.4 | 11.8 | 5.8 | 7.4 |
| 6 | D576D | 1.8 | 0.9 | 3.1 | 3.1 | 2.3 |
| 6 | D576G | 0.8 | 1.3 | 1.9 | 6.7 | 7.1 |
| 6 | S577R | 1.3 | 2.8 | 4.6 | 3.1 | 3.9 |
| 6 | S577D | 1.1 | 10.4 | 6.4 | 2.2 | 2.8 |
| 6 | S577G | 2.0 | 3.2 | 6.0 | 6.0 | 5.6 |
| 6 | V578V | 0.9 | 0.3 | 2.7 | 3.4 | 3.5 |
| 6 | E579T | 2.5 | 3.5 | 0.2 | 3.3 | 5.8 |
| 6 | E579L | 2.3 | 1.8 | 2.9 | 6.2 | 6.5 |
| 6 | E579A | 1.8 | 2.2 | 4.8 | 5.6 | 8.8 |
| 6 | E579A | 0.9 | 1.1 | 3.0 | 1.1 | 1.0 |
| 6 | E579G | 0.6 | 0.1 | 1.6 | 1.3 | 1.7 |
| 6 | E579S | 1.5 | 3.6 | 8.4 | 4.2 | 4.3 |
| 6 | I580L | 2.7 | 2.3 | 7.8 | 6.8 | 5.9 |
| 6 | I580V | 1.3 | 1.8 | 4.1 | 2.4 | 1.9 |
| 6 | I580C | 0.9 | 2.6 | 1.1 | 3.3 | 1.6 |
| 6 | I580L | 0.6 | 1.3 | 4.7 | 2.8 | 2.5 |
| 6 | S581K | 0.8 | 2.5 | 2.0 | 1.3 | 1.8 |
| 6 | S581N | 1.0 | 1.1 | 0.7 | 1.4 | 1.7 |
| 6 | S581M | 0.4 | 0.2 | 0.1 | 2.5 | 2.6 |
| 6 | S581Q | 1.5 | 1.7 | 1.4 | 6.1 | 6.7 |
| 6 | S581L | 1.1 | 1.1 | 2.7 | 1.9 | 3.1 |
| 6 | S581D | 1.1 | 2.6 | 3.3 | 1.9 | 2.4 |
| 6 | S581A | 1.4 | 3.8 | 2.9 | 4.8 | 4.1 |
| 6 | S581Y | 1.4 | 1.3 | 1.7 | 0.8 | 1.2 |
| 6 | G582A | 0.7 | 1.7 | 1.7 | 2.4 | 1.4 |
| 6 | G582G | 1.7 | 1.5 | 3.9 | 5.4 | 4.9 |
| 6 | V583N | 0.8 | 0.3 | 2.4 | 3.6 | 3.2 |
| 6 | V583T | 1.1 | 1.5 | 2.0 | 1.9 | 1.2 |
| 6 | V583M | 1.2 | 1.3 | 6.3 | 7.2 | 7.7 |
| 6 | V583R | 0.5 | 0.5 | 3.7 | 3.6 | 3.8 |
| 6 | V583G | 1.2 | 0.2 | 1.6 | 1.6 | 1.1 |
| 6 | V583V | 0.6 | 1.9 | 1.3 | 1.6 | 3.0 |
| 6 | E584K | 1.2 | 1.2 | 1.0 | 1.3 | 1.3 |
| 6 | E584I | 0.9 | 1.2 | 1.2 | 2.6 | 2.3 |
| 6 | E584A | 0.8 | 0.5 | 3.2 | 3.4 | 2.8 |
| 6 | E584G | 0.8 | 2.0 | 2.4 | 3.9 | 3.2 |
| 6 | E584G | 0.7 | 1.1 | 2.1 | 2.6 | 2.2 |
| 6 | E584V | 1.1 | 0.9 | 1.7 | 2.3 | 1.3 |
| 6 | D585L | 1.2 | 0.9 | 4.4 | 1.2 | 1.3 |
| 6 | D585E | 1.0 | 2.1 | 2.2 | 4.1 | 3.5 |
| 6 | D585G | 0.9 | 0.6 | 2.4 | 4.4 | 2.7 |
| 6 | D585C | 0.5 | 2.3 | 1.6 | 1.6 | 1.7 |
| 6 | D585F | 1.6 | 2.0 | 4.5 | 3.6 | 4.5 |
| 6 | R586N | 0.7 | 0.5 | 1.1 | 0.6 | 1.0 |
| 6 | R586T | 1.3 | 3.9 | 10.1 | 11.8 | 14.6 |
| 6 | R586L | 0.7 | 1.5 | 1.9 | 3.2 | 2.0 |
| 6 | R586A | 1.2 | 1.5 | 5.3 | 20.0 | 12.9 |
| 6 | R586L | 1.2 | 1.8 | 5.5 | 9.7 | 7.2 |
| 6 | F587A | 0.3 | 0.6 | 1.1 | 1.5 | 1.3 |
| 6 | F587V | 0.8 | 0.7 | 2.5 | 1.9 | 2.1 |
| 6 | F587C | 1.3 | 1.4 | 1.3 | 1.4 | 1.3 |
| 6 | N588N | 2.0 | 2.3 | 4.1 | 5.8 | 6.9 |
| 6 | N588R | 1.0 | 0.7 | 3.0 | 7.6 | 5.9 |
| 6 | N588L | 2.1 | 2.9 | 7.1 | 2.8 | 3.4 |
| 6 | N588G | 1.5 | 1.4 | 2.5 | 0.7 | 1.1 |
| 6 | N588L | 0.7 | 0.6 | 1.7 | 2.3 | 1.9 |
| 6 | A589N | 0.9 | 2.0 | 3.2 | 2.7 | 1.7 |
| 6 | A589S | 1.2 | 2.3 | 1.9 | 1.7 | 1.8 |
| 6 | A589A | 1.4 | 0.7 | 1.8 | 3.1 | 3.7 |
| 6 | A589S | 1.6 | 1.4 | 5.9 | 22.6 | 16.2 |
| 6 | S590T | 0.8 | 1.4 | 3.9 | 4.1 | 4.3 |
| 6 | S590E | 1.7 | 3.6 | 4.8 | 2.6 | 1.1 |
| 6 | S590A | 2.2 | 3.7 | 8.5 | 9.4 | 10.1 |
| 6 | S590S | 1.5 | 1.4 | 2.6 | 4.8 | 4.0 |
| 6 | S590C | 1.3 | 2.0 | 1.8 | 2.1 | 1.5 |
| 6 | S590F | 1.3 | 1.1 | 1.6 | 0.9 | 1.4 |
| 6 | G592N | 1.8 | 3.6 | 2.9 | 10.9 | 7.8 |
| 6 | G592M | 2.6 | 1.9 | 5.7 | 6.9 | 4.9 |
| 6 | G592A | 0.8 | 0.6 | 0.0 | 0.7 | 1.3 |
| 6 | G592C | 1.0 | 1.4 | 1.8 | 0.8 | 1.0 |
| 6 | T593T | 0.7 | 0.5 | 1.3 | 3.3 | 1.2 |
| 6 | T593G | 1.2 | 1.8 | 3.9 | 1.8 | 1.6 |
| 6 | T593S | 1.9 | 1.7 | 8.6 | 1.1 | 3.1 |
| 6 | Y594F | 1.4 | 2.5 | 3.0 | 3.6 | 3.7 |
| 6 | H595N | 0.8 | 0.7 | 2.7 | 4.1 | 5.0 |
| 6 | H595Q | 1.0 | 2.3 | 0.2 | 1.1 | 1.9 |
| 6 | H595V | 0.6 | 0.3 | 3.8 | 3.1 | 5.3 |
| 6 | L597F | 0.9 | 1.6 | 5.0 | 11.1 | 5.5 |
| 6 | L598M | 1.4 | 0.8 | 3.3 | 4.0 | 3.2 |
| 6 | L598Q | 1.1 | 1.5 | 2.6 | 8.0 | 9.7 |
| 6 | L598H | 1.0 | 2.5 | 4.1 | 6.7 | 4.4 |
| 6 | L598R | 1.4 | 2.4 | 5.6 | 4.8 | 5.4 |
| 6 | L598L | 0.9 | 2.5 | 10.7 | 8.4 | 7.8 |
| 6 | L598G | 0.8 | 1.6 | 4.5 | 1.5 | 2.7 |
| 6 | L598L | 1.7 | 1.4 | 2.8 | 5.1 | 3.4 |
| 6 | L598F | 0.6 | 1.4 | 1.2 | 4.5 | 3.6 |
| 6 | K599T | 1.8 | 2.7 | 2.9 | 8.8 | 5.4 |
| 6 | K599L | 1.7 | 3.1 | 2.0 | 28.1 | 47.4 |
| 6 | K599G | 1.4 | 0.7 | 2.2 | 2.9 | 1.7 |
| 6 | K599W | 1.4 | 0.9 | 3.4 | 1.9 | 2.2 |
| 6 | I600I | 2.2 | 2.5 | 3.0 | 2.8 | 3.6 |
| 6 | I600D | 1.8 | 1.8 | 2.6 | 12.8 | 18.2 |
| 6 | I600V | 1.4 | 1.0 | 3.4 | 4.0 | 4.3 |
| 7 | I601T | 1.4 | 0.4 | 0.6 | 0.5 | 1.0 |
| 7 | I601V | 1.4 | 1.3 | 1.0 | 0.4 | 1.6 |
| 7 | I601C | 0.8 | 0.8 | 0.7 | 1.2 | 2.8 |
| 7 | K602M | 1.3 | 1.7 | 1.7 | 1.2 | 1.9 |
| 7 | K602Q | 0.5 | 1.1 | 2.1 | 3.5 | 1.3 |
| 7 | K602P | 0.9 | 1.1 | 1.2 | 2.7 | 4.1 |
| 7 | K602E | 1.0 | 1.5 | 2.0 | 2.4 | 3.8 |
| 7 | K602A | 1.2 | 1.2 | 1.0 | 0.8 | 1.5 |
| 7 | K602L | 1.0 | 0.9 | 1.0 | 1.4 | 2.7 |
| 7 | K604K | 1.0 | 1.6 | 3.3 | 2.8 | 3.5 |
| 7 | K604A | 2.0 | 4.1 | 20.2 | 9.7 | 3.9 |
| 7 | D605R | 1.4 | 2.3 | 0.7 | 12.9 | 14.7 |
| 7 | D605S | 1.1 | 3.2 | 7.0 | 6.6 | 4.5 |
| 7 | D605D | 0.6 | 0.2 | 0.2 | 0.6 | 1.3 |
| 7 | D605A | 1.6 | 4.5 | 3.4 | 2.4 | 1.0 |
| 7 | F606Q | 4.7 | 10.8 | 2.1 | 12.4 | 35.6 |
| 7 | L607L | 1.9 | 2.3 | 14.2 | 13.2 | 21.9 |
| 7 | D608Y | 0.9 | 1.0 | 1.0 | 0.9 | 1.1 |
| 7 | N609K | 1.3 | 1.6 | 2.1 | 2.5 | 1.9 |
| 7 | N609T | 1.8 | 17.0 | 20.0 | 5.1 | 1.7 |
| 7 | N609R | 1.7 | 4.3 | 4.9 | 9.2 | 5.4 |
| 7 | N609I | 1.3 | 2.9 | 2.0 | 2.7 | 1.2 |
| 7 | N609E | 1.3 | 0.7 | 2.3 | 3.3 | 1.5 |
| 7 | N609V | 1.7 | 3.2 | 3.9 | 2.7 | 6.3 |
| 7 | E610A | 2.6 | 3.3 | 5.3 | 3.2 | 10.3 |
| 7 | E610G | 1.7 | 1.3 | 0.7 | 3.7 | 3.8 |
| 7 | E611N | 0.5 | 0.1 | 0.5 | 1.1 | 1.2 |
| 7 | E611V | 1.1 | 1.7 | 3.5 | 1.7 | 1.1 |
| 7 | E611Y | 2.1 | 2.7 | 23.9 | 42.0 | 34.4 |
| 7 | E611S | 1.1 | 2.3 | 1.3 | 0.7 | 1.9 |
| 7 | E611F | 1.6 | 4.0 | 0.1 | 3.1 | 6.0 |
| 7 | N612R | 0.7 | 0.8 | 0.0 | 1.1 | 3.5 |
| 7 | E613S | 1.0 | 1.4 | 5.0 | 15.3 | 10.5 |
| 7 | E613R | 1.2 | 2.1 | 2.1 | 5.7 | 3.3 |
| 7 | E613E | 1.0 | 1.1 | 2.4 | 1.8 | 1.0 |
| 7 | D614N | 1.1 | 1.7 | 1.7 | 4.2 | 4.6 |
| 7 | D614S | 0.9 | 2.9 | 7.9 | 8.2 | 6.7 |
| 7 | D614I | 1.9 | 1.7 | 3.0 | 3.3 | 5.1 |
| 7 | D614P | 2.3 | 5.7 | 10.4 | 8.3 | 2.7 |
| 7 | D614G | 0.8 | 1.0 | 0.7 | 4.0 | 3.7 |
| 7 | D614F | 1.4 | 3.6 | 0.9 | 3.5 | 2.1 |
| 7 | I615T | 0.7 | 1.0 | 0.2 | 1.2 | 1.3 |
| 7 | I615A | 0.9 | 0.4 | 1.4 | 4.6 | 4.3 |
| 7 | I615Y | 0.7 | 4.0 | 5.0 | 4.8 | 1.6 |
| 7 | L616R | 0.3 | 4.1 | 3.7 | 4.3 | 2.9 |
| 7 | L616L | 1.2 | 1.8 | 1.6 | 0.5 | 4.7 |
| 7 | D618T | 1.2 | 1.3 | 2.4 | 2.4 | 8.3 |
| 7 | D618R | 0.1 | 0.3 | 0.8 | 0.6 | 1.0 |
| 7 | D618E | 0.7 | 1.8 | 1.7 | 2.6 | 5.3 |
| 7 | D618D | 1.4 | 2.2 | 1.6 | 3.1 | 3.1 |
| 7 | D618Y | 1.3 | 1.3 | 3.9 | 3.1 | 2.6 |
| 7 | I619M | 1.1 | 1.3 | 0.9 | 3.8 | 6.7 |
| 7 | I619V | 1.5 | 1.5 | 1.8 | 1.6 | 3.2 |
| 7 | V620C | 0.6 | 0.1 | 0.0 | 2.1 | 2.1 |
| 7 | V620L | 0.9 | 1.1 | 3.4 | 7.1 | 4.2 |
| 7 | L621T | 1.0 | 1.2 | 2.7 | 4.6 | 3.5 |
| 7 | L621E | 1.0 | 1.1 | 0.6 | 1.4 | 3.9 |
| 7 | L621V | 0.9 | 1.1 | 1.4 | 0.8 | 1.6 |
| 7 | L621S | 1.4 | 2.8 | 3.3 | 2.2 | 1.0 |
| 7 | T622S | 0.8 | 1.0 | 3.0 | 1.9 | 3.4 |
| 7 | T622I | 2.4 | 3.3 | 3.7 | 2.2 | 2.1 |
| 7 | L623N | 1.0 | 1.1 | 1.4 | 2.3 | 9.7 |
| 7 | L623L | 0.9 | 1.2 | 2.1 | 1.3 | 1.9 |
| 7 | L623L | 1.0 | 1.5 | 1.5 | 0.8 | 2.7 |
| 7 | T624T | 1.3 | 0.8 | 1.6 | 2.3 | 1.2 |
| 7 | L625R | 1.2 | 2.3 | 6.7 | 4.9 | 4.2 |
| 7 | L625A | 1.7 | 1.5 | 0.8 | 2.5 | 1.5 |
| 7 | L625A | 1.5 | 2.6 | 2.3 | 4.2 | 5.8 |
| 7 | L625G | 1.1 | 1.0 | 0.7 | 2.7 | 2.8 |
| 7 | L625Y | 0.8 | 0.6 | 0.3 | 1.0 | 2.1 |
| 7 | L625C | 1.0 | 0.5 | 0.2 | 0.2 | 1.5 |
| 7 | F626C | 0.6 | 0.4 | 0.7 | 0.6 | 2.1 |
| 7 | L625H | 1.5 | 6.4 | 8.3 | 2.2 | 1.5 |
| 7 | L625R | 1.5 | 1.9 | 2.7 | 1.9 | 1.9 |
| 7 | L625A | 0.8 | 0.6 | 0.4 | 0.8 | 1.1 |
| 7 | L625G | 1.9 | 2.7 | 0.2 | 2.6 | 1.2 |
| 7 | L625G | 2.2 | 2.3 | 1.7 | 23.9 | 27.6 |
| 7 | L625C | 1.3 | 1.0 | 0.5 | 0.5 | 2.9 |
| 7 | R629I | 0.8 | 0.2 | 0.5 | 0.3 | 1.0 |
| 7 | R629R | 1.5 | 2.7 | 4.6 | 10.0 | 16.0 |
| 7 | R629L | 2.5 | 4.9 | 11.2 | 25.0 | 35.2 |
| 7 | R629L | 2.4 | 1.1 | 1.1 | 1.0 | 2.3 |
| 7 | R629G | 2.6 | 5.2 | 12.9 | 3.3 | 3.1 |
| 7 | R629Y | 1.8 | 1.7 | 2.4 | 3.1 | 4.0 |
| 7 | R629S | 1.3 | 0.9 | 4.3 | 5.7 | 12.0 |
| 7 | R629W | 0.8 | 3.0 | 6.0 | 3.0 | 2.9 |
| 7 | E630E | 2.7 | 12.9 | 10.1 | 12.3 | 8.1 |
| 7 | E630G | 1.5 | 2.4 | 4.3 | 6.6 | 2.4 |
| 7 | E630V | 1.4 | 19.3 | 5.7 | 5.7 | 1.8 |
| 7 | I632L | 1.9 | 5.9 | 1.5 | 2.7 | 4.4 |
| 7 | I632F | 1.0 | 0.9 | 0.8 | 1.1 | 1.0 |
| 7 | E633A | 0.9 | 0.2 | 6.3 | 6.7 | 5.5 |
| 7 | E633G | 3.0 | 4.6 | 2.4 | 5.8 | 2.2 |
| 7 | E634K | 2.0 | 5.3 | 27.2 | 54.9 | 7.7 |
| 7 | E634S | 1.0 | 1.8 | 17.6 | 21.9 | 36.7 |
| 7 | E634E | 2.6 | 3.6 | 9.0 | 8.7 | 5.4 |
| 7 | R635R | 1.9 | 2.1 | 2.8 | 3.1 | 1.7 |
| 7 | R635G | 0.8 | 1.6 | 2.1 | 3.3 | 3.3 |
| 7 | L636L | 1.7 | 0.4 | 0.5 | 1.0 | 1.9 |
| 7 | K637K | 1.3 | 1.7 | 4.0 | 2.5 | 2.5 |
| 7 | K637T | 1.9 | 0.1 | 5.1 | 9.3 | 37.8 |
| 7 | K637R | 1.0 | 2.1 | 2.5 | 2.8 | 1.6 |
| 7 | K637G | 1.3 | 3.8 | 9.1 | 5.3 | 11.7 |
| 7 | K637C | 2.3 | 3.0 | 3.8 | 2.8 | 1.1 |
| 7 | T638N | 1.4 | 1.5 | 3.9 | 1.4 | 2.7 |
| 7 | T638S | 1.4 | 1.6 | 2.2 | 1.7 | 1.0 |
| 7 | T638M | 1.4 | 0.7 | 2.9 | 4.4 | 1.3 |
| 7 | T638P | 2.6 | 5.6 | 7.8 | 6.0 | 9.5 |
| 7 | T638L | 1.7 | 1.8 | 2.4 | 3.1 | 4.5 |
| 7 | T638D | 1.2 | 1.8 | 4.7 | 1.8 | 1.5 |
| 7 | T638A | 2.5 | 2.2 | 6.4 | 5.1 | 8.4 |
| 7 | T638G | 2.0 | 1.9 | 2.4 | 1.0 | 2.3 |
| 7 | T638V | 1.7 | 2.3 | 0.2 | 3.1 | 1.8 |
| 7 | T638C | 1.5 | 2.2 | 3.9 | 5.8 | 9.6 |
| 7 | Y639V | 0.8 | 0.4 | 0.5 | 1.1 | 1.6 |
| 7 | Y639S | 3.4 | 4.4 | 5.2 | 1.5 | 3.7 |
| 7 | Y639S | 1.1 | 0.6 | 2.4 | 2.4 | 2.0 |
| 7 | Y639C | 1.4 | 2.0 | 3.5 | 4.8 | 4.4 |
| 7 | A640T | 3.2 | 7.0 | 9.7 | 9.3 | 8.1 |
| 7 | A640L | 2.3 | 3.1 | 7.0 | 8.4 | 11.3 |
| 7 | A640E | 2.9 | 3.0 | 3.4 | 5.0 | 11.1 |
| 7 | A640A | 1.2 | 1.0 | 2.7 | 5.0 | 3.0 |
| 7 | A640V | 1.9 | 1.3 | 2.0 | 4.2 | 5.2 |
| 7 | A640V | 1.7 | 3.4 | 0.4 | 3.8 | 2.1 |
| 7 | A640W | 1.3 | 2.3 | 1.8 | 28.3 | 19.0 |
| 7 | A640C | 4.1 | 3.3 | 6.0 | 2.2 | 1.7 |
| 7 | H641V | 5.1 | 22.8 | 71.1 | 37.4 | 90.1 |
| 7 | L642K | 3.6 | 2.5 | 6.9 | 4.8 | 3.4 |
| 7 | L642L | 2.5 | 3.8 | 1.3 | 2.3 | 3.3 |
| 7 | L642G | 1.1 | 1.4 | 5.1 | 2.0 | 1.8 |
| 7 | L642L | 1.3 | 1.9 | 2.1 | 5.5 | 6.3 |
| 7 | F643Y | 0.6 | 1.7 | 4.7 | 8.3 | 10.5 |
| 7 | D644K | 2.2 | 5.1 | 8.4 | 12.1 | 30.6 |
| 7 | D644N | 2.1 | 6.6 | 9.6 | 8.3 | 1.1 |
| 7 | D644P | 2.2 | 4.7 | 19.1 | 11.0 | 5.6 |
| 7 | D644V | 1.7 | 3.8 | 18.0 | 13.4 | 12.4 |
| 7 | D645V | 0.9 | 2.4 | 0.9 | 2.0 | 1.5 |
| 7 | D645Y | 1.7 | 2.0 | 3.0 | 18.6 | 16.4 |
| 7 | K646T | 2.7 | 4.4 | 1.2 | 0.8 | 2.8 |
| 7 | K646S | 1.9 | 3.4 | 1.5 | 25.5 | 9.3 |
| 7 | K646STOP | 0.6 | 0.9 | 1.1 | 1.4 | 1.3 |
| 7 | K646W | 1.1 | 1.0 | 2.7 | 4.7 | 4.3 |
| 7 | K646L | 1.0 | 1.5 | 2.3 | 0.9 | 1.4 |
| 7 | V647V | 1.7 | 1.5 | 2.8 | 6.4 | 3.6 |
| 7 | V647C | 2.2 | 1.0 | 6.5 | 3.7 | 4.3 |
| 7 | M648G | 2.0 | 1.2 | 5.4 | 10.6 | 1.8 |
| 7 | M648Y | 2.7 | 2.7 | 9.2 | 142.6 | 157.2 |
| 7 | M648S | 3.4 | 0.6 | 1.1 | 0.8 | 1.9 |
| 7 | M648L | 0.8 | 0.9 | 0.8 | 1.0 | 1.0 |
| 7 | K649N | 1.1 | 2.4 | 16.9 | 32.0 | 37.8 |
| 7 | K649Q | 1.7 | 4.0 | 23.6 | 7.5 | 5.3 |
| 7 | K649E | 1.7 | 4.8 | 9.6 | 17.8 | 42.3 |
| 7 | K649C | 9.1 | 20.9 | 57.4 | 48.7 | 64.0 |
| 7 | K652R | 3.5 | 10.4 | 9.5 | 16.6 | 25.2 |
| 7 | K652A | 0.6 | 0.9 | 1.7 | 2.2 | 2.0 |
| 7 | K652Y | 2.4 | 5.8 | 13.2 | 22.9 | 20.5 |
| 7 | K652L | 2.0 | 0.9 | 7.4 | 26.5 | 23.1 |
| 7 | K652F | 0.7 | 1.6 | 0.1 | 5.2 | 6.5 |
| 7 | R653N | 3.5 | 7.0 | 40.0 | 33.3 | 8.0 |
| 7 | R653S | 1.4 | 1.9 | 5.0 | 5.1 | 12.8 |
| 7 | R653G | 1.0 | 1.1 | 1.1 | 1.2 | 1.1 |
| 7 | R653C | 1.3 | 1.8 | 3.2 | 15.5 | 11.7 |
| 7 | R654K | 1.2 | 5.3 | 20.1 | 31.4 | 33.4 |
| 7 | R654T | 0.6 | 1.8 | 8.2 | 25.6 | 31.9 |
| 7 | R654R | 1.6 | 2.4 | 3.6 | 1.4 | 2.9 |
| 7 | R654Q | 4.6 | 4.8 | 4.7 | 3.5 | 3.3 |
| 7 | R654G | 1.7 | 1.0 | 6.3 | 6.1 | 7.5 |
| 7 | R655Q | 4.7 | 6.5 | 1.7 | 16.3 | 1.5 |
| 7 | R655R | 0.7 | 3.3 | 6.5 | 10.3 | 22.0 |
| 7 | R655L | 1.0 | 0.1 | 18.4 | 23.5 | 18.4 |
| 7 | Y656W | 1.2 | 0.6 | 2.4 | 2.8 | 1.6 |
| 7 | T657S | 1.1 | 1.7 | 3.0 | 0.9 | 2.2 |
| 7 | T657I | 1.0 | 2.6 | 1.0 | 1.5 | 4.0 |
| 7 | T657A | 2.2 | 5.0 | 6.9 | 9.1 | 10.0 |
| 7 | T657V | 0.7 | 0.4 | 0.5 | 1.2 | 1.7 |
| 7 | T657W | 2.1 | 1.3 | 2.4 | 6.9 | 5.6 |
| 7 | W659M | 0.5 | 1.4 | 1.1 | 1.1 | 2.3 |
| 7 | G660G | 1.0 | 6.0 | 6.8 | 15.9 | 7.8 |
| 7 | R661P | 0.8 | 4.4 | 10.8 | 6.3 | 26.4 |
| 7 | R661Y | 3.5 | 7.4 | 29.2 | 7.2 | 6.8 |
| 7 | R661C | 1.1 | 1.9 | 6.9 | 15.7 | 16.4 |
| 7 | L662T | 1.1 | 1.2 | 1.8 | 0.7 | 1.1 |
| 7 | L662S | 1.5 | 2.4 | 1.4 | 2.6 | 2.2 |
| 7 | L662L | 1.3 | 1.6 | 3.7 | 12.9 | 6.0 |
| 7 | L662L | 2.8 | 2.5 | 0.4 | 3.6 | 4.7 |
| 7 | L662A | 2.6 | 5.8 | 4.6 | 5.5 | 6.8 |
| 7 | L662F | 1.5 | 1.3 | 5.1 | 9.2 | 7.8 |
| 7 | S663G | 0.9 | 1.7 | 1.4 | 1.4 | 1.2 |
| 7 | S663S | 2.1 | 3.2 | 1.6 | 3.2 | 6.3 |
| 7 | R664R | 0.8 | 1.1 | 2.8 | 1.8 | 6.8 |
| 7 | R664H | 1.8 | 3.8 | 7.6 | 22.0 | 12.1 |
| 7 | R664R | 2.2 | 1.9 | 4.0 | 2.1 | 3.5 |
| 7 | R664G | 1.4 | 1.1 | 0.8 | 3.2 | 2.4 |
| 7 | K665R | 1.7 | 2.5 | 3.6 | 4.4 | 2.7 |
| 7 | K665F | 0.2 | 0.9 | 9.3 | 18.7 | 21.8 |
| 7 | L666S | 0.9 | 0.2 | 7.3 | 6.7 | 4.1 |
| 7 | L666M | 1.4 | 1.9 | 10.5 | 8.6 | 10.9 |
| 7 | N668H | 9.6 | 15.2 | 31.7 | 41.7 | 91.0 |
| 7 | G669N | 7.3 | 19.7 | 26.5 | 3.4 | 16.6 |
| 7 | G669G | 2.5 | 3.8 | 6.1 | 3.6 | 1.6 |
| 7 | G669G | 1.5 | 1.7 | 2.6 | 2.5 | 1.7 |
| 7 | I670M | 2.2 | 10.8 | 4.7 | 3.8 | 7.8 |
| 7 | R671K | 1.9 | 2.7 | 2.7 | 6.6 | 10.0 |
| 7 | R671R | 1.7 | 3.9 | 5.1 | 4.5 | 4.6 |
| 7 | K673N | 0.9 | 1.1 | 1.0 | 0.9 | 1.1 |
| 7 | K673R | 1.4 | 3.0 | 2.6 | 9.2 | 17.2 |
| 7 | K673M | 1.2 | 1.0 | 4.5 | 2.1 | 2.4 |
| 7 | K673I | 1.5 | 2.8 | 6.3 | 5.0 | 3.6 |
| 7 | Q674S | 3.8 | 6.8 | 19.7 | 100.6 | 61.8 |
| 7 | G676K | 0.2 | 0.4 | 2.0 | 1.1 | 1.1 |
| 7 | K677L | 0.7 | 3.4 | 18.5 | 5.7 | 16.5 |
| 7 | K677F | 2.0 | 3.7 | 23.2 | 23.6 | 14.7 |
| 7 | T678S | 0.3 | 2.8 | 9.5 | 11.1 | 13.0 |
| 7 | T678S | 1.4 | 1.1 | 0.9 | 0.9 | 1.0 |
| 7 | I679N | 0.7 | 1.6 | 0.5 | 0.6 | 1.1 |
| 7 | L680V | 0.6 | 0.5 | 0.9 | 1.1 | 1.0 |
| 7 | K684STOP | 0.9 | 1.0 | 1.1 | 1.1 | 1.2 |
| 7 | S685N | 10.1 | 42.0 | 44.2 | 88.9 | 14.5 |
| 7 | S685T | 4.6 | 5.8 | 47.7 | 59.7 | 36.5 |
| 7 | S685G | 3.2 | 5.0 | 4.8 | 5.3 | 5.6 |
| 7 | S685S | 1.4 | 1.8 | 1.8 | 2.0 | 2.1 |
| 7 | G687T | 0.4 | 0.7 | 2.4 | 3.4 | 4.1 |
| 7 | G687M | 1.7 | 5.0 | 6.9 | 10.7 | 3.4 |
| 7 | G687I | 1.2 | 0.6 | 6.6 | 0.3 | 5.6 |
| 7 | G687R | 0.4 | 6.0 | 20.3 | 18.0 | 28.8 |
| 7 | G687G | 1.2 | 0.7 | 2.2 | 2.4 | 3.1 |
| 7 | G687W | 1.8 | 3.2 | 9.8 | 13.3 | 8.5 |
| 7 | F688N | 0.8 | 2.8 | 9.8 | 11.1 | 4.4 |
| 7 | F688L | 3.8 | 4.9 | 2.5 | 1.6 | 3.9 |
| 7 | F688E | 0.9 | 1.1 | 2.0 | 5.5 | 4.6 |
| 7 | F688A | 1.2 | 0.5 | 1.5 | 0.9 | 3.4 |
| 7 | F688V | 1.9 | 2.4 | 6.4 | 6.4 | 21.3 |
| 7 | F688V | 2.1 | 10.1 | 26.9 | 25.5 | 19.8 |
| 7 | A689P | 0.5 | 3.4 | 2.6 | 12.2 | 21.4 |
| 7 | A689L | 2.8 | 6.7 | 11.2 | 131.9 | 228.2 |
| 7 | A689E | 0.3 | 0.2 | 1.9 | 1.5 | 1.0 |
| 7 | A689D | 1.0 | 2.4 | 3.3 | 5.1 | 2.2 |
| 7 | A689G | 2.7 | 5.3 | 14.1 | 7.7 | 7.3 |
| 7 | A689G | 1.2 | 1.3 | 1.4 | 0.5 | 1.1 |
| 7 | A689W | 1.3 | 2.2 | 12.9 | 13.5 | 9.4 |
| 7 | M694S | 4.3 | 94.6 | 78.0 | 122.3 | 87.0 |
| 7 | M694S | 3.7 | 47.5 | 42.0 | 55.5 | 35.3 |
| 7 | H698V | 1.1 | 1.8 | 1.2 | 2.5 | 1.5 |
| 7 | H698Y | 1.1 | 1.0 | 0.6 | 2.3 | 1.0 |
| 7 | D699A | 1.3 | 0.5 | 6.9 | 10.3 | 23.5 |
| 7 | D699V | 0.7 | 1.1 | 1.4 | 5.2 | 4.0 |
| 7 | D700R | 0.8 | 0.8 | 3.0 | 2.6 | 3.3 |
| 7 | D700P | 1.2 | 3.7 | 11.5 | 33.1 | 23.4 |
| 7 | D700G | 0.8 | 0.8 | 4.8 | 2.4 | 1.0 |
| 7 | D700V | 0.3 | 0.4 | 2.9 | 1.0 | 1.7 |
| 8 | S701G | 1.5 | 1.9 | 4.0 | 8.6 | 5.1 |
| 8 | S701Y | 1.9 | 2.9 | 1.6 | 0.3 | 2.3 |
| 8 | S701S | 1.0 | 0.7 | 0.8 | 0.5 | 1.3 |
| 8 | S701S | 2.4 | 1.4 | 4.3 | 7.1 | 12.2 |
| 8 | S701W | 1.1 | 1.9 | 3.6 | 7.6 | 5.9 |
| 8 | L702K | 1.2 | 1.7 | 0.8 | 1.1 | 2.1 |
| 8 | L702L | 1.1 | 1.6 | 3.1 | 1.2 | 4.0 |
| 8 | L702V | 1.0 | 0.8 | 1.7 | 0.7 | 3.2 |
| 8 | L702V | 1.3 | 1.7 | 4.3 | 4.8 | 1.9 |
| 8 | L702L | 1.5 | 1.0 | 2.9 | 9.5 | 5.7 |
| 8 | T703G | 0.5 | 0.8 | 0.8 | 1.6 | 3.8 |
| 8 | T703S | 2.6 | 2.1 | 0.6 | 0.9 | 5.1 |
| 8 | K705N | 1.2 | 1.7 | 3.2 | 4.2 | 3.7 |
| 8 | K705R | 1.7 | 2.0 | 1.8 | 2.3 | 3.5 |
| 8 | K705R | 2.3 | 1.9 | 7.0 | 9.6 | 10.8 |
| 8 | E706T | 1.5 | 5.9 | 9.2 | 18.4 | 1.4 |
| 8 | E706R | 3.0 | 1.5 | 3.4 | 8.4 | 1.8 |
| 8 | E706R | 3.8 | 3.6 | 6.4 | 7.8 | 16.6 |
| 8 | E706G | 1.7 | 2.8 | 2.1 | 4.2 | 1.5 |
| 8 | E706S | 4.3 | 5.2 | 12.1 | 13.7 | 9.7 |
| 8 | E706C | 1.7 | 2.6 | 5.7 | 9.4 | 4.7 |
| 8 | D707N | 1.6 | 1.8 | 1.4 | 1.5 | 1.5 |
| 8 | D707T | 1.0 | 7.4 | 11.3 | 5.3 | 4.0 |
| 8 | I708V | 3.3 | 3.4 | 10.4 | 7.7 | 15.0 |
| 8 | I708L | 2.2 | 5.1 | 3.5 | 1.4 | 1.7 |
| 8 | I708F | 0.7 | 1.6 | 0.5 | 1.3 | 1.3 |
| 8 | Q709R | 2.5 | 6.9 | 11.8 | 12.9 | 10.8 |
| 8 | Q709H | 2.3 | 2.5 | 4.9 | 11.0 | 11.6 |
| 8 | Q709R | 1.2 | 0.6 | 1.9 | 3.1 | 6.6 |
| 8 | Q709A | 3.2 | 4.2 | 4.2 | 35.3 | 18.9 |
| 8 | K710S | 0.9 | 1.2 | 0.7 | 0.5 | 2.2 |
| 8 | K710Q | 0.7 | 1.2 | 0.7 | 1.0 | 1.1 |
| 8 | K710G | 1.6 | 3.1 | 4.2 | 18.0 | 6.0 |
| 8 | K710S | 1.1 | 3.9 | 12.1 | 12.4 | 25.5 |
| 8 | K710W | 2.0 | 2.2 | 2.4 | 2.6 | 6.3 |
| 8 | A711K | 4.1 | 4.3 | 6.4 | 4.7 | 1.8 |
| 8 | A711E | 1.0 | 2.3 | 6.2 | 4.6 | 17.6 |
| 8 | A711A | 2.0 | 3.2 | 9.2 | 10.2 | 13.7 |
| 8 | A711Y | 4.8 | 12.7 | 17.5 | 31.5 | 25.4 |
| 8 | Q712S | 1.5 | 1.6 | 3.6 | 3.5 | 2.8 |
| 8 | Q712I | 0.7 | 0.6 | 5.0 | 8.7 | 2.3 |
| 8 | Q712H | 1.4 | 2.0 | 0.5 | 1.6 | 4.4 |
| 8 | Q712G | 1.6 | 2.8 | 1.7 | 1.5 | 2.7 |
| 8 | V713N | 1.9 | 2.8 | 1.4 | 2.8 | 5.8 |
| 8 | V713S | 1.7 | 1.2 | 1.1 | 0.5 | 1.2 |
| 8 | V713R | 1.7 | 1.9 | 6.6 | 9.6 | 5.8 |
| 8 | V713D | 1.3 | 1.5 | 2.8 | 1.9 | 1.2 |
| 8 | V713V | 1.9 | 1.8 | 2.6 | 1.4 | 1.1 |
| 8 | V713W | 2.4 | 3.2 | 2.0 | 0.4 | 1.0 |
| 8 | V713C | 1.0 | 1.8 | 1.6 | 1.5 | 2.2 |
| 8 | V713F | 1.1 | 1.8 | 1.8 | 4.6 | 1.0 |
| 8 | S714K | 2.3 | 4.4 | 6.2 | 6.4 | 4.7 |
| 8 | S714M | 1.5 | 3.8 | 5.7 | 3.6 | 1.5 |
| 8 | S714I | 1.5 | 1.8 | 2.8 | 2.8 | 1.3 |
| 8 | S714R | 1.6 | 2.1 | 4.5 | 5.4 | 4.1 |
| 8 | G715R | 2.1 | 3.7 | 2.7 | 0.7 | 1.4 |
| 8 | G715H | 4.5 | 20.5 | 24.4 | 56.0 | 110.5 |
| 8 | G715R | 2.0 | 2.1 | 7.0 | 8.7 | 18.8 |
| 8 | G715R | 0.5 | 1.0 | 3.6 | 2.6 | 2.6 |
| 8 | G715A | 3.1 | 2.4 | 1.5 | 3.6 | 2.5 |
| 8 | G715G | 1.7 | 1.1 | 1.5 | 1.3 | 1.2 |
| 8 | G715V | 1.8 | 2.7 | 2.1 | 0.6 | 1.6 |
| 8 | G715L | 4.6 | 5.1 | 1.9 | 30.6 | 79.0 |
| 8 | Q716N | 1.9 | 0.9 | 3.2 | 9.7 | 4.8 |
| 8 | Q716Q | 2.1 | 4.2 | 8.9 | 4.7 | 5.5 |
| 8 | Q716R | 3.9 | 1.0 | 1.0 | 1.2 | 1.0 |
| 8 | Q716E | 0.7 | 0.4 | 2.8 | 1.8 | 3.6 |
| 8 | G717M | 0.9 | 2.6 | 1.6 | 1.0 | 15.5 |
| 8 | G717R | 0.7 | 2.3 | 3.2 | 4.4 | 2.8 |
| 8 | G717G | 1.7 | 3.4 | 7.2 | 6.6 | 4.2 |
| 8 | G717V | 1.5 | 1.7 | 2.0 | 1.7 | 3.6 |
| 8 | G717Y | 0.9 | 2.1 | 6.6 | 3.1 | 14.2 |
| 8 | G717L | 1.3 | 2.8 | 0.9 | 2.3 | 12.5 |
| 8 | D718K | 3.1 | 3.9 | 6.4 | 7.8 | 2.1 |
| 8 | D718N | 3.4 | 4.7 | 2.7 | 5.0 | 2.6 |
| 8 | D718T | 0.7 | 2.3 | 1.5 | 1.8 | 5.8 |
| 8 | D718S | 2.5 | 3.9 | 9.1 | 2.1 | 1.0 |
| 8 | D718P | 2.8 | 4.0 | 6.5 | 4.8 | 3.7 |
| 8 | D718L | 1.8 | 3.0 | 2.0 | 3.0 | 3.1 |
| 8 | D718A | 1.9 | 3.0 | 3.3 | 6.8 | 41.8 |
| 8 | D718G | 3.2 | 4.8 | 9.0 | 5.6 | 5.5 |
| 8 | D718V | 2.7 | 3.6 | 4.8 | 3.0 | 2.1 |
| 8 | D718Y | 1.3 | 1.7 | 2.3 | 11.8 | 7.8 |
| 8 | D718S | 3.5 | 5.1 | 4.9 | 34.6 | 43.9 |
| 8 | D718L | 1.3 | 1.3 | 2.5 | 2.5 | 6.6 |
| 8 | D718F | 1.8 | 4.5 | 2.6 | 10.0 | 4.5 |
| 8 | S719N | 1.0 | 1.7 | 2.4 | 3.4 | 2.3 |
| 8 | S719T | 2.1 | 4.5 | 7.3 | 23.2 | 14.4 |
| 8 | S719S | 2.1 | 0.9 | 2.0 | 0.9 | 1.9 |
| 8 | H721Q | 1.9 | 1.3 | 1.2 | 1.4 | 1.3 |
| 8 | H721Y | 1.3 | 2.5 | 3.1 | 6.4 | 8.4 |
| 8 | E722T | 2.2 | 4.5 | 6.3 | 22.9 | 10.7 |
| 8 | E722R | 3.2 | 4.9 | 4.7 | 4.4 | 1.8 |
| 8 | E722G | 3.6 | 7.0 | 2.4 | 2.5 | 2.8 |
| 8 | H723Q | 1.6 | 2.1 | 3.0 | 1.1 | 1.0 |
| 8 | H723H | 1.9 | 2.5 | 6.1 | 12.2 | 8.0 |
| 8 | H723R | 0.8 | 3.2 | 5.9 | 4.7 | 13.8 |
| 8 | H723L | 1.5 | 2.2 | 2.9 | 4.0 | 1.0 |
| 8 | A725H | 5.1 | 4.1 | 16.7 | 9.5 | 40.3 |
| 8 | A725Y | 4.8 | 10.2 | 20.3 | 38.2 | 10.2 |
| 8 | N726T | 1.3 | 2.1 | 1.3 | 4.8 | 3.4 |
| 8 | N726S | 2.2 | 6.7 | 0.4 | 2.5 | 4.5 |
| 8 | N726I | 1.0 | 1.6 | 1.4 | 1.7 | 1.6 |
| 8 | L727W | 1.1 | 1.6 | 1.1 | 0.9 | 2.9 |
| 8 | A728S | 0.9 | 1.7 | 1.3 | 1.9 | 2.1 |
| 8 | G729G | 0.9 | 1.9 | 3.5 | 2.1 | 2.7 |
| 8 | G729V | 1.3 | 2.7 | 1.3 | 3.1 | 4.6 |
| 8 | P731R | 2.0 | 2.2 | 9.3 | 18.6 | 30.8 |
| 8 | P731R | 2.4 | 1.2 | 3.6 | 20.2 | 18.1 |
| 8 | A732K | 2.3 | 2.8 | 8.3 | 13.7 | 36.4 |
| 8 | A732T | 2.6 | 3.1 | 2.4 | 6.8 | 12.2 |
| 8 | A732R | 4.1 | 2.2 | 15.2 | 15.7 | 31.9 |
| 8 | A732A | 3.4 | 6.8 | 5.5 | 13.1 | 16.1 |
| 8 | G736A | 1.7 | 3.1 | 3.1 | 19.6 | 7.1 |
| 8 | G736G | 1.3 | 1.5 | 1.2 | 3.4 | 1.8 |
| 8 | L738T | 1.1 | 2.4 | 11.2 | 14.8 | 10.0 |
| 8 | L738R | 1.4 | 1.8 | 5.8 | 9.0 | 12.2 |
| 8 | L738R | 1.1 | 1.2 | 5.1 | 4.8 | 8.7 |
| 8 | L738L | 2.5 | 6.3 | 6.0 | 15.3 | 2.0 |
| 8 | L738F | 1.6 | 1.8 | 3.9 | 7.4 | 3.8 |
| 8 | Q739L | 1.6 | 1.7 | 1.8 | 1.6 | 2.1 |
| 8 | Q739Y | 1.6 | 4.3 | 17.7 | 42.9 | 4.0 |
| 8 | Q739L | 1.4 | 2.0 | 10.5 | 27.3 | 39.2 |
| 8 | T740T | 2.0 | 2.2 | 2.6 | 3.6 | 4.3 |
| 8 | T740T | 1.8 | 1.7 | 0.8 | 1.2 | 1.0 |
| 8 | T740A | 3.5 | 3.1 | 2.9 | 7.9 | 3.1 |
| 8 | V741G | 1.0 | 0.9 | 1.0 | 1.0 | 1.1 |
| 8 | V741V | 1.7 | 1.6 | 1.8 | 1.6 | 1.8 |
| 8 | K742N | 1.7 | 1.7 | 1.9 | 1.6 | 1.9 |
| 8 | K742R | 3.1 | 6.3 | 0.4 | 4.0 | 4.7 |
| 8 | V743V | 3.5 | 3.3 | 2.2 | 0.8 | 1.4 |
| 8 | V744V | 2.3 | 3.7 | 4.0 | 4.3 | 3.1 |
| 8 | D745H | 1.5 | 0.9 | 1.8 | 1.3 | 1.2 |
| 8 | D745E | 2.3 | 1.6 | 3.4 | 1.0 | 1.4 |
| 8 | D745V | 1.3 | 1.5 | 1.5 | 1.6 | 1.7 |
| 8 | E746Q | 2.0 | 0.8 | 2.2 | 0.3 | 1.4 |
| 8 | E746V | 1.2 | 1.5 | 1.4 | 2.2 | 1.8 |
| 8 | L747I | 1.5 | 1.8 | 2.3 | 1.4 | 1.6 |
| 8 | L747V | 1.4 | 1.4 | 1.7 | 1.5 | 1.6 |
| 8 | V748I | 3.3 | 5.5 | 16.8 | 9.9 | 2.3 |
| 8 | V748V | 1.4 | 1.1 | 2.0 | 1.0 | 1.4 |
| 8 | K749M | 1.6 | 1.9 | 1.9 | 2.1 | 2.0 |
| 8 | K749S | 0.9 | 0.4 | 0.6 | 0.6 | 3.0 |
| 8 | M751L | 1.6 | 2.1 | 2.3 | 1.8 | 2.1 |
| 8 | G752G | 2.4 | 0.6 | 0.1 | 0.3 | 1.4 |
| 8 | G752V | 1.3 | 0.9 | 0.9 | 0.9 | 1.0 |
| 8 | G752Y | 0.4 | 0.4 | 3.2 | 10.4 | 2.1 |
| 8 | R753R | 1.7 | 0.7 | 1.3 | 4.4 | 2.3 |
| 8 | R753H | 1.7 | 5.5 | 3.0 | 6.7 | 7.8 |
| 8 | R753P | 1.2 | 1.4 | 1.5 | 1.6 | 1.8 |
| 8 | R753L | 1.5 | 1.6 | 1.7 | 1.8 | 2.0 |
| 8 | R753C | 0.9 | 1.2 | 2.4 | 1.7 | 3.0 |
| 8 | H754R | 1.3 | 1.8 | 1.1 | 0.9 | 2.8 |
| 8 | H754S | 3.8 | 6.7 | 12.6 | 7.5 | 18.5 |
| 8 | H754R | 4.0 | 2.2 | 3.4 | 5.2 | 4.7 |
| 8 | H754L | 1.8 | 1.7 | 1.6 | 1.5 | 2.1 |
| 8 | H754C | 1.3 | 0.9 | 5.2 | 18.3 | 5.5 |
| 8 | K755R | 1.7 | 3.4 | 5.9 | 6.7 | 7.8 |
| 8 | K755M | 1.3 | 1.5 | 1.6 | 1.4 | 1.6 |
| 8 | P756P | 0.8 | 0.9 | 1.5 | 2.2 | 2.2 |
| 8 | E757R | 4.2 | 3.6 | 2.0 | 5.6 | 10.8 |
| 8 | E757Q | 1.1 | 2.1 | 8.9 | 29.5 | 11.6 |
| 8 | E757E | 2.7 | 5.1 | 11.4 | 5.1 | 16.0 |
| 8 | N758N | 1.0 | 0.4 | 1.1 | 2.3 | 8.7 |
| 8 | N758T | 1.9 | 2.0 | 7.5 | 9.5 | 14.7 |
| 8 | N758M | 2.2 | 7.2 | 13.5 | 25.9 | 65.2 |
| 8 | N758I | 1.3 | 2.3 | 8.4 | 34.4 | 31.2 |
| 8 | N758H | 0.7 | 1.7 | 6.0 | 1.8 | 4.3 |
| 8 | N758R | 0.8 | 1.0 | 6.0 | 4.3 | 8.5 |
| 8 | N758A | 1.4 | 0.6 | 1.0 | 1.5 | 2.3 |
| 8 | N758Y | 3.3 | 14.5 | 79.0 | 181.6 | 640.5 |
| 8 | N758L | 1.1 | 1.0 | 2.1 | 1.4 | 6.4 |
| 8 | N758F | 1.8 | 7.0 | 5.6 | 9.9 | 2.2 |
| 8 | V760V | 1.5 | 2.4 | 2.5 | 2.8 | 1.4 |
| 8 | V760F | 3.7 | 7.8 | 13.6 | 13.4 | 18.4 |
| 8 | I761F | 1.6 | 1.9 | 2.0 | 2.0 | 2.2 |
| 8 | M763V | 1.1 | 1.2 | 1.2 | 0.8 | 2.0 |
| 8 | M763L | 1.9 | 1.9 | 1.8 | 1.8 | 1.8 |
| 8 | M763V | 1.3 | 1.5 | 1.6 | 1.3 | 2.6 |
| 8 | E766S | 0.9 | 0.5 | 2.8 | 8.5 | 6.0 |
| 8 | E766D | 1.4 | 0.9 | 2.7 | 1.5 | 2.5 |
| 8 | N767S | 1.0 | 1.3 | 1.5 | 2.4 | 3.6 |
| 8 | N767D | 1.0 | 1.7 | 1.3 | 1.7 | 3.5 |
| 8 | N767V | 1.2 | 1.3 | 2.2 | 2.7 | 4.3 |
| 8 | N767Y | 1.1 | 1.8 | 2.1 | 2.3 | 2.6 |
| 8 | N767F | 1.4 | 1.6 | 2.2 | 1.4 | 2.6 |
| 8 | Q768R | 0.7 | 0.7 | 1.3 | 1.1 | 5.5 |
| 8 | Q768L | 2.1 | 2.6 | 6.0 | 14.3 | 4.2 |
| 8 | Q768L | 1.8 | 3.9 | 2.0 | 1.9 | 2.0 |
| 8 | Q768E | 1.0 | 3.5 | 3.5 | 22.6 | 11.1 |
| 8 | Q768G | 0.8 | 1.2 | 1.8 | 0.7 | 3.8 |
| 8 | T769R | 0.8 | 0.9 | 1.0 | 0.4 | 1.1 |
| 8 | T769S | 1.2 | 1.4 | 2.7 | 3.9 | 1.0 |
| 8 | T769E | 1.0 | 1.5 | 2.8 | 3.3 | 4.0 |
| 8 | T769G | 1.5 | 1.6 | 1.5 | 2.8 | 2.4 |
| 8 | K772G | 0.7 | 0.6 | 1.1 | 0.8 | 2.6 |
| 8 | Q774K | 1.2 | 0.2 | 0.2 | 0.1 | 2.1 |
| 8 | Q774N | 0.5 | 1.5 | 10.0 | 6.3 | 1.0 |
| 8 | Q774R | 1.3 | 1.5 | 2.8 | 1.7 | 4.3 |
| 8 | K775Q | 1.5 | 1.5 | 2.0 | 1.1 | 1.3 |
| 8 | K775L | 1.8 | 3.7 | 2.2 | 5.9 | 7.2 |
| 8 | N776K | 0.5 | 0.4 | 1.4 | 5.4 | 2.6 |
| 8 | N776S | 1.1 | 1.1 | 1.2 | 1.2 | 1.3 |
| 8 | S777V | 2.4 | 3.9 | 4.6 | 1.1 | 2.7 |
| 8 | R778R | 2.2 | 2.6 | 2.4 | 4.8 | 2.0 |
| 8 | R778L | 1.5 | 1.5 | 3.9 | 1.2 | 1.7 |
| 8 | R778V | 0.7 | 3.3 | 8.2 | 3.7 | 2.0 |
| 8 | E779L | 0.5 | 2.4 | 0.1 | 3.2 | 4.2 |
| 8 | M781L | 1.2 | 1.3 | 6.1 | 11.6 | 4.5 |
| 8 | K782R | 1.8 | 1.1 | 1.6 | 6.5 | 2.4 |
| 8 | E785T | 2.2 | 3.1 | 7.0 | 8.8 | 19.0 |
| 8 | E785R | 3.5 | 5.0 | 15.8 | 26.2 | 5.9 |
| 8 | E785R | 2.6 | 6.6 | 6.1 | 4.3 | 1.9 |
| 8 | E785V | 1.6 | 1.5 | 3.1 | 4.9 | 6.9 |
| 8 | E786R | 1.1 | 1.3 | 3.3 | 7.0 | 4.9 |
| 8 | E786S | 2.5 | 2.0 | 2.9 | 2.7 | 1.7 |
| 8 | E786M | 1.7 | 1.7 | 2.2 | 3.7 | 3.4 |
| 8 | E786R | 2.1 | 3.2 | 3.7 | 3.9 | 10.3 |
| 8 | E786E | 1.1 | 1.8 | 2.1 | 6.4 | 4.7 |
| 8 | E786A | 2.5 | 3.7 | 2.5 | 0.8 | 3.4 |
| 8 | E786Y | 1.2 | 1.3 | 1.1 | 1.1 | 5.0 |
| 8 | E786W | 0.8 | 0.1 | 0.4 | 0.6 | 1.6 |
| 8 | I788L | 0.9 | 1.5 | 2.0 | 5.5 | 8.6 |
| 8 | I788V | 0.9 | 0.6 | 1.0 | 2.0 | 5.8 |
| 8 | K789L | 0.4 | 0.8 | 2.1 | 1.4 | 3.9 |
| 8 | K789C | 1.2 | 0.3 | 0.6 | 1.3 | 3.5 |
| 8 | E790K | 1.2 | 0.3 | 4.3 | 4.2 | 6.1 |
| 8 | E790T | 1.9 | 4.9 | 7.0 | 10.8 | 22.1 |
| 8 | E790M | 1.2 | 2.4 | 11.0 | 9.4 | 6.6 |
| 8 | E790A | 2.4 | 1.9 | 6.8 | 15.1 | 11.6 |
| 8 | E790V | 1.5 | 2.7 | 2.2 | 11.9 | 31.4 |
| 8 | E790Y | 2.6 | 1.9 | 7.1 | 2.6 | 7.3 |
| 8 | L791I | 0.1 | 0.0 | 5.4 | 6.9 | 1.4 |
| 8 | G792G | 1.4 | 1.0 | 2.3 | 2.9 | 4.6 |
| 8 | G792S | 1.0 | 2.2 | 10.2 | 14.2 | 1.5 |
| 8 | Q794V | 1.9 | 2.4 | 1.0 | 1.7 | 21.6 |
| 8 | L796M | 0.9 | 0.7 | 0.5 | 0.3 | 1.6 |
| 8 | K797T | 3.1 | 0.3 | 29.0 | 36.4 | 1.6 |
| 8 | E798N | 1.2 | 1.8 | 2.2 | 5.5 | 12.6 |
| 9 | V801C | 2.1 | 1.6 | 2.9 | 2.0 | 1.0 |
| 9 | E802T | 1.4 | 3.3 | 5.0 | 6.2 | 4.6 |
| 9 | E802Q | 1.6 | 1.3 | 0.8 | 2.6 | 5.3 |
| 9 | E802D | 1.8 | 1.7 | 2.0 | 2.2 | 1.0 |
| 9 | E802V | 1.4 | 1.3 | 0.5 | 0.5 | 2.1 |
| 9 | E802V | 1.0 | 0.1 | 1.0 | 0.8 | 3.1 |
| 9 | E802S | 1.8 | 4.5 | 7.8 | 5.8 | 21.3 |
| 9 | N803N | 0.9 | 1.6 | 3.4 | 4.6 | 5.7 |
| 9 | N803P | 3.6 | 8.9 | 34.5 | 39.6 | 25.2 |
| 9 | N803D | 1.1 | 1.5 | 7.9 | 7.1 | 4.2 |
| 9 | N803A | 0.8 | 1.9 | 2.4 | 7.7 | 8.0 |
| 9 | T804R | 1.8 | 2.5 | 1.7 | 2.4 | 3.1 |
| 9 | T804M | 1.2 | 2.5 | 1.1 | 0.8 | 1.8 |
| 9 | T804A | 1.2 | 1.9 | 2.9 | 2.8 | 2.0 |
| 9 | T804A | 1.6 | 2.2 | 1.2 | 1.9 | 2.7 |
| 9 | T804G | 2.1 | 2.2 | 2.5 | 2.0 | 1.6 |
| 9 | T804Y | 1.3 | 2.9 | 6.8 | 6.0 | 1.6 |
| 9 | Q805D | 2.1 | 5.7 | 9.3 | 13.0 | 10.2 |
| 9 | Q805A | 1.3 | 1.3 | 3.1 | 5.7 | 11.1 |
| 9 | Q805S | 2.1 | 2.5 | 4.1 | 3.2 | 1.8 |
| 9 | L806L | 1.1 | 1.3 | 0.5 | 0.9 | 2.4 |
| 9 | Q807I | 1.3 | 2.6 | 1.5 | 0.7 | 11.1 |
| 9 | Q807R | 1.7 | 1.6 | 1.3 | 2.5 | 1.7 |
| 9 | Q807G | 1.0 | 1.0 | 1.0 | 0.7 | 2.0 |
| 9 | N808R | 1.7 | 0.9 | 1.6 | 3.2 | 1.2 |
| 9 | N808D | 2.3 | 3.4 | 3.8 | 4.8 | 5.0 |
| 9 | N808S | 1.2 | 0.4 | 1.1 | 0.7 | 1.1 |
| 9 | E809D | 1.2 | 1.1 | 1.0 | 1.3 | 1.2 |
| 9 | K810N | 1.3 | 0.9 | 0.9 | 0.9 | 1.0 |
| 9 | K810R | 1.2 | 1.1 | 0.9 | 0.3 | 1.0 |
| 9 | K810R | 3.5 | 2.8 | 3.3 | 7.0 | 3.4 |
| 9 | L811L | 1.8 | 3.2 | 5.5 | 7.1 | 7.4 |
| 9 | L811V | 2.1 | 3.3 | 2.5 | 2.9 | 1.5 |
| 9 | Y814F | 2.0 | 0.4 | 3.0 | 3.2 | 2.7 |
| 9 | Y815N | 1.6 | 2.2 | 2.3 | 2.4 | 2.2 |
| 9 | Y815STOP | 1.3 | 1.5 | 1.7 | 1.9 | 1.7 |
| 9 | G819G | 1.7 | 4.3 | 8.6 | 8.6 | 7.7 |
| 9 | R820R | 1.4 | 1.8 | 1.0 | 1.4 | 1.8 |
| 9 | Y823Y | 1.5 | 1.5 | 1.3 | 1.5 | 1.8 |
| 9 | V824T | 0.8 | 4.2 | 4.4 | 8.7 | 15.0 |
| 9 | V824E | 2.2 | 1.2 | 1.6 | 2.5 | 2.3 |
| 9 | V824V | 1.4 | 1.8 | 3.5 | 4.0 | 2.2 |
| 9 | D825D | 1.2 | 2.3 | 2.9 | 3.7 | 3.8 |
| 9 | D825S | 3.4 | 1.1 | 2.2 | 1.0 | 2.0 |
| 9 | Q826M | 1.5 | 3.8 | 2.8 | 0.9 | 1.7 |
| 9 | E827N | 2.6 | 4.9 | 5.0 | 5.9 | 5.4 |
| 9 | E827R | 3.1 | 1.7 | 5.1 | 8.0 | 14.0 |
| 9 | E827S | 0.7 | 1.3 | 5.0 | 3.4 | 3.2 |
| 9 | E827Q | 1.6 | 5.9 | 6.0 | 8.4 | 16.4 |
| 9 | E827C | 1.2 | 0.2 | 1.6 | 3.0 | 3.5 |
| 9 | L828L | 1.6 | 4.6 | 2.9 | 1.9 | 1.6 |
| 9 | L828V | 1.4 | 1.1 | 1.5 | 1.1 | 1.1 |
| 9 | N831T | 4.6 | 4.1 | 2.7 | 3.0 | 8.3 |
| 9 | R832Q | 3.7 | 4.0 | 14.5 | 32.3 | 70.3 |
| 9 | R832L | 1.9 | 2.5 | 3.0 | 1.3 | 1.1 |
| 9 | L833L | 2.3 | 2.4 | 2.8 | 1.5 | 2.9 |
| 9 | S834T | 0.7 | 0.5 | 0.4 | 0.5 | 1.5 |
| 9 | S834C | 2.7 | 3.3 | 2.9 | 4.0 | 5.0 |
| 9 | L833V | 0.8 | 1.3 | 0.4 | 1.3 | 1.0 |
| 9 | L833L | 2.8 | 0.8 | 1.2 | 1.1 | 2.1 |
| 9 | S834T | 0.6 | 3.3 | 4.5 | 5.1 | 20.1 |
| 9 | S834C | 1.2 | 0.4 | 0.5 | 0.4 | 1.0 |
| 9 | D835S | 0.8 | 0.7 | 0.0 | 1.9 | 2.5 |
| 9 | D835E | 1.0 | 1.6 | 4.8 | 4.6 | 3.9 |
| 9 | D837N | 1.8 | 3.3 | 2.5 | 2.0 | 2.7 |
| 9 | D837L | 1.1 | 3.2 | 3.7 | 6.1 | 3.1 |
| 9 | V838V | 0.8 | 2.4 | 5.3 | 4.0 | 3.2 |
| 9 | D839G | 0.8 | 0.8 | 0.1 | 0.8 | 1.0 |
| 9 | A840T | 0.5 | 1.9 | 6.7 | 3.8 | 2.1 |
| 9 | A840R | 1.7 | 1.9 | 3.0 | 4.3 | 2.9 |
| 9 | A840R | 2.2 | 1.5 | 5.6 | 5.9 | 4.6 |
| 9 | A840D | 1.1 | 1.6 | 1.8 | 3.6 | 1.7 |
| 9 | A840V | 1.5 | 3.4 | 3.1 | 1.8 | 1.6 |
| 9 | A840Y | 2.6 | 4.6 | 24.7 | 11.3 | 23.7 |
| 9 | A840W | 1.3 | 0.2 | 4.0 | 5.3 | 8.7 |
| 9 | A840L | 0.9 | 1.9 | 5.4 | 5.7 | 2.6 |
| 9 | I841I | 1.2 | 1.0 | 2.1 | 2.5 | 2.2 |
| 9 | Q844E | 2.8 | 6.8 | 12.6 | 14.0 | 10.9 |
| 9 | S845S | 2.1 | 2.4 | 3.4 | 4.9 | 4.0 |
| 9 | S845A | 1.8 | 3.4 | 6.9 | 14.1 | 15.4 |
| 9 | S845A | 2.6 | 2.3 | 5.0 | 2.4 | 5.3 |
| 9 | L847V | 1.8 | 1.3 | 1.5 | 1.9 | 1.7 |
| 9 | L847L | 2.3 | 2.6 | 3.5 | 3.1 | 2.4 |
| 9 | L847F | 1.7 | 2.2 | 4.9 | 2.2 | 1.4 |
| 9 | K848K | 1.4 | 0.9 | 2.0 | 3.0 | 3.0 |
| 9 | K848S | 1.3 | 3.4 | 4.2 | 2.1 | 5.3 |
| 9 | K848Q | 1.2 | 1.0 | 0.4 | 0.2 | 1.1 |
| 9 | I852F | 1.9 | 1.0 | 1.0 | 1.1 | 1.1 |
| 9 | D853A | 1.7 | 0.2 | 0.4 | 0.3 | 1.1 |
| 9 | K855R | 3.2 | 4.2 | 10.3 | 9.8 | 13.1 |
| 9 | K855L | 5.4 | 8.3 | 0.2 | 6.0 | 4.3 |
| 9 | K855E | 1.1 | 1.2 | 1.2 | 1.2 | 1.1 |
| 9 | K855STOP | 1.5 | 1.5 | 1.5 | 1.7 | 1.5 |
| 9 | L857Q | 1.5 | 0.9 | 1.9 | 1.4 | 1.2 |
| 9 | R859H | 0.9 | 4.7 | 4.0 | 6.3 | 5.0 |
| 9 | R859A | 1.9 | 6.2 | 10.3 | 5.4 | 12.4 |
| 9 | R859G | 0.8 | 1.0 | 4.9 | 2.8 | 1.1 |
| 9 | S860T | 1.2 | 1.6 | 1.5 | 1.5 | 1.5 |
| 9 | S860A | 1.4 | 1.4 | 1.3 | 1.4 | 1.3 |
| 9 | S860Y | 1.5 | 1.7 | 2.4 | 1.9 | 1.7 |
| 9 | S860S | 1.1 | 1.3 | 4.5 | 7.4 | 5.8 |
| 9 | S860W | 2.2 | 1.5 | 3.7 | 2.3 | 1.7 |
| 9 | S860C | 1.6 | 2.0 | 2.9 | 2.7 | 1.4 |
| 9 | D861N | 1.7 | 0.7 | 1.4 | 1.6 | 1.4 |
| 9 | D861S | 1.2 | 2.0 | 0.6 | 0.7 | 1.6 |
| 9 | D861I | 2.1 | 1.0 | 2.2 | 1.1 | 2.1 |
| 9 | D861Q | 1.1 | 0.2 | 6.9 | 9.2 | 14.2 |
| 9 | D861H | 0.5 | 0.4 | 0.6 | 1.3 | 2.5 |
| 9 | D861R | 2.0 | 2.2 | 7.4 | 5.4 | 4.8 |
| 9 | D861R | 1.4 | 2.3 | 6.9 | 12.8 | 15.9 |
| 9 | D861A | 3.0 | 6.3 | 14.7 | 17.4 | 16.3 |
| 9 | D861A | 2.0 | 4.4 | 8.8 | 8.1 | 5.3 |
| 9 | D861G | 2.6 | 2.8 | 3.1 | 3.4 | 2.0 |
| 9 | D861V | 0.6 | 1.7 | 6.6 | 12.3 | 17.2 |
| 9 | D861V | 0.8 | 0.7 | 1.6 | 4.1 | 8.0 |
| 9 | D861Y | 0.8 | 0.6 | 0.9 | 1.5 | 1.4 |
| 9 | D861S | 0.7 | 3.5 | 3.1 | 2.6 | 2.0 |
| 9 | D861C | 2.2 | 7.3 | 21.1 | 35.8 | 37.5 |
| 9 | D861F | 1.7 | 2.3 | 3.1 | 5.4 | 3.5 |
| 9 | K862N | 1.9 | 4.3 | 0.9 | 2.7 | 2.5 |
| 9 | K862I | 2.2 | 5.1 | 5.8 | 2.8 | 2.5 |
| 9 | K862Y | 1.1 | 1.7 | 2.2 | 1.1 | 7.8 |
| 9 | K862L | 1.0 | 1.1 | 2.6 | 1.8 | 1.4 |
| 9 | G865G | 1.1 | 1.1 | 0.3 | 0.5 | 3.3 |
| 9 | K866N | 1.2 | 1.3 | 3.8 | 7.5 | 3.0 |
| 9 | K866T | 2.0 | 3.5 | 11.0 | 4.3 | 5.3 |
| 9 | K866R | 1.5 | 17.4 | 12.7 | 10.7 | 4.3 |
| 9 | K866A | 0.4 | 0.9 | 2.2 | 2.4 | 3.6 |
| 9 | S867T | 1.7 | 2.0 | 1.3 | 0.7 | 1.1 |
| 9 | S867S | 0.7 | 0.1 | 3.6 | 1.4 | 4.5 |
| 9 | S867S | 1.9 | 1.7 | 3.2 | 3.3 | 2.5 |
| 9 | D868D | 1.3 | 1.6 | 0.6 | 1.4 | 1.5 |
| 9 | N869T | 1.7 | 6.3 | 4.2 | 2.4 | 1.3 |
| 9 | S872T | 0.6 | 0.0 | 1.7 | 1.3 | 4.9 |
| 9 | E873R | 3.9 | 4.4 | 5.8 | 1.9 | 4.0 |
| 9 | E873A | 2.3 | 1.7 | 6.6 | 7.2 | 12.1 |
| 9 | E873V | 1.5 | 2.9 | 1.3 | 0.7 | 3.2 |
| 9 | E873Y | 0.2 | 0.9 | 2.9 | 3.4 | 2.0 |
| 9 | E873W | 1.7 | 7.7 | 16.7 | 10.9 | 1.0 |
| 9 | E874K | 1.4 | 3.2 | 3.6 | 5.6 | 3.9 |
| 9 | E874M | 1.8 | 8.1 | 6.4 | 12.0 | 13.7 |
| 9 | E874G | 0.3 | 0.3 | 0.8 | 0.5 | 1.1 |
| 9 | E874Y | 0.6 | 2.7 | 2.6 | 2.9 | 8.8 |
| 9 | E874S | 2.5 | 1.4 | 6.3 | 8.0 | 7.2 |
| 9 | V875I | 1.3 | 1.6 | 1.6 | 1.8 | 1.6 |
| 9 | V875V | 1.1 | 3.1 | 1.7 | 0.6 | 1.9 |
| 9 | K877N | 1.2 | 2.8 | 1.1 | 0.8 | 1.0 |
| 9 | K878T | 2.1 | 1.7 | 1.1 | 0.6 | 1.5 |
| 9 | K878T | 1.9 | 3.7 | 14.9 | 11.9 | 2.3 |
| 9 | K878S | 1.7 | 0.8 | 4.1 | 2.3 | 1.7 |
| 9 | K878C | 1.1 | 1.4 | 5.9 | 0.8 | 2.3 |
| 9 | M879R | 0.8 | 1.0 | 1.4 | 1.3 | 1.0 |
| 9 | M879R | 0.9 | 2.1 | 0.1 | 1.1 | 1.2 |
| 9 | K880N | 1.3 | 1.5 | 1.6 | 1.2 | 1.4 |
| 9 | N881I | 2.2 | 1.4 | 1.7 | 1.6 | 1.6 |
| 9 | N881Y | 1.2 | 1.3 | 1.4 | 1.2 | 1.3 |
| 9 | Y882N | 1.6 | 1.6 | 1.6 | 1.4 | 1.3 |
| 9 | Y882F | 2.1 | 1.3 | 1.7 | 1.9 | 1.6 |
| 9 | R884L | 9.4 | 1.3 | 9.5 | 7.3 | 12.0 |
| 9 | R884E | 0.5 | 1.8 | 4.0 | 3.7 | 3.5 |
| 9 | R884S | 3.0 | 6.1 | 0.3 | 14.6 | 3.6 |
| 9 | Q885A | 4.6 | 6.0 | 5.1 | 12.9 | 6.9 |
| 9 | Q885S | 1.2 | 1.7 | 4.6 | 10.1 | 10.0 |
| 9 | L886P | 0.9 | 1.3 | 0.9 | 1.2 | 1.1 |
| 9 | L886L | 1.6 | 0.8 | 1.6 | 1.3 | 7.6 |
| 9 | L887C | 2.1 | 4.0 | 9.5 | 6.5 | 4.2 |
| 9 | N888K | 1.6 | 3.0 | 6.6 | 4.5 | 2.6 |
| 9 | N888S | 2.9 | 5.5 | 9.4 | 10.9 | 10.3 |
| 9 | N888C | 1.6 | 4.1 | 16.6 | 26.3 | 3.5 |
| 9 | K890R | 3.1 | 13.4 | 36.0 | 56.6 | 54.7 |
| 9 | K890A | 1.3 | 2.4 | 1.5 | 0.7 | 2.6 |
| 9 | K890G | 2.1 | 2.6 | 8.1 | 9.6 | 11.9 |
| 9 | L891Q | 1.5 | 1.3 | 1.4 | 1.2 | 1.2 |
| 9 | T893T | 1.9 | 1.4 | 1.2 | 1.1 | 1.1 |
| 9 | T893S | 1.0 | 4.1 | 2.6 | 3.9 | 1.9 |
| 9 | Q894R | 5.1 | 9.8 | 42.1 | 19.0 | 5.6 |
| 9 | Q894Q | 1.4 | 1.6 | 1.4 | 1.2 | 1.3 |
| 9 | Q894H | 1.5 | 1.8 | 1.9 | 2.0 | 1.8 |
| 9 | Q894A | 6.5 | 21.0 | 14.9 | 20.7 | 16.0 |
| 9 | R895V | 2.1 | 5.8 | 29.2 | 113.1 | 381.2 |
| 9 | R895V | 0.6 | 0.1 | 4.8 | 4.8 | 12.7 |
| 9 | F897F | 2.5 | 1.9 | 2.2 | 2.8 | 1.9 |
| 9 | N899H | 2.9 | 9.7 | 8.5 | 11.6 | 19.2 |
| 9 | N899L | 2.4 | 9.0 | 7.4 | 8.1 | 3.3 |
| 9 | N899Y | 3.2 | 5.7 | 6.1 | 9.4 | 4.6 |
| 9 | L900STOP | 1.2 | 1.2 | 1.4 | 1.3 | 1.3 |
| 10 | E904D | 1.1 | 0.8 | 0.8 | 0.9 | 1.4 |
| 10 | G907G | 0.8 | 0.5 | 1.7 | 6.5 | 6.6 |
| 10 | S909S | 2.5 | 2.7 | 5.1 | 5.5 | 1.3 |
| 10 | S909S | 1.8 | 2.1 | 21.2 | 6.2 | 59.5 |
| 10 | E910M | 0.3 | 0.5 | 3.4 | 4.1 | 16.2 |
| 10 | E910A | 1.1 | 1.4 | 6.1 | 6.3 | 3.7 |
| 10 | E910G | 1.8 | 0.3 | 5.2 | 6.8 | 31.8 |
| 10 | E910V | 1.4 | 6.7 | 7.2 | 18.8 | 8.2 |
| 10 | L911T | 2.0 | 18.6 | 61.7 | 52.6 | 35.2 |
| 10 | L911E | 0.4 | 1.2 | 2.3 | 2.1 | 4.5 |
| 10 | K913STOP | 1.1 | 1.0 | 1.0 | 0.4 | 1.1 |
| 10 | G915M | 0.9 | 7.3 | 21.5 | 13.3 | 1.2 |
| 10 | 1917V | 1.1 | 1.1 | 1.1 | 1.0 | 1.0 |
| 10 | K918R | 2.3 | 95.3 | 114.6 | 171.1 | 58.0 |
| 10 | R919R | 1.0 | 1.8 | 2.7 | 7.0 | 9.3 |
| 10 | T924S | 0.9 | 1.3 | 0.6 | 4.2 | 4.8 |
| 10 | R925R | 0.5 | 1.3 | 2.7 | 0.1 | 1.4 |
| 10 | Q926Q | 0.9 | 2.0 | 4.1 | 5.1 | 5.4 |
| 10 | Q926L | 2.7 | 12.0 | 6.2 | 28.8 | 1.2 |
| 10 | Q926V | 3.5 | 6.4 | 2.9 | 2.7 | 5.5 |
| 10 | T928A | 1.7 | 3.9 | 9.1 | 3.2 | 28.9 |
| 10 | T928F | 0.7 | 3.1 | 20.5 | 4.1 | 6.7 |
| 10 | H930S | 1.6 | 13.4 | 25.2 | 21.2 | 10.6 |
| 10 | I934V | 1.6 | 1.0 | 2.1 | 2.6 | 1.6 |
| 10 | L935I | 3.8 | 17.8 | 25.8 | 154.4 | 194.1 |
| 10 | D936S | 2.2 | 33.8 | 34.0 | 8.4 | 5.5 |
| 10 | S937N | 0.6 | 3.5 | 6.0 | 6.7 | 13.2 |
| 10 | S937R | 3.5 | 2.8 | 9.6 | 3.5 | 8.6 |
| 10 | S937A | 0.7 | 2.3 | 15.6 | 31.0 | 27.6 |
| 10 | T941T | 3.5 | 3.5 | 14.5 | 0.7 | 14.3 |
| 10 | K942R | 1.6 | 0.6 | 7.5 | 7.2 | 1.3 |
| 10 | K942S | 1.0 | 0.8 | 3.3 | 14.7 | 23.3 |
| 10 | K942M | 0.3 | 1.9 | 3.8 | 2.1 | 4.9 |
| 10 | E945R | 0.8 | 4.9 | 34.6 | 28.9 | 7.2 |
| 10 | E945L | 2.6 | 11.5 | 109.3 | 63.5 | 38.8 |
| 10 | N946K | 1.2 | 1.4 | 1.3 | 1.1 | 1.1 |
| 10 | D947L | 1.8 | 3.0 | 15.5 | 4.9 | 7.2 |
| 10 | K948N | 1.3 | 1.3 | 1.4 | 1.1 | 1.0 |
| 10 | L949Q | 1.1 | 1.4 | 1.1 | 1.1 | 1.0 |
| 10 | L949L | 0.4 | 0.6 | 2.8 | 5.2 | 13.7 |
| 10 | R951R | 1.1 | 1.5 | 1.0 | 1.1 | 1.2 |
| 10 | E952G | 1.8 | 1.4 | 6.2 | 26.3 | 23.3 |
| 10 | K954N | 1.0 | 1.3 | 1.3 | 1.1 | 1.1 |
| 10 | 1956N | 1.0 | 1.2 | 1.0 | 1.1 | 1.0 |
| 10 | K961K | 1.1 | 6.1 | 5.0 | 22.8 | 11.3 |
| 10 | K961R | 1.5 | 27.0 | 13.9 | 31.6 | 44.2 |
| 10 | K961E | 7.0 | 8.3 | 5.4 | 36.2 | 36.8 |
| 10 | K961S | 100.6 | 98.6 | 87.6 | 195.9 | 251.2 |
| 10 | V963G | 1.0 | 1.2 | 1.3 | 1.2 | 1.1 |
| 10 | V963V | 1.1 | 1.1 | 1.1 | 1.0 | 1.0 |
| 10 | S964C | 1.1 | 1.5 | 1.3 | 1.3 | 1.1 |
| 10 | K968T | 2.0 | 1.5 | 10.1 | 8.8 | 20.4 |
| 10 | K968Q | 2.2 | 4.1 | 13.5 | 25.3 | 23.4 |
| 10 | V975V | 2.3 | 3.7 | 9.5 | 7.4 | 5.1 |
| 10 | V975L | 3.7 | 14.8 | 12.8 | 31.7 | 5.4 |
| 10 | N980K | 1.2 | 1.0 | 0.9 | 1.0 | 1.0 |
| 10 | H985L | 1.2 | 0.9 | 0.1 | 0.5 | 1.2 |
| 10 | L989Q | 1.1 | 1.1 | 1.0 | 1.0 | 1.1 |
| 10 | N990T | 6.9 | 19.4 | 64.4 | 31.1 | 18.9 |
| 10 | N990T | 1.3 | 1.2 | 0.4 | 1.1 | 1.1 |
| 10 | V992E | 1.0 | 1.0 | 0.9 | 0.9 | 1.0 |
| 10 | T995T | 1.1 | 4.3 | 5.6 | 9.5 | 1.0 |
| 10 | A996V | 1.0 | 7.5 | 4.1 | 11.4 | 16.2 |
| 10 | L997L | 1.2 | 1.3 | 1.8 | 3.6 | 3.3 |
| 10 | I998R | 1.9 | 1.7 | 11.5 | 14.2 | 13.4 |
| 11 | P1002P | 1.3 | 2.9 | 2.9 | 3.0 | 5.1 |
| 11 | K1003M | 4.3 | 1.8 | 1.4 | 9.1 | 1.7 |
| 11 | K1003L | 6.0 | 13.6 | 8.7 | 4.9 | 1.4 |
| 11 | L1004L | 0.4 | 2.7 | 2.0 | 2.8 | 2.5 |
| 11 | E1005K | 2.1 | 3.9 | 4.5 | 5.9 | 2.1 |
| 11 | E1005R | 2.5 | 2.6 | 4.8 | 7.3 | 9.3 |
| 11 | E1005M | 2.0 | 4.9 | 7.6 | 5.9 | 6.3 |
| 11 | E1005I | 1.2 | 3.6 | 3.2 | 2.7 | 1.2 |
| 11 | E1005R | 2.8 | 3.8 | 10.3 | 21.0 | 22.5 |
| 11 | E1005L | 0.8 | 1.1 | 0.9 | 1.0 | 1.0 |
| 11 | E1005A | 2.6 | 3.8 | 3.9 | 3.9 | 2.4 |
| 11 | E1005G | 1.7 | 1.5 | 2.7 | 3.8 | 1.9 |
| 11 | E1005V | 0.8 | 1.1 | 3.1 | 2.5 | 1.8 |
| 11 | E1005S | 3.0 | 4.8 | 9.1 | 4.1 | 3.3 |
| 11 | E1005S | 4.9 | 12.5 | 11.0 | 7.9 | 4.8 |
| 11 | E1005W | 1.7 | 2.6 | 3.2 | 2.7 | 2.3 |
| 11 | S1006S | 0.4 | 1.7 | 3.1 | 3.2 | 2.4 |
| 11 | E1007N | 1.6 | 2.3 | 5.8 | 3.4 | 2.6 |
| 11 | E1007T | 4.6 | 2.8 | 4.5 | 5.5 | 1.9 |
| 11 | E1007M | 2.2 | 5.1 | 5.1 | 5.6 | 7.3 |
| 11 | E1007Q | 1.0 | 1.3 | 1.2 | 0.7 | 1.1 |
| 11 | E1007L | 3.4 | 9.1 | 19.0 | 26.6 | 35.4 |
| 11 | E1007L | 3.8 | 10.1 | 6.2 | 1.9 | 2.8 |
| 11 | E1007A | 0.6 | 1.7 | 2.0 | 2.0 | 1.3 |
| 11 | E1007V | 1.8 | 2.4 | 2.2 | 2.0 | 1.1 |
| 11 | E1007V | 1.4 | 2.1 | 2.3 | 3.4 | 2.8 |
| 11 | E1007W | 1.5 | 1.6 | 1.3 | 4.0 | 1.7 |
| 11 | E1007C | 2.5 | 6.1 | 7.0 | 7.5 | 7.2 |
| 11 | E1007L | 3.1 | 7.9 | 10.6 | 15.2 | 16.3 |
| 11 | F1008T | 2.0 | 4.4 | 5.7 | 5.3 | 1.0 |
| 11 | F1008M | 1.4 | 2.3 | 2.7 | 2.0 | 1.4 |
| 11 | F1008I | 2.0 | 5.3 | 6.6 | 7.2 | 9.1 |
| 11 | F1008L | 3.1 | 4.2 | 5.4 | 8.9 | 7.1 |
| 11 | F1008L | 2.4 | 1.4 | 2.4 | 4.1 | 4.2 |
| 11 | F1008A | 1.5 | 4.8 | 5.1 | 5.2 | 3.9 |
| 11 | F1008V | 1.8 | 3.4 | 4.9 | 6.7 | 6.6 |
| 11 | F1008V | 1.9 | 5.5 | 11.2 | 22.8 | 50.8 |
| 11 | F1008C | 1.5 | 3.4 | 4.7 | 5.5 | 3.2 |
| 11 | F1008L | 1.9 | 5.7 | 11.0 | 21.2 | 31.8 |
| 11 | Y1010Q | 0.4 | 2.6 | 3.9 | 6.7 | 5.1 |
| 11 | Y1010H | 3.6 | 2.3 | 4.9 | 8.7 | 10.2 |
| 11 | Y1010V | 1.0 | 2.7 | 3.1 | 2.9 | 1.7 |
| 11 | Y1010W | 1.0 | 0.8 | 1.3 | 2.1 | 2.4 |
| 11 | G1011G | 1.5 | 1.2 | 2.8 | 2.0 | 1.2 |
| 11 | D1012K | 1.0 | 1.6 | 1.7 | 1.4 | 1.8 |
| 11 | D1012M | 0.4 | 3.1 | 5.1 | 5.4 | 4.6 |
| 11 | D1012L | 0.2 | 3.5 | 2.9 | 1.5 | 1.3 |
| 11 | D1012V | 2.2 | 3.5 | 4.5 | 3.2 | 2.1 |
| 11 | Y1013N | 0.8 | 2.9 | 6.3 | 7.8 | 10.9 |
| 11 | K1014R | 2.6 | 9.7 | 8.6 | 3.2 | 2.6 |
| 11 | K1014G | 2.4 | 1.4 | 4.3 | 2.7 | 1.8 |
| 11 | K1014V | 0.3 | 4.1 | 4.9 | 8.5 | 3.5 |
| 11 | K1014C | 3.7 | 6.6 | 5.6 | 2.7 | 1.0 |
| 11 | V1015S | 1.8 | 3.9 | 6.5 | 9.4 | 8.7 |
| 11 | V1015Q | 0.2 | 3.4 | 4.3 | 1.9 | 1.1 |
| 11 | V1015H | 7.8 | 9.5 | 16.2 | 2.2 | 2.5 |
| 11 | V1015G | 1.2 | 2.1 | 2.9 | 2.6 | 1.0 |
| 11 | Y1016S | 2.4 | 6.6 | 5.9 | 4.1 | 2.1 |
| 11 | D1017A | 0.8 | 1.3 | 3.9 | 6.8 | 4.5 |
| 11 | V1018I | 3.1 | 2.9 | 1.5 | 2.2 | 3.1 |
| 11 | R1019K | 0.2 | 3.3 | 3.2 | 0.8 | 1.0 |
| 11 | R1019S | 4.4 | 1.7 | 1.8 | 2.6 | 2.4 |
| 11 | R1019C | 1.2 | 1.2 | 2.4 | 3.3 | 1.3 |
| 11 | K1020K | 6.4 | 5.7 | 4.1 | 3.7 | 4.7 |
| 11 | K1020N | 3.5 | 1.4 | 1.1 | 1.1 | 1.0 |
| 11 | K1020T | 5.2 | 21.8 | 50.4 | 44.8 | 26.8 |
| 11 | K1020S | 0.2 | 3.4 | 6.2 | 9.8 | 8.2 |
| 11 | K1020L | 6.1 | 8.8 | 17.0 | 8.0 | 3.2 |
| 11 | K1020E | 0.2 | 11.7 | 15.4 | 44.3 | 53.8 |
| 11 | K1020S | 3.6 | 14.8 | 15.2 | 45.6 | 13.7 |
| 11 | K1020F | 0.7 | 1.1 | 2.0 | 3.1 | 2.0 |
| 11 | M1021I | 1.4 | 0.4 | 0.7 | 1.2 | 1.4 |
| 11 | M1021H | 1.1 | 9.0 | 13.1 | 8.4 | 3.0 |
| 11 | M1021L | 3.6 | 6.2 | 3.5 | 12.2 | 8.4 |
| 11 | I1022F | 8.4 | 10.7 | 21.7 | 30.7 | 23.7 |
| 11 | K1024E | 5.3 | 8.0 | 3.1 | 4.5 | 1.9 |
| 11 | K1024A | 0.2 | 3.9 | 3.5 | 5.4 | 7.4 |
| 11 | K1024V | 7.4 | 33.1 | 46.1 | 32.7 | 13.5 |
| 11 | K1024C | 0.3 | 1.0 | 0.8 | 1.8 | 1.3 |
| 11 | E1026S | 0.4 | 0.8 | 3.6 | 7.5 | 5.5 |
| 11 | Q1027K | 1.5 | 2.0 | 8.0 | 5.0 | 1.2 |
| 11 | Q1027W | 2.6 | 2.0 | 4.1 | 5.0 | 7.2 |
| 11 | I1029S | 0.6 | 1.0 | 2.1 | 1.4 | 2.0 |
| 11 | I1029M | 2.4 | 2.9 | 5.1 | 4.3 | 1.8 |
| 11 | I1029Q | 5.7 | 7.7 | 6.8 | 9.3 | 8.3 |
| 11 | I1029L | 0.2 | 4.5 | 6.9 | 5.0 | 1.7 |
| 11 | I1029E | 2.7 | 7.1 | 10.9 | 6.9 | 6.7 |
| 11 | I1029A | 3.9 | 10.4 | 27.1 | 33.9 | 32.5 |
| 11 | K1031N | 1.3 | 1.3 | 1.3 | 1.2 | 1.1 |
| 11 | K1031M | 0.9 | 2.2 | 2.6 | 4.1 | 3.6 |
| 11 | K1031L | 0.2 | 0.7 | 1.4 | 2.0 | 1.1 |
| 11 | K1031G | 2.9 | 9.0 | 7.9 | 2.6 | 1.1 |
| 11 | K1031S | 0.2 | 8.7 | 7.1 | 7.7 | 4.0 |
| 11 | A1034T | 3.7 | 21.9 | 21.9 | 7.9 | 12.2 |
| 11 | A1034T | 6.7 | 20.4 | 26.1 | 39.4 | 39.5 |
| 11 | A1034M | 2.5 | 8.3 | 18.3 | 25.3 | 19.3 |
| 11 | A1034E | 0.7 | 10.1 | 11.6 | 25.9 | 34.8 |
| 11 | A1034G | 2.5 | 2.4 | 9.0 | 12.8 | 20.3 |
| 11 | A1034G | 1.0 | 2.8 | 5.0 | 6.0 | 7.0 |
| 11 | K1035N | 1.6 | 1.7 | 4.0 | 11.6 | 7.9 |
| 11 | K1035D | 0.2 | 2.6 | 2.0 | 0.8 | 1.9 |
| 11 | Y1036N | 1.1 | 1.3 | 1.1 | 1.0 | 1.0 |
| 11 | Y1036H | 3.0 | 0.7 | 1.0 | 1.9 | 1.9 |
| 11 | Y1036E | 1.9 | 5.3 | 4.4 | 0.6 | 1.1 |
| 11 | F1037E | 0.5 | 1.1 | 1.9 | 2.3 | 1.2 |
| 11 | S1040T | 1.6 | 3.6 | 5.5 | 2.7 | 3.1 |
| 11 | I1042L | 5.1 | 5.2 | 2.7 | 2.0 | 1.4 |
| 11 | M1043L | 1.9 | 3.1 | 3.6 | 1.5 | 1.6 |
| 11 | T1051M | 3.6 | 5.8 | 5.4 | 7.3 | 3.3 |
| 11 | N1054N | 1.3 | 2.6 | 6.4 | 2.6 | 2.3 |
| 11 | N1054A | 5.5 | 4.2 | 3.5 | 2.7 | 2.4 |
| 11 | I1057T | 2.0 | 2.3 | 2.9 | 8.2 | 6.9 |
| 11 | I1057R | 1.6 | 1.3 | 0.6 | 0.8 | 1.0 |
| 11 | R1058R | 0.2 | 0.4 | 1.4 | 2.7 | 3.6 |
| 11 | R1058L | 1.5 | 2.7 | 4.4 | 1.4 | 2.8 |
| 11 | R1058G | 0.8 | 0.8 | 0.7 | 7.2 | 2.7 |
| 11 | R1058W | 3.5 | 4.8 | 2.1 | 7.6 | 8.0 |
| 11 | R1058C | 2.5 | 2.8 | 2.1 | 3.2 | 2.3 |
| 11 | K1059T | 2.3 | 6.6 | 11.2 | 8.5 | 9.0 |
| 11 | K1059S | 1.6 | 5.7 | 7.4 | 1.8 | 3.1 |
| 11 | R1060T | 2.3 | 8.3 | 6.4 | 5.9 | 4.4 |
| 11 | R1060S | 2.0 | 4.7 | 4.2 | 7.8 | 4.8 |
| 11 | R1060H | 1.5 | 5.1 | 13.8 | 14.1 | 3.9 |
| 11 | R1060L | 4.5 | 2.7 | 8.4 | 27.2 | 26.8 |
| 11 | R1060G | 1.9 | 5.9 | 9.4 | 11.8 | 8.4 |
| 11 | R1060Y | 3.3 | 4.1 | 3.2 | 2.0 | 1.0 |
| 11 | R1060W | 0.9 | 1.2 | 1.2 | 1.0 | 1.1 |
| 11 | R1060L | 1.5 | 3.9 | 3.6 | 10.4 | 14.6 |
| 11 | R1060F | 1.3 | 0.2 | 2.5 | 2.1 | 1.4 |
| 11 | L1062N | 0.4 | 8.4 | 11.7 | 4.8 | 2.7 |
| 11 | I1063K | 0.2 | 7.3 | 2.4 | 16.9 | 30.5 |
| 11 | I1063T | 1.4 | 4.5 | 2.9 | 3.9 | 2.9 |
| 11 | I1063R | 0.5 | 2.2 | 4.5 | 4.3 | 7.3 |
| 11 | I1063M | 1.0 | 4.2 | 5.5 | 4.0 | 3.8 |
| 11 | I1063V | 3.9 | 15.6 | 26.5 | 16.1 | 12.6 |
| 11 | T1065R | 9.2 | 54.6 | 137.2 | 121.6 | 159.0 |
| 11 | T1065V | 3.9 | 4.6 | 5.5 | 3.2 | 1.3 |
| 11 | N1066K | 2.2 | 2.8 | 6.6 | 3.4 | 2.4 |
| 11 | N1066T | 0.2 | 0.1 | 0.3 | 1.2 | 1.2 |
| 11 | N1066R | 2.0 | 4.7 | 8.8 | 5.8 | 4.2 |
| 11 | G1067T | 14.5 | 36.9 | 16.1 | 7.8 | 6.2 |
| 11 | E1068A | 2.8 | 9.6 | 12.4 | 11.6 | 11.2 |
| 11 | E1068W | 3.5 | 5.8 | 5.2 | 6.1 | 3.7 |
| 11 | T1069K | 2.9 | 14.8 | 40.7 | 82.8 | 131.6 |
| 11 | T1069N | 3.4 | 14.1 | 16.6 | 12.4 | 11.1 |
| 11 | T1069T | 3.4 | 3.5 | 5.6 | 2.8 | 3.1 |
| 11 | T1069R | 1.8 | 7.7 | 25.0 | 35.2 | 45.3 |
| 11 | T1069S | 2.5 | 3.6 | 3.0 | 3.5 | 1.3 |
| 11 | T1069R | 0.2 | 1.8 | 1.6 | 3.1 | 1.4 |
| 11 | T1069L | 9.4 | 14.5 | 13.7 | 4.3 | 1.1 |
| 11 | T1069G | 1.1 | 2.1 | 2.2 | 1.9 | 2.3 |
| 11 | T1069Y | 2.6 | 7.8 | 18.0 | 30.7 | 36.2 |
| 11 | T1069S | 3.1 | 3.6 | 3.6 | 3.4 | 2.3 |
| 11 | G1070K | 7.3 | 10.8 | 23.6 | 31.7 | 33.9 |
| 11 | G1070Q | 4.0 | 7.8 | 11.3 | 7.1 | 2.8 |
| 11 | G1070R | 0.8 | 1.9 | 3.3 | 1.3 | 1.3 |
| 11 | G1070R | 0.1 | 2.8 | 9.6 | 7.7 | 3.3 |
| 11 | G1070D | 1.6 | 7.4 | 13.3 | 15.5 | 7.7 |
| 11 | G1070A | 1.8 | 1.8 | 0.9 | 1.2 | 1.5 |
| 11 | G1070L | 0.8 | 2.0 | 3.8 | 4.4 | 3.6 |
| 11 | E1071K | 2.5 | 13.3 | 46.1 | 110.3 | 202.3 |
| 11 | E1071N | 3.8 | 8.4 | 12.0 | 18.7 | 16.7 |
| 11 | E1071T | 2.7 | 8.4 | 21.7 | 23.6 | 23.3 |
| 11 | E1071R | 0.5 | 2.3 | 8.2 | 17.3 | 24.1 |
| 11 | E1071M | 1.8 | 4.1 | 2.5 | 3.1 | 2.8 |
| 11 | E1071Q | 2.0 | 4.1 | 9.0 | 14.3 | 23.7 |
| 11 | E1071H | 1.6 | 6.1 | 8.5 | 13.7 | 6.5 |
| 11 | E1071P | 1.9 | 8.1 | 15.6 | 10.8 | 2.5 |
| 11 | E1071R | 3.3 | 4.0 | 10.5 | 20.3 | 52.0 |
| 11 | E1071R | 3.2 | 12.3 | 26.7 | 46.0 | 45.6 |
| 11 | E1071L | 4.1 | 5.3 | 9.6 | 9.1 | 5.2 |
| 11 | E1071L | 2.3 | 5.0 | 10.3 | 7.4 | 4.2 |
| 11 | E1071A | 3.0 | 1.5 | 6.3 | 8.6 | 8.3 |
| 11 | E1071A | 2.0 | 0.8 | 2.3 | 2.2 | 2.7 |
| 11 | E1071G | 1.2 | 4.0 | 5.9 | 3.2 | 1.3 |
| 11 | E1071G | 1.4 | 3.6 | 3.6 | 1.6 | 1.0 |
| 11 | E1071V | 2.3 | 4.5 | 5.4 | 5.9 | 3.5 |
| 11 | E1071Y | 2.9 | 5.3 | 5.4 | 7.4 | 5.8 |
| 11 | E1071S | 3.8 | 5.0 | 8.3 | 9.2 | 8.0 |
| 11 | E1071S | 5.7 | 10.5 | 26.8 | 54.8 | 97.3 |
| 11 | E1071W | 2.9 | 7.9 | 13.6 | 24.4 | 20.3 |
| 11 | E1071L | 3.6 | 5.4 | 5.5 | 4.4 | 3.2 |
| 11 | E1071F | 1.1 | 2.8 | 4.1 | 8.1 | 10.2 |
| 11 | I1072K | 3.1 | 3.0 | 5.9 | 9.0 | 10.8 |
| 11 | I1072N | 0.2 | 4.5 | 5.9 | 19.5 | 11.0 |
| 11 | I1072T | 0.3 | 2.0 | 8.8 | 19.4 | 14.7 |
| 11 | I1072R | 1.3 | 5.7 | 6.0 | 5.7 | 3.6 |
| 11 | I1072C | 0.4 | 6.1 | 10.1 | 9.6 | 9.0 |
| 11 | W1074V | 0.2 | 5.0 | 9.0 | 29.9 | 5.2 |
| 11 | D1075N | 0.3 | 5.2 | 2.3 | 2.8 | 7.3 |
| 11 | D1075H | 0.2 | 16.6 | 28.3 | 40.1 | 20.0 |
| 11 | D1075D | 1.3 | 4.2 | 4.3 | 2.5 | 2.2 |
| 11 | K1076K | 2.0 | 3.0 | 1.8 | 0.3 | 1.0 |
| 11 | K1076G | 2.4 | 4.1 | 8.8 | 11.5 | 8.1 |
| 11 | G1077R | 0.2 | 2.1 | 4.7 | 2.5 | 2.9 |
| 11 | G1077R | 8.0 | 13.0 | 14.3 | 15.1 | 15.1 |
| 11 | G1077L | 0.1 | 15.1 | 13.4 | 12.7 | 10.6 |
| 11 | R1078R | 2.6 | 2.7 | 3.1 | 5.5 | 2.9 |
| 11 | A1081K | 3.8 | 4.4 | 3.9 | 2.8 | 1.2 |
| 11 | A1081T | 2.9 | 3.5 | 8.4 | 11.1 | 6.2 |
| 11 | A1081D | 0.8 | 0.9 | 1.3 | 1.3 | 1.3 |
| 11 | A1081G | 3.6 | 3.0 | 2.9 | 2.7 | 1.1 |
| 11 | A1081C | 4.0 | 6.3 | 5.0 | 4.2 | 1.0 |
| 11 | K1085Q | 3.2 | 4.8 | 5.0 | 2.0 | 1.7 |
| 11 | K1085R | 0.3 | 0.6 | 2.0 | 4.1 | 2.8 |
| 11 | K1085D | 3.0 | 2.8 | 4.6 | 3.8 | 1.5 |
| 11 | K1085G | 1.0 | 2.1 | 2.5 | 4.2 | 1.8 |
| 11 | V1086V | 1.6 | 5.6 | 8.0 | 3.5 | 1.9 |
| 11 | P1090S | 3.5 | 11.6 | 3.2 | 0.5 | 1.2 |
| 11 | Q1091H | 3.8 | 3.2 | 3.1 | 2.2 | 2.3 |
| 11 | V1095R | 1.4 | 2.2 | 4.2 | 2.1 | 2.2 |
| 11 | T1098S | 0.2 | 4.3 | 5.2 | 1.9 | 1.0 |
| 11 | V1100E | 4.7 | 2.3 | 3.5 | 4.1 | 1.6 |
| 12 | Q1101K | 1.6 | 3.2 | 2.4 | 2.8 | 2.7 |
| 12 | Q1101L | 1.0 | 1.2 | 1.1 | 0.9 | 1.4 |
| 12 | Q1101A | 1.5 | 2.4 | 1.3 | 1.0 | 1.8 |
| 12 | Q1101V | 1.4 | 2.3 | 1.9 | 1.4 | 1.4 |
| 12 | Q1101L | 1.1 | 1.3 | 1.0 | 0.9 | 1.7 |
| 12 | T1102K | 1.0 | 1.0 | 1.4 | 2.1 | 2.4 |
| 12 | T1102T | 1.2 | 1.4 | 1.8 | 2.5 | 1.5 |
| 12 | T1102R | 1.5 | 1.7 | 2.3 | 2.2 | 1.6 |
| 12 | T1102S | 1.3 | 0.9 | 1.9 | 2.2 | 2.1 |
| 12 | T1102R | 1.8 | 3.7 | 4.4 | 5.9 | 5.9 |
| 12 | T1102R | 1.3 | 1.5 | 2.2 | 2.8 | 6.8 |
| 12 | T1102G | 0.9 | 0.8 | 1.2 | 1.4 | 1.4 |
| 12 | T1102S | 1.5 | 1.7 | 1.0 | 0.7 | 1.1 |
| 12 | G1104R | 1.6 | 1.8 | 1.6 | 2.0 | 1.9 |
| 12 | G1104M | 1.2 | 1.7 | 1.9 | 3.2 | 3.1 |
| 12 | G1104Q | 1.5 | 1.6 | 1.8 | 2.3 | 2.6 |
| 12 | G1104P | 0.9 | 0.2 | 0.8 | 2.9 | 1.4 |
| 12 | G1104R | 1.6 | 2.5 | 3.4 | 5.0 | 5.0 |
| 12 | G1104R | 1.5 | 1.8 | 0.5 | 1.4 | 3.5 |
| 12 | G1104L | 1.1 | 1.6 | 2.1 | 1.9 | 1.5 |
| 12 | G1104A | 1.4 | 1.6 | 1.5 | 1.7 | 1.0 |
| 12 | G1104V | 1.1 | 1.4 | 2.6 | 3.7 | 3.7 |
| 12 | G1104S | 1.8 | 1.4 | 5.6 | 6.2 | 4.4 |
| 12 | G1104F | 0.5 | 0.5 | 1.5 | 1.4 | 2.4 |
| 12 | S1106T | 0.6 | 0.5 | 0.6 | 1.1 | 2.6 |
| 12 | S1106T | 0.8 | 1.0 | 1.0 | 1.1 | 1.4 |
| 12 | S1106S | 1.1 | 1.4 | 1.2 | 1.3 | 2.7 |
| 12 | S1106H | 0.9 | 1.5 | 1.7 | 1.4 | 1.0 |
| 12 | S1106A | 1.4 | 1.2 | 0.9 | 0.8 | 1.2 |
| 12 | S1106A | 1.1 | 1.8 | 1.4 | 1.7 | 4.5 |
| 12 | S1106G | 1.4 | 1.6 | 1.6 | 1.3 | 1.1 |
| 12 | S1106Y | 0.9 | 0.8 | 0.9 | 1.5 | 1.6 |
| 12 | S1106F | 1.0 | 1.1 | 0.8 | 0.6 | 1.0 |
| 12 | G1103G | 2.0 | 1.9 | 1.5 | 1.8 | 1.3 |
| 12 | G1104K | 3.9 | 6.6 | 1.2 | 2.1 | 2.1 |
| 12 | G1104R | 2.8 | 2.8 | 2.8 | 2.5 | 2.4 |
| 12 | G1104M | 2.3 | 4.5 | 5.7 | 11.2 | 11.2 |
| 12 | G1104Q | 2.5 | 4.5 | 5.4 | 6.7 | 7.4 |
| 12 | G1104H | 2.5 | 1.0 | 7.2 | 9.0 | 2.9 |
| 12 | G1104P | 1.7 | 0.3 | 2.4 | 8.1 | 4.1 |
| 12 | G1104R | 2.7 | 4.9 | 7.8 | 12.2 | 14.1 |
| 12 | G1104L | 2.6 | 3.9 | 5.2 | 5.3 | 3.8 |
| 12 | G1104L | 2.0 | 4.7 | 6.3 | 5.3 | 2.2 |
| 12 | G1104V | 2.1 | 2.9 | 5.2 | 7.7 | 8.0 |
| 12 | G1104S | 3.0 | 3.0 | 10.9 | 10.6 | 6.5 |
| 12 | G1104F | 1.3 | 1.6 | 4.9 | 4.9 | 8.1 |
| 12 | F1105H | 4.4 | 4.3 | 6.6 | 12.5 | 3.1 |
| 12 | S1106T | 1.2 | 0.8 | 1.1 | 0.8 | 2.6 |
| 12 | S1106T | 0.9 | 1.2 | 1.1 | 0.9 | 1.7 |
| 12 | S1106S | 1.9 | 3.5 | 3.6 | 4.6 | 10.2 |
| 12 | S1106H | 1.4 | 3.3 | 3.2 | 2.8 | 2.6 |
| 12 | S1106A | 2.1 | 2.9 | 2.1 | 1.7 | 3.1 |
| 12 | S1106A | 1.4 | 3.8 | 3.1 | 4.2 | 15.2 |
| 12 | S1106G | 2.2 | 1.8 | 2.1 | 1.8 | 1.3 |
| 12 | S1106G | 2.6 | 3.5 | 2.2 | 1.6 | 1.4 |
| 12 | S1106Y | 1.4 | 1.6 | 1.4 | 2.0 | 2.8 |
| 12 | S1106F | 1.9 | 2.5 | 2.1 | 1.5 | 1.3 |
| 12 | K1107R | 1.6 | 1.8 | 1.4 | 1.0 | 3.5 |
| 12 | K1107R | 2.1 | 3.3 | 3.1 | 2.9 | 1.2 |
| 12 | K1107R | 0.8 | 1.3 | 2.3 | 4.5 | 2.0 |
| 12 | E1108N | 1.0 | 3.7 | 3.1 | 9.4 | 11.5 |
| 12 | E1108E | 2.6 | 7.3 | 9.5 | 8.3 | 3.9 |
| 12 | E1108D | 1.1 | 2.1 | 1.7 | 2.1 | 3.1 |
| 12 | E1108V | 3.7 | 2.6 | 0.9 | 1.1 | 2.6 |
| 12 | E1108V | 1.4 | 1.4 | 2.4 | 0.9 | 1.1 |
| 12 | E1108L | 1.8 | 1.7 | 7.9 | 4.1 | 5.4 |
| 12 | S1109R | 2.1 | 2.3 | 2.3 | 5.7 | 6.2 |
| 12 | S1109I | 1.0 | 2.7 | 6.6 | 4.6 | 1.3 |
| 12 | S1109Q | 2.7 | 3.9 | 4.8 | 4.6 | 4.9 |
| 12 | S1109A | 2.0 | 2.7 | 6.3 | 8.1 | 5.1 |
| 12 | S1109A | 2.1 | 3.6 | 5.4 | 4.2 | 8.6 |
| 12 | S1109G | 2.7 | 4.5 | 5.1 | 8.2 | 6.8 |
| 12 | I1110V | 2.4 | 4.3 | 17.6 | 21.7 | 40.7 |
| 12 | L1111T | 5.7 | 6.5 | 26.5 | 17.8 | 24.6 |
| 12 | L1111Q | 0.2 | 0.6 | 6.4 | 2.8 | 2.9 |
| 12 | L1111V | 2.6 | 2.5 | 10.0 | 6.2 | 4.3 |
| 12 | P1112G | 1.7 | 3.5 | 3.7 | 5.8 | 2.1 |
| 12 | P1112C | 2.2 | 0.7 | 0.7 | 0.7 | 2.2 |
| 12 | R1114M | 1.4 | 2.5 | 3.9 | 5.3 | 11.2 |
| 12 | R1114R | 2.9 | 2.3 | 3.4 | 6.8 | 2.7 |
| 12 | R1114A | 5.3 | 7.8 | 11.0 | 16.7 | 5.8 |
| 12 | R1114S | 3.3 | 2.4 | 16.2 | 11.1 | 3.6 |
| 12 | N1115K | 0.9 | 2.7 | 7.7 | 10.3 | 8.4 |
| 12 | N1115T | 3.8 | 2.2 | 12.2 | 25.9 | 19.8 |
| 12 | N1115Q | 7.6 | 8.5 | 18.5 | 23.6 | 23.8 |
| 12 | N1115R | 3.0 | 2.6 | 6.1 | 8.0 | 10.4 |
| 12 | N1115C | 0.9 | 3.6 | 3.1 | 3.2 | 3.8 |
| 12 | S1116D | 4.2 | 6.7 | 3.7 | 3.4 | 9.5 |
| 12 | S1116G | 0.9 | 0.5 | 0.2 | 0.3 | 1.2 |
| 12 | S1116S | 3.9 | 8.4 | 11.0 | 8.4 | 15.6 |
| 12 | S1116S | 2.9 | 6.7 | 13.7 | 13.2 | 17.9 |
| 12 | D1117N | 3.6 | 6.7 | 4.5 | 9.4 | 8.7 |
| 12 | D1117I | 4.7 | 12.7 | 20.7 | 14.8 | 6.5 |
| 12 | D1117E | 3.0 | 4.5 | 4.7 | 2.6 | 3.4 |
| 12 | K1118N | 0.4 | 0.6 | 1.9 | 0.6 | 1.1 |
| 12 | K1118S | 5.7 | 25.1 | 16.1 | 25.0 | 1.5 |
| 12 | K1118C | 6.8 | 33.6 | 32.6 | 100.1 | 224.0 |
| 12 | L1119H | 0.7 | 0.8 | 0.8 | 0.9 | 1.0 |
| 12 | L1119L | 3.3 | 12.2 | 41.0 | 48.9 | 45.9 |
| 12 | I1120N | 0.7 | 0.9 | 1.2 | 1.2 | 1.3 |
| 12 | I1120T | 1.3 | 0.9 | 0.8 | 1.0 | 1.1 |
| 12 | A1121G | 2.4 | 5.8 | 3.3 | 2.2 | 6.2 |
| 12 | K1124T | 0.7 | 0.6 | 0.7 | 0.7 | 1.1 |
| 12 | K1124I | 0.1 | 5.3 | 9.2 | 12.6 | 12.4 |
| 12 | K1124V | 2.9 | 3.0 | 7.0 | 11.7 | 11.1 |
| 12 | D1125T | 5.9 | 7.2 | 16.5 | 19.5 | 45.4 |
| 12 | D1125S | 0.5 | 1.3 | 4.2 | 2.3 | 1.6 |
| 12 | D1125A | 0.9 | 1.7 | 2.4 | 3.0 | 1.4 |
| 12 | D1125G | 3.2 | 3.3 | 19.1 | 31.2 | 32.3 |
| 12 | W1126L | 4.8 | 17.0 | 17.4 | 9.5 | 12.7 |
| 12 | D1127G | 1.8 | 3.1 | 6.7 | 1.3 | 1.5 |
| 12 | D1127V | 1.0 | 1.4 | 1.5 | 1.8 | 1.2 |
| 12 | P1128V | 0.9 | 2.5 | 4.5 | 3.9 | 2.0 |
| 12 | K1129K | 2.4 | 2.2 | 4.9 | 9.7 | 2.1 |
| 12 | K1129T | 1.2 | 0.6 | 1.9 | 3.6 | 1.7 |
| 12 | K1129R | 0.4 | 0.3 | 1.2 | 2.9 | 4.0 |
| 12 | K1129V | 3.6 | 9.1 | 13.8 | 10.5 | 12.2 |
| 12 | K1129S | 0.7 | 2.1 | 3.4 | 11.7 | 12.2 |
| 12 | K1130R | 1.3 | 1.1 | 3.7 | 5.3 | 14.2 |
| 12 | Y1131N | 1.1 | 1.3 | 1.6 | 1.3 | 1.6 |
| 12 | Y1131F | 0.9 | 1.3 | 1.1 | 1.5 | 1.5 |
| 12 | G1133G | 0.9 | 2.3 | 1.9 | 1.7 | 1.5 |
| 12 | G1133G | 2.6 | 2.6 | 0.2 | 1.2 | 1.0 |
| 12 | F1134L | 1.6 | 1.6 | 2.1 | 1.9 | 2.1 |
| 12 | D1135N | 1.5 | 0.6 | 2.3 | 3.1 | 5.8 |
| 12 | D1135T | 1.6 | 5.8 | 6.3 | 5.0 | 7.8 |
| 12 | D1135R | 0.9 | 1.0 | 2.6 | 1.5 | 1.7 |
| 12 | D1135S | 4.4 | 10.5 | 15.7 | 30.3 | 33.1 |
| 12 | D1135R | 0.4 | 0.6 | 3.6 | 5.2 | 5.2 |
| 12 | D1135D | 2.2 | 7.7 | 3.5 | 3.0 | 2.4 |
| 12 | D1135A | 0.6 | 1.2 | 3.3 | 3.7 | 8.1 |
| 12 | D1135A | 3.2 | 10.0 | 11.0 | 10.3 | 14.6 |
| 12 | D1135V | 4.2 | 7.7 | 6.2 | 7.2 | 7.3 |
| 12 | D1135Y | 1.2 | 0.9 | 3.0 | 5.6 | 8.5 |
| 12 | S1136K | 3.4 | 7.8 | 11.3 | 4.8 | 10.1 |
| 12 | S1136Q | 2.6 | 3.0 | 9.2 | 4.6 | 2.8 |
| 12 | P1137A | 1.7 | 1.2 | 8.8 | 8.5 | 5.0 |
| 12 | T1138K | 2.8 | 10.6 | 5.2 | 4.4 | 7.5 |
| 12 | T1138S | 1.9 | 1.3 | 2.9 | 3.0 | 1.5 |
| 12 | T1138R | 4.2 | 18.7 | 26.1 | 22.2 | 28.5 |
| 12 | T1138A | 1.7 | 3.3 | 19.6 | 13.1 | 8.9 |
| 12 | T1138S | 3.5 | 4.1 | 2.7 | 5.8 | 6.2 |
| 12 | T1138C | 2.1 | 1.9 | 2.7 | 2.2 | 2.7 |
| 12 | V1139N | 3.2 | 2.9 | 1.9 | 2.1 | 1.6 |
| 12 | V1139T | 1.8 | 1.8 | 4.1 | 6.7 | 22.3 |
| 12 | V1139T | 2.0 | 1.3 | 7.1 | 7.6 | 7.5 |
| 12 | V1139Q | 1.4 | 2.4 | 4.4 | 0.5 | 1.7 |
| 12 | V1139H | 1.1 | 3.3 | 12.2 | 21.3 | 34.9 |
| 12 | V1139E | 3.1 | 3.5 | 7.9 | 9.1 | 2.8 |
| 12 | V1139D | 1.0 | 1.3 | 1.4 | 1.5 | 1.2 |
| 12 | V1139V | 2.0 | 0.6 | 4.1 | 4.8 | 3.9 |
| 12 | Y1141F | 0.5 | 1.6 | 5.1 | 2.6 | 8.9 |
| 12 | S1142S | 1.3 | 0.9 | 1.8 | 1.5 | 11.5 |
| 12 | V1143I | 4.0 | 19.0 | 2.6 | 7.5 | 8.8 |
| 12 | V1143A | 3.3 | 4.8 | 18.0 | 19.0 | 10.5 |
| 12 | V1145I | 2.0 | 3.4 | 4.1 | 7.0 | 11.3 |
| 12 | V1146L | 5.4 | 15.7 | 27.6 | 8.0 | 4.9 |
| 12 | V1146A | 9.8 | 8.0 | 13.0 | 57.8 | 19.4 |
| 12 | A1147E | 3.0 | 3.4 | 3.4 | 3.9 | 4.2 |
| 12 | A1147A | 1.9 | 2.1 | 2.6 | 2.8 | 2.9 |
| 12 | A1147G | 3.7 | 9.1 | 8.0 | 8.5 | 11.6 |
| 12 | K1148N | 1.7 | 2.1 | 2.7 | 2.5 | 3.2 |
| 12 | K1148C | 1.2 | 6.3 | 19.1 | 11.1 | 37.6 |
| 12 | V1149T | 0.1 | 3.1 | 14.5 | 15.2 | 17.7 |
| 12 | V1149V | 1.4 | 1.7 | 1.5 | 1.6 | 1.7 |
| 12 | V1149L | 0.6 | 0.6 | 1.1 | 1.0 | 1.0 |
| 12 | E1150M | 4.2 | 7.8 | 22.6 | 41.3 | 1.3 |
| 12 | E1150E | 2.3 | 1.1 | 0.9 | 0.9 | 1.0 |
| 12 | E1150D | 1.5 | 1.6 | 1.8 | 2.0 | 2.1 |
| 12 | E1150L | 2.1 | 1.3 | 5.9 | 2.0 | 2.9 |
| 12 | E1150F | 3.0 | 4.9 | 14.8 | 26.3 | 34.6 |
| 12 | K1151N | 0.7 | 0.7 | 0.8 | 1.0 | 1.0 |
| 12 | K1151M | 1.7 | 7.3 | 9.6 | 17.1 | 19.8 |
| 12 | K1151R | 0.0 | 16.0 | 50.1 | 58.7 | 100.7 |
| 12 | K1151STOP | 1.5 | 1.6 | 1.9 | 2.0 | 2.0 |
| 12 | G1152S | 0.8 | 2.7 | 3.4 | 1.6 | 2.3 |
| 12 | G1152C | 0.9 | 1.0 | 1.5 | 1.4 | 1.4 |
| 12 | G1152F | 1.1 | 3.2 | 0.3 | 2.2 | 3.6 |
| 12 | K1153N | 2.6 | 2.0 | 2.0 | 2.9 | 2.4 |
| 12 | K1153R | 7.6 | 11.5 | 12.0 | 4.6 | 13.0 |
| 12 | K1153S | 11.1 | 19.2 | 72.9 | 135.9 | 201.6 |
| 12 | S1154G | 3.6 | 4.6 | 19.2 | 18.3 | 13.6 |
| 12 | K1155N | 3.2 | 3.1 | 2.1 | 2.6 | 2.6 |
| 12 | K1155M | 1.6 | 1.5 | 1.9 | 2.1 | 1.8 |
| 12 | K1155Q | 2.8 | 10.8 | 38.6 | 27.1 | 27.0 |
| 12 | K1155E | 5.6 | 9.2 | 1.8 | 0.8 | 1.1 |
| 12 | K1155STOP | 2.3 | 2.2 | 2.8 | 2.7 | 3.1 |
| 12 | K1156N | 3.4 | 1.8 | 2.1 | 2.3 | 2.5 |
| 12 | K1156R | 1.6 | 11.9 | 12.2 | 6.2 | 3.5 |
| 12 | K1156A | 2.5 | 1.3 | 2.1 | 2.2 | 1.2 |
| 12 | L1157Q | 1.2 | 2.6 | 3.9 | 2.2 | 1.5 |
| 12 | L1157R | 1.9 | 5.3 | 2.2 | 0.6 | 1.0 |
| 12 | L1157L | 1.9 | 2.2 | 1.1 | 1.1 | 1.1 |
| 12 | K1158M | 1.9 | 3.7 | 10.0 | 1.1 | 2.6 |
| 12 | S1159T | 2.2 | 2.6 | 3.5 | 3.5 | 3.2 |
| 12 | S1159P | 4.5 | 1.9 | 1.3 | 1.1 | 1.6 |
| 12 | S1159C | 1.6 | 1.8 | 1.0 | 1.2 | 1.1 |
| 12 | S1159L | 2.1 | 9.2 | 6.5 | 9.7 | 16.8 |
| 12 | V1160T | 2.7 | 0.5 | 10.0 | 15.0 | 12.3 |
| 12 | V1160C | 3.4 | 0.6 | 12.3 | 17.0 | 14.6 |
| 12 | K1161STOP | 0.9 | 1.0 | 0.8 | 1.0 | 1.0 |
| 12 | E1162R | 2.4 | 4.5 | 8.0 | 16.7 | 24.8 |
| 12 | E1162V | 1.2 | 1.2 | 1.3 | 1.7 | 1.6 |
| 12 | E1162V | 1.1 | 3.1 | 3.5 | 3.4 | 1.5 |
| 12 | L1163L | 1.2 | 4.6 | 9.6 | 18.5 | 16.9 |
| 12 | L1164L | 6.5 | 15.0 | 45.2 | 96.1 | 110.7 |
| 12 | G1165G | 3.1 | 2.9 | 1.7 | 1.7 | 1.7 |
| 12 | I1166I | 1.1 | 3.0 | 3.6 | 3.6 | 3.8 |
| 12 | T1167T | 5.4 | 5.1 | 9.7 | 2.3 | 6.3 |
| 12 | T1167S | 5.0 | 12.6 | 18.5 | 26.8 | 57.7 |
| 12 | I1168N | 1.1 | 0.6 | 0.7 | 1.0 | 1.0 |
| 12 | I1168T | 0.9 | 0.8 | 1.0 | 0.9 | 1.0 |
| 12 | I1168F | 1.7 | 1.7 | 2.0 | 2.2 | 2.4 |
| 12 | M1169K | 1.2 | 1.7 | 1.6 | 1.8 | 1.5 |
| 12 | M1169I | 0.8 | 1.0 | 1.4 | 1.1 | 1.4 |
| 12 | M1169R | 3.0 | 2.6 | 24.1 | 19.4 | 7.7 |
| 12 | M1169L | 2.1 | 2.5 | 2.9 | 3.1 | 3.1 |
| 12 | E1170G | 1.2 | 0.9 | 0.8 | 1.1 | 1.1 |
| 12 | E1170V | 0.9 | 1.2 | 1.3 | 1.6 | 1.3 |
| 12 | E1170STOP | 1.1 | 1.6 | 2.7 | 2.0 | 2.3 |
| 12 | R1171S | 1.0 | 1.0 | 1.3 | 1.4 | 1.6 |
| 12 | R1171H | 0.4 | 0.7 | 0.9 | 0.5 | 1.0 |
| 12 | S1172R | 2.1 | 5.5 | 9.0 | 16.5 | 19.2 |
| 12 | S1172G | 1.0 | 0.9 | 3.9 | 8.1 | 6.8 |
| 12 | S1172S | 7.2 | 7.9 | 7.3 | 9.0 | 9.4 |
| 12 | S1172L | 1.1 | 10.0 | 8.2 | 12.4 | 41.5 |
| 12 | S1173T | 8.8 | 7.5 | 8.9 | 10.3 | 8.7 |
| 12 | S1173R | 6.4 | 15.7 | 38.3 | 55.6 | 80.5 |
| 12 | S1173L | 2.8 | 2.4 | 10.0 | 19.7 | 32.2 |
| 12 | S1173V | 7.1 | 12.3 | 14.9 | 6.8 | 13.7 |
| 12 | S1173Y | 2.0 | 0.9 | 0.0 | 1.9 | 3.7 |
| 12 | S1173C | 0.7 | 1.7 | 4.7 | 5.9 | 14.1 |
| 12 | S1173L | 2.5 | 1.4 | 4.9 | 5.0 | 5.1 |
| 12 | F1174F | 1.8 | 1.8 | 6.3 | 4.5 | 5.3 |
| 12 | S1173T | 2.7 | 3.7 | 4.4 | 5.4 | 3.8 |
| 12 | S1173R | 2.6 | 3.4 | 7.1 | 10.1 | 13.9 |
| 12 | S1173L | 2.8 | 2.2 | 9.4 | 19.1 | 27.7 |
| 12 | S1173V | 2.2 | 3.4 | 3.1 | 2.9 | 3.2 |
| 12 | S1173C | 0.5 | 1.1 | 2.6 | 3.6 | 8.1 |
| 12 | S1173L | 1.0 | 0.7 | 4.3 | 5.2 | 4.6 |
| 12 | F1174F | 1.7 | 1.3 | 2.4 | 2.0 | 2.2 |
| 12 | E1175L | 1.3 | 1.3 | 4.8 | 4.1 | 1.4 |
| 12 | K1176M | 0.9 | 0.6 | 1.0 | 2.7 | 1.5 |
| 12 | K1176G | 0.9 | 0.8 | 2.8 | 1.1 | 1.4 |
| 12 | N1177Y | 0.4 | 2.7 | 0.5 | 1.2 | 4.2 |
| 12 | I1179N | 1.1 | 1.0 | 1.1 | 1.0 | 1.1 |
| 12 | I1179L | 1.8 | 2.0 | 0.7 | 0.6 | 1.4 |
| 12 | D1180T | 2.1 | 1.7 | 7.3 | 13.6 | 3.4 |
| 12 | D1180H | 2.4 | 6.9 | 5.6 | 3.7 | 5.5 |
| 12 | D1180G | 1.7 | 1.8 | 3.4 | 5.1 | 3.5 |
| 12 | D1180V | 1.7 | 1.4 | 1.6 | 2.2 | 2.1 |
| 12 | E1183H | 0.9 | 1.4 | 1.3 | 0.8 | 2.4 |
| 12 | E1183A | 1.7 | 1.4 | 1.2 | 1.1 | 1.9 |
| 12 | A1184R | 0.4 | 1.5 | 2.1 | 2.0 | 1.8 |
| 12 | A1184A | 1.0 | 0.4 | 1.3 | 2.1 | 3.1 |
| 12 | A1184G | 0.5 | 1.0 | 1.8 | 1.4 | 2.9 |
| 12 | A1184L | 1.2 | 4.3 | 1.4 | 2.9 | 2.1 |
| 12 | K1185C | 0.9 | 0.2 | 3.6 | 4.3 | 1.9 |
| 12 | G1186S | 2.4 | 3.3 | 2.7 | 6.0 | 18.0 |
| 12 | G1186G | 1.7 | 2.2 | 2.1 | 1.6 | 2.6 |
| 12 | Y1187D | 1.0 | 1.0 | 1.0 | 1.1 | 1.1 |
| 12 | Y1187F | 2.2 | 2.2 | 5.5 | 2.9 | 1.3 |
| 12 | K1188T | 1.6 | 0.3 | 1.4 | 1.1 | 2.2 |
| 12 | K1188R | 0.6 | 0.6 | 1.9 | 2.1 | 4.7 |
| 12 | K1188R | 1.6 | 2.3 | 10.3 | 8.1 | 6.7 |
| 12 | K1188L | 1.2 | 2.6 | 3.9 | 0.8 | 1.2 |
| 12 | K1188A | 1.4 | 1.4 | 2.0 | 7.0 | 23.1 |
| 12 | E1189A | 0.4 | 3.1 | 6.2 | 5.3 | 4.9 |
| 12 | V1190V | 0.7 | 1.3 | 2.2 | 5.1 | 7.1 |
| 12 | V1190V | 1.9 | 0.6 | 2.9 | 1.8 | 2.0 |
| 12 | K1191Q | 2.2 | 4.4 | 3.3 | 7.3 | 5.5 |
| 12 | K1192R | 1.4 | 0.9 | 3.7 | 6.6 | 4.0 |
| 12 | K1192L | 0.9 | 0.8 | 1.4 | 6.5 | 6.4 |
| 12 | K1192G | 2.0 | 3.1 | 2.7 | 1.8 | 2.2 |
| 12 | D1193H | 1.5 | 1.5 | 6.5 | 11.1 | 9.5 |
| 12 | L1194S | 1.4 | 0.3 | 2.6 | 4.4 | 3.3 |
| 12 | L1194Q | 2.0 | 3.1 | 3.8 | 6.1 | 2.6 |
| 12 | L1194H | 0.8 | 4.3 | 5.9 | 7.7 | 5.3 |
| 12 | L1194Y | 2.0 | 2.5 | 0.8 | 1.7 | 7.9 |
| 12 | L1194L | 1.7 | 0.3 | 4.0 | 3.5 | 4.4 |
| 12 | I1195L | 0.9 | 0.6 | 0.5 | 1.0 | 3.6 |
| 12 | I1196F | 1.5 | 1.7 | 4.1 | 6.1 | 3.8 |
| 12 | L1198M | 1.3 | 2.4 | 3.5 | 2.3 | 1.0 |
| 13 | S1202T | 2.4 | 2.8 | 3.5 | 5.2 | 4.0 |
| 13 | S1202S | 3.7 | 5.1 | 4.1 | 1.9 | 2.2 |
| 13 | S1202S | 2.6 | 3.8 | 2.6 | 3.0 | 3.1 |
| 13 | S1202S | 3.1 | 3.7 | 1.6 | 2.5 | 4.8 |
| 13 | L1203L | 3.5 | 3.3 | 5.9 | 2.2 | 2.0 |
| 13 | F1204F | 2.6 | 3.7 | 1.7 | 1.5 | 1.7 |
| 13 | L1206M | 1.7 | 2.4 | 6.0 | 4.3 | 4.9 |
| 13 | E1207E | 1.7 | 2.1 | 0.5 | 4.9 | 4.0 |
| 13 | N1208F | 0.7 | 1.8 | 20.7 | 7.4 | 8.3 |
| 13 | G1209A | 0.8 | 0.5 | 2.4 | 1.3 | 2.5 |
| 13 | G1209G | 3.0 | 3.9 | 1.3 | 1.1 | 1.4 |
| 13 | R1210R | 2.2 | 3.1 | 3.7 | 1.4 | 2.8 |
| 13 | K1211R | 3.0 | 2.6 | 5.7 | 5.5 | 4.5 |
| 13 | R1212R | 1.9 | 3.4 | 3.5 | 4.4 | 3.0 |
| 13 | R1212V | 1.4 | 2.9 | 0.2 | 9.5 | 3.6 |
| 13 | M1213L | 2.0 | 3.7 | 1.1 | 1.1 | 1.6 |
| 13 | M1213L | 2.3 | 3.4 | 7.9 | 1.4 | 1.0 |
| 13 | L1214L | 1.6 | 3.6 | 6.8 | 3.9 | 3.0 |
| 13 | L1214L | 1.6 | 1.7 | 5.5 | 10.5 | 7.5 |
| 13 | A1215S | 2.9 | 2.5 | 6.1 | 1.5 | 1.1 |
| 13 | A1215A | 1.3 | 1.5 | 2.2 | 5.2 | 5.7 |
| 13 | A1215G | 2.3 | 2.9 | 2.9 | 5.0 | 3.6 |
| 13 | A1215G | 1.2 | 1.5 | 2.1 | 1.0 | 1.2 |
| 13 | S1216G | 2.6 | 3.5 | 2.2 | 1.1 | 2.5 |
| 13 | S1216S | 2.5 | 3.8 | 2.8 | 2.7 | 1.4 |
| 13 | A1217A | 2.3 | 3.6 | 12.2 | 16.8 | 6.9 |
| 13 | G1218N | 5.3 | 10.2 | 25.0 | 32.4 | 40.7 |
| 13 | G1218R | 1.6 | 1.7 | 3.6 | 7.7 | 6.1 |
| 13 | G1218G | 2.1 | 4.4 | 11.8 | 31.2 | 77.7 |
| 13 | L1220L | 1.1 | 1.4 | 3.1 | 3.6 | 1.4 |
| 13 | Q1221M | 1.1 | 0.9 | 2.0 | 1.2 | 5.1 |
| 13 | Q1221H | 3.1 | 4.2 | 6.2 | 2.2 | 1.0 |
| 13 | Q1221L | 1.1 | 1.8 | 1.8 | 1.0 | 1.0 |
| 13 | G1223G | 2.3 | 2.8 | 4.2 | 2.0 | 2.1 |
| 13 | G1223G | 2.2 | 2.1 | 8.3 | 6.8 | 9.3 |
| 13 | N1224Y | 1.4 | 1.7 | 2.0 | 2.0 | 2.3 |
| 13 | A1227S | 2.8 | 3.5 | 6.1 | 2.5 | 1.4 |
| 13 | A1227M | 2.4 | 1.6 | 11.5 | 13.5 | 10.2 |
| 13 | A1227Q | 2.1 | 2.2 | 2.5 | 3.4 | 2.1 |
| 13 | A1227A | 1.1 | 1.9 | 2.5 | 0.2 | 1.2 |
| 13 | A1227V | 2.4 | 3.9 | 11.1 | 13.0 | 18.8 |
| 13 | A1227V | 2.8 | 3.9 | 3.8 | 3.7 | 6.0 |
| 13 | A1227C | 1.9 | 4.0 | 7.4 | 22.6 | 32.6 |
| 13 | L1228L | 4.5 | 4.5 | 3.8 | 10.1 | 7.2 |
| 13 | L1228V | 2.2 | 2.6 | 4.3 | 8.6 | 4.1 |
| 13 | P1229K | 4.8 | 6.2 | 7.5 | 9.6 | 7.0 |
| 13 | P1229N | 2.0 | 2.0 | 3.5 | 9.5 | 15.5 |
| 13 | P1229R | 2.1 | 0.6 | 2.9 | 2.7 | 1.2 |
| 13 | P1229S | 0.9 | 0.3 | 3.1 | 6.6 | 3.0 |
| 13 | P1229Q | 3.6 | 3.0 | 5.7 | 3.5 | 3.2 |
| 13 | P1229P | 2.4 | 1.7 | 4.2 | 2.8 | 2.3 |
| 13 | P1229E | 4.5 | 8.3 | 24.1 | 35.6 | 38.1 |
| 13 | P1229D | 2.0 | 4.0 | 4.1 | 6.5 | 8.1 |
| 13 | P1229A | 3.0 | 4.1 | 8.7 | 14.5 | 6.6 |
| 13 | P1229G | 1.9 | 1.9 | 2.6 | 4.6 | 2.0 |
| 13 | P1229C | 2.2 | 3.2 | 1.2 | 2.4 | 3.5 |
| 13 | S1230M | 1.2 | 1.3 | 5.5 | 12.7 | 3.9 |
| 13 | S1230I | 4.0 | 3.5 | 0.7 | 1.9 | 1.8 |
| 13 | S1230L | 0.4 | 0.1 | 1.6 | 1.9 | 3.0 |
| 13 | S1230V | 1.0 | 0.8 | 0.1 | 0.0 | 3.2 |
| 13 | S1230Y | 2.2 | 3.0 | 5.3 | 6.2 | 3.8 |
| 13 | S1230S | 1.8 | 1.9 | 5.8 | 5.6 | 4.1 |
| 13 | S1230C | 4.5 | 7.6 | 11.6 | 8.7 | 9.7 |
| 13 | K1231STOP | 1.1 | 1.4 | 1.6 | 1.4 | 1.6 |
| 13 | Y1232L | 0.3 | 0.4 | 1.1 | 0.6 | 1.2 |
| 13 | V1233L | 1.9 | 2.7 | 2.6 | 2.7 | 2.8 |
| 13 | V1233G | 1.1 | 0.8 | 1.0 | 1.0 | 1.0 |
| 13 | V1233F | 1.8 | 1.6 | 1.7 | 1.8 | 1.9 |
| 13 | N1234K | 3.1 | 3.7 | 14.8 | 18.7 | 18.5 |
| 13 | N1234R | 3.4 | 5.0 | 1.7 | 2.6 | 2.3 |
| 13 | F1235Y | 1.1 | 3.3 | 0.3 | 5.5 | 9.5 |
| 13 | F1235F | 1.7 | 2.2 | 4.9 | 2.6 | 3.3 |
| 13 | L1236L | 2.5 | 3.1 | 5.6 | 2.6 | 5.0 |
| 13 | L1236L | 0.9 | 0.8 | 7.3 | 0.3 | 1.9 |
| 13 | Y1237N | 1.7 | 1.9 | 2.0 | 2.0 | 2.2 |
| 13 | Y1237F | 1.0 | 1.0 | 1.4 | 1.5 | 3.6 |
| 13 | L1238Y | 0.5 | 0.4 | 1.0 | 2.6 | 1.5 |
| 13 | S1240R | 2.9 | 6.5 | 13.0 | 6.3 | 1.6 |
| 13 | S1240W | 3.4 | 2.8 | 6.4 | 6.3 | 5.3 |
| 13 | S1240C | 4.7 | 6.6 | 5.6 | 8.0 | 2.8 |
| 13 | S1240L | 0.8 | 0.1 | 0.5 | 0.8 | 2.2 |
| 13 | H1241K | 1.0 | 1.3 | 0.6 | 0.7 | 1.0 |
| 13 | H1241R | 2.7 | 4.1 | 6.0 | 3.1 | 5.6 |
| 13 | H1241S | 3.6 | 7.8 | 12.0 | 10.1 | 6.7 |
| 13 | H1241S | 3.4 | 3.1 | 9.1 | 9.8 | 22.5 |
| 13 | H1241C | 2.6 | 1.6 | 7.8 | 7.6 | 3.0 |
| 13 | E1243T | 3.5 | 2.4 | 3.0 | 1.0 | 3.2 |
| 13 | E1243D | 2.7 | 3.0 | 3.6 | 7.3 | 5.1 |
| 13 | E1243G | 1.6 | 2.6 | 1.9 | 0.2 | 1.1 |
| 13 | E1243H | 1.0 | 1.3 | 3.4 | 12.2 | 7.2 |
| 13 | E1243D | 1.5 | 1.9 | 3.0 | 4.4 | 2.7 |
| 13 | K1244K | 1.5 | 1.1 | 0.6 | 1.4 | 4.0 |
| 13 | K1244N | 1.8 | 1.7 | 2.0 | 2.4 | 4.9 |
| 13 | K1244I | 1.1 | 0.9 | 3.8 | 1.7 | 3.1 |
| 13 | K1244L | 1.0 | 1.4 | 2.0 | 1.0 | 1.1 |
| 13 | L1245Q | 1.1 | 0.7 | 1.8 | 22.5 | 22.3 |
| 13 | K1246R | 0.9 | 1.2 | 0.3 | 1.9 | 2.6 |
| 13 | K1246D | 0.8 | 1.4 | 10.6 | 17.3 | 28.2 |
| 13 | K1246A | 2.1 | 1.8 | 2.7 | 5.6 | 4.5 |
| 13 | K1246L | 1.5 | 2.3 | 0.2 | 4.5 | 5.3 |
| 13 | G1247R | 0.9 | 0.8 | 5.8 | 2.2 | 2.3 |
| 13 | G1247L | 4.0 | 7.4 | 0.4 | 30.7 | 37.2 |
| 13 | G1247E | 1.4 | 2.6 | 2.9 | 1.2 | 1.3 |
| 13 | G1247A | 1.3 | 0.7 | 0.1 | 4.3 | 7.3 |
| 13 | G1247G | 1.3 | 1.1 | 2.7 | 3.2 | 2.0 |
| 13 | S1248T | 1.7 | 4.1 | 6.8 | 0.9 | 1.0 |
| 13 | S1248G | 2.0 | 3.7 | 4.4 | 2.2 | 2.2 |
| 13 | S1248G | 0.7 | 2.3 | 2.0 | 2.9 | 3.9 |
| 13 | S1248F | 1.5 | 1.5 | 2.2 | 5.9 | 5.6 |
| 13 | P1249M | 0.8 | 1.0 | 3.5 | 10.3 | 18.5 |
| 13 | P1249R | 1.4 | 4.1 | 5.8 | 16.6 | 16.5 |
| 13 | P1249D | 1.2 | 0.6 | 1.9 | 2.3 | 2.1 |
| 13 | P1249A | 1.4 | 2.4 | 4.1 | 9.9 | 10.5 |
| 13 | P1249G | 1.2 | 1.1 | 5.4 | 0.6 | 3.1 |
| 13 | E1250T | 0.9 | 1.1 | 2.9 | 5.3 | 5.2 |
| 13 | E1250R | 2.1 | 2.5 | 8.6 | 5.0 | 5.0 |
| 13 | E1250S | 1.6 | 1.4 | 3.2 | 4.5 | 6.7 |
| 13 | E1250I | 0.9 | 0.2 | 3.2 | 4.9 | 7.2 |
| 13 | E1250H | 1.1 | 0.6 | 2.4 | 12.2 | 11.1 |
| 13 | E1250V | 1.4 | 2.4 | 2.5 | 3.3 | 5.2 |
| 13 | E1250C | 1.6 | 1.0 | 3.5 | 5.1 | 4.5 |
| 13 | E1250F | 2.5 | 2.2 | 13.8 | 3.6 | 3.7 |
| 13 | D1251K | 1.5 | 1.6 | 2.2 | 1.8 | 1.9 |
| 13 | D1251R | 1.5 | 0.6 | 4.3 | 1.6 | 1.0 |
| 13 | D1251S | 2.0 | 1.9 | 0.5 | 3.3 | 3.5 |
| 13 | D1251M | 0.5 | 0.9 | 5.5 | 1.0 | 3.1 |
| 13 | D1251R | 0.9 | 0.7 | 3.0 | 3.0 | 2.0 |
| 13 | D1251D | 0.9 | 1.0 | 0.5 | 0.8 | 1.6 |
| 13 | D1251A | 1.8 | 1.9 | 1.3 | 2.3 | 2.9 |
| 13 | D1251W | 2.5 | 4.4 | 9.7 | 12.8 | 13.2 |
| 13 | N1252N | 1.8 | 5.0 | 2.8 | 10.1 | 8.7 |
| 13 | N1252D | 1.8 | 2.2 | 2.6 | 3.8 | 6.3 |
| 13 | N1252V | 0.9 | 1.3 | 2.8 | 3.6 | 6.5 |
| 13 | N1252C | 1.3 | 1.6 | 2.8 | 7.2 | 4.2 |
| 13 | N1252L | 2.6 | 1.6 | 3.8 | 2.0 | 1.8 |
| 13 | E1253K | 3.1 | 4.0 | 5.4 | 5.3 | 2.0 |
| 13 | E1253I | 1.3 | 2.2 | 6.4 | 10.3 | 12.6 |
| 13 | E1253R | 1.7 | 2.9 | 7.1 | 7.8 | 6.1 |
| 13 | E1253R | 2.3 | 5.1 | 7.6 | 1.5 | 2.0 |
| 13 | E1253L | 3.2 | 4.9 | 6.1 | 1.4 | 2.6 |
| 13 | E1253E | 1.1 | 1.7 | 4.8 | 2.9 | 1.7 |
| 13 | E1253A | 3.0 | 3.2 | 2.6 | 6.0 | 9.7 |
| 13 | E1253A | 0.9 | 0.8 | 1.5 | 3.8 | 9.9 |
| 13 | E1253G | 1.5 | 3.4 | 2.8 | 4.3 | 2.9 |
| 13 | E1253S | 0.7 | 2.9 | 5.6 | 2.1 | 2.8 |
| 13 | E1253W | 2.1 | 2.0 | 6.9 | 2.4 | 3.1 |
| 13 | Q1254N | 3.0 | 8.4 | 19.2 | 21.2 | 21.4 |
| 13 | Q1254T | 3.0 | 3.3 | 0.2 | 4.5 | 3.7 |
| 13 | Q1254L | 0.9 | 0.7 | 3.3 | 7.4 | 9.1 |
| 13 | Q1254D | 0.7 | 1.3 | 0.1 | 5.0 | 2.3 |
| 13 | Q1254S | 2.2 | 6.0 | 6.2 | 17.8 | 24.1 |
| 13 | Q1254L | 0.5 | 0.6 | 6.9 | 2.6 | 3.4 |
| 13 | K1255K | 1.4 | 3.8 | 4.3 | 3.6 | 2.9 |
| 13 | K1255N | 1.9 | 2.3 | 3.3 | 3.3 | 1.7 |
| 13 | K1255T | 1.5 | 4.4 | 9.6 | 2.2 | 4.6 |
| 13 | K1255R | 2.6 | 1.7 | 4.3 | 5.0 | 1.8 |
| 13 | Q1256N | 1.1 | 1.9 | 10.1 | 2.6 | 2.8 |
| 13 | Q1256Q | 1.8 | 2.8 | 8.9 | 5.4 | 18.3 |
| 13 | Q1256L | 2.2 | 3.2 | 12.8 | 19.4 | 32.0 |
| 13 | L1257K | 2.0 | 5.0 | 2.8 | 6.1 | 13.9 |
| 13 | L1257N | 2.2 | 5.2 | 9.4 | 7.8 | 11.4 |
| 13 | L1257T | 1.8 | 2.6 | 4.1 | 10.6 | 13.1 |
| 13 | L1257M | 1.3 | 1.9 | 5.5 | 3.1 | 5.9 |
| 13 | L1257P | 2.3 | 1.8 | 14.1 | 36.5 | 10.0 |
| 13 | L1257W | 0.6 | 0.6 | 5.5 | 5.8 | 4.9 |
| 13 | V1259V | 1.0 | 0.8 | 1.5 | 2.0 | 2.4 |
| 13 | V1259V | 1.3 | 2.5 | 8.1 | 7.3 | 4.6 |
| 13 | E1260K | 2.0 | 0.9 | 5.1 | 2.7 | 2.1 |
| 13 | E1260Q | 1.2 | 3.9 | 33.8 | 7.6 | 2.9 |
| 13 | Q1261D | 2.4 | 6.9 | 13.9 | 72.0 | 54.8 |
| 13 | H1262S | 2.5 | 6.7 | 15.3 | 16.1 | 5.9 |
| 13 | K1263S | 0.2 | 0.3 | 0.0 | 0.4 | 1.1 |
| 13 | K1263L | 2.3 | 6.8 | 27.7 | 29.3 | 48.3 |
| 13 | K1263G | 6.0 | 6.2 | 23.4 | 53.5 | 27.0 |
| 13 | K1263V | 1.8 | 0.4 | 1.8 | 11.9 | 13.7 |
| 13 | K1263F | 1.8 | 3.2 | 0.3 | 18.2 | 6.6 |
| 13 | H1264Y | 1.4 | 3.1 | 15.8 | 28.4 | 15.2 |
| 13 | H1264F | 3.0 | 2.5 | 3.6 | 25.0 | 4.0 |
| 13 | Y1265M | 2.2 | 4.5 | 5.0 | 76.4 | 67.9 |
| 13 | Y1265F | 1.4 | 1.6 | 2.4 | 4.2 | 5.4 |
| 13 | L1266T | 1.8 | 5.6 | 1.3 | 0.7 | 1.1 |
| 13 | E1268E | 1.6 | 0.7 | 0.8 | 1.3 | 1.3 |
| 13 | I1270M | 1.6 | 0.8 | 5.3 | 2.6 | 5.2 |
| 13 | E1271K | 1.4 | 1.2 | 1.4 | 1.9 | 1.9 |
| 13 | Q1272A | 1.2 | 1.0 | 11.6 | 49.2 | 58.3 |
| 13 | I1273S | 1.0 | 1.1 | 1.1 | 1.2 | 1.2 |
| 13 | I1273V | 1.5 | 3.4 | 9.3 | 3.8 | 3.0 |
| 13 | S1274K | 2.8 | 7.5 | 6.6 | 9.8 | 5.8 |
| 13 | S1274F | 0.7 | 0.4 | 4.1 | 1.8 | 1.2 |
| 13 | E1275I | 1.0 | 1.3 | 12.5 | 30.4 | 31.4 |
| 13 | E1275G | 1.4 | 0.9 | 4.7 | 4.5 | 4.7 |
| 13 | E1275C | 0.7 | 0.1 | 2.7 | 1.0 | 1.0 |
| 13 | F1276F | 0.8 | 1.0 | 1.9 | 2.8 | 2.8 |
| 13 | S1277A | 1.1 | 1.3 | 11.7 | 5.1 | 2.2 |
| 13 | S1277C | 1.1 | 1.4 | 2.5 | 1.7 | 2.2 |
| 13 | K1278E | 1.0 | 0.8 | 1.1 | 0.4 | 1.6 |
| 13 | R1279K | 1.2 | 0.7 | 1.0 | 0.4 | 1.0 |
| 13 | R1279R | 1.7 | 0.5 | 3.4 | 13.5 | 7.1 |
| 13 | R1279A | 1.6 | 2.4 | 3.5 | 8.7 | 5.5 |
| 13 | V1280V | 0.9 | 1.7 | 1.1 | 1.2 | 1.4 |
| 13 | V1280V | 0.6 | 0.8 | 2.2 | 0.5 | 1.0 |
| 13 | V1280Y | 1.3 | 2.5 | 6.6 | 1.6 | 1.5 |
| 13 | I1281I | 1.2 | 1.6 | 2.5 | 2.6 | 1.1 |
| 13 | L1282Q | 1.1 | 1.0 | 5.8 | 5.6 | 9.2 |
| 13 | L1282R | 1.3 | 2.6 | 3.3 | 2.2 | 3.4 |
| 13 | L1282L | 2.1 | 2.7 | 3.5 | 3.7 | 3.6 |
| 13 | D1284G | 1.3 | 1.6 | 2.6 | 4.0 | 3.6 |
| 13 | A1285R | 1.6 | 4.2 | 6.9 | 19.7 | 28.4 |
| 13 | A1285R | 2.9 | 4.4 | 3.6 | 2.3 | 2.9 |
| 13 | N1286N | 1.6 | 1.7 | 1.6 | 2.2 | 4.4 |
| 13 | N1286T | 1.5 | 1.4 | 3.0 | 6.2 | 8.2 |
| 13 | N1286E | 1.6 | 1.8 | 4.4 | 4.1 | 5.4 |
| 13 | N1286A | 1.6 | 0.9 | 0.2 | 2.4 | 3.4 |
| 13 | N1286G | 0.4 | 0.4 | 7.4 | 5.0 | 1.8 |
| 13 | N1286V | 1.4 | 0.5 | 6.2 | 3.5 | 4.1 |
| 13 | D1288L | 5.0 | 5.7 | 14.7 | 17.2 | 20.6 |
| 13 | K1289I | 1.1 | 0.5 | 0.0 | 4.0 | 2.9 |
| 13 | V1290A | 3.8 | 7.5 | 24.9 | 24.5 | 37.4 |
| 13 | L1291R | 0.8 | 1.3 | 5.7 | 2.1 | 4.1 |
| 13 | S1292A | 3.4 | 5.3 | 10.7 | 6.5 | 8.6 |
| 13 | A1293L | 2.1 | 1.4 | 0.8 | 3.5 | 1.3 |
| 13 | A1293C | 0.3 | 0.6 | 1.9 | 17.1 | 22.1 |
| 13 | Y1294Y | 1.1 | 1.1 | 0.4 | 2.7 | 6.7 |
| 13 | Y1294F | 0.7 | 1.4 | 2.7 | 30.4 | 27.1 |
| 13 | N1295T | 0.3 | 0.2 | 3.0 | 4.4 | 6.0 |
| 13 | N1295L | 3.6 | 4.7 | 11.8 | 9.4 | 6.6 |
| 13 | N1295Y | 1.1 | 1.5 | 0.9 | 1.3 | 1.1 |
| 13 | K1296T | 1.4 | 1.9 | 4.0 | 3.6 | 1.2 |
| 13 | K1296S | 0.5 | 1.0 | 0.5 | 4.9 | 1.3 |
| 13 | K1296M | 2.1 | 1.7 | 1.9 | 4.7 | 6.5 |
| 13 | K1296R | 0.8 | 1.6 | 4.0 | 5.9 | 1.1 |
| 13 | K1296L | 1.6 | 2.5 | 3.2 | 4.1 | 3.3 |
| 13 | K1296L | 3.2 | 7.0 | 8.3 | 5.6 | 17.6 |
| 13 | K1296L | 1.4 | 1.5 | 5.9 | 6.8 | 9.0 |
| 13 | H1297T | 1.2 | 1.8 | 35.8 | 5.9 | 4.4 |
| 13 | H1297Q | 2.9 | 2.8 | 1.9 | 8.2 | 1.3 |
| 13 | R1298Q | 0.4 | 0.0 | 3.7 | 55.9 | 49.1 |
| 13 | D1299K | 0.6 | 2.6 | 16.3 | 12.1 | 14.2 |
| 13 | D1299S | 1.3 | 0.4 | 4.4 | 1.9 | 2.2 |
| 13 | D1299R | 0.5 | 1.1 | 1.7 | 2.7 | 3.5 |
| 13 | D1299G | 2.0 | 1.2 | 0.7 | 3.3 | 3.5 |
| 13 | D1299S | 1.2 | 1.9 | 1.2 | 1.3 | 2.3 |
| 13 | K1300R | 1.7 | 2.1 | 0.2 | 2.0 | 3.3 |
| 14 | P1301H | 0.6 | 0.4 | 1.1 | 1.4 | 2.1 |
| 14 | P1301P | 1.3 | 1.8 | 1.5 | 2.0 | 2.3 |
| 14 | P1301V | 0.3 | 0.1 | 0.4 | 1.2 | 2.2 |
| 14 | R1303N | 1.3 | 1.3 | 2.2 | 1.8 | 2.2 |
| 14 | R1303M | 1.9 | 2.4 | 2.8 | 5.5 | 3.2 |
| 14 | R1303H | 0.8 | 1.5 | 2.8 | 2.7 | 3.2 |
| 14 | R1303P | 1.0 | 3.1 | 4.4 | 5.2 | 5.1 |
| 14 | R1303V | 1.1 | 0.7 | 1.0 | 0.5 | 1.0 |
| 14 | R1303V | 0.7 | 1.1 | 1.6 | 1.1 | 1.4 |
| 14 | A1306A | 1.1 | 1.3 | 1.8 | 2.1 | 2.1 |
| 14 | A1306V | 0.6 | 0.8 | 0.8 | 0.8 | 1.0 |
| 14 | E1307L | 0.7 | 2.0 | 1.2 | 0.9 | 1.2 |
| 14 | E1307C | 0.9 | 0.4 | 0.4 | 1.0 | 1.7 |
| 14 | N1308M | 1.6 | 0.8 | 2.7 | 2.2 | 1.7 |
| 14 | N1308E | 1.1 | 0.9 | 2.6 | 4.1 | 4.1 |
| 14 | N1308A | 1.9 | 2.7 | 1.6 | 3.9 | 1.0 |
| 14 | I1309M | 1.1 | 2.2 | 3.6 | 3.2 | 4.9 |
| 14 | I1309I | 0.7 | 0.9 | 0.9 | 1.7 | 1.0 |
| 14 | I1309L | 0.8 | 0.9 | 4.1 | 8.3 | 5.5 |
| 14 | I1309L | 0.4 | 0.8 | 3.0 | 3.3 | 2.5 |
| 14 | I1310M | 1.2 | 1.1 | 0.9 | 0.9 | 1.1 |
| 14 | I1310L | 1.3 | 2.9 | 3.1 | 2.3 | 3.2 |
| 14 | I1310V | 1.2 | 1.3 | 1.5 | 1.8 | 1.5 |
| 14 | H1311K | 1.2 | 0.8 | 1.0 | 1.4 | 1.0 |
| 14 | H1311S | 1.5 | 2.5 | 3.7 | 3.0 | 3.7 |
| 14 | H1311R | 1.7 | 3.5 | 6.9 | 10.3 | 11.5 |
| 14 | H1311R | 1.0 | 0.7 | 3.2 | 3.7 | 3.6 |
| 14 | H1311A | 1.5 | 1.1 | 3.7 | 1.2 | 1.7 |
| 14 | H1311V | 0.5 | 1.5 | 1.7 | 0.5 | 1.7 |
| 14 | H1311T | 1.8 | 2.9 | 3.2 | 2.3 | 1.2 |
| 14 | H1311S | 2.0 | 1.9 | 5.1 | 1.6 | 1.7 |
| 14 | H1311H | 1.5 | 2.6 | 2.9 | 1.8 | 1.7 |
| 14 | H1311R | 2.6 | 5.1 | 9.8 | 17.3 | 19.9 |
| 14 | H1311R | 1.9 | 1.8 | 6.2 | 7.2 | 7.1 |
| 14 | H1311A | 2.4 | 1.8 | 5.9 | 1.9 | 2.7 |
| 14 | H1311G | 1.5 | 2.5 | 3.1 | 2.5 | 1.1 |
| 14 | H1311V | 0.7 | 2.4 | 2.6 | 0.8 | 2.7 |
| 14 | H1311S | 0.7 | 2.9 | 3.0 | 9.2 | 8.5 |
| 14 | F1313F | 1.3 | 1.6 | 2.2 | 1.4 | 2.6 |
| 14 | T1314K | 0.7 | 1.2 | 3.1 | 6.2 | 2.1 |
| 14 | T1314T | 1.5 | 1.3 | 3.8 | 5.3 | 2.7 |
| 14 | L1315M | 2.6 | 8.8 | 31.3 | 20.1 | 18.5 |
| 14 | L1318F | 0.5 | 0.0 | 1.2 | 1.9 | 1.0 |
| 14 | P1321A | 1.2 | 0.5 | 0.0 | 1.5 | 1.2 |
| 14 | A1322R | 2.6 | 4.1 | 4.9 | 2.1 | 2.5 |
| 14 | A1323S | 2.1 | 3.7 | 17.4 | 1.0 | 1.4 |
| 14 | A1323A | 1.4 | 1.6 | 1.5 | 1.8 | 1.8 |
| 14 | A1323V | 0.9 | 3.7 | 1.8 | 4.1 | 2.4 |
| 14 | A1323S | 2.0 | 4.3 | 4.9 | 6.0 | 7.0 |
| 14 | Y1326N | 1.4 | 1.6 | 1.7 | 1.9 | 2.0 |
| 14 | Y1326M | 2.7 | 6.8 | 2.2 | 5.8 | 2.9 |
| 14 | Y1326F | 2.3 | 3.9 | 9.0 | 12.8 | 15.6 |
| 14 | F1327W | 1.2 | 1.7 | 1.4 | 1.8 | 2.9 |
| 14 | D1328D | 1.9 | 1.4 | 2.3 | 5.1 | 3.0 |
| 14 | T1329K | 1.4 | 0.9 | 1.3 | 1.1 | 1.4 |
| 14 | T1329Q | 2.2 | 6.4 | 1.0 | 3.2 | 2.1 |
| 14 | T1329L | 2.0 | 2.2 | 4.7 | 4.4 | 2.9 |
| 14 | T1329V | 1.6 | 1.9 | 4.7 | 3.1 | 1.4 |
| 14 | T1329S | 0.7 | 1.2 | 3.9 | 2.2 | 7.1 |
| 14 | T1329L | 1.1 | 1.9 | 1.5 | 2.6 | 2.5 |
| 14 | T1330H | 1.7 | 1.0 | 1.8 | 1.4 | 1.4 |
| 14 | I1331I | 1.8 | 2.3 | 4.5 | 4.6 | 1.4 |
| 14 | D1332P | 10.0 | 32.1 | 19.2 | 46.5 | 42.6 |
| 14 | R1333H | 0.9 | 1.0 | 1.1 | 1.2 | 1.3 |
| 14 | R1333P | 1.2 | 1.5 | 1.5 | 1.6 | 1.9 |
| 14 | R1333R | 1.8 | 2.4 | 1.9 | 1.8 | 2.2 |
| 14 | K1334N | 1.2 | 0.9 | 1.0 | 1.0 | 1.2 |
| 14 | K1334STOP | 1.1 | 1.1 | 1.2 | 1.2 | 1.4 |
| 14 | Y1336F | 1.1 | 1.2 | 1.3 | 1.3 | 1.3 |
| 14 | T1337V | 1.0 | 0.6 | 2.3 | 4.0 | 1.3 |
| 14 | T1337L | 2.9 | 4.7 | 2.1 | 4.0 | 4.4 |
| 14 | S1338N | 1.5 | 1.5 | 1.5 | 1.7 | 1.8 |
| 14 | S1338T | 1.7 | 2.6 | 3.6 | 1.3 | 1.2 |
| 14 | S1338R | 1.8 | 3.3 | 2.2 | 2.4 | 2.0 |
| 14 | S1338Q | 3.3 | 5.8 | 24.0 | 39.0 | 31.6 |
| 14 | T1339S | 2.9 | 6.4 | 13.0 | 7.5 | 1.8 |
| 14 | T1339M | 0.9 | 0.9 | 1.3 | 0.9 | 2.0 |
| 14 | T1339S | 1.2 | 1.0 | 3.8 | 4.9 | 2.7 |
| 14 | K1340K | 1.9 | 1.7 | 2.3 | 2.4 | 4.6 |
| 14 | K1340N | 1.5 | 6.9 | 10.1 | 8.7 | 3.2 |
| 14 | L1343L | 1.3 | 5.2 | 8.0 | 12.3 | 27.9 |
| 14 | L1343C | 1.3 | 2.7 | 16.7 | 2.5 | 1.3 |
| 14 | D1344V | 1.6 | 1.7 | 1.5 | 1.7 | 1.3 |
| 14 | A1345G | 1.6 | 4.0 | 11.3 | 13.8 | 18.7 |
| 14 | L1347H | 1.1 | 1.0 | 1.1 | 1.1 | 1.3 |
| 14 | L1347L | 1.1 | 2.8 | 5.3 | 5.2 | 1.1 |
| 14 | I1348N | 1.3 | 1.1 | 1.2 | 1.3 | 1.1 |
| 14 | I1348V | 1.2 | 1.4 | 4.3 | 6.8 | 2.8 |
| 14 | H1349K | 2.4 | 4.0 | 2.9 | 3.1 | 2.9 |
| 14 | H1349I | 2.0 | 3.0 | 15.8 | 15.4 | 5.6 |
| 14 | H1349H | 1.3 | 3.0 | 0.3 | 5.3 | 19.5 |
| 14 | H1349R | 2.6 | 5.3 | 6.5 | 5.7 | 9.1 |
| 14 | H1349R | 1.6 | 0.8 | 2.1 | 4.9 | 4.2 |
| 14 | H1349L | 1.6 | 4.7 | 8.2 | 20.1 | 7.8 |
| 14 | Q1350Y | 2.0 | 6.2 | 15.4 | 19.8 | 11.0 |
| 14 | S1351S | 2.2 | 1.7 | 2.0 | 3.4 | 2.9 |
| 14 | I1352N | 1.3 | 1.3 | 1.5 | 1.5 | 1.7 |
| 14 | I1352V | 2.4 | 4.5 | 2.4 | 6.3 | 7.3 |
| 14 | T1353T | 1.4 | 2.8 | 5.2 | 2.3 | 4.0 |
| 14 | G1354G | 1.6 | 3.3 | 4.2 | 7.4 | 3.9 |
| 14 | Y1356N | 1.6 | 2.1 | 2.2 | 2.1 | 2.5 |
| 14 | Y1356F | 1.9 | 1.7 | 1.7 | 1.1 | 1.0 |
| 14 | T1358T | 1.1 | 4.7 | 2.8 | 4.5 | 7.3 |
| 14 | I1360V | 1.9 | 4.1 | 1.1 | 1.6 | 1.1 |
| 14 | I1360F | 1.8 | 4.9 | 12.9 | 6.9 | 3.9 |
| 14 | L1362L | 2.7 | 5.9 | 7.7 | 5.6 | 5.5 |
| 14 | S1363S | 1.6 | 2.9 | 6.1 | 3.6 | 1.9 |
| 14 | S1363Q | 0.6 | 1.8 | 4.1 | 4.9 | 1.5 |
| 14 | Q1364S | 1.2 | 2.4 | 8.0 | 12.5 | 23.7 |
| 14 | Q1364Q | 1.8 | 2.3 | 3.9 | 1.2 | 1.0 |
| 14 | Q1364L | 2.1 | 1.9 | 7.2 | 7.3 | 6.5 |
| 14 | L1365L | 4.5 | 6.6 | 21.7 | 8.8 | 5.7 |
| 14 | G1366C | 2.3 | 0.8 | 2.1 | 2.1 | 1.3 |
| 14 | G1367R | 1.4 | 1.1 | 2.5 | 2.4 | 2.9 |
| 14 | G1367Q | 1.0 | 0.4 | 5.3 | 4.4 | 3.5 |
| 14 | G1367R | 1.1 | 2.8 | 3.4 | 5.2 | 7.7 |
| 14 | G1367L | 1.5 | 4.2 | 18.5 | 12.8 | 4.5 |
| 14 | G1367L | 1.9 | 3.4 | 14.7 | 9.8 | 3.9 |
| 14 | G1367A | 1.0 | 1.7 | 7.4 | 11.5 | 13.3 |
| 14 | G1367G | 2.3 | 2.2 | 3.4 | 8.7 | 2.4 |
| 14 | G1367V | 1.6 | 2.5 | 2.6 | 2.5 | 2.4 |
| 14 | D1368S | 2.1 | 4.2 | 4.8 | 4.9 | 1.3 |
| 14 | D1368A | 2.0 | 3.1 | 2.0 | 2.9 | 1.5 |
| 14 | D1368V | 1.9 | 2.1 | 1.9 | 2.0 | 2.0 |
| 14 | D1368W | 1.0 | 1.7 | 14.2 | 4.1 | 3.8 |
| 14 | D1368L | 1.5 | 4.6 | 6.5 | 19.4 | 18.0 |

TABLE 3

| Exemplary | Sequence | |
|---|---|---|
| Sequences | Identifier | Sequence |
| Cas9 nickase | SEQ ID NO: 1401 | MDKKYSIGLDIGTNSVGWAVITDEYKVPSKKFKVLGNTDRHSIKK |
| (H840A), | | NLIGALLFDSGETAEATRLKRTARRRYTRRKNRICYLQEIFSNEMA |
| Streptococcus | | KVDDSFFHRLEESFLVEEDKKHERHPIFGNIVDEVAYHEKYPTIY |
| pyogenes | | HLRKKLVDSTDKADLRLIYLALAHMIKFRGHFLIEGDLNPDNSDV |
| | | DKLFIQLVQTYNQLFEENPINASGVDAKAILSARLSKSRRLENLIA |
| | | QLPGEKKNGLFGNLIALSLGLTPNFKSNFDLAEDAKLQLSKDTY |
| | | DDDLDNLLAQIGDQYADLFLAAKNLSDAILLSDILRVNTEITKAPL |
| | | SASMIKRYDEHHQDLTLLKALVRQQLPEKYKEIFFDQSKNGYAG |
| | | YIDGGASQEEFYKFIKPILEKMDGTEELLVKLNREDLLRKQRTFD |
| | | NGSIPHQIHLGELHAILRRQEDFYPFLKDNREKIEKILTFRIPYYVG |
| | | PLARGNSRFAWMTRKSEETITPWNFEEVVDKGASAQSFIERMTN |
| | | FDKNLPNEKVLPKHSLLYEYFTVYNELTKVKYVTEGMRKPAFLSG |
| | | EQKKAIVDLLFKTNRKVTVKQLKEDYFKKIECFDSVEISGVEDRFN |
| | | ASLGTYHDLLKIIKDKDFLDNEENEDILEDIVLTLTLFEDREMIEER |
| | | LKTYAHLFDDKVMKQLKRRRYTGWGRLSRKLINGIRDKQSGKTIL |
| | | DFLKSDGFANRNFMQLIHDDSLTFKEDIQKAQVSGQGDSLHEH |
| | | IANLAGSPAIKKGILQTVKVVDELVKVMGRHKPENIVIEMARENQ |
| | | TTQKGQKNSRERMKRIEEGIKELGSQILKEHPVENTQLQNEKLYL |
| | | YYLQNGRDMYVDQELDINRLSDYDVDAIVPQSFLKDDSIDNKVL |
| | | TRSDKNRGKSDNVPSEEVVKKMKNYWRQLLNAKLITQRKFDNL |
| | | TKAERGGLSELDKAGFIKRQLVETRQITKHVAQILDSRMNTKYDE |
| | | NDKLIREVKVITLKSKLVSDFRKDFQFYKVREINNYHHAHDAYLN |
| | | AVVGTALIKKYPKLESEFVYGDYKVYDVRKMIAKSEQEIGKATAKY |
| | | FFYSNIMNFFKTEITLANGEIRKRPLIETNGETGEIVWDKGRDFAT |
| | | VRKVLSMPQVNIVKKTEVQTGGFSKESILPKRNSDKLIARKKDW |
| | | DPKKYGGFDSPTVAYSVLVVAKVEKGKSKKLKSVKELLGITIMERS |
| | | SFEKNPIDFLEAKGYKEVKKDLIIKLPKYSLFELENGRKRMLASAG |
| | | ELQKGNELALPSKYVNFLYLASHYEKLKGSPEDNEQKQLFVEQH |
| | | KHYLDEIIEQISEFSKRVILADANLDKVLSAYNKHRDKPIREQAEN |
| | | IIHLFTLTNLGAPAAFKYFDTTIDRKRYTSTKEVLDATLIHQSITGLYE |
| | | TRIDLSQLGGD |
| Cas9 Wild- | SEQ ID NO: 1402 | MDKKYSIGLDIGTNSVGWAVITDEYKVPSKKFKVLGNTDRHSIKK |
| Type, | | NLIGALLFDSGETAEATRLKRTARRRYTRRKNRICYLQEIFSNEMA |
| Streptococcus | | KVDDSFFHRLEESFLVEEDKKHERHPIFGNIVDEVAYHEKYPTIY |
| pyogenes | | HLRKKLVDSTDKADLRLIYLALAHMIKFRGHFLIEGDLNPDNSDV |
| | | DKLFIQLVQTYNQLFEENPINASGVDAKAILSARLSKSRRLENLIA |
| | | QLPGEKKNGLFGNLIALSLGLTPNFKSNFDLAEDAKLQLSKDTY |
| | | DDDLDNLLAQIGDQYADLFLAAKNLSDAILLSDILRVNTEITKAPL |
| | | SASMIKRYDEHHQDLTLLKALVRQQLPEKYKEIFFDQSKNGYAG |
| | | YIDGGASQEEFYKFIKPILEKMDGTEELLVKLNREDLLRKQRTFD |
| | | NGSIPHQIHLGELHAILRRQEDFYPFLKDNREKIEKILTFRIPYYVG |
| | | PLARGNSRFAWMTRKSEETITPWNFEEVVDKGASAQSFIERMTN |
| | | FDKNLPNEKVLPKHSLLYEYFTVYNELTKVKYVTEGMRKPAFLSG |
| | | EQKKAIVDLLFKTNRKVTVKQLKEDYFKKIECFDSVEISGVEDRFN |
| | | ASLGTYHDLLKIIKDKDFLDNEENEDILEDIVLTLTLFEDREMIEER |
| | | LKTYAHLFDDKVMKQLKRRRYTGWGRLSRKLINGIRDKQSGKTIL |
| | | DFLKSDGFANRNFMQLIHDDSLTFKEDIQKAQVSGQGDSLHEH |
| | | IANLAGSPAIKKGILQTVKVVDELVKVMGRHKPENIVIEMARENQ |
| | | TTQKGQKNSRERMKRIEEGIKELGSQILKEHPVENTQLQNEKLYL |
| | | YYLQNGRDMYVDQELDINRLSDYDVDHIVPQSFLKDDSIDNKV |
| | | LTRSDKNRGKSDNVPSEEVVKKMKNYWRQLLNAKLITQRKFDN |
| | | LTKAERGGLSELDKAGFIKRQLVETRQITKHVAQILDSRMNTKYD |
| | | ENDKLIREVKVITLKSKLVSDFRKDFQFYKVREINNYHHAHDAYL |
| | | NAVVGTALIKKYPKLESEFVYGDYKVYDVRKMIAKSEQEIGKATAK |
| | | YFFYSNIMNFFKTEITLANGEIRKRPLIETNGETGEIVWDKGRDFA |
| | | TVRKVLSMPQVNIVKKTEVQTGGFSKESILPKRNSDKLIARKKDW |
| | | DPKKYGGFDSPTVAYSVLVVAKVEKGKSKKLKSVKELLGITIMERS |
| | | SFEKNPIDFLEAKGYKEVKKDLIIKLPKYSLFELENGRKRMLASAG |
| | | ELQKGNELALPSKYVNFLYLASHYEKLKGSPEDNEQKQLFVEQH |
| | | KHYLDEIIEQISEFSKRVILADANLDKVLSAYNKHRDKPIREQAEN |
| | | IIHLFTLTNLGAPAAFKYFDTTIDRKRYTSTKEVLDATLIHQSITGLYE |
| | | TRIDLSQLGGD |
| sgRNA | SEQ ID NO: 1403 | GUUUUAGAGCUAGAAAUAGCAAGUUAAAAUAAGGCUAGUC |
| scaffold | | CGUUAUCAACUUGAAAAAGUGGCACCGAGUCGGUGC |
| domain of a | | |
| full length | | |
| pegRNA | | |
| shortened | SEQ ID NO: 1404 | GUUUUAGAGCUAGAAAUAGCAAGUUAAAAUAAGGCUAGUC |
| sgRNA | | CGUU |
| scaffold | | |
| domain of a | | |
| truncated | | |
| pegRNA | | |
| representative | SEQ ID NO: 1405 | NNNNNNNNNNNNNNNNNNNNGUUUUAGAGCUAGAAAUA |
| full-length | | GCAAGUUAAAAUAAGGCUAGUCCGUUAUCAACUUGAAAAA |
| pegRNA (″N″ | | GUGGCACCGAGUCGGUGCNNNNNNNNNNNNNNNNNNN |
| denotes any | | NNNN |
| base) | | |
| representative | SEQ ID NO: 1406 | NNNNNNNNNNNNNNNNNNNNGUUUUAGAGCUAGAAAUA |
| truncated | | GCAAGUUAAAAUAAGGCUAGUCCGUUNNNNNNNNNNNN |
| pegRNA (″N″ | | NNNNNNNNNNN |
| denotes any | | |
| base) | | |

While the invention has been described in detail and with reference to specific examples thereof, it will be apparent to one skilled in the art that various changes and modifications can be made therein without departing from the spirit and scope thereof.

### REFERENCES

1. Jinek, M., Et Al., A Programmable Dual-Rna-Guided Dna Endonuclease In Adaptive Bacterial Immunity. Science, 2012. 337(6096): P. 816-21.
2. Komor, A. C., Et Al., Programmable Editing Of A Target Base In Genomic Dna Without Double-Stranded Dna Cleavage. Nature, 2016. 533(7603): P. 420-4.
3. Gaudelli, N. M., Et Al., Programmable Base Editing Of A*T To G*C In Genomic Dna Without Dna Cleavage. Nature, 2017. 551(7681): P. 464-471.
4. Anzalone, A. V., Et Al., Search-And-Replace Genome Editing Without Double-Strand Breaks Or Donor Dna. Nature, 2019. 576(7785): P. 149-157.
5. Tong, Y., Et Al., A Versatile Genetic Engineering Toolkit For E. Coli Based On Crispr-Prime Editing. Nat Commun, 2021. 12(1): P. 5206.
6. Chen, S., Et Al., Fastp: An Ultra-Fast All-In-One Fastq Preprocessor. Bioinformatics, 2018. 34(17): P. 884-890.
7. Martin M. Cutadapt Removes Adapter Sequences From High-Throughput Sequencing Reads. Embnet. Journal. 2011. 17(1): P. 10-2.

1. A prime editor variant, comprising:
a. a modified Cas9 having one or more mutations listed in Table 2; and
b. an engineered reverse transcriptase,
wherein the prime editor variant binds to a truncated pegRNA, and exhibits enhanced prime editing activity relative to wild-type Cas9 with a full-length pegRNA.

2. The prime editor variant of claim 1, wherein the one or more mutations comprises at least one mutation selected from Table 2 that exhibits greater than 2-fold enrichment relative to the wild-type Cas9 amino acid sequence of SEQ ID NO: 1402.

3. The prime editor variant of claim 1, wherein the one or more mutations comprises at least one mutation selected from the group consisting of: E1071R, E1071K, T1065R, N758Y, A1034E, D180H, H420Q, Q894A, R40L, I122P, R215L, K961E, E1007L, K31Y, D364S, K382S, R457S, S42E, N193M, K649N, Q1364S, and F1008V relative to the wild-type Cas9 amino acid sequence of SEQ ID NO: 1402.

4. The prime editor variant of claim 1, the modified Cas9 further comprises a H840A mutation relative to the wild-type Cas9 amino acid sequence of SEQ ID NO: 1402.

5. The prime editor variant of claim 1, wherein the modified Cas9 comprises a Cas protein selected from the group consisting of wild-type Cas9, dCas9, and Cas9 nickase.

6. The prime editor variant of claim 1, wherein the truncated pegRNA comprises a target sequence, sgRNA scaffold, reverse transcriptase template (RTT), and a primer binding site (PBS) domains, wherein the truncated pegRNA comprises the sgRNA scaffold domain that is shorter from the 3′ end by at least the number of bases selected from the group consisting of 30 bases, 31 bases, 32 bases, 33 bases, 34 bases, 35 bases, 36 bases, 37 bases, 38 bases, 39 bases, and 40 bases relative to the sgRNA scaffold domain of a full-length pegRNA represented by SEQ ID NO: 1403.

7. The prime editor variant of claim 1, wherein the truncated pegRNA comprises the sgRNA scaffold domain that is 31 bases shorter from the 3′ end relative to the sgRNA scaffold domain of a full-length pegRNA represented by SEQ ID NO: 1403.

8. The prime editor variant of claim 1, the modified Cas9 further comprises at least one nuclear localization signal.

9. An isolated nucleic acid encoding the prime editor variant of claim 1.

10. An isolated nucleic acid encoding the modified Cas9 of claim 1.

11. An mRNA encoding the prime editor variant of claim 1.

12. A prime editing system, comprising:
a. a prime editor variant of claim 1; and
b. a truncated pegRNA,
wherein the prime editing system exhibits enhanced prime editing activity relative to wild-type Cas9 with a full-length pegRNA.

13-29. (canceled)

30. A modified Cas9, comprising one or more mutations selected from Table 2 relative to the wild-type Cas9 amino acid sequence of SEQ ID NO: 1402, wherein the modified Cas9 binds to a truncated pegRNA to form a complex, and wherein the complex exhibits enhanced prime editing activity relative to wild-type Cas9 with a full-length pegRNA.

31. The modified Cas9 of claim 30, wherein the one or more mutations comprises at least one mutation selected from the group consisting of: E1071R, E1071K, T1065R, N758Y, A1034E, D180H, H420Q, Q894A, R40L, I122P, R215L, K961E, E1007L, K31Y, D364S, K382S, R457S, S42E, N193M, K649N, Q1364S, and F1008V relative to the wild-type Cas9 amino acid sequence of SEQ ID NO: 1402.

32-40. (canceled)

41. A method of delivering a Cas9 truncated pegRNA system of claim 1 to a mammalian cell, the method comprising the steps of:
(a) providing a first viral vector component encoding a truncated pegRNA that hybridizes with a target sequence;
(b) providing a second viral vector component encoding a Cas9 variant;
wherein components (a) and (b) are located on same or different vectors of the system; and
(c) transducing the mammalian cell with the viral vector(s) under conditions sufficient to express the Cas9-Truncated pegRNA,
wherein the Cas9-Truncated pegRNA complex form a complex that binds to and edits the target sequence.

42. A method for delivering a Cas9 truncated pegRNA system of claim 1 to a mammalian cell, comprising:
(a) providing lipid nanoparticles encapsulating a first mRNA which is a truncated pegRNA that hybridizes with a target sequence;
(b) providing lipid nanoparticles encapsulating a second mRNA encoding a Cas9 variant of any one of claims 18-24;
wherein components (a) and (b) are located on same or different vectors of the system,
(c) transducing the mammalian cell with the lipid nanoparticles under conditions sufficient to express the Cas9-truncated pegRNA,
wherein the Cas9-truncated pegRNA are expressed and form a complex to edit a specific target sequence in the cell's genome.

43. A method of performing gene editing having reduced off-target editing activity and/or increased on-target editing activity, comprising:
(a) providing a ribonucleoprotein (RNP) complex comprising the Cas9-truncated pegRNA of claim 1 and a truncated pegRNA; and
(b) introducing the RNP complex into the mammalian cell using electroporation, wherein the Cas9-truncated pegRNA system binds to and edits a target sequence within the genome of the mammalian cell.

44. A method of delivering the Cas9-truncated pegRNA system of claim 1 to a mammalian cell, comprising:
preparing a lipofection reagent comprising a first regulatory element operable in a eukaryotic cell operably linked to at least one nucleotide sequence encoding a truncated pegRNA that hybridizes with a target sequence;
a second regulatory element operable in a eukaryotic cell operably linked to a nucleotide sequence encoding a Cas9 variant;
wherein components (a) and (b) are located on the same or different vectors of the system, and
applying the lipofection reagent to the mammalian cell,
wherein the Cas9-truncated pegRNA system are expressed and form a complex to edit a specific target sequence in the cell's genome.

45. A method of targeted delivery of the Cas9-truncated pegRNA system of claim 1 to a mammalian cell, comprising:
(a) preparing exosomes encapsulating a first nucleotide sequence encoding a truncated pegRNA that hybridizes with a target sequence; and
(b) preparing exosomes encapsulating a second nucleotide sequence encoding a Cas9 variant;
wherein components (a) and (b) are located on same or different vectors of the system; and
(c) delivering the engineered exosomes to the mammalian cell under conditions that allow the Cas9-truncated pegRNA system to edit the target sequence.
